(19) United States
(12) Patent Application Publication
(10) Pub. No.: US 2025/0002910 A1
(43) Pub. Date: Jan. 2, 2025

(54) TREATMENT OF COMPLEMENT-MEDIATED DISORDERS

(71) Applicant: Apellis Pharmaceuticals, Inc., Waltham, MA (US)

(72) Inventor: Lukas Scheibler, Telluride, CO (US)

(21) Appl. No.: 18/563,184

(22) PCT Filed: May 26, 2022
(86) PCT No.: PCT/US22/31115

§ 371 Date: Nov. 21, 2023

Related U.S. Application Data
(60) Provisional application No. 63/193,573, filed on May 26, 2021.

(51) Int. Cl. C12N 15/113; A61P 27/02

(52) U.S. Cl. CPC C12N 15/113; A61P 27/02; C12N 2310/11; C12N 2310/14; C12N 2310/321; C12N 2310/322; C12N 2310/351; C12N 2320/32

(57) ABSTRACT

Inappropriate or excessive complement activation is an underlying cause or contributing factor to a number of serious diseases and conditions, and considerable effort has been devoted over the past several decades to exploring various complement inhibitors as therapeutic agents. RNAs, such as miRNA and siRNA, and their use in treating complement-mediated disorders, are described.

### CROSS REFERENCE TO RELATED APPLICATIONS

This application claims the benefit of U.S. Provisional Application No. 63/193,573, filed May 26, 2021, the contents of which is hereby incorporated by reference in its entirety.

### BACKGROUND

Complement is a system consisting of more than 30 plasma and cell-bound proteins that plays a significant role in both innate and adaptive immunity. The proteins of the complement system act in a series of enzymatic cascades through a variety of protein interactions and cleavage events. Complement activation occurs via three main pathways: the antibody-dependent classical pathway, the alternative pathway, and the mannose-binding lectin (MBL) pathway. Inappropriate or excessive complement activation is an underlying cause or contributing factor to a number of serious diseases and conditions, and considerable effort has been devoted over the past several decades to exploring various complement inhibitors as therapeutic agents.

### SUMMARY

In one aspect, the disclosure features a method of treating a complement-mediated eye disorder, the method comprising reducing level of C3 in the liver of a subject, thereby treating the eye disorder. In some embodiments, the method comprises systemically administering to a subject an siRNA that targets C3 mRNA in the subject. In some embodiments, the siRNA comprises a liver-targeting moiety. In some embodiments, the liver-targeting moiety is a GalNAc moiety.

In some embodiments, the siRNA comprises an antisense strand comprising a sequence listed in Tables 2A, 2B, 4, 5, 6, 10, 15, 16, 17, 18, 24-70, or 72-73 and/or comprises a sense strand comprising a sequence listed in Tables 1, 3A, 3B, 10, 15, 16, 17, 18, or 21-71.

In some embodiments, the siRNA comprises an antisense strand and a sense strand, wherein the antisense strand is complementary to a nucleotide sequence that is at least 90% identical to any one of SEQ ID NOs: 76-100 and/or the sense strand comprises a nucleotide sequence that is at least 90% identical to any one of SEQ ID NOs: 76-100. In some embodiments, the antisense strand is complementary to a nucleotide sequence comprising a sequence that differs by no more than 1, 2, 3, or 4 nucleotides from any one of SEQ ID NOs: 76-100 and/or the sense strand comprises a nucleotide sequence that differs by no more than 1, 2, 3, or 4 nucleotides from any one of SEQ ID NOs: 76-100. In some embodiments, the antisense strand is complementary to a nucleotide sequence comprising any one of SEQ ID NOs: 76-100. In some embodiments, the antisense strand comprises a nucleotide sequence comprising any one of SEQ ID NOs: 101-125.

In some embodiments, one or both of the sense strand and the antisense strand comprises at least one overhang region. In some embodiments, the at least one overhang region comprises a 1, 2, 3, 4, or 5, nucleotide overhang. In some embodiments, the at least one overhang region comprises a 3′ overhang. In some embodiments, the at least one overhang region is complementary to a fragment of SEQ ID NO: 75. In some embodiments, the 3′ overhang comprises a 2-nucleotide overhang.

In some embodiments, one or both of the sense strand and the antisense strand comprises at least one additional nucleotide on the 5′ end, the 3′ end, or both the 5′ end and the 3′ end, which is not complementary to a fragment of SEQ ID NO: 75.

In some embodiments, one or both of the sense stand and the antisense strand comprises at least one modified nucleotide. In some embodiments, the at least one modified nucleotide comprises a nucleotide that includes a 2′-O-Methyl group, a nucleotide that includes a 2′-Fluoro group, and/or a phosphorothioate bond with an adjacent nucleotide. In some embodiments, the at least one modified nucleotide comprises a phosphorothioate bond between the last two, three, or four nucleotides of (i) the 5′ terminus of the sense strand; (ii) the 3′ terminus of the sense strand; (iii) the 5′ terminus of the antisense strand, and/or (iv) the 3′ terminus of the antisense strand. In some embodiments, the at least one modified nucleotide comprises a phosphorothioate bond between the last three nucleotides of (i) the 5′ terminus of the sense strand; (ii) the 3′ terminus of the sense strand; (iii) the 5′ terminus of the antisense strand, and/or (iv) the 3′ terminus of the antisense strand. In some embodiments, the at least one modified nucleotide comprises a phosphorothioate bond between the last two, three, or four nucleotides of (i) the 5′ terminus of the sense strand; (ii) the 3′ terminus of the sense strand; (iii) the 5′ terminus of the antisense strand, and (iv) the 3′ terminus of the antisense strand.

In some embodiments, the sense strand comprises the nucleotide sequence of any one of SEQ ID NOs: 76-100, 126-150, 201, 203, 205, 207, 209, 211, 213, 215, 217, 219, 221, 223, 225, 227, 229, 231, 233, 235, 237, 239, 241, 243, 245, 247, 249, 255, 259, 264, 268, 272, 276, 325, 326, and 327. In some embodiments, the antisense strand comprises the nucleotide sequence of any one of SEQ ID NOs: 101-125, 151-200, 202, 204, 206, 208, 210, 212, 214, 216, 218, 220, 222, 224, 226, 228, 230, 232, 234, 236, 238, 240, 242, 244, 246, 248, 250, 252, 254, 256, 257, 258, 260, 261, 262, 263, 265, 266, 267, 269, 270, 271, 273, 274, 275, 277, 278, and 300-324. In some embodiments, the siRNA comprises a sense strand nucleotide sequence/antisense strand nucleotide sequence of any one of the following sets of sense/antisense SEQ ID NOs: 201/202, 203/204, 205/206, 207/208, 209/210, 211/212, 213/214, 215/216, 217/218, 219/220, 221/222, 223/224, 225/226, 227/228, 229/230, 231/232, 233/234, 235/236, 237/238, 239/240, 241/242, 243/244, 245/246, 247/248, 249/250, 251/252, 253/254, 201/256, 255/256, 255/257, 201/258, 255/258, 207/260, 259/260, 259/261, 207/262, 259/262, 217/263, 264/263, 264/265, 217/266, 264/266, 219/267, 268/267, 268/269, 219/270, 268/270, 231/271, 272/271, 272/273, 231/274, 272/274, 243/275, 276/275, 276/277, 243/278, 276/278, 325/275, 326/260, and 327/258.

In some embodiments, the siRNA comprises at least one ligand attached to one or more of the 5′ end of the sense strand, the 3′ end of the sense strand, the 5′ end of the antisense strand, and the 3′ end of the antisense strand. In some embodiments, the ligand comprises at least one GalNAc moiety. In some embodiments, the ligand comprises three GalNAc moieties.

In some embodiments, the method comprises administering to the subject a composition comprising a nucleic acid encoding the siRNA.

In some embodiments, after the administration of the siRNA or the composition, a level of C3 transcript or C3 protein in the subject or in a biological sample from the subject is reduced relative to a level before the administration of the siRNA or the composition. In some embodiments, the level of C3 transcript or C3 protein is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%, relative to a level before the administration.

In some embodiments, the siRNA or the composition is administered intravenously or subcutaneously to the subject. In some embodiments, the siRNA or the composition is administered to a hepatocyte of the subject. In some embodiments, the siRNA or the composition is administered to the hepatocyte ex vivo. In some embodiments, siRNA or the composition is administered to the hepatocyte in vivo.

In some embodiments, the method further comprises systemically administering to the subject a second agent. In some embodiments, the second agent is an anti-C3 antibody or a compstatin analog.

In some embodiments, the eye disorder is geographic atrophy or intermediate AMD.

In another aspect, the disclosure features a method of inhibiting or reducing, relative to a control, level of complement C3 in the eye of a subject, the method comprising reducing level of C3 in the liver of a subject, thereby reducing level of C3 in the eye. In some embodiments, the method comprises systemically administering to the subject an siRNA that targets C3 mRNA in the subject. In some embodiments, the siRNA comprises a liver-targeting moiety. In some embodiments, the liver-targeting moiety is a GalNAc moiety.

In some embodiments, the siRNA comprises an antisense strand comprising a sequence listed in Tables 2A, 2B, 4, 5, 6, 10, 15, 16, 17, 18, 24-70, or 72-73, and/or comprises a sense strand comprising a sequence listed in Tables 1, 3A, 3B, 10, 15, 16, 17, 18, or 21-71.

In some embodiments, the siRNA comprises an antisense strand and a sense strand, wherein the antisense strand is complementary to a nucleotide sequence that is at least 90% identical to any one of SEQ ID NOs: 76-100 and/or the sense strand comprises a nucleotide sequence that is at least 90% identical to any one of SEQ ID NOs: 76-100. In some embodiments, the antisense strand is complementary to a nucleotide sequence comprising a sequence that differs by no more than 1, 2, 3, or 4 nucleotides from any one of SEQ ID NOs: 76-100 and/or the sense strand comprises a nucleotide sequence that differs by no more than 1, 2, 3, or 4 nucleotides from any one of SEQ ID NOs: 76-100. In some embodiments, the antisense strand is complementary to a nucleotide sequence comprising any one of SEQ ID NOs: 76-100. In some embodiments, the antisense strand comprises a nucleotide sequence comprising any one of SEQ ID NOs: 101-125.

In some embodiments, one or both of the sense strand and the antisense strand comprises at least one overhang region. In some embodiments, the at least one overhang region comprises a 1, 2, 3, 4, or 5, nucleotide overhang. In some embodiments, the at least one overhang region comprises a 3′ overhang. In some embodiments, the at least one overhang region is complementary to a fragment of SEQ ID NO: 75. In some embodiments, the 3′ overhang comprises a 2-nucleotide overhang.

In some embodiments, one or both of the sense strand and the antisense strand comprises at least one additional nucleotide on the 5′ end, the 3′ end, or both the 5′ end and the 3′ end, which is not complementary to a fragment of SEQ ID NO:75.

In some embodiments, one or both of the sense stand and the antisense strand comprises at least one modified nucleotide. In some embodiments, the at least one modified nucleotide comprises a nucleotide that includes a 2′-O-Methyl group, a nucleotide that includes a 2′-Fluoro group, and/or a phosphorothioate bond with an adjacent nucleotide. In some embodiments, the at least one modified nucleotide comprises a phosphorothioate bond between the last two, three, or four nucleotides of (i) the 5′ terminus of the sense strand; (ii) the 3′ terminus of the sense strand; (iii) the 5′ terminus of the antisense strand, and/or (iv) the 3′ terminus of the antisense strand. In some embodiments, the at least one modified nucleotide comprises a phosphorothioate bond between the last three nucleotides of (i) the 5′ terminus of the sense strand; (ii) the 3′ terminus of the sense strand; (iii) the 5′ terminus of the antisense strand, and/or (iv) the 3′ terminus of the antisense strand. In some embodiments, the at least one modified nucleotide comprises a phosphorothioate bond between the last two, three, or four nucleotides of (i) the 5′ terminus of the sense strand; (ii) the 3′ terminus of the sense strand; (iii) the 5′ terminus of the antisense strand, and (iv) the 3′ terminus of the antisense strand. In some embodiments, the sense strand comprises the nucleotide sequence of any one of SEQ ID NOs: 76-100, 126-150, 201, 203, 205, 207, 209, 211, 213, 215, 217, 219, 221, 223, 225, 227, 229, 231, 233, 235, 237, 239, 241, 243, 245, 247, 249, 255, 259, 264, 268, 272, 276, 325, 326, and 327. In some embodiments, the antisense strand comprises the nucleotide sequence of any one of SEQ ID NOs: 101-125, 151-200, 202, 204, 206, 208, 210, 212, 214, 216, 218, 220, 222, 224, 226, 228, 230, 232, 234, 236, 238, 240, 242, 244, 246, 248, 250, 252, 254, 256, 257, 258, 260, 261, 262, 263, 265, 266, 267, 269, 270, 271, 273, 274, 275, 277, 278, and 300-324. In some embodiments, the siRNA comprises a sense strand nucleotide sequence/antisense strand nucleotide sequence of any one of the following sets of sense/antisense SEQ ID NOs: 201/202, 203/204, 205/206, 207/208, 209/210, 211/212, 213/214, 215/216, 217/218, 219/220, 221/222, 223/224, 225/226, 227/228, 229/230, 231/232, 233/234, 235/236, 237/238, 239/240, 241/242, 243/244, 245/246, 247/248, 249/250, 251/252, 253/254, 201/256, 255/256, 255/257, 201/258, 255/258, 207/260, 259/260, 259/261, 207/262, 259/262, 217/263, 264/263, 264/265, 217/266, 264/266, 219/267, 268/267, 268/269, 219/270, 268/270, 231/271, 272/271, 272/273, 231/274, 272/274, 243/275, 276/275, 276/277, 243/278, 276/278, 325/275, 326/260, and 327/258.

In some embodiments, the siRNA further comprises at least one ligand attached to one or more of the 5′ end of the sense strand, the 3′ end of the sense strand, the 5′ end of the antisense strand, and the 3′ end of the antisense strand. In some embodiments, the ligand comprises at least one GalNAc moiety. In some embodiments, the ligand comprises three GalNAc moieties.

In some embodiments, the method comprises administering to the subject a composition comprising a nucleic acid encoding the siRNA.

In some embodiments, after the administering step, the level of C3 transcript or C3 protein is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%, relative to a control level of C3 in the subject before the administering step.

In some embodiments, the method further comprises systemically administering to the subject a second agent. In some embodiments, the second agent is an anti-C3 antibody or a compstatin analog.

In some embodiments, the subject suffers from a complement-mediated eye disorder. In some embodiments, the eye disorder is geographic atrophy or intermediate AMD.

In some embodiments of any of the aspects described herein, the siRNA, the composition, or the second agent is not locally administered to the eye of the subject.

### Definitions

Antibody: As used herein, the term “antibody” refers to an immunoglobulin or a derivative thereof containing an immunoglobulin domain capable of binding to an antigen. The antibody can be of any species, e.g., human, rodent, rabbit, goat, chicken, etc. The antibody may be a member of any immunoglobulin class, including any of the human classes: IgG, IgM, IgA, IgD, and IgE, or subclasses thereof such as IgG1, IgG2, etc. In various embodiments of the invention the antibody is a fragment such as an Fab′, F(ab′)₂, scFv (single-chain variable) or other fragment that retains an antigen binding site, or a recombinantly produced scFv fragment, including recombinantly produced fragments. See, e.g., Allen, T., Nature Reviews Cancer, Vol. 2, 750-765, 2002, and references therein. The antibody can be monovalent, bivalent or multivalent. The antibody may be a chimeric or “humanized” antibody in which, for example, a variable domain of rodent origin is fused to a constant domain of human origin, thus retaining the specificity of the rodent antibody. The domain of human origin need not originate directly from a human in the sense that it is first synthesized in a human being. Instead, “human” domains may be generated in rodents whose genome incorporates human immunoglobulin genes. See, e.g., Vaughan, et al., (1998), Nature Biotechnology, 16:535-539. The antibody may be partially or completely humanized. An antibody may be polyclonal or monoclonal, though for purposes of the present invention monoclonal antibodies are generally preferred. Methods for producing antibodies that specifically bind to virtually any molecule of interest are known in the art. For example, monoclonal or polyclonal antibodies can be purified from blood or ascites fluid of an animal that produces the antibody (e.g., following natural exposure to or immunization with the molecule or an antigenic fragment thereof), can be produced using recombinant techniques in cell culture or transgenic organisms, or can be made at least in part by chemical synthesis.

Approximately: As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

Complementary: As used herein, in accordance with its art-accepted meaning, “complementary” refers to the capacity for precise pairing between particular bases, nucleosides, nucleotides or nucleic acids. For example, adenine (A) and uridine (U) are complementary; adenine (A) and thymidine (T) are complementary; and guanine (G) and cytosine (C), are complementary and are referred to in the art as Watson-Crick base pairings. If a nucleotide at a certain position of a first nucleic acid sequence is complementary to a nucleotide located opposite in a second nucleic acid sequence when the strands are aligned in anti-parallel orientation, the nucleotides form a complementary base pair, and the nucleic acids are complementary at that position. The percent complementarity of a first nucleic acid to a second nucleic acid may be evaluated by aligning them in antiparallel orientation for maximum complementarity over a window of evaluation, determining the total number of nt in both strands that form complementary base pairs within the window, dividing by the total number of nt within the window, and multiplying by 100. For example, AAAAAAAA and TTTGTTAT are 75% complementary since there are 12 nt in complementary base pairs out of a total of 16 nt. When computing the number of complementary nt needed to achieve a particular % complementarity, fractions are rounded to the nearest whole number. A position occupied by non-complementary nucleotides constitutes a mismatch, i.e., the position is occupied by a non-complementary base pair. In certain embodiments a window of evaluation has the length described herein for duplex portions or target portions. Complementary sequences include base-pairing of a polynucleotide comprising a first nucleotide sequence to a polynucleotide comprising a second nucleotide sequence over the entire length of both nucleotide sequences (if the same length) or over the entire length of the shorter sequence (if different lengths). Such sequences can be referred to as “perfectly complementary” (100% complementarity) with respect to each other herein. Nucleic acids that are at least 70% complementary over a window of evaluation are considered “substantially complementary” over that window. In certain embodiments complementary nucleic acids are at least 75%, at least 80%, at least 85%, at least 90%, or at least 95% complementary over the window of evaluation. Where a first sequence is referred to as “substantially complementary” with respect to a second sequence herein, the two sequences may be perfectly complementary or they may comprise one or more unmatched bases upon hybridization, e.g., up to about 5%, 10%, 15%, 20%, or 25% unmatched bases upon hybridization, e.g., 1, 2, 3, 4, 5, or 6 mismatched base pairs upon hybridization for a duplex up to 30 base pairs, while retaining the ability to hybridize under the conditions most relevant to their intended use. It should be understood that where two oligonucleotides are designed to form, upon hybridization, one or more single stranded overhangs, such overhangs are not regarded as mismatches or unpaired nucleotides with regard to the determination of percent complementarity. For example, the two strands of a dsRNA comprising one oligonucleotide 21 nucleotides in length and another oligonucleotide 23 nucleotides in length, wherein the longer oligonucleotide comprises a sequence of 21 nucleotides that is perfectly complementary to the shorter oligonucleotide and a 2 nucleotide overhang, may be referred to as “perfectly complementary” herein. “Complementary” sequences, as used herein may include one or more non-Watson-Crick base pairs and/or base pairs formed from non-natural and other modified nucleotides, in so far as the requirements with respect to their ability to hybridize are fulfilled. Such non-Watson-Crick base pairs include, but are not limited to, G:U Wobble or Hoogsteen base pairing. Those of ordinary skill in the art are aware that guanine, cytosine, adenine, and uracil can be replaced by other bases without substantially altering the base pairing properties of a polynucleotide comprising a nucleotide bearing such bases, according to the so-called “wobble” rules (see, e.g., Murphy, FV IV & V Ramakrishnan, V., Nature Structural and Molecular Biology 11:1251-1252 (2004)). For example, a nucleotide comprising inosine as its base can base pair with nucleotides containing adenine, cytosine, or uracil. Thus, nucleotides containing uracil, guanine, or adenine can be replaced in the nucleotide sequences of an Inhibitory RNA described herein by a nucleotide containing, for example, inosine. It will be understood that the terms “complementary”, “perfectly complementary”, and “substantially complementary” can be used with respect to the base matching between any two nucleic acids, e.g., the base matching between the sense strand and the antisense strand of a dsRNA, or between the antisense strand of a ds inhibitory RNA (e.g., an siRNA) and a target sequence, or between an antisense oligonucleotide and a target sequence, as will be evident from the context. “Hybridize”, as used herein, refers to the interaction between two nucleic acid sequences comprising or consisting of complementary portions such that a duplex structure is formed that is stable under the particular conditions of interest, as will be understood by the ordinary skilled artisan.

Complement component: As used herein, the terms “complement component” or “complement protein” is a molecule that is involved in activation of the complement system or participates in one or more complement-mediated activities. Components of the classical complement pathway include, e.g., C1q, C1r, C1s, C2, C3, C4, C5, C6, C7, C8, C9, and the C5b-9 complex, also referred to as the membrane attack complex (MAC) and active fragments or enzymatic cleavage products of any of the foregoing (e.g., C3a, C3b, C4a, C4b, C5a, etc.). Components of the alternative pathway include, e.g., factors B, D, H, and I, and properdin, with factor H being a negative regulator of the pathway. Components of the lectin pathway include, e.g., MBL2, MASP-1, and MASP-2. Complement components also include cell-bound receptors for soluble complement components. Such receptors include, e.g., C5a receptor (C5aR), C3a receptor (C3aR), Complement Receptor 1 (CR1), Complement Receptor 2 (CR2), Complement Receptor 3 (CR3), etc. It will be appreciated that the term “complement component” is not intended to include those molecules and molecular structures that serve as “triggers” for complement activation, e.g., antigen-antibody complexes, foreign structures found on microbial or artificial surfaces, etc.

Host cell: As used herein, the term “host cell” refers to a cell into which exogenous DNA (recombinant or otherwise) has been introduced. Persons of skill upon reading this disclosure will understand that such terms refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. In some embodiments, host cells include prokaryotic and eukaryotic cells selected from any of the Kingdoms of life that are suitable for expressing an exogenous DNA (e.g., a recombinant nucleic acid sequence). Exemplary cells include those of prokaryotes and eukaryotes (single-cell or multiple-cell), bacterial cells (e.g., strains of E. coli, Bacillus spp., Streptomyces spp., etc.), mycobacteria cells, fungal cells, yeast cells (e.g., S. cerevisiae, S. pombe, P. pastoris, P. methanolica, etc.), plant cells, insect cells (e.g., SF-9, SF-21, baculovirus-infected insect cells, Trichoplusia ni, etc.), non-human animal cells, human cells, or cell fusions such as, for example, hybridomas or quadromas. In some embodiments, the cell is a human, monkey, ape, hamster, rat, or mouse cell. In some embodiments, the cell is eukaryotic and is selected from the following cells: CHO (e.g., CHO KI, DXB-1 1 CHO, Veggie-CHO), COS (e.g., COS-7), retinal cell, Vero, CV1, kidney (e.g., HEK293, 293 EBNA, MSR 293, MDCK, HaK, BHK), HeLa, HepG2, WI38, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3 A cell, HT1080 cell, myeloma cell, tumor cell, and a cell line derived from an aforementioned cell. In some embodiments, the cell comprises one or more viral genes.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4:11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Linked: As used herein, the term “linked”, when used with respect to two or more moieties, means that the moieties are physically associated or connected with one another to form a molecular structure that is sufficiently stable so that the moieties remain associated under the conditions in which the linkage is formed and, preferably, under the conditions in which the new molecular structure is used, e.g., physiological conditions. In certain preferred embodiments of the invention the linkage is a covalent linkage. In other embodiments the linkage is noncovalent. Moieties may be linked either directly or indirectly. When two moieties are directly linked, they are either covalently bonded to one another or are in sufficiently close proximity such that intermolecular forces between the two moieties maintain their association. When two moieties are indirectly linked, they are each linked either covalently or noncovalently to a third moiety, which maintains the association between the two moieties. In general, when two moieties are referred to as being linked by a “linker” or “linking moiety” or “linking portion”, the linkage between the two linked moieties is indirect, and typically each of the linked moieties is covalently bonded to the linker. The linker can be any suitable moiety that reacts with the two moieties to be linked within a reasonable period of time, under conditions consistent with stability of the moieties (which may be protected as appropriate, depending upon the conditions), and in sufficient amount, to produce a reasonable yield.

MicroRNA (miRNA): As used herein, the term “microRNA” or “miRNA” refers to a small non-coding RNA molecule that can function in transcriptional and/or post-transcriptional regulation of target gene expression. The terms encompass a mature miRNA sequence or a precursor miRNA sequence, including a primary transcript (pri-miRNA) and a stem-loop precursor (pre-miRNA). The biogenesis of a naturally occurring miRNA initiates in the nucleus by RNA polymerase II transcription, generating a primary transcript (pri-miRNA). The primary transcript is cleaved by Drosha ribonuclease III enzyme to produce an approximately 70 nt stem-loop precursor miRNA (pre-miRNA). The pre-miRNA is then actively exported to the cytoplasm where it is cleaved by Dicer ribonuclease to form the mature miRNA, which includes an “antisense strand” or “guide strand” (that includes a region that is substantially complementary to a target sequence) and a “sense strand” or “passenger strand” (that includes a region that is substantially complementary to a region of the antisense strand). Those of ordinary skill in the art will appreciate that a guide strand may be perfectly complementary to a target region of a target RNA or may have less than perfect complementarity to a target region of a target RNA. The guide strand of this miRNA is incorporated into an RNA-induced silencing complex (RISC) that recognizes target mRNAs through base pairing with the miRNA, and commonly results in translational inhibition or destabilization of the target mRNA. As is understood in the field, for naturally occurring miRNAs, target mRNA recognition occurs through imperfect base pairing with the mRNA. In some embodiments, an miRNA is synthetic or engineered, and target mRNA recognition occurs through perfect base pairing with the mRNA. Typically, the target mRNA contains a sequence complementary to a “seed” sequence of the miRNA, which usually corresponds to nucleotides 2-8 of the miRNA. Information concerning miRNAs and associated pri-miRNA and pre-miRNA sequences is available in miRNA databases such as miRBase (Griffiths-Jones et al. 2008 Nucl Acids Res 36, (Database Issue: D154-D158) and the NCBI human genome database.

Operably linked: As used herein, the term “operably linked” refers to a juxtaposition wherein the components described are in a relationship permitting them to function in their intended manner. A control element “operably linked” to a functional element is associated in such a way that expression and/or activity of the functional element is achieved under conditions compatible with the control element. In some embodiments, “operably linked” control elements are contiguous (e.g., covalently linked) with the coding elements of interest; in some embodiments, control elements act in trans to or otherwise at a from the functional element of interest.

Recombinant: As used herein, the term “recombinant” is intended to refer to polypeptides that are designed, engineered, prepared, expressed, created, manufactured, and/or or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof; and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc.).

RNA interference: As used herein, the term “RNA interference” or “RNAi” refers generally to a process in which a double-stranded RNA molecule or a short hairpin RNA molecule reduces or inhibits expression of a nucleic acid sequence with which the double-stranded or short hairpin RNA molecule shares substantial or total homology. Without wishing to be bound by any theory, it is believed that, in nature, the RNAi pathway is initiated by a Type III endonuclease known as Dicer, which cleaves long double-stranded RNA (dsRNA) into double-stranded fragments typically of 21-23 base pairs with 2-base 3′ overhangs (although variations in length and overhangs are also contemplated), referred to as “short interfering RNAs” (“siRNAs”). Such siRNAs comprise two single-stranded RNAs (ssRNAs), with an “antisense strand” or “guide strand” that includes a region that is substantially complementary to a target sequence, and a “sense strand” or “passenger strand” that includes a region that is substantially complementary to a region of the antisense strand. Those of ordinary skill in the art will appreciate that a guide strand may be perfectly complementary to a target region of a target RNA or may have less than perfect complementarity to a target region of a target RNA.

Subject: As used herein, the term “subject” or “test subject” refers to any organism to which a provided compound or composition is administered in accordance with the present invention e.g., for experimental, diagnostic, prophylactic, and/or therapeutic purposes. Typical subjects include animals (e.g., mammals such as mice, rats, rabbits, non-human primates, and humans; insects; worms; etc.) and plants. In some embodiments, a subject may be suffering from, and/or susceptible to a disease, disorder, and/or condition.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Suffering from: An individual who is “suffering from” a disease, disorder, and/or condition has been diagnosed with and/or displays one or more symptoms of a disease, disorder, and/or condition.

Target gene: A “target gene”, as used herein, refers to a gene whose expression is to be modulated, e.g., inhibited. As used herein, the term “target RNA” refers to an RNA to be degraded or translationally repressed or otherwise inhibited using one or more miRNAs. A target RNA may also be referred to as a target sequence or target transcript. The RNA may be a primary RNA transcript transcribed from the target gene (e.g., a pre-mRNA) or a processed transcript, e.g., mRNA encoding a polypeptide. As used herein, the term “target portion” or “target region” refers to a contiguous portion of the nucleotide sequence of a target RNA. In some embodiments, a target portion an mRNA is at least long enough to serve as a substrate for RNA interference (RNAi)-mediated cleavage within that portion in the presence of a suitable inhibitory RNA. A target portion may be from about 8-36 nucleotides in length, e.g., about 10-20 or about 15-30 nucleotides in length. A target portion length may have specific value or subrange within the afore-mentioned ranges. For example, in certain embodiments a target portion may be between about 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length.

Therapeutic agent: As used herein, the phrase “therapeutic agent” refers to any agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or signs of the disease, disorder, and/or condition. In some embodiments, a therapeutically effective amount is administered in a single dose; in some embodiments, multiple unit doses are required to deliver a therapeutically effective amount.

Treating: As used herein, the term “treating” refers to providing treatment, i.e., providing any type of medical or surgical management of a subject. The treatment can be provided in order to reverse, alleviate, inhibit the progression of, prevent or reduce the likelihood of a disease, disorder, or condition, or in order to reverse, alleviate, inhibit or prevent the progression of, prevent or reduce the likelihood of one or more symptoms or manifestations of a disease, disorder or condition. “Prevent” refers to causing a disease, disorder, condition, or symptom or manifestation of such not to occur for at least a period of time in at least some individuals. Treating can include administering an agent to the subject following the development of one or more symptoms or manifestations indicative of a complement-mediated condition, e.g., in order to reverse, alleviate, reduce the severity of, and/or inhibit or prevent the progression of the condition and/or to reverse, alleviate, reduce the severity of, and/or inhibit or one or more symptoms or manifestations of the condition. A composition of the disclosure can be administered to a subject who has developed a complement-mediated disorder or is at increased risk of developing such a disorder relative to a member of the general population. A composition of the disclosure can be administered prophylactically, i.e., before development of any symptom or manifestation of the condition. Typically in this case the subject will be at risk of developing the condition.

Nucleic acid: The term “nucleic acid” includes any nucleotides, analogs thereof, and polymers thereof. The term “polynucleotide” as used herein refer to a polymeric form of nucleotides of any length, either ribonucleotides (RNA) or deoxyribonucleotides (DNA). These terms refer to the primary structure of the molecules and, thus, include double- and single-stranded DNA, and double- and single-stranded RNA. These terms include, as equivalents, analogs of either RNA or DNA made from nucleotide analogs and modified polynucleotides such as, though not limited to, methylated, protected and/or capped nucleotides or polynucleotides. The terms encompass poly- or oligo-ribonucleotides (RNA) and poly- or oligo-deoxyribonucleotides (DNA); RNA or DNA derived from N-glycosides or C-glycosides of nucleobases and/or modified nucleobases; nucleic acids derived from sugars and/or modified sugars; and nucleic acids derived from phosphate bridges and/or modified phosphorus-atom bridges (also referred to herein as “internucleotide linkages”). The term encompasses nucleic acids containing any combinations of nucleobases, modified nucleobases, sugars, modified sugars, phosphate bridges or modified phosphorus atom bridges. Examples include, and are not limited to, nucleic acids containing ribose moieties, the nucleic acids containing deoxy-ribose moieties, nucleic acids containing both ribose and deoxyribose moieties, nucleic acids containing ribose and modified ribose moieties. In some embodiments, the prefix poly-refers to a nucleic acid containing 2 to about 10,000, 2 to about 50,000, or 2 to about 100,000 nucleotide monomer units. In some embodiments, the prefix oligo-refers to a nucleic acid containing 2 to about 200 nucleotide monomer units.

Vector: As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.”

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose.

### BRIEF DESCRIPTION OF THE DRAWING

FIG. 1 shows a chart that discloses example modification patterns 1-5 of the sense and antisense strands of the duplex of an inhibitory RNA (e.g., an siRNA). In FIG. 1, “2OM” represents a 2′-O-methyl modification, “2F” represents a 2′-Fluoro modification, and “PS” represents a phosphorothioate bond with the adjacent 3′ nucleotide.

FIG. 2 shows the structure of pegcetacoplan (“APL-2”), assuming n of about 800 to about 1100 and a PEG of about 40 kD.

FIG. 3 presents results from an in vivo study in non-human primates. siRNA 58 at dosages of 3 mg/kg, 10 mg/kg, 30 mg/kg, or vehicle, was administered by subcutaneous injection. The graph shows the time course for the levels of serum C3 protein for up to 67 days post-dose for each group. The level of C3 protein in serum was measured using an ELISA assay. Values at day −1 value were used as the baseline.

FIG. 4 presents data from an in vivo study in non-human primates. siRNA 58 at dosages of 3 mg/kg, 10 mg/kg, 30 mg/kg, or vehicle, was administered by subcutaneous injection. The graph depicts C3 mRNA expression in liver biopsies taken from the non-human primates on day 15 post-injection. The level of C3 mRNA in the samples was measured using a quantitative PCR assay, C3 mRNA level was normalized to the level of ActB mRNA in these experiments.

FIG. 5 presents data from an in vivo study in non-human primates. siRNA 58 at dosages of 3 mg/kg, 10 mg/kg, 30 mg/kg, or vehicle, was administered by subcutaneous injection. The graph depicts C3 mRNA expression in liver biopsies taken from the non-human primates on day 46 post-injection. The level of C3 mRNA in the samples was measured using a quantitative PCR assay, C3 mRNA level was normalized to the level of ActB mRNA in these experiments.

FIG. 6 presents a time course for levels of alternative pathway (AH50) activity in serum from the non-human primates injected with varying doses of siRNA 58 (3 mg/kg, 10 mg/kg, 30 mg/kg, or vehicle), collected up to day 29. Alternative pathway activity (AH50) was determined using an ELISA assay. Values at day −1 value were used as the baseline.

FIG. 7 shows the percentage change in plasma C3 concentration from baseline following subcutaneous administration of siRNA 59 as a single bolus (A) or 3× daily bolus (B). Values depicted as zero were below LLOQ of assay. Data represents mean±SEM (n=3).

FIG. 8 shows a measurement of alternative pathway activity, by detection and quantification of soluble C5b-9 complex using ELISA (measurement of optical density; OD), in serum from animals subcutaneously treated with vehicle (A), siRNA (−) control (B) siRNA 59 at 3 mg/kg (C), siRNA 59 at 10 mg/kg (D) and siRNA 59 at 30 mg/kg (E). Data represents mean #SEM (n=3).

FIG. 9 shows levels of C3 mRNA in liver tissue at 3 days (A) and 30 days (B and C) following a single administration (A and B) or three daily administrations (C) of siRNA 59. Data represents mean±SEM (n=3).

FIG. 10 shows levels of C3 in serum from non-human primates at various timepoints after administration of either siRNA (100 mg/kg) or vehicle. Panel A shows average serum C3 concentration (ng/ml). Panel B shows average serum C3 reduction as a percent reduction from levels before siRNA or vehicle were administered (i.e., baseline levels).

FIG. 11 shows levels of C3a in plasma from non-human primates at various timepoints after administration of either siRNA (100 mg/kg) or vehicle. Panel A shows average plasma C3a concentration (ng/ml). Panel B shows average plasma C3a reduction as a percent reduction from levels before siRNA or vehicle were administered (i.e., baseline levels).

FIG. 12 shows C3 (Panel A) and C3a (Panel B) levels (ng/ml) in vitreous humor (VH) of non-human primates 44 days after administration of either siRNA (100 mg/kg) or vehicle.

FIG. 13 shows C3 (Panel A) and C3a (Panel B) levels (ng/ml) in aqueous humor (AH) of non-human primates 44 days after administration of either siRNA (100 mg/kg) or vehicle.

### DETAILED DESCRIPTION OF CERTAIN EMBODIMENTS

The disclosure is based, in part, on the insight that eye disorders (e.g., complement-mediated eye disorders) can be treated by targeted reduction of complement in the liver without local administration of a complement inhibitor to the eye. In some embodiments, complement-mediated eye disorders can be treated by systemic administration of one or more complement inhibitors (e.g., a complement inhibitor described herein, e.g., a complement inhibitor targeted to the liver) and without local administration of a complement inhibitor, e.g., a complement inhibitor described herein, to the eye.

### I. COMPLEMENT SYSTEM

To facilitate understanding of the disclosure, and without intending to limit the invention in any way, this section provides an overview of complement and its pathways of activation. Further details are found, e.g., in Kuby Immunology, 6th ed., 2006; Paul, W. E., Fundamental Immunology, Lippincott Williams & Wilkins; 6th ed., 2008; and Walport M J., Complement. First of two parts. N Engl J Med., 344 (14): 1058-66, 2001.

Complement is an arm of the innate immune system that plays an important role in defending the body against infectious agents. The complement system comprises more than 30 serum and cellular proteins that are involved in three major pathways, known as the classical, alternative, and lectin pathways. The classical pathway is usually triggered by binding of a complex of antigen and IgM or IgG antibody to C1 (though certain other activators can also initiate the pathway). Activated C1 cleaves C4 and C2 to produce C4a and C4b, in addition to C2a and C2b, C4b and C2a combine to form C3 convertase, which cleaves C3 to form C3a and C3b. Binding of C3b to C3 convertase produces C5 convertase, which cleaves C5 into C5a and C5b, C3a, C4a, and C5a are anaphylotoxins and mediate multiple reactions in the acute inflammatory response, C3a and C5a are also chemotactic factors that attract immune system cells such as neutrophils. It will be understood that the names “C2a” and “C2b” used initially were subsequently reversed in the scientific literature.

The alternative pathway is initiated by and amplified at, e.g., microbial surfaces and various complex polysaccharides. In this pathway, hydrolysis of C3 to C3 (H₂O), which occurs spontaneously at a low level, leads to binding of factor B, which is cleaved by factor D, generating a fluid phase C3 convertase that activates complement by cleaving C3 into C3a and C3b, C3b binds to targets such as cell surfaces and forms a complex with factor B, which is later cleaved by factor D, resulting in a C3 convertase. Surface-bound C3 convertases cleave and activate additional C3 molecules, resulting in rapid C3b deposition in close proximity to the site of activation and leading to formation of additional C3 convertase, which in turn generates additional C3b. This process results in a cycle of C3 cleavage and C3 convertase formation that significantly amplifies the response. Cleavage of C3 and binding of another molecule of C3b to the C3 convertase gives rise to a C5 convertase, C3 and C5 convertases of this pathway are regulated by cellular molecules CR1, DAF, MCP, CD59, and fH. The mode of action of these proteins involves either decay accelerating activity (i.e., ability to dissociate convertases), ability to serve as cofactors in the degradation of C3b or C4b by factor I, or both. Normally the presence of complement regulatory proteins on cell surfaces prevents significant complement activation from occurring thereon.

The C5 convertases produced in both pathways cleave C5 to produce C5a and C5b. C5b then binds to C6, C7, and C8 to form C5b-8, which catalyzes polymerization of C9 to form the C5b-9 membrane attack complex (MAC). The MAC inserts itself into target cell membranes and causes cell lysis. Small amounts of MAC on the membrane of cells may have a variety of consequences other than cell death.

The lectin complement pathway is initiated by binding of mannose-binding lectin (MBL) and MBL-associated serine protease (MASP) to carbohydrates. The MB1-1 gene (known as LMAN-1 in humans) encodes a type I integral membrane protein localized in the intermediate region between the endoplasmic reticulum and the Golgi. The MBL-2 gene encodes the soluble mannose-binding protein found in serum. In the human lectin pathway, MASP-1 and MASP-2 are involved in the proteolysis of C4 and C2, leading to a C3 convertase described above.

Complement activity is regulated by various mammalian proteins referred to as complement control proteins (CCPs) or regulators of complement activation (RCA) proteins (U.S. Pat. No. 6,897,290). These proteins differ with respect to ligand specificity and mechanism(s) of complement inhibition. They may accelerate the normal decay of convertases and/or function as cofactors for factor I, to enzymatically cleave C3b and/or C4b into smaller fragments. CCPs are characterized by the presence of multiple (typically 4-56) homologous motifs known as short consensus repeats (SCR), complement control protein (CCP) modules, or SUSHI domains, about 50-70 amino acids in length that contain a conserved motif including four disulfide-bonded cysteines (two disulfide bonds), proline, tryptophan, and many hydrophobic residues. The CCP family includes complement receptor type 1 (CR1; C3b: C4b receptor), complement receptor type 2 (CR2), membrane cofactor protein (MCP; CD46), decay-accelerating factor (DAF), complement factor H (fH), and C4b-binding protein (C4 bp). CD59 is a membrane-bound complement regulatory protein unrelated structurally to the CCPs. Complement regulatory proteins normally serve to limit complement activation that might otherwise occur on cells and tissues of the mammalian, e.g., human host. Thus, “self” cells are normally protected from the deleterious effects that would otherwise ensue were complement activation to proceed on these cells. Deficiencies or defects in complement regulatory protein(s) are involved in the pathogenesis of a variety of complement-mediated disorders, e.g., as discussed herein.

Complement components (including C3 protein or C3 mRNA) have been reported to be expressed in eye tissues (including the retina, RPE, and choroid) and cell types (including microglia, astrocytes, myeloid cells and vascular cells) (see, e.g., Jong et al., Prog. Retinal and Eye Research, https://doi.org/10.1016/j.preteyeres.2021.100952 (2021)), C3 mRNA expression by microglia/monocytes in the retina was reported to contribute to activation of complement in the aging retina in rats (see, e.g., Rutar et al., PLOS ONE PLOS ONE 9 (4): e93343. doi: 10.1371/journal.pone.0093343 (2014)). Additionally, local complement dysregulation was reported in neovascular age-related macular degeneration (see, e.g., Schick et al., Eye 31:810-813 (2017)). Using a mouse model of retinal degeneration, intravitreal injection of C3 siRNA was reported to inhibit complement activation and deposition and to reduce cell death, whereas systemic depletion of serum complement was reported to have no effect (see, e.g., Natoli et al., Invest. Ophthalmol. Vis. Sci. 58:2977-2990 (2017)).

### II. INHIBITORY RNAS FOR C3

The disclosure includes compositions and methods related to one or more nucleotide sequences that are, comprise, or encode an inhibitory RNA that binds to and inhibits expression of messenger RNA (mRNA) produced by a target gene (e.g., C3). Inhibitory RNAs can be single stranded (e.g., an antisense oligonucleotide) or double stranded nucleic acid. In some embodiments, an inhibitory RNA comprises a double stranded RNA duplex such as microRNA (miRNA) or small interfering RNA (siRNA). In some embodiments, in inhibitory RNA is an siRNA or miRNA, or a vector comprising a nucleotide sequence encoding an siRNA or miRNA.

In some embodiments, an inhibitory RNA is capable of inhibiting expression of C3 of one or more non-human species, e.g., a non-human primate C3, e.g., Macaca fascicularis C3, or e.g., chlorocebus sabaeus in addition to human C3. The Macaca fascicularis C3 gene has been assigned NCBI Gene ID: 102131458 and the predicted amino acid and nucleotide sequence of Macaca fascicularis C3 are listed under NCBI RefSeq accession numbers XP_005587776.1 and XM_005587719.2, respectively. In some embodiments, an inhibitory RNA comprises an antisense strand that is complementary to a target portion that is identical in the human and Macaca fascicularis C3 transcripts. In some embodiments, an inhibitory RNA comprises an antisense strand that is complementary to a target portion of a human C3 transcript that differs by 1, 2, or 3 nucleotides from a sequence in a Macaca fascicularis C3 transcript. It will be appreciated that an inhibitory RNA that inhibits expression of human C3 may also inhibit expression of non-primate C3, e.g., rat or mouse C3, particularly if conserved regions of C3 transcript are targeted.

The amino acid and nucleotide sequences of human C3 are known in the art and can be found in publicly available databases, for example, the National Center for Biotechnology Information (NCBI) Reference Sequence (RefSeq) database, where they are listed under RefSeq accession numbers NP_000055 (accession.version number NP_000055.2) and NM_000064 (accession.version number NM_000064.4), respectively (where “amino acid sequence” refers to the sequence of the C3 polypeptide and “nucleotide sequence” in this context refers to the C3 mRNA sequence as represented in genomic DNA, it being understood that the actual mRNA nucleotide sequence contains U rather than T). One of ordinary skill in the art will appreciate that the afore-mentioned sequences are for the complement C3 preproprotein, which includes a signal sequence that is cleaved off and is therefore not present in the mature protein. The human C3 gene has been assigned NCBI Gene ID: 718, and the genomic C3 sequence has RefSeq accession number NG_009557 (accession.version number NG_009557.1). The nucleotide sequence of human C3 mRNA is presented below (from RefSeq accession number NM_000064.3 with T replaced by U; AUG initiation codon underlined starting at position 94).

(SEQ ID NO: 75)
AGAUAAAAAGCCAGCUCCAGCAGGCGCUGCUCACUCCUCCCCAUCCUCUCCCUCUGU
CCCUCUGUCCCUCUGACCCUGCACUGUCCCAGCACCAUGGGACCCACCUCAGGUCCCAGCCUGC
UGCUCCUGCUACUAACCCACCUCCCCCUGGCUCUGGGGAGUCCCAUGUACUCUAUCAUCACCCC
CAACAUCUUGCGGCUGGAGAGCGAGGAGACCAUGGUGCUGGAGGCCCACGACGCGCAAGGGGAU
GUUCCAGUCACUGUUACUGUCCACGACUUCCCAGGCAAAAAACUAGUGCUGUCCAGUGAGAAGA
CUGUGCUGACCCCUGCCACCAACCACAUGGGCAACGUCACCUUCACGAUCCCAGCCAACAGGGA
GUUCAAGUCAGAAAAGGGGCGCAACAAGUUCGUGACCGUGCAGGCCACCUUCGGGACCCAAGUG
GUGGAGAAGGUGGUGCUGGUCAGCCUGCAGAGCGGGUACCUCUUCAUCCAGACAGACAAGACCA
UCUACACCCCUGGCUCCACAGUUCUCUAUCGGAUCUUCACCGUCAACCACAAGCUGCUACCCGU
GGGCCGGACGGUCAUGGUCAACAUUGAGAACCCGGAAGGCAUCCCGGUCAAGCAGGACUCCUUG
UCUUCUCAGAACCAGCUUGGCGUCUUGCCCUUGUCUUGGGACAUUCCGGAACUCGUCAACAUGG
GCCAGUGGAAGAUCCGAGCCUACUAUGAAAACUCACCACAGCAGGUCUUCUCCACUGAGUUUGA
GGUGAAGGAGUACGUGCUGCCCAGUUUCGAGGUCAUAGUGGAGCCUACAGAGAAAUUCUACUAC
AUCUAUAACGAGAAGGGCCUGGAGGUCACCAUCACCGCCAGGUUCCUCUACGGGAAGAAAGUGG
AGGGAACUGCCUUUGUCAUCUUCGGGAUCCAGGAUGGCGAACAGAGGAUUUCCCUGCCUGAAUC
CCUCAAGCGCAUUCCGAUUGAGGAUGGCUCGGGGGAGGUUGUGCUGAGCCGGAAGGUACUGCUG
GACGGGGUGCAGAACCCCCGAGCAGAAGACCUGGUGGGGAAGUCUUUGUACGUGUCUGCCACCG
UCAUCUUGCACUCAGGCAGUGACAUGGUGCAGGCAGAGCGCAGCGGGAUCCCCAUCGUGACCUC
UCCCUACCAGAUCCACUUCACCAAGACACCCAAGUACUUCAAACCAGGAAUGCCCUUUGACCUC
AUGGUGUUCGUGACGAACCCUGAUGGCUCUCCAGCCUACCGAGUCCCCGUGGCAGUCCAGGGCG
AGGACACUGUGCAGUCUCUAACCCAGGGAGAUGGCGUGGCCAAACUCAGCAUCAACACACACCC
CAGCCAGAAGCCCUUGAGCAUCACGGUGCGCACGAAGAAGCAGGAGCUCUCGGAGGCAGAGCAG
GCUACCAGGACCAUGCAGGCUCUGCCCUACAGCACCGUGGGCAACUCCAACAAUUACCUGCAUC
UCUCAGUGCUACGUACAGAGCUCAGACCCGGGGAGACCCUCAACGUCAACUUCCUCCUGCGAAU
GGACCGCGCCCACGAGGCCAAGAUCCGCUACUACACCUACCUGAUCAUGAACAAGGGCAGGCUG
UUGAAGGCGGGACGCCAGGUGCGAGAGCCCGGCCAGGACCUGGUGGUGCUGCCCCUGUCCAUCA
CCACCGACUUCAUCCCUUCCUUCCGCCUGGUGGCGUACUACACGCUGAUCGGUGCCAGCGGCCA
GAGGGAGGUGGUGGCCGACUCCGUGUGGGUGGACGUCAAGGACUCCUGCGUGGGCUCGCUGGUG
GUAAAAAGCGGCCAGUCAGAAGACCGGCAGCCUGUACCUGGGCAGCAGAUGACCCUGAAGAUAG
AGGGUGACCACGGGGCCCGGGUGGUACUGGUGGCCGUGGACAAGGGCGUGUUCGUGCUGAAUAA
GAAGAACAAACUGACGCAGAGUAAGAUCUGGGACGUGGUGGAGAAGGCAGACAUCGGCUGCACC
CCGGGCAGUGGGAAGGAUUACGCCGGUGUCUUCUCCGACGCAGGGCUGACCUUCACGAGCAGCA
GUGGCCAGCAGACCGCCCAGAGGGCAGAACUUCAGUGCCCGCAGCCAGCCGCCCGCCGACGCCG
UUCCGUGCAGCUCACGGAGAAGCGAAUGGACAAAGUCGGCAAGUACCCCAAGGAGCUGCGCAAG
UGCUGCGAGGACGGCAUGCGGGAGAACCCCAUGAGGUUCUCGUGCCAGCGCCGGACCCGUUUCA
UCUCCCUGGGCGAGGCGUGCAAGAAGGUCUUCCUGGACUGCUGCAACUACAUCACAGAGCUGCG
GCGGCAGCACGCGCGGGCCAGCCACCUGGGCCUGGCCAGGAGUAACCUGGAUGAGGACAUCAUU
GCAGAAGAGAACAUCGUUUCCCGAAGUGAGUUCCCAGAGAGCUGGCUGUGGAACGUUGAGGACU
UGAAAGAGCCACCGAAAAAUGGAAUCUCUACGAAGCUCAUGAAUAUAUUUUUGAAAGACUCCAU
CACCACGUGGGAGAUUCUGGCUGUGAGCAUGUCGGACAAGAAAGGGAUCUGUGUGGCAGACCCC
UUCGAGGUCACAGUAAUGCAGGACUUCUUCAUCGACCUGCGGCUACCCUACUCUGUUGUUCGAA
ACGAGCAGGUGGAAAUCCGAGCCGUUCUCUACAAUUACCGGCAGAACCAAGAGCUCAAGGUGAG
GGUGGAACUACUCCACAAUCCAGCCUUCUGCAGCCUGGCCACCACCAAGAGGCGUCACCAGCAG
ACCGUAACCAUCCCCCCCAAGUCCUCGUUGUCCGUUCCAUAUGUCAUCGUGCCGCUAAAGACCG
GCCUGCAGGAAGUGGAAGUCAAGGCUGCUGUCUACCAUCAUUUCAUCAGUGACGGUGUCAGGAA
GUCCCUGAAGGUCGUGCCGGAAGGAAUCAGAAUGAACAAAACUGUGGCUGUUCGCACCCUGGAU
CCAGAACGCCUGGGCCGUGAAGGAGUGCAGAAAGAGGACAUCCCACCUGCAGACCUCAGUGACC
AAGUCCCGGACACCGAGUCUGAGACCAGAAUUCUCCUGCAAGGGACCCCAGUGGCCCAGAUGAC
AGAGGAUGCCGUCGACGCGGAACGGCUGAAGCACCUCAUUGUGACCCCCUCGGGCUGCGGGGAA
CAGAACAUGAUCGGCAUGACGCCCACGGUCAUCGCUGUGCAUUACCUGGAUGAAACGGAGCAGU
GGGAGAAGUUCGGCCUAGAGAAGCGGCAGGGGGCCUUGGAGCUCAUCAAGAAGGGGUACACCCA
GCAGCUGGCCUUCAGACAACCCAGCUCUGCCUUUGCGGCCUUCGUGAAACGGGCACCCAGCACC
UGGCUGACCGCCUACGUGGUCAAGGUCUUCUCUCUGGCUGUCAACCUCAUCGCCAUCGACUCCC
AAGUCCUCUGCGGGGCUGUUAAAUGGCUGAUCCUGGAGAAGCAGAAGCCCGACGGGGUCUUCCA
GGAGGAUGCGCCCGUGAUACACCAAGAAAUGAUUGGUGGAUUACGGAACAACAACGAGAAAGAC
AUGGCCCUCACGGCCUUUGUUCUCAUCUCGCUGCAGGAGGCUAAAGAUAUUUGCGAGGAGCAGG
UCAACAGCCUGCCAGGCAGCAUCACUAAAGCAGGAGACUUCCUUGAAGCCAACUACAUGAACCU
ACAGAGAUCCUACACUGUGGCCAUUGCUGGCUAUGCUCUGGCCCAGAUGGGCAGGCUGAAGGGG
CCUCUUCUUAACAAAUUUCUGACCACAGCCAAAGAUAAGAACCGCUGGGAGGACCCUGGUAAGC
AGCUCUACAACGUGGAGGCCACAUCCUAUGCCCUCUUGGCCCUACUGCAGCUAAAAGACUUUGA
CUUUGUGCCUCCCGUCGUGCGUUGGCUCAAUGAACAGAGAUACUACGGUGGUGGCUAUGGCUCU
ACCCAGGCCACCUUCAUGGUGUUCCAAGCCUUGGCUCAAUACCAAAAGGACGCCCCUGACCACC
AGGAACUGAACCUUGAUGUGUCCCUCCAACUGCCCAGCCGCAGCUCCAAGAUCACCCACCGUAU
CCACUGGGAAUCUGCCAGCCUCCUGCGAUCAGAAGAGACCAAGGAAAAUGAGGGUUUCACAGUC
ACAGCUGAAGGAAAAGGCCAAGGCACCUUGUCGGUGGUGACAAUGUACCAUGCUAAGGCCAAAG
AUCAACUCACCUGUAAUAAAUUCGACCUCAAGGUCACCAUAAAACCAGCACCGGAAACAGAAAA
GAGGCCUCAGGAUGCCAAGAACACUAUGAUCCUUGAGAUCUGUACCAGGUACCGGGGAGACCAG
GAUGCCACUAUGUCUAUAUUGGACAUAUCCAUGAUGACUGGCUUUGCUCCAGACACAGAUGACC
UGAAGCAGCUGGCCAAUGGUGUUGACAGAUACAUCUCCAAGUAUGAGCUGGACAAAGCCUUCUC
CGAUAGGAACACCCUCAUCAUCUACCUGGACAAGGUCUCACACUCUGAGGAUGACUGUCUAGCU
UUCAAAGUUCACCAAUACUUUAAUGUAGAGCUUAUCCAGCCUGGAGCAGUCAAGGUCUACGCCU
AUUACAACCUGGAGGAAAGCUGUACCCGGUUCUACCAUCCGGAAAAGGAGGAUGGAAAGCUGAA
CAAGCUCUGCCGUGAUGAACUGUGCCGCUGUGCUGAGGAGAAUUGCUUCAUACAAAAGUCGGAU
GACAAGGUCACCCUGGAAGAACGGCUGGACAAGGCCUGUGAGCCAGGAGUGGACUAUGUGUACA
AGACCCGACUGGUCAAGGUUCAGCUGUCCAAUGACUUUGACGAGUACAUCAUGGCCAUUGAGCA
GACCAUCAAGUCAGGCUCGGAUGAGGUGCAGGUUGGACAGCAGCGCACGUUCAUCAGCCCCAUC
AAGUGCAGAGAAGCCCUGAAGCUGGAGGAGAAGAAACACUACCUCAUGUGGGGUCUCUCCUCCG
AUUUCUGGGGAGAGAAGCCCAACCUCAGCUACAUCAUCGGGAAGGACACUUGGGUGGAGCACUG
GCCCGAGGAGGACGAAUGCCAAGACGAAGAGAACCAGAAACAAUGCCAGGACCUCGGCGCCUUC
ACCGAGAGCAUGGUUGUCUUUGGGUGCCCCAACUGACCACACCCCCAUUCCCCCACUCCAGAUA
AAGCUUCAGUUAUAUCUCAAAAAAAAAAAAAAAAA

In some embodiments, an inhibitory RNA comprises a nucleic acid strand that is complementary to a target portion of a C3 transcript, e.g., C3 mRNA (e.g., complementary to a nucleotide sequence that is at least 90%, 91%, 92%, 93%, 94%, 95%, 96%, 97%, 98%, 99%, or 100% identical to a target portion of SEQ ID NO:75). The target portion may be 15-30 nucleotides long, e.g., 15, 16, 17, 18, 19, 20, 21, 22, 23, 24, 25, 26, 27, 28, 29, or 30 nucleotides long, although shorter and longer target portions are also contemplated. In some embodiments, the target portion comprises a sequence that is at least 90%, 91%, 92%, 93%, 94%, 95%, 96%, 97%, 98%, 99%, or 100% identical to any one of the sequences listed below in Table 1.

TABLE 1

| SEQ ID NO: | Target Sequence (5′ TO 3′) |
|---|---|
| 76 | UCAACUCACCUGUAAUAA |
| 77 | AGGAUGCCACUAUGUCUA |
| 78 | CUUGAAGCCAACUACAUG |
| 79 | UCCAAGCCUUGGCUCAAU |
| 80 | AGUCAAGGUCUACGCCUA |
| 81 | AAACUGUGGCUGUUCGCA |
| 82 | UGAGAUCUGUACCAGGUA |
| 83 | CUUUGUUCUCAUCUCGCU |
| 84 | AUCGGAUCUUCACCGUCA |
| 85 | UCAACUUCCUCCUGCGAA |
| 86 | CGUGCUGCCCAGUUUCGA |
| 87 | AUAGGAACACCCUCAUCA |
| 88 | UGGUCAAGGUCUUCUCUC |
| 89 | GCAACUCCAACAAUUACC |
| 90 | CCUUUGUCAUCUUCGGGA |
| 91 | CAAUGACUUUGACGAGUA |
| 92 | ACGACUUCCCAGGCAAAA |
| 93 | GAACAGAGAUACUACGGU |
| 94 | GUUUCGAGGUCAUAGUGG |
| 95 | AAUGAACAGAGAUACUAC |
| 96 | AGCUAAAAGACUUUGACU |
| 97 | CCAACUACAUGAACCUAC |
| 98 | CUACUCUGUUGUUCGAAA |
| 99 | GUGCGUUGGCUCAAUGAA |
| 100 | CUCCUGCGAAUGGACCGC |

Administration of an inhibitory RNA can reduce the level of C3 transcript or C3 protein in the subject or in a biological sample (e.g., a blood, serum or plasma sample, a sample comprising hepatocytes) compared to a level before the administration of the composition. In some embodiments, the level of C3 transcript or C3 protein is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%, relative to a level before the administration. Level of C3 protein can be measured, for example, in a blood (serum or plasma) sample.

### MicroRNAs

The disclosure also includes compositions and methods related to one or more oligonucleotides that are, comprise, or encode, microRNAs. MicroRNAs (miRNAs) are a highly conserved class of small RNA molecules that are transcribed from DNA in the genomes of plants and animals, but are not translated into protein. Naturally occurring miRNAs are first transcribed as long hairpin-containing primary transcripts (pri-miRNAs). The primary transcript is cleaved by Drosha ribonuclease III enzyme to produce an approximately 70 nt stem-loop precursor miRNA (pre-miRNA), which includes an “antisense strand” or “guide strand” (that includes a region that is substantially complementary to a target sequence) and a “sense strand” or “passenger strand” (that includes a region that is substantially complementary to a region of the antisense strand). The pre-miRNA is then actively exported to the cytoplasm where it is cleaved by Dicer ribonuclease to form the mature miRNA. Processed microRNAs are incorporated into the RNA-induced silencing complex (RISC) to form mature gene-silencing complexes, which induce target mRNA degradation and/or translation repression. The number of miRNA sequences identified to date is large and growing, illustrative examples of which can be found, for example, in: “miRBase: microRIVA sequences, targets and gene nomenclature” Griffiths-Jones S, Grocock R J, van Dongen S, Bateman A, Enright A J. NAR, 2006, 34, Database Issue, D140-D144; “The microRNA Registry” Griffiths-Jones S. NAR, 2004, 32, Database Issue, D109-D111.

miRNAs can be designed and/or synthesized as mature molecules or precursors (e.g., pri- or pre-miRNAs). In some embodiments, a pre-miRNA includes a guide strand and a passenger strand that are the same length (e.g., about 15, 16, 17, 18, 19, 20, 21, 22, 23, 24, or 25 nucleotides). In some embodiments, a pre-miRNA includes a guide strand and a passenger strand that are different lengths (e.g., one strand is about 19 nucleotides, and the other is about 21 nucleotides). In some embodiments, an miRNA can target the coding region, the 5′ untranslated region, and/or 3′ untranslated region, of endogenous mRNA. In some embodiments, an miRNA comprises a guide strand comprising a nucleotide sequence having sufficient sequence complementary with an endogenous mRNA of a subject to hybridize with and inhibit expression of the endogenous mRNA.

In some embodiments, the miRNA comprises a nucleic acid strand that comprises a region that is perfectly complementary to at least 6, 7, 8, 9, 10, 11, 12, 13 14, 15, 16, 17, 18, 19, 20, 21, 22, 23, 24, 25, 26, 27, 28, 29, or 30 consecutive nucleotides of SEQ ID NO: 75 (e.g., any one of SEQ ID NOs: 76-100). In some embodiments, an miRNA comprises a mature guide strand having a nucleotide sequence that is perfectly complementary to a target portion comprising a sequence that is at least 90%, 91%, 92%, 93%, 94%, 95%, 96%, 97%, 98%, 99%, or 100% identical to any one of SEQ ID NOs: 76-100.

siRNAs

In some embodiments, an inhibitory RNA is a double stranded RNA (dsRNA), and inhibits C3 expression by RNA interference (RNAi). RNAi is a process of sequence-specific post-transcriptional gene silencing by which, e.g., double stranded RNA (dsRNA) homologous to a target locus can specifically inactivate gene function (Hammond et al., Nature Genet. 2001; 2:110-119; Sharp, Genes Dev. 1999; 13:139-141). This dsRNA-induced gene silencing can be mediated by short double-stranded small interfering RNAs (siRNAs) generated from longer dsRNAs by ribonuclease III cleavage (Bernstein et al., Nature 2001; 409:363-366 and Elbashir et al., Genes Dev. 2001; 15:188-200). RNAi-mediated gene silencing is thought to occur via sequence-specific RNA degradation, where sequence specificity is determined by the interaction of an siRNA with its complementary sequence within a target RNA (see, e.g., Tuschl, Chem. Biochem. 2001; 2:239-245). RNAi can involve the use of, e.g., siRNAs (Elbashir, et al., Nature 2001; 411:494-498) or short hairpin RNAs (shRNAs) bearing a fold back stem-loop structure (Paddison et al., Genes Dev. 2002; 16:948-958; Sui et al., Proc. Natl. Acad. Sci. USA 2002; 99:5515-5520; Brummelkamp et al., Science 2002; 296:550-553; Paul et al., Nature Biotechnol. 2002; 20:505-508).

The disclosure includes siRNA molecules targeting C3 transcript, e.g., C3 mRNA (SEQ ID NO: 75). In some embodiments, an siRNA molecule comprises a sequence that is complementary to a target region comprising any one of SEQ ID NOs: 76-100. In some embodiments, an siRNA molecule comprises (i) a nucleotide sequence that is at least 90%, 91%, 92%, 93%, 94%, 95%, 96%, 97%, 98%, 99%, or 100% identical to any one of SEQ ID NOs: 76-100 (or a portion thereof) and/or (ii) a nucleotide sequence that is complementary to a nucleotide sequence that is at least 90%, 91%, 92%, 93%, 94%, 95%, 96%, 97%, 98%, 99%, or 100% identical to any one of SEQ ID NOs: 76-100 (or a portion thereof).

In some embodiments, siRNAs of the disclosure are double stranded nucleic acid duplexes (of, e.g., 15, 16, 17, 18, 19, 20, 21, 22, 23, 24, 25, 26, or 27 base pairs) comprising annealed complementary single stranded nucleic acid molecules. In some embodiments, the siRNAs are short dsRNAs comprising annealed complementary single strand RNAs. In some embodiments, the siRNAs comprise an annealed RNA: DNA duplex, wherein the sense strand of the duplex is a DNA molecule and the antisense strand of the duplex is a RNA molecule. In some embodiments, an siRNA comprises a sense strand having a nucleotide sequence that is at least 90%, 91%, 92%, 93%, 94%, 95%, 96%, 97%, 98%, 99%, or 100% identical to any one of SEQ ID NOs: 76-100 (or a portion thereof).

In some embodiments, an siRNA comprises an antisense strand having a nucleotide sequence that is at least 90%, 91%, 92%, 93%, 94%, 95%, 96%, 97%, 98%, 99%, or 100% identical to any one of SEQ ID NOs: 101-125 in the following Table 2A:

TABLE 2A

| SEQ ID NO: | Antisense Sequence (5′ TO 3′) |
|---|---|
| 101 | UUAUUACAGGUGAGUUGA |
| 102 | UAGACAUAGUGGCAUCCU |
| 103 | CAUGUAGUUGGCUUCAAG |
| 104 | AUUGAGCCAAGGCUUGGA |
| 105 | UAGGCGUAGACCUUGACU |
| 106 | UGCGAACAGCCACAGUUU |
| 107 | UACCUGGUACAGAUCUCA |
| 108 | AGCGAGAUGAGAACAAAG |
| 109 | UGACGGUGAAGAUCCGAU |
| 110 | UUCGCAGGAGGAAGUUGA |
| 111 | UCGAAACUGGGCAGCACG |
| 112 | UGAUGAGGGUGUUCCUAU |
| 113 | GAGAGAAGACCUUGACCA |
| 114 | GGUAAUUGUUGGAGUUGC |
| 115 | UCCCGAAGAUGACAAAGG |
| 116 | UACUCGUCAAAGUCAUUG |
| 117 | UUUUGCCUGGGAAGUCGU |
| 118 | ACCGUAGUAUCUCUGUUC |
| 119 | CCACUAUGACCUCGAAAC |
| 120 | GUAGUAUCUCUGUUCAUU |
| 121 | AGUCAAAGUCUUUUAGCU |
| 122 | GUAGGUUCAUGUAGUUGG |
| 123 | UUUCGAACAACAGAGUAG |
| 124 | UUCAUUGAGCCAACGCAC |
| 125 | GCGGUCCAUUCGCAGGAG |

In some embodiments, an siRNA comprises an antisense strand having a nucleotide sequence that is at least 90%, 91%, 92%, 93%, 94%, 95%, 96%, 97%, 98%, 99%, or 100% identical to any one of the sequences in the following Table 2B:

TABLE 2B
Antisense Sequence (5′ to 3′)

AGAGGAUGGGGAGGAGUGA
AGAGGGACAGAGGGACAGA
UGUGGGUCCCAUGGUGCUG
AGCAGGCUGGGACCUGAGG
AGCAGGAGCAGCAGGCUGG
AGGGGGAGGUGGGUUAGUA
UGGGGGUGAUGAUAGAGUA
UUCCAGCCGCAAGAUGUUG
ACCAUGGUCUCCUCGCUCU
ACAGUAACAGUGACUGGAA
UUAGUUUUUUGCCUGGGAA
UUUCUCACUGGACAGCACU
UCAGGGGUCAGCACAGUCU
UUCUGACUUGAACUCCCUG
UCCCCUUUUCUGACUUGAA
UGGCCUGCACGGUCACGAA
UGGUCCCGAAGGUGGCCUG
UCACCUUCUCCACCACUUG
UCUGCAGGCUGACCAGCAC
UGAUGAAGAGGUACCCGCU
AGAUGGUCUUGUCUGUCUG
UGUGGAGCCAGGGGUGUAG
AGAUCCGAUAGAGAACUGU
UCUUGUGGUUGACGGUGAA
AUGUUGACCAUGACCGUCC
AUGCCUUCCGGGUUCUCAA
AGUCCUGCUUGACCGGGAU
UUUCUGAGAAGACAAGGAG
UCUGGUUCUGAGAAGACAA
UAAGGGCAAGACGCCAAGC
AUGUCCCAAGACAAGGGCA
UGAUCUUCCACUGGCCCAU
UCUCGGAUCUUCCACUGGC
UGUGAGUUUUCAUAGUAGG
UUUCACCUCAAACUCAGUG
UGUACUCCUUCACCUCAAA
UUCGAAACUGGGCAGCACG
UUAGGCUCCACUAUGACCU
AUGUAGUAGAAUUUCUCUG
AGGCCCUUCUCGUUAUAGA
UGGUGAUGGUGACCUCCAG
UCCCGUAGAGGAACCUGGC
UAGUUCCCUCCACUUUCUU
AGAUGACAAAGGCAGUUCC
UGCCAUCCUGGAUCCCGAA
UAGGGAAAUCCUCUGUUCG
AGGGAUUCAGGCAGGGAAA
UAAUCGGAAUGCGCUUGAG
UUCCCCCGAGCCAUCCUCA
UUCCGGCUCAGCACAACCU
AGUACCUUCCGGCUCAGCA
UACCCCGUCCAGCAGUACC
UUCUGCUCGGGGGUUCUGC
UGACGGUGGCAGACACGUA
UGUCACUGCCUGAGUGCAA
AUCCCGCUGCGCUCUGCCU
ACUUGGGUGUCUUGGUGAA
AGUACUUGGGUGUCUUGGU
AGGGCAUUCCUGGUUUGAA
UCAUCAGGGUUCGUCACGA
ACAGUGUCCUCGCCCUGGA
UAGACUGCACAGUGUCCUC
UGCCAUCUCCCUGGGUUAG
UAUGCUGAGUUUGGCCACG
UGGCUGGGGUGUGUGUUGA
UGAUGCUCAAGGGCUUCUG
UGCGCACCGUGAUGCUCAA
UCUCUGCCUCCGAGAGCUC
UCAUGGUCCUGGUAGCCUG
UCUGUAGGGCAGAGCCUGC
AGUUGCCCACGGUGCUGUA
AUGCAGGUAAUUGUUGGAG
AGCUCUGUACGUAGCACUG
AGGGUCUCCCCGGGUCUGA
AUCUUGGCCUCGUGGGCGC
AGGUAGGUGUAGUAGCGGA
UGCCCUUGUUCAUGAUCAG
UCCCGCCUUCAACAGCCUG
UGCUCUCGCACCUGGCGUC
UCACCAGGUCCUGGCCGGG
AGUACGCCACCAGGCGGAA
UGCACCGAUCAGCGUGUAG
AGUCCUUGACGUCCACCCA
ACCAGCGAGCCCACGCAGG
UACUGGCCGCUUUUUACCA
ACAGGCUGCCGGUCUUCUG
UGGUCAUCUGCUGCCCAGG
UGUCACCCUCUAUCUUCAG
UACCACCCGGGCCCCGUGG
UUUGUCCACGGCCACCAGU
UGUCAGUUUGUUCUUCUUA
UCCCAGAUCUUACUCUGCG
UUGCCUUCUCCACCACGUC
UCUUCCCACUGCCCGGGGU
AGAAGACACCGGCGUAAUC
AGGUCAGCCCUGCGUCGGA
UGCCACUGCUGCUCGUGAA
UCUCUGGGCGGUCUGCUGG
AGUUCUGCCCUCUGGGCGG
UCUGGCUGCGGGCACUGAA
AACGGCGUCGGCGGGCGGC
UGUCCAUUCGCUUCUCCGU
UGGGGUACUUGCCGACUUU
UCACUUGCGCAGCUCCUUG
UGCAUGCCGUCCUCGCAGC
UGGCACGAGAACCUCAUGG
UCCGGCGCUGGCACGAGAA
UGCCCAGGGAGAUGAAACG
AGACCUUCUUGCACGCCUC
AGUUGCAGCAGUCCAGGAA
UCGCAGCUCUGUGAUGUAG
AGGUGGCUGGCCCGCGCGU
UUACUCCUGGCCAGGCCCA
AUGAUGUCCUCAUCCAGGU
ACGAUGUUCUCUUCUGCAA
UGAACUCACUUCGGGAAAC
ACAGCCAGCUCUCUGGGAA
UAAGUCCUCAACGUUCCAC
UGCUCUUUCAAGUCCUCAA
UGUGGCUCUUUCAAGUCCU
UUAGAGAUUCCAUUUUUCG
AUUCAUGAGCUUCGUAGAG
UGAGUCUUUCAAAAAUAUA
AUCUCCCACGUGGUGAUGG
ACAUGCUCACAGCCAGAAU
AGAUCCCUUUCUUGUCCGA
UGACCUCGAAGGGGUCUGC
UUAGCCGCAGGUCGAUGAA
UCGAACAACAGAGUAGGGU
UGCUCGUUUCGAACAACAG
UGGCUCGGAUUUCCACCUG
UCGGUAAUUGUAGAGAACG
ACCCUCACCUUGAGCUCUU
UAUUGUGGAGUAGUUCCAC
AAGGCUGGAUUGUGGAGUA
UGGUGGCCAGGCUGCAGAA
UUGGUGACGCCUCUUGGUG
UGGAUGGUUACGGUCUGCU
AUGGAACGGACAACGAGGA
UUAGCGGCACGAUGACAUA
ACUUCCUGCAGGCCGGUCU
UAGCAGCCUUGACUUCCAC
UGACACCGUCACUGAUGAA
UACCUUCAGGGACUUCCUG
AGUUUUGUUCAUUCUGAUU
AGGGUGCGAACAGCCACAG
UCAGGCGUUCUGGAUCCAG
UCCUCUUUCUGCACUCCUU
UUGUCCGGGACUUGGUCAC
AUUCUGGUCUCAGACUCGG
UGGGUCCCUUGCAGGAGAA
UUGUCAUCUGGGCCACUGG
UAGCCGUUCCGCGUCGACG
UAUCAUGUUCUGUUCCCCG
ACCGUGGGCGUCAUGCCGA
UCUCCGUUUCAUCCAGGUA
UACUGCUCCGUUUCAUCCA
UCUAGGCCGAACUUCUCCC
AAGGCCCCCUGCCGCUUCU
UCUUCUUGAUGAGCUCCAA
ACCCCUUCUUGAUGAGCUC
AGGCCAGCUGCUGGGUGUA
AGAGCUGGGUUGUCUGAAG
AGGCCGCAAAGGCAGAGCU
UGGGUGCCCGUUUCACGAA
UGCGGUCAGCCAGGUGCUG
AGAAGACCUUGACCACGUA
UAGGUUGACAGCCAGAGAG
UCUUCUCCAGGAUCAGCCA
AGACCCCGUCGGGCUUCUG
UCGCAUCCUCCUGGAAGAC
UUUCUUGGUGUAUCACGGG
UCCGUAAUCCACCAAUCAU
UGUCUUUCUCGUUGUUGUU
UCAGCGAGAUGAGAACAAA
AAAUAUCUUUAGCCUCCUG
UUUGACCUGCUCCUCGCAA
AUGCUGCCUGGCAGGCUGU
UUGAUGCUGCCUGGCAGGC
AGGAAGUCUCCUGCUUUAG
UCAUGUAGUUGGCUUCAAG
AUCUCUGUAGGUUCAUGUA
AAUGGCCACAGUGUAGGAU
UCCAGAGCAUAGCCAGCAA
UUUAAGAAGAGGCCCCUUC
UGCUGUGGUCAGAAAUUUG
UCAGCGGUUCUUAUCUUUG
UGCUUACCAGGGUCCUCCC
AGAGCUGCUUACCAGGGUC
AUGUGGCCUCCACGUUGUA
AGGGCCAAGAGGGCAUAGG
AAGUCUUUUAGCUGCAGUA
AUUGAGCCAACGCACGACG
UUAGUAUCUCUGUUCAUUG
UAGCCAUAGCCACCACCGU
UGAAGGUGGCCUGGGUAGA
AGGCUUGGAACACCAUGAA
CUUUUGGUAUUGAGCCAAG
UGGUGGUCAGGGGCGUCCU
UAUCAAGGUUCAGUUCCUG
AGUUGGAGGGACACAUCAA
UGGAGCUGCGGCUGGGCAG
UGGUGAUCUUGGAGCUGCG
UUCCCAGUGGAUACGGUGG
AGAUUCCCAGUGGAUACGG
UGAUCGCAGGAGGCUGGCA
AUUUUCCUUGGUCUCUUCU
UUGACUGUGAAACCCUCAU
UGCCUUUUCCUUCAGCUGU
UACCGACAAGGUGCCUUGG
ACAUUGUCACCACCGACAA
UUUGGCCUUAGCAUGGUAC
AGUUGAUCUUUGGCCUUAG
AUUUAUUACAGGUGAGUUG
UGGUGACCUUGAGGUCGAA
UGUGCUGGUUUUAUGGUGA
UGCCUCUUUUCUGUUUCCG
AGUGUUCUUGGCAUCCUGA
ACAGAUCUCAAGGAUCAUA
UGGUCUCCCCGGUACCUGG
UAGACAUAGUGGCAUCCUG
AUCUGUGUCUGGAGCAAAG
UCCAGCUGCUUCAGGUCAU
UUGGAGAUGUAUCUGUCAA
CUUUGUCCAGCUCAUACUU
AGGCUUUGUCCAGCUCAUA
UGUGUUCCUAUCGGAGAAG
UCCAGGUAGAUGAUGAGGG
UAGAGUGUGAGACCUUGUC
AGUCAUCCUCAGAGUGUGA
AAGCUAGACAGUCAUCCUC
AGUAUUGGUGAACUUUGAA
UAUAAGCUCUACAUUAAAG
AAUAGGCGUAGACCUUGAC
AGCUUUCCUCCAGGUUGUA
AUGGUAGAACCGGGUACAG
UCAUCCUCCUUUUCCGGAU
AGCUUUCCAUCCUCCUUUU
UACGGCAGAGCUUGUUCAG
ACAGCGGCACAGUUCAUCA
AAGCAAUUCUCCUCAGCAC
ACUUUUGUAUGAAGCAAUU
UGUGACCUUGUCAUCCGAC
UCCAGCCGUUCUUCCAGGG
UUGGCUCACAGGCCUUGUC
ACUCCUGGCUCACAGGCCU
ACACAUAGUCCACUCCUGG
UGACCAGUCGGGUCUUGUA
AAGUCAUUGGACAGCUGAA
UCAUGAUGUACUCGUCAAA
UCUCAAUGGCCAUGAUGUA
AGCCUGACUUGAUGGUCUG
AACCUGCACCUCAUCCGAG
ACUUGAUGGGGCUGAUGAA
AGGGCUUCUCUGCACUUGA
UCUUCUCCUCCAGCUUCAG
GUUUCUUCUCCUCCAGCUU
ACCCCACAUGAGGUAGUGU
UGGGCUUCUCUCCCCAGAA
UGUCCUUCCCGAUGAUGUA
UCAGUGCUCCACCCAAGUG
UCGUCUUGGCAUUCGUCCU
UAUUGUUUCUGGUUCUCUU
AGGCGCCGAGGUCCUGGCA
AACCAUGCUCUCGGUGAAG
UUGGGGGAAUGGGGGUGUG
ACUGAAGCUUUAUCUGGAG
UUUUUUUUUUGAGAUAUAA
UAUGAAGCAAUUCUCCUCAGCAC
UUUUGUAUGAAGCAAUUCUCCUC
AAGUCAAAGUCUUUUAGCUGCAG
AAUGGUCUUGUCUGUCUGGAUGA
UUGUAGAUGGUCUUGUCUGUCUG
AGAGAGAAGACCUUGACCACGUA
AGAUGGUCUUGUCUGUCUGGAUG
AACAGCCACAGUUUUGUUCAUUC
AAGGCUUGGAAUACCAUGAAGGU
AUGGUCUUGUCUGUCUGGAUGAA
UAGAUGGUCUUGUCUGUCUGGAU
GUGUAGAUGGUCUUGUCUGUCUG
AGAGAAGACCUUGACCACGUAGG
UGUAGAUGGUCUUGUCUGUCUGG
GAGAGAAGACCUUGACCACGUAG
UCGUAGAGAUUCCAUUUUUCUCU
AACGAACACCAUGAGGUCAAAGG
AGGGGUGUAGAUGGUCUUGUCUG
UCUGCCACACAGAUCCCUUUCUU
GUCUUGGUGAAGUGGAUCUGGUA
CUUGAGGUCGAAUUUAUUACAGG
GAUGGUCUUGUCUGUCUGGAUGA
UGUGAGUUUGUUCUUCUUGUUCA
AAUUUGGGUGUCUUGGUGAAGUG
AUCACGAACACCAUGAGGUCAAA
AUUACUGUGACCUCGAAGGGGUC
UGUGUAGAUGGUCUUGUCUGUCU
ACCAAUCAUUUCUUGGUGAAUCA
AUCAUAGUGUUCUUGGCAUCCUG
UCUCUGUAGGUUCAUGUAGUUGG
GUCACGAACACCAUGAGGUCAAA
AAGUUUGCCACCACUGUCACGUA
UCCACUAUGACCUCGAAACUGGG
UGGUCUGCCACACAGAUCCCUUU
GGGUCUGCCACACAGAUCCCUUU
UAGAGAAGACCUUGACCACGUAG
AAUGUUCCAAGACAAAGGCAAGA
GGUUCUUUCAACUCUUCUAUGGU
AUUCUGAUUCCUUCCGGCACGAC
UUGACGUCCACCCACACGGAGUC
UCACGAACACCAUGAGGUCAAAG
AGAGUCAAAGUCUUUCAGCAGCA
UCCACACAGAUCCCUUUCUUGUC
UGCCACACAGAUCCCUUUCUUGU
CACGAACACCAUGAGGUCAAAGG
UUUAGGAAGUCUUGCACAGUGAC
UCUUCUAUGGUCCACAACCAGCU
UCCAGGAUCAGCCAUUUAACAGC
AGGGGUCUGCCACACAGAUCCCU
GGUGUAGAUGGUCUUGUCUGUCU
UUUGCCACCACUGUCACGUACUU
UUGAGAAAGAUGUUCAUGACCUU
UCUCAUUCCUUCUGGCACGACCU
GCCACACAGAUCCCUUUCUUGUC
UCUUUAGGAAGUCUUGCACAGUG
UUCUUGGUGAAGUGGAUCUGGUA
GAGUCAAAGUCUUUCAGCAGCAG
UGAGAGAAGACCUUGACCA
UCUUGGUGAAGUGGAUCUG
AGAGAGAAGACCUUGACCA
GUUGUAAUAGGCGUAGACC
AUUGUAAUAGGCGUAGACC
UUUCAUAGUAGGCUCGGAU
UUUCUCUGUAGGCUCCACU
UUAUAGAUGUAGUAGAAUU
AUGACAAAGGCAGUUCCCU
AUCUGGUAGGGAGAGGUCA
UGUGUGUUGAUGCUGAGUU
UAAUUGUUGGAGUUGCCCA
AGGAAGUUGACGUUGAGGG
AUGAAGUCGGUGGUGAUGG
UUUACCACCAGCGAGCCCA
UCUAUCUUCAGGGUCAUCU
AUGUAGUUGCAGCAGUCCA
AAUAUAUUCAUGAGCUUCG
AAAUAUAUUCAUGAGCUUC
UCCUGCAUUACUGUGACCU
CAACAGAGUAGGGUAGCCG
UCGAACAACAGAGUAGGGU
UUUCGAACAACAGAGUAGG
GUUUCAUCCAGGUAAUGCA
UUAUCUUUGGCUGUGGUCA
UGUUCAUUGAGCCAACGCA
UUGGUAUUGAGCCAAGGCU
UUUAUUACAGGUGAGUUGA
UUUCUGUUUCCGGUGCUGG
UCAAGGAUCAUAGUGUUCU
AUCUCAAGGAUCAUAGUGU
UCAUACUUGGAGAUGUAUC
UAUCGGAGAAGGCUUUGUC
AUGAUGAGGGUGUUCCUAU
UAUUGGUGAACUUUGAAAG
AGCUUGUUCAGCUUUCCAU
UUUGUAUGAAGCAAUUCUC
GUCUUGUACACAUAGUCCA
UGCUCAAUGGCCAUGAUGU
UUGGCAUUCGUCCUCCUCG
GUCUGGAUGAAGAGGUACC
UGUCUGGAUGAAGAGGUAC
UCUGUCUGGAUGAAGAGGU
CUUGUCUGUCUGGAUGAAG
AUGGUCUUGUCUGUCUGGA
AGAUGGUCUUGUCUGUCUG
GUAGAUGGUCUUGUCUGUC
GUGUAGAUGGUCUUGUCUG
GGGGUGUAGAUGGUCUUGU
UAGGCUCGGAUCUUCCACU
CUCGAAACUGGGCAGCACG
UUGGUGAAGUGGAUCUGGU
GUCUUGGUGAAGUGGAUCU
GGUGUCUUGGUGAAGUGGA
AACACCAUGAGGUCAAAGG
GAACACCAUGAGGUCAAAG
GUCACGAACACCAUGAGGU
ACACAGAUCCCUUUCUUGU
CCUUGCAGGAGAAUUCUGG
GAGUCGAUGGCGAUGAGGU
GCUUGGAACACCAUGAAGG
UCAUUUUCCUUGGUCUCUU
UGUGUCUGGAGCAAAGCCA
AGGUAGAUGAUGAGGGUGU
UUGUAAUAGGCGUAGACCU
GGGUCUUGUACACAUAGUC
CACUUGAUGGGGCUGAUGA
AUGUAGAUGGUCUUGUCUG
UGAGAGACGACCUUGACCA
UGAGAGAAUACCUUGACCA
UGAGAGAAGACCUUGACCG
AUGAGCUUCG UAGAGAUUC
UUGUAGUAGCGGAUCUUGG
UUCUGUCUGGAUGAAGAGG
UGAGAGAAGA CCUUGACCA
UGAGAGACGA CCUUGACCG
UGAGAGAAGA CCUUGACCA

In some embodiments, an siRNA comprises an antisense strand that is modified or unmodified, and that includes any one of the sequences disclosed in WO 2020/104669, WO 2021/037941, WO 2015/089368, WO 2019/089922, WO 2021/081026, WO 2021/178607, U.S. Pat. No. 7,582,746, WO 2007/089375, or WO 2003/066805, each of which are herein incorporated by reference in their entirety.

In some embodiments, an siRNA comprises mismatch(es) with the target, within the duplex, or combinations thereof. The mismatch may occur in an overhang region and/or the duplex portion. The base pair may be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G:C (I-inosine).

In some embodiments, an siRNA comprises at least one of the first 1, 2, 3, 4, or 5 base pairs within the duplex portions from the 5′-end of the antisense strand independently selected from the group of: A:U, G:U, I:C, and mismatched pairs. In some embodiments, the nucleotide at the 1 position within the duplex portion from the 5′-end in the antisense strand is selected from the group consisting of A, dA, dU, U, and dT. Additionally or alternatively, at least one of the first 1, 2 or 3 base pairs within the duplex portion from the 5′-end of the antisense strand is an AU base pair. For example, the first base pair within the duplex portion from the 5′-end of the antisense strand is an AU base pair.

In some embodiments, a sense strand can include one or more (e.g., 2, 3, 4, or 5) nucleotides on the 3′ and/or 5′ end that is not identical to the target sequence, and/or an antisense strand can include one or more (e.g., 2, 3, 4, or 5) nucleotides on the 3′ and/or 5′ end that is not complementary to the target sequence. For example, in some embodiments, a duplexed siRNA comprises a sense strand comprising a sequence listed in the following Table 3. The sequences in Table 3 contain an adenine (A) nucleotide at the 3′ end, which, in some of the sequences, is complementary to the target sequence (e.g., is complementary to the next contiguous nucleotide of the target sequence). In some of the sequences in Table 3A, the adenine (A) nucleotide at the 3′ end is not complementary to the target sequence (e.g., is not complementary to the next contiguous nucleotide of the target sequence).

TABLE 3A

| SEQ ID | Sense Sequence |
|---|---|
| NO: | (5′ to 3′) |
| 126 | UCAACUCACCUGUAAUAAA |
| 127 | AGGAUGCCACUAUGUCUAA |
| 128 | CUUGAAGCCAACUACAUGA |
| 129 | UCCAAGCCUUGGCUCAAUA |
| 130 | AGUCAAGGUCUACGCCUAA |
| 131 | AAACUGUGGCUGUUCGCAA |
| 132 | UGAGAUCUGUACCAGGUAA |
| 133 | CUUUGUUCUCAUCUCGCUA |
| 134 | AUCGGAUCUUCACCGUCAA |
| 135 | UCAACUUCCUCCUGCGAAA |
| 136 | CGUGCUGCCCAGUUUCGAA |
| 137 | AUAGGAACACCCUCAUCAA |
| 138 | UGGUCAAGGUCUUCUCUCA |
| 139 | GCAACUCCAACAAUUACCA |
| 140 | CCUUUGUCAUCUUCGGGAA |
| 141 | CAAUGACUUUGACGAGUAA |
| 142 | ACGACUUCCCAGGCAAAAA |
| 143 | GAACAGAGAUACUACGGUA |
| 144 | GUUUCGAGGUCAUAGUGGA |
| 145 | AAUGAACAGAGAUACUACA |
| 146 | AGCUAAAAGACUUUGACUA |
| 147 | CCAACUACAUGAACCUACA |
| 148 | CUACUCUGUUGUUCGAAAA |
| 149 | GUGCGUUGGCUCAAUGAAA |
| 150 | CUCCUGCGAAUGGACCGCA |

In some embodiments, an siRNA comprises a sense strand having a nucleotide sequence that is at least 90%, 91%, 92%, 93%, 94%, 95%, 96%, 97%, 98%, 99%, or 100% identical to any one of the sequences in the following Table 3B:

TABLE 3B
Sense Sequence
(5′ to 3′)
UCACUCCUCCCCAUCCUCU
UCUGUCCCUCUGUCCCUCU
CAGCACCAUGGGACCCACA
CCUCAGGUCCCAGCCUGCU
CCAGCCUGCUGCUCCUGCU
UACUAACCCACCUCCCCCU
UACUCUAUCAUCACCCCCA
CAACAUCUUGCGGCUGGAA
AGAGCGAGGAGACCAUGGU
UUCCAGUCACUGUUACUGU
UUCCCAGGCAAAAAACUAA
AGUGCUGUCCAGUGAGAAA
AGACUGUGCUGACCCCUGA
CAGGGAGUUCAAGUCAGAA
UUCAAGUCAGAAAAGGGGA
UUCGUGACCGUGCAGGCCA
CAGGCCACCUUCGGGACCA
CAAGUGGUGGAGAAGGUGA
GUGCUGGUCAGCCUGCAGA
AGCGGGUACCUCUUCAUCA
CAGACAGACAAGACCAUCU
CUACACCCCUGGCUCCACA
ACAGUUCUCUAUCGGAUCU
UUCACCGUCAACCACAAGA
GGACGGUCAUGGUCAACAU
UUGAGAACCCGGAAGGCAU
AUCCCGGUCAAGCAGGACU
CUCCUUGUCUUCUCAGAAA
UUGUCUUCUCAGAACCAGA
GCUUGGCGUCUUGCCCUUA
UGCCCUUGUCUUGGGACAU
AUGGGCCAGUGGAAGAUCA
GCCAGUGGAAGAUCCGAGA
CCUACUAUGAAAACUCACA
CACUGAGUUUGAGGUGAAA
UUUGAGGUGAAGGAGUACA
CGUGCUGCCCAGUUUCGAA
AGGUCAUAGUGGAGCCUAA
CAGAGAAAUUCUACUACAU
UCUAUAACGAGAAGGGCCU
CUGGAGGUCACCAUCACCA
GCCAGGUUCCUCUACGGGA
AAGAAAGUGGAGGGAACUA
GGAACUGCCUUUGUCAUCU
UUCGGGAUCCAGGAUGGCA
CGAACAGAGGAUUUCCCUA
UUUCCCUGCCUGAAUCCCU
CUCAAGCGCAUUCCGAUUA
UGAGGAUGGCUCGGGGGAA
AGGUUGUGCUGAGCCGGAA
UGCUGAGCCGGAAGGUACU
GGUACUGCUGGACGGGGUA
GCAGAACCCCCGAGCAGAA
UACGUGUCUGCCACCGUCA
UUGCACUCAGGCAGUGACA
AGGCAGAGCGCAGCGGGAU
UUCACCAAGACACCCAAGU
ACCAAGACACCCAAGUACU
UUCAAACCAGGAAUGCCCU
UCGUGACGAACCCUGAUGA
UCCAGGGCGAGGACACUGU
GAGGACACUGUGCAGUCUA
CUAACCCAGGGAGAUGGCA
CGUGGCCAAACUCAGCAUA
UCAACACACACCCCAGCCA
CAGAAGCCCUUGAGCAUCA
UUGAGCAUCACGGUGCGCA
GAGCUCUCGGAGGCAGAGA
CAGGCUACCAGGACCAUGA
GCAGGCUCUGCCCUACAGA
UACAGCACCGUGGGCAACU
CUCCAACAAUUACCUGCAU
CAGUGCUACGUACAGAGCU
UCAGACCCGGGGAGACCCU
GCGCCCACGAGGCCAAGAU
UCCGCUACUACACCUACCU
CUGAUCAUGAACAAGGGCA
CAGGCUGUUGAAGGCGGGA
GACGCCAGGUGCGAGAGCA
CCCGGCCAGGACCUGGUGA
UUCCGCCUGGUGGCGUACU
CUACACGCUGAUCGGUGCA
UGGGUGGACGUCAAGGACU
CCUGCGUGGGCUCGCUGGU
UGGUAAAAAGCGGCCAGUA
CAGAAGACCGGCAGCCUGU
CCUGGGCAGCAGAUGACCA
CUGAAGAUAGAGGGUGACA
CCACGGGGCCCGGGUGGUA
ACUGGUGGCCGUGGACAAA
UAAGAAGAACAAACUGACA
CGCAGAGUAAGAUCUGGGA
GACGUGGUGGAGAAGGCAA
ACCCCGGGCAGUGGGAAGA
GAUUACGCCGGUGUCUUCU
UCCGACGCAGGGCUGACCU
UUCACGAGCAGCAGUGGCA
CCAGCAGACCGCCCAGAGA
CCGCCCAGAGGGCAGAACU
UUCAGUGCCCGCAGCCAGA
GCCGCCCGCCGACGCCGUU
ACGGAGAAGCGAAUGGACA
AAAGUCGGCAAGUACCCCA
CAAGGAGCUGCGCAAGUGA
GCUGCGAGGACGGCAUGCA
CCAUGAGGUUCUCGUGCCA
UUCUCGUGCCAGCGCCGGA
CGUUUCAUCUCCCUGGGCA
GAGGCGUGCAAGAAGGUCU
UUCCUGGACUGCUGCAACU
CUACAUCACAGAGCUGCGA
ACGCGCGGGCCAGCCACCU
UGGGCCUGGCCAGGAGUAA
ACCUGGAUGAGGACAUCAU
UUGCAGAAGAGAACAUCGU
GUUUCCCGAAGUGAGUUCA
UUCCCAGAGAGCUGGCUGU
GUGGAACGUUGAGGACUUA
UUGAGGACUUGAAAGAGCA
AGGACUUGAAAGAGCCACA
CGAAAAAUGGAAUCUCUAA
CUCUACGAAGCUCAUGAAU
UAUAUUUUUGAAAGACUCA
CCAUCACCACGUGGGAGAU
AUUCUGGCUGUGAGCAUGU
UCGGACAAGAAAGGGAUCU
GCAGACCCCUUCGAGGUCA
UUCAUCGACCUGCGGCUAA
ACCCUACUCUGUUGUUCGA
CUGUUGUUCGAAACGAGCA
CAGGUGGAAAUCCGAGCCA
CGUUCUCUACAAUUACCGA
AAGAGCUCAAGGUGAGGGU
GUGGAACUACUCCACAAUA
UACUCCACAAUCCAGCCUU
UUCUGCAGCCUGGCCACCA
CACCAAGAGGCGUCACCAA
AGCAGACCGUAACCAUCCA
UCCUCGUUGUCCGUUCCAU
UAUGUCAUCGUGCCGCUAA
AGACCGGCCUGCAGGAAGU
GUGGAAGUCAAGGCUGCUA
UUCAUCAGUGACGGUGUCA
CAGGAAGUCCCUGAAGGUA
AAUCAGAAUGAACAAAACU
CUGUGGCUGUUCGCACCCU
CUGGAUCCAGAACGCCUGA
AAGGAGUGCAGAAAGAGGA
GUGACCAAGUCCCGGACAA
CCGAGUCUGAGACCAGAAU
UUCUCCUGCAAGGGACCCA
CCAGUGGCCCAGAUGACAA
CGUCGACGCGGAACGGCUA
CGGGGAACAGAACAUGAUA
UCGGCAUGACGCCCACGGU
UACCUGGAUGAAACGGAGA
UGGAUGAAACGGAGCAGUA
GGGAGAAGUUCGGCCUAGA
AGAAGCGGCAGGGGGCCUU
UUGGAGCUCAUCAAGAAGA
GAGCUCAUCAAGAAGGGGU
UACACCCAGCAGCUGGCCU
CUUCAGACAACCCAGCUCU
AGCUCUGCCUUUGCGGCCU
UUCGUGAAACGGGCACCCA
CAGCACCUGGCUGACCGCA
UACGUGGUCAAGGUCUUCU
CUCUCUGGCUGUCAACCUA
UGGCUGAUCCUGGAGAAGA
CAGAAGCCCGACGGGGUCU
GUCUUCCAGGAGGAUGCGA
CCCGUGAUACACCAAGAAA
AUGAUUGGUGGAUUACGGA
AACAACAACGAGAAAGACA
UUUGUUCUCAUCUCGCUGA
CAGGAGGCUAAAGAUAUUU
UUGCGAGGAGCAGGUCAAA
ACAGCCUGCCAGGCAGCAU
GCCUGCCAGGCAGCAUCAA
CUAAAGCAGGAGACUUCCU
CUUGAAGCCAACUACAUGA
UACAUGAACCUACAGAGAU
AUCCUACACUGUGGCCAUU
UUGCUGGCUAUGCUCUGGA
GAAGGGGCCUCUUCUUAAA
CAAAUUUCUGACCACAGCA
CAAAGAUAAGAACCGCUGA
GGGAGGACCCUGGUAAGCA
GACCCUGGUAAGCAGCUCU
UACAACGUGGAGGCCACAU
CCUAUGCCCUCUUGGCCCU
UACUGCAGCUAAAAGACUU
CGUCGUGCGUUGGCUCAAU
CAAUGAACAGAGAUACUAA
ACGGUGGUGGCUAUGGCUA
UCUACCCAGGCCACCUUCA
UUCAUGGUGUUCCAAGCCU
CUUGGCUCAAUACCAAAAG
AGGACGCCCCUGACCACCA
CAGGAACUGAACCUUGAUA
UUGAUGUGUCCCUCCAACU
CUGCCCAGCCGCAGCUCCA
CGCAGCUCCAAGAUCACCA
CCACCGUAUCCACUGGGAA
CCGUAUCCACUGGGAAUCU
UGCCAGCCUCCUGCGAUCA
AGAAGAGACCAAGGAAAAU
AUGAGGGUUUCACAGUCAA
ACAGCUGAAGGAAAAGGCA
CCAAGGCACCUUGUCGGUA
UUGUCGGUGGUGACAAUGU
GUACCAUGCUAAGGCCAAA
CUAAGGCCAAAGAUCAACU
CAACUCACCUGUAAUAAAU
UUCGACCUCAAGGUCACCA
UCACCAUAAAACCAGCACA
CGGAAACAGAAAAGAGGCA
UCAGGAUGCCAAGAACACU
UAUGAUCCUUGAGAUCUGU
CCAGGUACCGGGGAGACCA
CAGGAUGCCACUAUGUCUA
CUUUGCUCCAGACACAGAU
AUGACCUGAAGCAGCUGGA
UUGACAGAUACAUCUCCAA
AAGUAUGAGCUGGACAAAG
UAUGAGCUGGACAAAGCCU
CUUCUCCGAUAGGAACACA
CCCUCAUCAUCUACCUGGA
GACAAGGUCUCACACUCUA
UCACACUCUGAGGAUGACU
GAGGAUGACUGUCUAGCUU
UUCAAAGUUCACCAAUACU
CUUUAAUGUAGAGCUUAUA
GUCAAGGUCUACGCCUAUU
UACAACCUGGAGGAAAGCU
CUGUACCCGGUUCUACCAU
AUCCGGAAAAGGAGGAUGA
AAAAGGAGGAUGGAAAGCU
CUGAACAAGCUCUGCCGUA
UGAUGAACUGUGCCGCUGU
GUGCUGAGGAGAAUUGCUU
AAUUGCUUCAUACAAAAGU
GUCGGAUGACAAGGUCACA
CCCUGGAAGAACGGCUGGA
GACAAGGCCUGUGAGCCAA
AGGCCUGUGAGCCAGGAGU
CCAGGAGUGGACUAUGUGU
UACAAGACCCGACUGGUCA
UUCAGCUGUCCAAUGACUU
UUUGACGAGUACAUCAUGA
UACAUCAUGGCCAUUGAGA
CAGACCAUCAAGUCAGGCU
CUCGGAUGAGGUGCAGGUU
UUCAUCAGCCCCAUCAAGU
UCAAGUGCAGAGAAGCCCU
CUGAAGCUGGAGGAGAAGA
AAGCUGGAGGAGAAGAAAC
ACACUACCUCAUGUGGGGU
UUCUGGGGAGAGAAGCCCA
UACAUCAUCGGGAAGGACA
CACUUGGGUGGAGCACUGA
AGGACGAAUGCCAAGACGA
AAGAGAACCAGAAACAAUA
UGCCAGGACCUCGGCGCCU
CUUCACCGAGAGCAUGGUU
CACACCCCCAUUCCCCCAA
CUCCAGAUAAAGCUUCAGU
UUAUAUCUCAAAAAAAAAA
GCUGAGGAGAAUUGCUUCAUA
GGAGAAUUGCUUCAUACAAAA
GCAGCUAAAAGACUUUGACUU
AUCCAGACAGACAAGACCAUU
GACAGACAAGACCAUCUACAA
CGUGGUCAAGGUCUUCUCUCU
UCCAGACAGACAAGACCAUCU
AUGAACAAAACUGUGGCUGUU
CUUCAUGGUAUUCCAAGCCUU
CAUCCAGACAGACAAGACCAU
CCAGACAGACAAGACCAUCUA
GACAGACAAGACCAUCUACAC
UACGUGGUCAAGGUCUUCUCU
AGACAGACAAGACCAUCUACA
ACGUGGUCAAGGUCUUCUCUC
AGAAAAAUGGAAUCUCUACGA
UUUGACCUCAUGGUGUUCGUU
GACAAGACCAUCUACACCCCU
GAAAGGGAUCUGUGUGGCAGA
CCAGAUCCACUUCACCAAGAC
UGUAAUAAAUUCGACCUCAAG
AUCCAGACAGACAAGACCAUC
AACAAGAAGAACAAACUCACA
CUUCACCAAGACACCCAAAUU
UGACCUCAUGGUGUUCGUGAU
CCCCUUCGAGGUCACAGUAAU
ACAGACAAGACCAUCUACACA
AUUCACCAAGAAAUGAUUGGU
GGAUGCCAAGAACACUAUGAU
AACUACAUGAACCUACAGAGA
UGACCUCAUGGUGUUCGUGAC
CGUGACAGUGGUGGCAAACUU
CAGUUUCGAGGUCAUAGUGGA
AGGGAUCUGUGUGGCAGACCA
AGGGAUCUGUGUGGCAGACCC
ACGUGGUCAAGGUCUUCUCUA
UUGCCUUUGUCUUGGAACAUU
CAUAGAAGAGUUGAAAGAACC
CGUGCCGGAAGGAAUCAGAAU
CUCCGUGUGGGUGGACGUCAA
UUGACCUCAUGGUGUUCGUGA
CUGCUGAAAGACUUUGACUCU
CAAGAAAGGGAUCUGUGUGGA
AAGAAAGGGAUCUGUGUGGCA
UUUGACCUCAUGGUGUUCGUG
CACUGUGCAAGACUUCCUAAA
CUGGUUGUGGACCAUAGAAGA
UGUUAAAUGGCUGAUCCUGGA
GGAUCUGUGUGGCAGACCCCU
ACAGACAAGACCAUCUACACC
GUACGUGACAGUGGUGGCAAA
GGUCAUGAACAUCUUUCUCAA
GUCGUGCCAGAAGGAAUGAGA
CAAGAAAGGGAUCUGUGUGGC
CUGUGCAAGACUUCCUAAAGA
CCAGAUCCACUUCACCAAGAA
GCUGCUGAAAGACUUUGACUC
UGGUCAAGGUCUUCUCUCU
CAGAUCCACUUCACCAAGA
UGGUCAAGGUCUUCUCUCU
GGUCUACGCCUAUUACAAC
GGUCUACGCCUAUUACAAU
AUCCGAGCCUACUAUGAAA
AGUGGAGCCUACAGAGAAA
AAUUCUACUACAUCUAUAA
AGGGAACUGCCUUUGUCAU
UGACCUCUCCCUACCAGAU
AACUCAGCAUCAACACACA
UGGGCAACUCCAACAAUUA
CCCUCAACGUCAACUUCCU
CCAUCACCACCGACUUCAU
UGGGCUCGCUGGUGGUAAA
AGAUGACCCUGAAGAUAGA
UGGACUGCUGCAACUACAU
CGAAGCUCAUGAAUAUAUU
GAAGCUCAUGAAUAUAUUU
AGGUCACAGUAAUGCAGGA
CGGCUACCCUACUCUGUUG
ACCCUACUCUGUUGUUCGA
CCUACUCUGUUGUUCGAAA
UGCAUUACCUGGAUGAAAC
UGACCACAGCCAAAGAUAA
UGCGUUGGCUCAAUGAACA
AGCCUUGGCUCAAUACCAA
UCAACUCACCUGUAAUAAA
CCAGCACCGGAAACAGAAA
AGAACACUAUGAUCCUUGA
ACACUAUGAUCCUUGAGAU
GAUACAUCUCCAAGUAUGA
GACAAAGCCUUCUCCGAUA
AUAGGAACACCCUCAUCAU
CUUUCAAAGUUCACCAAUA
AUGGAAAGCUGAACAAGCU
GAGAAUUGCUUCAUACAAA
UGGACUAUGUGUACAAGAC
ACAUCAUGGCCAUUGAGCA
CGAGGAGGACGAAUGCCAA
GGUACCUCUUCAUCCAGAC
GUACCUCUUCAUCCAGACA
ACCUCUUCAUCCAGACAGA
CUUCAUCCAGACAGACAAG
UCCAGACAGACAAGACCAU
CAGACAGACAAGACCAUCU
GACAGACAAGACCAUCUAC
CAGACAAGACCAUCUACAC
ACAAGACCAUCUACACCCC
AGUGGAAGAUCCGAGCCUA
CGUGCUGCCCAGUUUCGAG
ACCAGAUCCACUUCACCAA
AGAUCCACUUCACCAAGAC
UCCACUUCACCAAGACACC
CCUUUGACCUCAUGGUGUU
CUUUGACCUCAUGGUGUUC
ACCUCAUGGUGUUCGUGAC
ACAAGAAAGGGAUCUGUGU
CCAGAAUUCUCCUGCAAGG
ACCUCAUCGCCAUCGACUC
CCUUCAUGGUGUUCCAAGC
AAGAGACCAAGGAAAAUGA
UGGCUUUGCUCCAGACACA
ACACCCUCAUCAUCUACCU
AGGUCUACGCCUAUUACAA
GACUAUGUGUACAAGACCC
UCAUCAGCCCCAUCAAGUG
CAGACAAGACCAUCUACAU
UGGUCAAGGUCGUCUCUCA
UGGUCAAGGUCUUCUCUCU
CGGUCAAGGUCUUCUCUCU
GAAUCUCUACGAAGCUCAU
CCAAGAUCCGCUACUACAA
CCUCUUCAUCCAGACAGAC
UGGUCAAGGUCUUCUCUCA
CGGUCAAGGUCGUCUCUCA
UGGUCAAGGUCUUCUCUCU
CCUCUUCAUCCAGACAGAA
CGGUCAAGGUCUUCUCUCA
CCAAGAUCCGCUACUACAC

In some embodiments, an siRNA comprises a sense strand that is modified or unmodified, and that includes any one of the sequences disclosed in WO 2020/104669, WO 2021/037941, WO 2015/089368, WO 2019/089922, WO 2021/081026, WO 2021/178607, U.S. Pat. No. 7,582,746, WO 2007/089375, or WO 2003/066805, each of which are herein incorporated by reference in their entirety.

In some embodiments, duplexed siRNAs comprise blunt ends on both ends. In some embodiments, duplexed siRNAs comprise at least one overhang region. In some embodiments, a duplexed siRNA comprises a 1, 2, 3, 4, 5, or 6 nucleotide 3′ overhang on the sense and/or antisense strand of the duplex. In some embodiments, a duplexed siRNA comprises a 1, 2, 3, 4, 5, or 6 nucleotide 5′ overhang on the sense and/or antisense strand of the duplex.

In some embodiments, an antisense strand comprises an overhang comprising one or more nucleotides that are complementary to the C3 mRNA transcript (SEQ ID NO: 75). In some embodiments, an antisense strand comprises an overhang comprising 1, 2, 3, 4, 5, or 6 nucleotides that are complementary to the C3 mRNA transcript (SEQ ID NO: 75). For example, in some embodiments, a duplexed siRNA comprises an antisense strand comprising the sequence of any one of SEQ ID NOs: 300-324.

TABLE 4

| SEQ ID | Antisense Sequence |
|---|---|
| NO: | (5′ TO 3′) |
| 300 | UUUAUUACAGGUGAGUUGAUC |
| 301 | UUAGACAUAGUGGCAUCCUGG |
| 302 | UCAUGUAGUUGGCUUCAAGGA |
| 303 | UAUUGAGCCAAGGCUUGGAAC |
| 304 | UUAGGCGUAGACCUUGACUGC |
| 305 | UUGCGAACAGCCACAGUUUUG |
| 306 | UUACCUGGUACAGAUCUCAAG |
| 307 | UAGCGAGAUGAGAACAAAGGC |
| 308 | UUGACGGUGAAGAUCCGAUAG |
| 309 | UUUCGCAGGAGGAAGUUGACG |
| 310 | UUCGAAACUGGGCAGCACGUA |
| 311 | UUGAUGAGGGUGUUCCUAUCG |
| 312 | UGAGAGAAGACCUUGACCACG |
| 313 | UGGUAAUUGUUGGAGUUGCCC |
| 314 | UUCCCGAAGAUGACAAAGGCA |
| 315 | UUACUCGUCAAAGUCAUUGGA |
| 316 | UUUUUGCCUGGGAAGUCGUGG |
| 317 | UACCGUAGUAUCUCUGUUCAU |
| 318 | UCCACUAUGACCUCGAAACUG |
| 319 | UGUAGUAUCUCUGUUCAUUGA |
| 320 | UAGUCAAAGUCUUUUAGCUGC |
| 321 | UGUAGGUUCAUGUAGUUGGCU |
| 322 | UUUUCGAACAACAGAGUAGGG |
| 323 | UUUCAUUGAGCCAACGCACGA |
| 324 | UGCGGUCCAUUCGCAGGAGGA |

In some embodiments, a duplexed siRNA comprises an antisense strand comprising the sequence of any one of SEQ ID NOs: 300-324, but lacking the “U” at the 5′ end.

In some embodiments, an antisense strand comprises an overhang comprising one or more nucleotides that are not complementary to the C3 mRNA transcript (SEQ ID NO: 75). In some embodiments, an antisense strand comprises an overhang comprising 1, 2, 3, 4, 5, or 6 nucleotides that are not complementary to the C3 mRNA transcript (SEQ ID NO: 75). In one example, an overhang comprises a 3′ overhang on the antisense and/or sense strand including 1, 2, or 3 uracil nucleotides. In one example, an overhang comprises a 3′ overhang on the antisense and/or sense strand including 1, 2, or 3 adenine nucleotides.

In some embodiments, a duplexed siRNA comprises an antisense strand comprising a sequence listed in the following Table 5:

TABLE 5

| SEQ ID | Antisense Sequence |
|---|---|
| NO: | (5′ TO 3′) |
| 151 | UUAUUACAGGUGAGUUGAUU |
| 152 | UAGACAUAGUGGCAUCCUUU |
| 153 | CAUGUAGUUGGCUUCAAGUU |
| 154 | AUUGAGCCAAGGCUUGGAUU |
| 155 | UAGGCGUAGACCUUGACUUU |
| 156 | UGCGAACAGCCACAGUUUUU |
| 157 | UACCUGGUACAGAUCUCAUU |
| 158 | AGCGAGAUGAGAACAAAGUU |
| 159 | UGACGGUGAAGAUCCGAUUU |
| 160 | UUCGCAGGAGGAAGUUGAUU |
| 161 | UCGAAACUGGGCAGCACGUU |
| 162 | UGAUGAGGGUGUUCCUAUUU |
| 163 | GAGAGAAGACCUUGACCAUU |
| 164 | GGUAAUUGUUGGAGUUGCUU |
| 165 | UCCCGAAGAUGACAAAGGUU |
| 166 | UACUCGUCAAAGUCAUUGUU |
| 167 | UUUUGCCUGGGAAGUCGUUU |
| 168 | ACCGUAGUAUCUCUGUUCUU |
| 169 | CCACUAUGACCUCGAAACUU |
| 170 | GUAGUAUCUCUGUUCAUUUU |
| 171 | AGUCAAAGUCUUUUAGCUUU |
| 172 | GUAGGUUCAUGUAGUUGGUU |
| 173 | UUUCGAACAACAGAGUAGUU |
| 174 | UUCAUUGAGCCAACGCACUU |
| 175 | GCGGUCCAUUCGCAGGAGUU |

In some embodiments, a duplexed siRNA comprises an antisense strand comprising a sequence listed in the following Table 6:

TABLE 6

| SEQ ID | Antisense Sequence |
|---|---|
| NO: | (5′ TO 3′) |
| 176 | UUUAUUACAGGUGAGUUGAUU |
| 177 | UUAGACAUAGUGGCAUCCUUU |
| 178 | UCAUGUAGUUGGCUUCAAGUU |
| 179 | UAUUGAGCCAAGGCUUGGAUU |
| 180 | UUAGGCGUAGACCUUGACUUU |
| 181 | UUGCGAACAGCCACAGUUUUU |
| 182 | UUACCUGGUACAGAUCUCAUU |
| 183 | UAGCGAGAUGAGAACAAAGUU |
| 184 | UUGACGGUGAAGAUCCGAUUU |
| 185 | UUUCGCAGGAGGAAGUUGAUU |
| 186 | UUCGAAACUGGGCAGCACGUU |
| 187 | UUGAUGAGGGUGUUCCUAUUU |
| 188 | UGAGAGAAGACCUUGACCAUU |
| 189 | UGGUAAUUGUUGGAGUUGCUU |
| 190 | UUCCCGAAGAUGACAAAGGUU |
| 191 | UUACUCGUCAAAGUCAUUGUU |
| 192 | UUUUUGCCUGGGAAGUCGUUU |
| 193 | UACCGUAGUAUCUCUGUUCUU |
| 194 | UCCACUAUGACCUCGAAACUU |
| 195 | UGUAGUAUCUCUGUUCAUUUU |
| 196 | UAGUCAAAGUCUUUUAGCUUU |
| 197 | UGUAGGUUCAUGUAGUUGGUU |
| 198 | UUUUCGAACAACAGAGUAGUU |
| 199 | UUUCAUUGAGCCAACGCACUU |
| 200 | UGCGGUCCAUUCGCAGGAGUU |

In some embodiments, siRNAs comprise 5′-phosphate and/or 3′-hydroxyl (e.g., on one or both ends of a sense strand and/or on one or both ends of an antisense strand) groups and/or may comprise one or more additional modifications described herein.

### Modifications

In some embodiments, an inhibitory RNA (e.g., an siRNA or miRNA) of the disclosure includes one or more natural nucleobase and/or one or more modified nucleobases derived from a natural nucleobase. Examples include, but are not limited to, uracil, thymine, adenine, cytosine, and guanine having their respective amino groups protected by acyl protecting groups, 2-fluorouracil, 2-fluorocytosine, 5-bromouracil, 5-iodouracil, 2,6-diaminopurine, azacytosine, pyrimidine analogs such as pseudoisocytosine and pseudouracil and other modified nucleobases such as 8-substituted purines, xanthine, or hypoxanthine (the latter two being the natural degradation products). Exemplary modified nucleobases are disclosed in Chiu and Rana, R N A, 2003, 9, 1034-1048, Limbach et al. Nucleic Acids Research, 1994, 22, 2183-2196 and Revankar and Rao, Comprehensive Natural Products Chemistry, vol. 7, 313.

Modified nucleobases also include expanded-size nucleobases in which one or more aryl rings, such as phenyl rings, have been added. Nucleic base replacements described in the Glen Research catalog (www.glenresearch.com); Krueger A T et al, Acc. Chem. Res., 2007, 40, 141-150; Kool, ET, Acc. Chem. Res., 2002, 35, 936-943; Benner S.A., et al., Nat. Rev. Genet., 2005, 6, 553-543; Romesberg, F. E., et al., Curr. Opin. Chem. Biol., 2003, 7, 723-733; Hirao, I., Curr. Opin. Chem. Biol., 2006, 10, 622-627, are contemplated as useful for siRNA molecules described herein. Modified nucleobases also encompass structures that are not considered nucleobases but are other moieties such as, but not limited to, corrin- or porphyrin-derived rings. Porphyrin-derived base replacements have been described in Morales-Rojas, H and Kool, ET, Org. Lett., 2002, 4, 4377-4380.

In some embodiments, modified nucleobases are of any one of the following structures, optionally substituted:

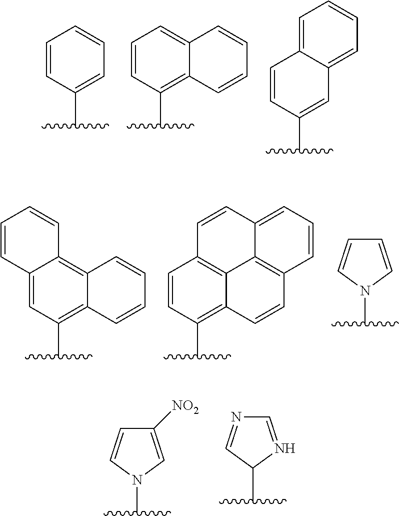

In some embodiments, a modified nucleobase is fluorescent. Exemplary such fluorescent modified nucleobases include phenanthrene, pyrene, stillbene, isoxanthine, isozanthopterin, terphenyl, terthiophene, benzoterthiophene, coumarin, lumazine, tethered stillbene, benzo-uracil, and naphtho-uracil, as shown below:

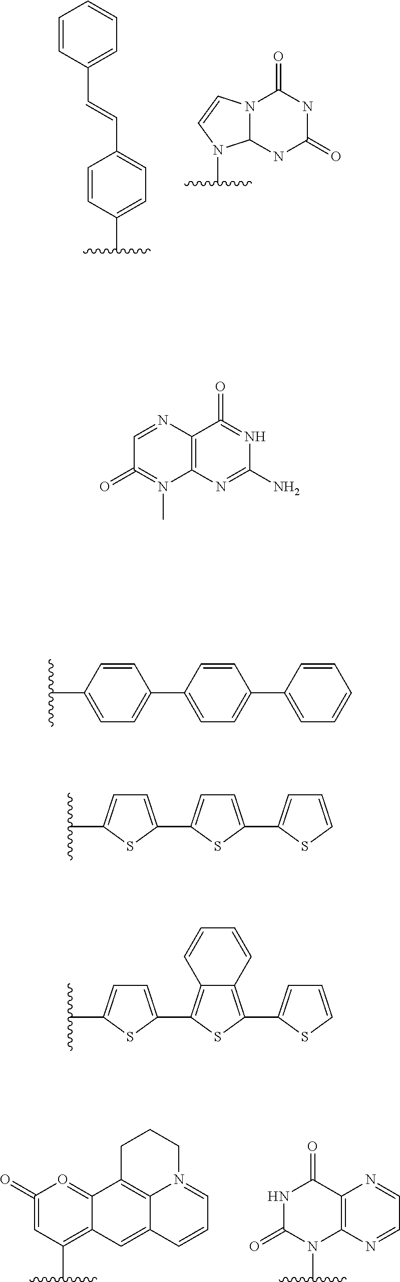

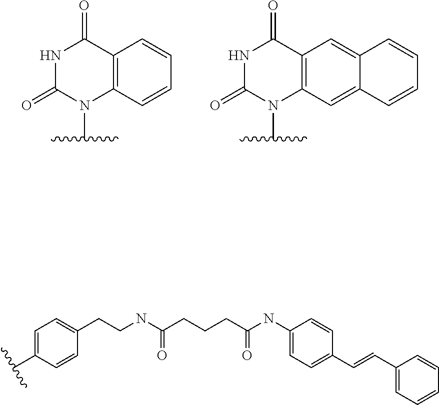 [SMILES: *n1c(=O)[nH]c(=O)c2ccccc21 ; *n1c(=O)[nH]c(=O)c2cc3ccccc3cc21 ; *c1ccc(CCNC(=O)CCCC(=O)Nc2ccc(/C=C/c3ccccc3)cc2)cc1]

In some embodiments, a modified nucleobase is unsubstituted. In some embodiments, a modified nucleobase is substituted. In some embodiments, a modified nucleobase is substituted such that it contains, e.g., heteroatoms, alkyl groups, or linking moieties connected to fluorescent moieties, biotin or avidin moieties, or other protein or peptides. In some embodiments, a modified nucleobase is a “universal base” that is not a nucleobase in the most classical sense, but that functions similarly to a nucleobase. One representative example of such a universal base is 3-nitropyrrole.

In some embodiments, an siRNA described herein includes nucleosides that incorporate modified nucleobases and/or nucleobases covalently bound to modified sugars. Some examples of nucleosides that incorporate modified nucleobases include 4-acetylcytidine; 5-(carboxyhydroxylmethyl) uridine; 2′-O-methylcytidine; 5-carboxymethylaminomethyl-2-thiouridine; 5-carboxymethylaminomethyluridine; dihydrouridine; 2′-O-methylpseudouridine; beta, D-galactosylqueosine; 2′-O-methylguanosine; N⁶-isopentenyladenosine; 1-methyladenosine; 1-methylpseudouridine; 1-methylguanosine; 1-methylinosine; 2,2-dimethylguanosine; 2-methyladenosine; 2-methylguanosine; N⁷-methylguanosine; 3-methyl-cytidine; 5-methylcytidine; 5-hydroxymethylcytidine; 5-formylcytosine; 5-carboxylcytosine; N⁶-methyladenosine; 7-methylguanosine; 5-methylaminoethyluridine; 5-methoxyaminomethyl-2-thiouridine; beta,D-mannosylqueosine; 5-methoxycarbonylmethyluridine; 5-methoxyuridine; 2-methylthio-N⁶-isopentenyladenosine; N-((9-beta,D-ribofuranosyl-2-methylthiopurine-6-yl) carbamoyl) threonine; N-((9-beta,D-ribofuranosylpurine-6-yl)-N-methylcarbamoyl) threonine; uridine-5-oxyacetic acid methylester; uridine-5-oxyacetic acid (v); pseudouridine; queosine; 2-thiocytidine; 5-methyl-2-thiouridine; 2-thiouridine; 4-thiouridine; 5-methyluridine; 2′-O-methyl-5-methyluridine; and 2′-O-methyluridine.

In some embodiments, nucleosides include 6′-modified bicyclic nucleoside analogs that have either (R) or(S)-chirality at the 6′-position and include the analogs described in U.S. Pat. No. 7,399,845. In other embodiments, nucleosides include 5′-modified bicyclic nucleoside analogs that have either (R) or(S)-chirality at the 5′-position and include the analogs described in U.S. Publ. No. 20070287831. In some embodiments, a nucleobase or modified nucleobase is 5-bromouracil, 5-iodouracil, or 2,6-diaminopurine. In some embodiments, a nucleobase or modified nucleobase is modified by substitution with a fluorescent moiety.

Methods of preparing modified nucleobases are described in, e.g., U.S. Pat. Nos. 3,687,808; 4,845,205; 5,130,30; 5,134,066; 5,175,273; 5,367,066; 5,432,272; 5,457,187; 5,457,191; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088.

In some embodiments, an siRNA described herein includes one or more modified nucleotides wherein a phosphate group or linkage phosphorus in the nucleotides are linked to various positions of a sugar or modified sugar. As non-limiting examples, the phosphate group or linkage phosphorus can be linked to the 2′, 3′, 4′ or 5′ hydroxyl moiety of a sugar or modified sugar. Nucleotides that incorporate modified nucleobases as described herein are also contemplated in this context.

Other modified sugars can also be incorporated within an siRNA molecule. In some embodiments, a modified sugar contains one or more substituents at the 2′ position including one of the following: —F; —CF₃, —CN, —N₃, —NO, —NO₂, —OR′, —SR′, or —N(R′)₂, wherein each R′ is independently as defined above and described herein; —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C1-C10 alkylene)-NH—(C₁-C₁₀ alkyl) or —O—(C₁-C₁₀ alkylene)-NH(C₁-C₁₀ alkyl)₂, —NH—(C₁-C₁₀ alkylene)-O—(C₁-C₁₀ alkyl), or —N(C₁-C₁₀ alkyl)-(C₁-C₁₀ alkylene)-O—(C₁-C₁₀ alkyl), wherein the alkyl, alkylene, alkenyl and alkynyl may be substituted or unsubstituted. Examples of substituents include, and are not limited to, —O(CH₂)ₙOCH₃, and —O(CH₂)ₙNH₂, wherein n is from 1 to about 10, MOE, DMAOE, DMAEOE. Also contemplated herein are modified sugars described in WO 2001/088198; and Martin et al., Helv. Chim. Acta, 1995, 78, 486-504. In some embodiments, a modified sugar comprises one or more groups selected from a substituted silyl group, an RNA cleaving group, a reporter group, a fluorescent label, an intercalator, a group for improving the pharmacokinetic properties of a nucleic acid, a group for improving the pharmacodynamic properties of a nucleic acid, or other substituents having similar properties. In some embodiments, modifications are made at one or more of the 2′, 3′, 4′, 5′, or 6′ positions of the sugar or modified sugar, including the 3′ position of the sugar on the 3′-terminal nucleotide or in the 5′ position of the 5′-terminal nucleotide.

In some embodiments, the 2′-OH of a ribose is replaced with a substituent including one of the following: —H, —F; CF₃, —CN, —N₃, —NO, —NO₂, —OR′, —SR′, or —N(R′) 2, wherein each R′ is independently as defined above and described herein; —O—(C₁-C₁₀ alkyl), —S(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C₁-C₁₀ alkylene)-NH—(C₁-C₁₀ alkyl) or —O—(C₁-C₁₀ alkylene)-NH (C₁-C₁₀ alkyl)₂, —NH—(C₁-C₁₀ alkylene)-O—(C₁-C₁₀ alkyl), or —N(C₁-C₁₀ alkyl)-(C₁-C₁₀ alkylene)-O—(C₁-C₁₀ alkyl), wherein the alkyl, alkylene, alkenyl and alkynyl may be substituted or unsubstituted. In some embodiments, the 2′ OH is replaced with —H (deoxyribose). In some embodiments, the 2′-OH is replaced with —F. In some embodiments, the 2′-OH is replaced with —OR′. In some embodiments, the 2′-OH is replaced with —OMe. In some embodiments, the 2′-OH is replaced with —OCH₂CH₂OMe.

Modified sugars also include locked nucleic acids (LNAs). In some embodiments, the locked nucleic acid has the structure indicated below. A locked nucleic acid of the structure below is indicated, wherein Ba represents a nucleobase or modified nucleobase as described herein, and wherein R^{2s} is —OCH₂C4′-

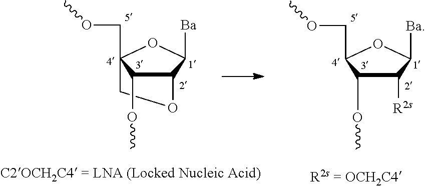

In some embodiments, a modified sugar is an ENA such as those described in, e.g., Seth et al., J Am Chem Soc. 2010 Oct. 27; 132 (42): 14942-14950. In some embodiments, a modified sugar is any of those found in an XNA (xenonucleic acid), for instance, arabinose, anhydrohexitol, threose, 2′fluoroarabinose, or cyclohexene.

Modified sugars include sugar mimetics such as cyclobutyl or cyclopentyl moieties in place of the pentofuranosyl sugar (see, e.g., U.S. Pat. Nos. 4,981,957; 5,118,800; 5,319,080; and 5,359,044). Some modified sugars that are contemplated include sugars in which the oxygen atom within the ribose ring is replaced by nitrogen, sulfur, selenium, or carbon. In some embodiments, a modified sugar is a modified ribose wherein the oxygen atom within the ribose ring is replaced with nitrogen, and wherein the nitrogen is optionally substituted with an alkyl group (e.g., methyl, ethyl, isopropyl, etc.).

Non-limiting examples of modified sugars include glycerol, which form glycerol nucleic acid (GNA) analogues. One example of a GNA analogue is described in Zhang, R et al., J. Am. Chem. Soc., 2008, 130, 5846-5847; Zhang L, et al., J. Am. Chem. Soc., 2005, 127, 4174-4175 and Tsai C H et al., PNAS, 2007, 14598-14603. Another example of a GNA derived analogue, flexible nucleic acid (FNA) based on the mixed acetal aminal of formyl glycerol, is described in Joyce G F et al., PNAS, 1987, 84, 4398-4402 and Heuberger B D and Switzer C, J. Am. Chem. Soc., 2008, 130, 412-413. Additional non-limiting examples of modified sugars include hexopyranosyl (6′ to 4′), pentopyranosyl (4′ to 2′), pentopyranosyl (4′ to 3′), or tetrofuranosyl (3′ to 2′) sugars.

Modified sugars and sugar mimetics can be prepared by methods known in the art, including, but not limited to: A. Eschenmoser, Science (1999), 284:2118; M. Bohringer et al, Helv. Chim. Acta (1992), 75:1416-1477; M. Egli et al, J. Am. Chem. Soc. (2006), 128 (33): 10847-56; A. Eschenmoser in Chemical Synthesis: Gnosis to Prognosis, C. Chatgilialoglu and V. Sniekus, Ed., (Kluwer Academic, Netherlands, 1996), p. 293; K.-U. Schoning et al, Science (2000), 290:1347-1351; A. Eschenmoser et al, Helv. Chim. Acta (1992), 75:218; J. Hunziker et al, Helv. Chim. Acta (1993), 76:259; G. Otting et al, Helv. Chim. Acta (1993), 76:2701; K. Groebke et al, Helv. Chim. Acta (1998), 81:375; and A. Eschenmoser, Science (1999), 284:2118. Modifications to the 2′ modifications can be found in Verma, S. et al. Annu. Rev. Biochem. 1998, 67, 99-134 and all references therein. Specific modifications to the ribose can be found in the following references: 2′-fluoro (Kawasaki et. al., J. Med. Chem., 1993, 36, 831-841), 2′-MOE (Martin, P. Helv. Chim. Acta 1996, 79, 1930-1938), “LNA” (Wengel, J. Acc. Chem. Res. 1999, 32, 301-310); PCT Publication No. WO2012/030683.

According to certain embodiments various nucleotide modifications or nucleotide modification patterns may be used selectively in either the sense or antisense strand of an inhibitory RNA (e.g., siRNA) described herein. For example, in some embodiments one may utilize unmodified ribonucleotides in the antisense strand (at least within the duplex portion thereof) while employing modified nucleotides and/or modified or unmodified deoxyribonucleotides at some or all positions in the sense strand. In some embodiments, particular patterns of modifications are employed throughout part or all of either or both strands of an siRNA. Nucleotide modifications may occur in any of a variety of patterns. For example, an alternating pattern may be used. For example, the antisense, sense strand, or both, may have 2′-O-methyl or 2′-fluoro modifications on every other nucleotide. In some embodiments, an inhibitory RNA (e.g., siRNA) comprises a sense and/or antisense strand with at least one unmodified nucleotide.

In some embodiments, a sense and/or antisense strand comprises one or more motifs of three identical modifications on three consecutive nucleotides. For example, in some embodiments a double-stranded siRNA comprises one or more motifs of three identical modifications on three consecutive nucleotides in a sense strand, antisense strand, or both. In some embodiments such a motif may occur at or near the cleavage site in either or both strands. Examples of such motifs are described in US Pat. App. Pubs. 20150197746, 20150247143, and 20160298124.

In some embodiments, an inhibitory RNA (e.g., siRNA) is a bluntmer of 19 nucleotides in length, wherein the sense strand contains at least one motif of three 2′-F modifications on three consecutive nucleotides at positions 7, 8, 9 from the 5′end, and where the antisense strand contains at least one motif of three 2′-O-methyl modifications on three consecutive nucleotides at positions 11, 12, 13 from the 5′end. In some embodiments, an inhibitory RNA (e.g., siRNA) is a double ended bluntmer of 20 nucleotides in length, wherein the sense strand contains at least one motif of three 2′-F modifications on three consecutive nucleotides at positions 8, 9, 10 from the 5′end, and where the antisense strand contains at least one motif of three 2′-O-methyl modifications on three consecutive nucleotides at positions 11, 12, 13 from the 5′end. In some embodiments an inhibitory RNA (e.g., siRNA) is a double ended bluntmer of 21 nucleotides in length, wherein the sense strand contains at least one motif of three 2′-F modifications on three consecutive nucleotides at positions 9, 10, 11 from the 5′end, and where the antisense strand contains at least one motif of three 2′-O-methyl modifications on three consecutive nucleotides at positions 11, 12, 13 from the 5′end.

In some embodiments, an inhibitory RNA (e.g., siRNA) comprises a 19 nucleotide sense strand and a 21 nucleotide antisense strand, wherein the sense strand contains at least one motif of three 2′-F modifications on three consecutive nucleotides at positions 7, 8, 9 from the 5′end; the antisense strand contains at least one motif of three 2′-O-methyl modifications on three consecutive nucleotides at positions 11, 12, 13 from the 5′end, wherein one end of the inhibitory RNA (e.g., siRNA) is blunt, while the other end comprises a 2 nucleotide overhang. Preferably, the 2 nucleotide overhang is at the 3′-end of the antisense strand. When the 2 nucleotide overhang is at the 3′-end of the antisense strand, there may be two phosphorothioate internucleotide linkages between the terminal three nucleotides, wherein two of the three nucleotides are the overhang nucleotides, and the third nucleotide is a paired nucleotide next to the overhang nucleotide. In some embodiments, the inhibitory RNA (e.g., siRNA) additionally has two phosphorothioate internucleotide linkages between the terminal three nucleotides at both the 5′-end of the sense strand and at the 5′-end of the antisense strand. In some embodiments, every nucleotide in the sense strand and the antisense strand of an inhibitory RNA (e.g., siRNA), including the nucleotides that are part of the motifs are modified nucleotides. In some embodiments each residue is independently modified with a 2′-O-methyl or 3′-fluoro, e.g., in an alternating motif.

In some embodiments, an inhibitory RNA (e.g., siRNA) comprises a 19 nucleotide sense strand and a 21 nucleotide antisense strand, wherein (i) the sense strand contains 2′-F modifications at positions 3, 7, 8, 9, 12, and 17 from the 5′end; (ii) the sense strand contains 2′-O-methyl modifications at positions 1, 2, 4, 5, 6, 10, 11, 13, 14, 15, 16, 18, and 19 from the 5′end; (iii) the antisense strand contains 2′-F modifications at positions 2 and 14 from the 5′end; and (iv) the antisense strand contains 2′-O-methyl modifications at positions 1, 3, 4, 5, 6, 7, 8, 9, 10, 11, 12, 13, 15, 16, 17, 18, 19, 20, and 21 from the 5′end; wherein one end of the inhibitory RNA (e.g., siRNA) is blunt, while the other end comprises a 2 nucleotide overhang at the 3′-end of the antisense strand. In some embodiments, the inhibitory RNA (e.g., siRNA) includes an antisense strand comprising two phosphorothioate internucleotide linkages between the terminal three nucleotides at the 3′ end, wherein two of the three nucleotides are the overhang nucleotides, and the third nucleotide is a paired nucleotide next to the overhang nucleotide. In some embodiments, an inhibitory RNA (e.g., siRNA) additionally has two phosphorothioate internucleotide linkages between the terminal three nucleotides at both the 5′-end of the sense strand and at the 5′-end of the antisense strand.

In some embodiments, every nucleotide in the sense strand and antisense strand of an inhibitory RNA (e.g., siRNA), including the nucleotides that are part of the motifs, may be modified. Each nucleotide may be modified with the same or different modification which can include one or more alteration of one or both of the non-linking phosphate oxygens and/or of one or more of the linking phosphate oxygens; alteration of a constituent of the ribose sugar, e.g., of the 2′ hydroxyl on the ribose sugar; wholesale replacement of the phosphate moiety with “dephospho” linkers; modification or replacement of a naturally occurring base; and replacement or modification of the ribose-phosphate backbone.

In some embodiments at least 50%, 60%, 70%, 80%, 90%, or more, e.g., 100% of the residues of the sense strand and antisense strand is independently modified with LNA, CRN, CET, UNA, HNA (1,5-anhydrohexitol nucleic acid), CeNA (cyclohexenyl nucleic acid—a DNA mimic in which the deoxyribose is replaced by a six-membered cyclohexene ring), 2′-methoxyethyl, 2′-O-methyl, 2′-O-allyl, 2′-C-allyl, 2′-deoxy, 2′-hydroxyl, or 2′-fluoro. The strands can contain more than one modification. In some embodiments at least 50%, 60%, 70%, 80%, 90%, or more, e.g., 100% of the residues of the sense strand and antisense strand is independently modified with 2′-O-methyl or 2′-fluoro. In some embodiments at least two different modifications are present on the sense strand and antisense strand. Those two modifications may be the 2′-O-methyl or 2′-fluoro modifications, or others.

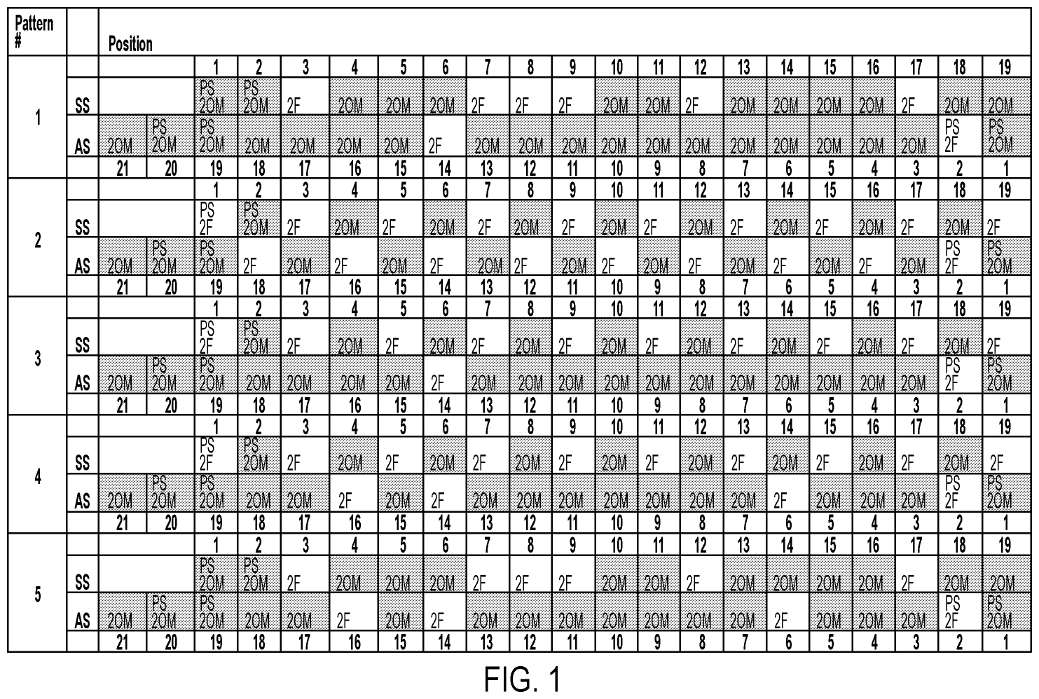

In some embodiments, the sense and antisense strands of the duplex of an inhibitory RNA (e.g., an siRNA), comprise any one of the modification patterns depicted as patterns 1-5 in FIG. 1. In FIG. 1, at any given position, a “2OM” represents a 2′-O-methyl modification and a “2F” represents a 2′-Fluoro modification. A “PS” represents a phosphorothioate bond between the nucleotide at a position noted with a “PS” and the adjacent nucleotide that is 3′ to the position noted with a “PS”. In some embodiments, any one of the antisense strands disclosed in SEQ ID NOs: 176-200 and 300-324, can be modified according to any one of the modification patterns 1-5 of the antisense strand (“AS”) disclosed in FIG. 1. In some embodiments, any one of the sense strands disclosed in SEQ ID NOs: 126-150, can be modified according to any one of the modification patterns 1-5 of the sense strand (“SS”) disclosed in FIG. 1. In some embodiments, the sense and/or antisense strands of the duplex of an inhibitory RNA (e.g., an siRNA), comprises any one of the modification patterns depicted as patterns 1-5 in FIG. 1, but where any 1, 2, 3, or 4 positions of the sense and/or antisense strands do not include the modification depicted at such 1, 2, 3, or 4 positions in one of patterns 1-5.

In some embodiments, an siRNA comprises any one of the modification patterns 1-5 (depicted in FIG. 1), and also includes a phosphorothioate bond between the last two, three, or four nucleotides of (i) the 5′ terminus of the sense strand; (ii) the 3′ terminus of the sense strand; (iii) the 5′ terminus of the antisense strand, and/or (iv) the 3′ terminus of the antisense strand. For example, in some embodiments, an siRNA includes (i) a sense strand that includes a phosphorothioate bond between the nucleotides at positions 1 and 2 from the 5′ end, and between the nucleotides at positions 2 and 3 from the 5′ end; (ii) a sense strand that includes a phosphorothioate bond between the nucleotides at positions 1 and 2 from the 3′ end, and between the nucleotides at positions 2 and 3 from the 3′ end; (iii) an antisense strand that includes a phosphorothioate bond between the nucleotides at positions 1 and 2 from the 5′ end, and between the nucleotides at positions 2 and 3 from the 5′ end; and/or (iv) an antisense strand that includes a phosphorothioate bond between the nucleotides at positions 1 and 2 from the 3′ end, and between the nucleotides at positions 2 and 3 from the 3′ end.

In some embodiments, an siRNA can be modified according to any one of the modification patterns 1-5 in FIG. 1 and can also be conjugated to a ligand, e.g., as described herein. In some such cases, a ligand can be attached to any of the 3′ or 5′ terminus of the sense or antisense strand. In some embodiments, an siRNA (e.g., any of the siRNAs: 1-57 listed in Tables 10 and 15, e.g., siRNA 22, 32, and 53) comprises a ligand (e.g., a GalNAc ligand, e.g., a GalNAc of Formula XD or XE described herein) conjugated to a terminus (e.g., 3′ or 5′ terminus of a sense or antisense strand), and said siRNA does not include a phosphorothioate bond between the two, three, or four nucleotides at the end of terminus that is conjugated to a ligand. For example, in some embodiments, an siRNA (e.g., any of the siRNAs: 1-57 listed in Tables 10 and 15, e.g., siRNA 22, 32, and 53) includes a ligand (e.g., a GalNAc ligand, e.g., a GalNAc of Formula XD or XE described herein) conjugated to the 5′ end of the sense strand, and the siRNA includes (i) a sense strand that does not includes a phosphorothioate bond between the nucleotides at positions 1, 2, 3, or 4 from the 5′ end; (ii) a sense strand that includes a phosphorothioate bond between the nucleotides at positions 1 and 2 from the 3′ end, and between the nucleotides at positions 2 and 3 from the 3′ end; (iii) an antisense strand that includes a phosphorothioate bond between the nucleotides at positions 1 and 2 from the 5′ end, and between the nucleotides at positions 2 and 3 from the 5′ end; and (iv) an antisense strand that includes a phosphorothioate bond between the nucleotides at positions 1 and 2 from the 3′ end, and between the nucleotides at positions 2 and 3 from the 3′ end.

In some embodiments, an siRNA (e.g., any of the siRNAs: 1-57 listed in Tables 10 and 15, e.g., siRNA 22, 32, and 53) includes a ligand (e.g., a GalNAc ligand, e.g., a GalNAc of Formula XD or XE described herein) conjugated to the 3′ end of the sense strand, and the siRNA includes (i) a sense strand that includes a phosphorothioate bond between the nucleotides at positions 1 and 2 from the 5′ end, and between the nucleotides at positions 2 and 3 from the 5′ end; (ii) a sense strand that does not include a phosphorothioate bond between the nucleotides at positions 1, 2, 3, or 4 from the 3′ end; (iii) an antisense strand that includes a phosphorothioate bond between the nucleotides at positions 1 and 2 from the 5′ end, and between the nucleotides at positions 2 and 3 from the 5′ end; and (iv) an antisense strand that includes a phosphorothioate bond between the nucleotides at positions 1 and 2 from the 3′ end, and between the nucleotides at positions 2 and 3 from the 3′ end.

In some embodiments, an siRNA (e.g., any of the siRNAs: 1-57 listed in Tables 10 and 15, e.g., siRNA 22, 32, and 53) includes a ligand (e.g., a GalNAc ligand, e.g., a GalNAc of Formula XD or XE described herein) conjugated to the 5′ end of the antisense strand, and the siRNA includes (i) a sense strand that includes a phosphorothioate bond between the nucleotides at positions 1 and 2 from the 5′ end, and between the nucleotides at positions 2 and 3 from the 5′ end; (ii) a sense strand that includes a phosphorothioate bond between the nucleotides at positions 1 and 2 from the 3′ end, and between the nucleotides at positions 2 and 3 from the 3′ end; (iii) an antisense strand that does not include a phosphorothioate bond between the nucleotides at positions 1, 2, 3, or 4 from the 5′ end; and (iv) an antisense strand that includes a phosphorothioate bond between the nucleotides at positions 1 and 2 from the 3′ end, and between the nucleotides at positions 2 and 3 from the 3′ end.

In some embodiments, an siRNA (e.g., any of the siRNAs: 1-57 listed in Tables 10 and 15, e.g., siRNA 22, 32, and 53) includes a ligand (e.g., a GalNAc ligand, e.g., a GalNAc of Formula XD or XE described herein) conjugated to the 3′ end of the antisense strand, and the siRNA includes (i) a sense strand that includes a phosphorothioate bond between the nucleotides at positions 1 and 2 from the 5′ end, and between the nucleotides at positions 2 and 3 from the 5′ end; (ii) a sense strand that includes a phosphorothioate bond between the nucleotides at positions 1 and 2 from the 3′ end, and between the nucleotides at positions 2 and 3 from the 3′ end; (iii) an antisense strand that includes a phosphorothioate bond between the nucleotides at positions 1 and 2 from the 5′ end, and between the nucleotides at positions 2 and 3 from the 5′ end; and (iv) an antisense strand that does not include a phosphorothioate bond between the nucleotides at positions 1, 2, 3, or 4 from the 3′ end.

In some embodiments, an siRNA (e.g., any of the siRNAs: 1-57 listed in Tables 10 and 15, e.g., siRNA 22, 32, and 53) comprises a ligand (e.g., a GalNAc ligand, e.g., a GalNAc of Formula XD or XE described herein) conjugated to a terminus (e.g., 3′ or 5′ terminus of a sense or antisense strand), and said siRNA includes a phosphorothioate bond between the two, three, or four nucleotides at the end of terminus that is conjugated to a ligand.

For example, in some embodiments, an siRNA (e.g., any of the siRNAs: 1-57 listed in Tables 10 and 15, e.g., siRNA 22, 32, and 53) includes a ligand (e.g., a GalNAc ligand, e.g., a GalNAc of Formula XD or XE described herein) conjugated to the 5′ end of the sense strand, and the siRNA includes (i) a sense strand that includes a phosphorothioate bond between the nucleotides at positions 1, 2, 3, or 4 from the 5′ end; (ii) a sense strand that includes a phosphorothioate bond between the nucleotides at positions 1 and 2 from the 3′ end, and between the nucleotides at positions 2 and 3 from the 3′ end; (iii) an antisense strand that includes a phosphorothioate bond between the nucleotides at positions 1 and 2 from the 5′ end, and between the nucleotides at positions 2 and 3 from the 5′ end; and (iv) an antisense strand that includes a phosphorothioate bond between the nucleotides at positions 1 and 2 from the 3′ end, and between the nucleotides at positions 2 and 3 from the 3′ end.

In some embodiments, an siRNA (e.g., any of the siRNAs: 1-57 listed in Tables 10 and 15, e.g., siRNA 22, 32, and 53) includes a ligand (e.g., a GalNAc ligand, e.g., a GalNAc of Formula XD or XE described herein) conjugated to the 3′ end of the sense strand, and the siRNA includes (i) a sense strand that includes a phosphorothioate bond between the nucleotides at positions 1 and 2 from the 5′ end, and between the nucleotides at positions 2 and 3 from the 5′ end; (ii) a sense strand that includes a phosphorothioate bond between the nucleotides at positions 1, 2, 3, or 4 from the 3′ end; (iii) an antisense strand that includes a phosphorothioate bond between the nucleotides at positions 1 and 2 from the 5′ end, and between the nucleotides at positions 2 and 3 from the 5′ end; and (iv) an antisense strand that includes a phosphorothioate bond between the nucleotides at positions 1 and 2 from the 3′ end, and between the nucleotides at positions 2 and 3 from the 3′ end.

In some embodiments, an siRNA (e.g., any of the siRNAs: 1-57 listed in Tables 10 and 15, e.g., siRNA 22, 32, and 53) includes a ligand (e.g., a GalNAc ligand, e.g., a GalNAc of Formula XD or XE described herein) conjugated to the 5′ end of the antisense strand, and the siRNA includes (i) a sense strand that includes a phosphorothioate bond between the nucleotides at positions 1 and 2 from the 5′ end, and between the nucleotides at positions 2 and 3 from the 5′ end; (ii) a sense strand that includes a phosphorothioate bond between the nucleotides at positions 1 and 2 from the 3′ end, and between the nucleotides at positions 2 and 3 from the 3′ end; (iii) an antisense strand that includes a phosphorothioate bond between the nucleotides at positions 1, 2, 3, or 4 from the 5′ end; and (iv) an antisense strand that includes a phosphorothioate bond between the nucleotides at positions 1 and 2 from the 3′ end, and between the nucleotides at positions 2 and 3 from the 3′ end.

In some embodiments, an siRNA (e.g., any of the siRNAs: 1-57 listed in Tables 10 and 15, e.g., siRNA 22, 32, and 53) includes a ligand (e.g., a GalNAc ligand, e.g., a GalNAc of Formula XD or XE described herein) conjugated to the 3′ end of the antisense strand, and the siRNA includes (i) a sense strand that includes a phosphorothioate bond between the nucleotides at positions 1 and 2 from the 5′ end, and between the nucleotides at positions 2 and 3 from the 5′ end; (ii) a sense strand that includes a phosphorothioate bond between the nucleotides at positions 1 and 2 from the 3′ end, and between the nucleotides at positions 2 and 3 from the 3′ end; (iii) an antisense strand that includes a phosphorothioate bond between the nucleotides at positions 1 and 2 from the 5′ end, and between the nucleotides at positions 2 and 3 from the 5′ end; and (iv) an antisense strand that includes a phosphorothioate bond between the nucleotides at positions 1, 2, 3, or 4 from the 3′ end.

In some embodiments, the sense and/or antisense strand comprises modifications of an alternating pattern. The term “alternating motif” as used herein refers to a motif having one or more modifications, each modification occurring on alternating groups of one or more nucleotides of one strand. For example, an alternating nucleotide may refer to one per every other nucleotide or one per every three nucleotides, or a similar pattern. For example, if A, B and C each represent one type of modification to the nucleotide, the alternating motif can be “ABABABABABAB . . . ,” “AABBAABBAABB . . . ,” “AABAABAABAAB . . . ,” “AAABAAABAAAB . . . ,” “AAABBBAAABBB . . . ,” or “ABCABCABCABC . . . ,” etc.

The type of modifications contained in the alternating motif may be the same or different. For example, if A, B, C, D each represent one type of modification on the nucleotide, the alternating pattern, i.e., modifications on every other nucleotide, may be the same, but each of the sense strand or antisense strand can be selected from several possibilities of modifications within the alternating motif such as “ABABAB . . . ”, “ACACAC . . . ” “BDBDBD . . . ” or “CDCDCD . . . ,” etc.

In some embodiments, an inhibitory RNA (e.g., siRNA) comprises the modification pattern for the alternating motif on the sense strand that is shifted relative to the modification pattern for the alternating motif on the antisense strand. The shift may be such that the modified group of nucleotides of the sense strand corresponds to a differently modified group of nucleotides of the antisense strand and vice versa. For example, when paired with the antisense strand in the dsRNA duplex, the alternating motif in the sense strand may start with “ABABAB” from 5′-3′ of the strand and the alternating motif in the antisense strand may start with “BAB ABA” from 5′-3 of the strand, within the duplex portion. As another example, the alternating motif in the sense strand may start with “AABBAABB” from 5′-3′ of the strand and the alternating motif in the antisense strand may start with “BBAABBAA” from 5′-3′ of the strand, within the duplex portion, so that there is a complete or partial shift of the modification patterns between the sense strand and the antisense strand.

In some embodiments, an inhibitory RNA (e.g., siRNA) comprises the pattern of the alternating motif of 2′-O-methyl modification and 2′-F modification on the sense strand has a shift relative to the pattern of the alternating motif of 2′-O-methyl modification and 2′-F modification on the antisense strand, i.e., the 2′-O-methyl modified nucleotide on the sense strand base pairs with a 2′-F modified nucleotide on the antisense strand and vice versa. The 1 position of the sense strand may start with the 2′-F modification, and the 1 position of the antisense strand may start with the 2′-O-methyl modification.

In some embodiments, one or more motifs of three identical modifications can be introduced on three consecutive nucleotides of the sense strand and/or antisense strand to interrupt the initial modification pattern present in the sense strand and/or antisense strand. In some embodiments, when the motif of three identical modifications on three consecutive nucleotides is introduced to any of the strands, the modification of the nucleotide next to the motif is a different modification than the modification of the motif. For example, the portion of the sequence containing the motif is “ . . . . NaYYYNb . . . ,” where “Y” represents the modification of the motif of three identical modifications on three consecutive nucleotide, and “Na” and “Nb” represent a modification to the nucleotide next to the motif “YYY” that is different than the modification of Y, and where Na and Nb can be the same or different modifications.

An inhibitory RNA (e.g., siRNA) may further comprise at least one phosphorothioate or methylphosphonate internucleotide linkage. In some embodiments, the internucleotide linkage modification may occur on every nucleotide on the sense strand and/or antisense strand; each internucleotide linkage modification may occur in an alternating pattern on the sense strand and/or antisense strand; or the sense strand or antisense strand may contain both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand. In some embodiments, an inhibitory RNA (e.g., siRNA) comprises 6-8 phosphorothioate internucleotide linkages. In some embodiments, the antisense strand comprises two phosphorothioate internucleotide linkages at the 5′-terminus and two phosphorothioate internucleotide linkages at the 3′-terminus, and the sense strand comprises at least two phosphorothioate internucleotide linkages at either the 5′-terminus or the 3 ′-terminus.

In certain embodiments, an inhibitory RNA (e.g., siRNA) may have any of the configurations and/or modification patterns described from p. 59 (line 20) to p. 65 (line 15) of WO/2015/089368, or corresponding paragraphs [0469]-[0537] of US Pat. App. Pub. No. 20160298124 or in the claims of either or both of said publications. For example, in some embodiments an inhibitory RNA (e.g., siRNA) comprises a sense strand and an antisense strand, wherein said sense strand is complementary to said antisense strand, wherein said antisense strand comprises a region complementary to part of an mRNA encoding C3 (e.g., a target region described herein), wherein each strand is about 14 to about 30 nucleotides in length, wherein said agent is represented by formula (III):

| | |
|---|---|
| sense: | 5′ nₚ-Nₐ-(X X X)ᵢ- N_{b}- Y Y Y-N_{b}-(Z Z Z)ⱼ-Nₐ- n_{q} 3′ |
| antisense: | 3′ nₚ′ Nₐ′ (X′X′X′)ₖ N_{b}′ Y′Y′Y′ N_{b}′ (Z′Z′Z′)ₗ Nₐ′ n_{q}′ 5′ |

wherein: i, j, k, and 1 are each independently 0 or 1; p, p′, q, and q′ are each independently 0-6; each Na and Na′ independently represents an oligonucleotide sequence comprising 0-25 nucleotides which are either modified or unmodified or combinations thereof, each sequence comprising at least two differently modified nucleotides; each No and No′ independently represents an oligonucleotide sequence comprising 0-10 nucleotides which are either modified or unmodified or combinations thereof; each np, np′, nq, and nq′, each of which may or may not be present, independently represents an overhang nucleotide; XXX, YYY, ZZZ, X′X′X′, Y′Y′Y′, and Z′Z′Z′ each independently represent one motif of three identical modifications on three consecutive nucleotides; modifications on Nb differ from the modification on Y and modifications on Nb′ differ from the modification on Y′; and wherein the sense strand is conjugated to at least one ligand. In some embodiments i is 0; j is 0; i is 1; j is 1; both i and j are 0; or both i and j are 1. In some embodiments XXX is complementary to X′X′X′, YYY is complementary to Y′Y′Y′, and ZZZ is complementary to Z′Z′Z′. It should be understood that each X may comprise a different base, so long as each X comprises the same modification. For example, XXX could represent AGC where each nucleotide comprises a 2-F modification. Similarly, each X′, each Y, each Y′, each Z, and each Z may be different.

In some embodiments formula (III) is represented by formula (IIIa):

| | |
|---|---|
| sense: | 5′ nₚ-Nₐ- Y Y Y-Nₐ- n_{q} 3′ |
| antisense: | 3′ nₚ′-Nₐ′-Y′Y′Y′-Nₐ′-n_{q}′ 5′ |

or wherein formula (III) is represented by formula (IIIb):

| | |
|---|---|
| sense: | 5′ nₚ-Nₐ-Y Y Y-N_{b}-Z Z Z-Nₐ-n_{q} 3′ |
| antisense: | 3′ nₚ′-Nₐ′-Y′Y′Y′-N_{b}-Z′Z′Z′-Nₐ′-n_{q}′ 5′ |

wherein each Nb and Nb′ independently represents an oligonucleotide sequence comprising 1-5 modified nucleotides; or wherein formula (III) is represented by formula (IIIc):

| | |
|---|---|
| sense: | 5′ nₚ-Nₐ- X X X-N_{b}-Y Y Y-Nₐ-n_{q} 3′ |
| antisense: | 3′ nₚ′-Nₐ′-X′X′X′-N_{b}-Y′Y′Y′-Nₐ′-n_{q}′ 5′ |

wherein each Nb and Nb′ independently represents an oligonucleotide sequence comprising 1-5 modified nucleotides; or wherein formula (III) is represented by formula (IIId):

| | |
|---|---|
| sense: | 5′ nₚ-Nₐ- X X X-N_{b}-Y Y Y- N_{b}-Z Z Z- |
| | Nₐ-n_{q} 3′ |
| antisense: | 3′ nₚ′-Nₐ′-X′X′X′-N_{b}-Y′Y′Y′-N_{b}-Z′Z′Z′- |
| | Nₐ′-n_{q}′ 5 |

wherein each Nb and Nb′ independently represents an oligonucleotide sequence comprising 1-5 modified nucleotides and each Na and Na′ independently represents an oligonucleotide sequence comprising 2-10 modified nucleotides.

In some embodiments, the modifications on the nucleotides are selected from the group consisting of LNA, CRN, cET, UNA, HNA, CeNA, 2′-methoxyethyl, 2′-O-methyl, 2′-O-alkyl, 2′-O-allyl, 2′-C-allyl, 2′-fluoro, 2′-deoxy, 2′-hydroxyl, and combinations thereof.

In some embodiments, the modifications on the nucleotides are 2′-O-methyl or 2′-fluoro modifications. In some embodiments the ligand is one or more GalNAc derivatives attached through a bivalent or trivalent branched linker. In some embodiments the ligand is depicted in Formula XA, XB, or XC, or another GalNAc structure described herein.

In some embodiments the ligand is attached to the 3′ end of the sense strand. In some embodiments the attachment is as depicted in Formula XD shown below.

In some embodiments, an inhibitory RNA (e.g., siRNA) further comprises at least one phosphorothioate or methylphosphonate internucleotide linkage.

In some embodiments p′>0; or p′=2.

In some embodiments q′=0, p=0, q=0, and p′ overhang nucleotides are complementary to C3 mRNA. In some embodiments q′=0, p=0, q=0, and p′ overhang nucleotides are non-complementary to C3 mRNA.

In some embodiments at least one np′ is linked to a neighboring nucleotide via a phosphorothioate linkage

In some embodiments the ligand targets the nucleic acid molecule to hepatocytes. For example, in some embodiments the ligand binds to hepatocyte-specific asialoglycoprotein receptor (ASGPR), e.g., the ligand comprises a galactose derivative, e.g., GalNAc.

In some embodiments an inhibitory RNA (e.g., siRNA) is conjugated to or otherwise physically associated with one or more moieties that modulate, e.g., enhance, the activity, stability, cellular distribution, and/or cellular uptake of the inhibitory RNA (e.g., siRNA) and/or alter one or more physical properties of the inhibitory RNA (e.g., siRNA), such as charge or solubility. In some embodiments, a moiety may comprise an antibody or ligand. A ligand may be a carbohydrate, lectin, protein, glycoprotein, lipid, cholesterol, steroid, bile acid, nucleic acid hormone, growth factor, or receptor. In some embodiments a biologically inactive variant of a naturally occurring hormone, growth factor, or other ligand may be used. In some embodiments, the moiety comprises a targeting moiety that targets the inhibitory RNA (e.g., siRNA) to a specified cell type, e.g., a hepatocyte. In some embodiments a targeting moiety binds to hepatocyte-specific asialoglycoprotein receptor (ASGPR).

In some embodiments a moiety is attached to an inhibitory RNA (e.g., siRNA) via a reversible linkage. A “reversible linkage” is a linkage that comprises a reversible bond. A “reversible bond” (also referred to as a labile bond or cleavable bond) is a covalent bond other than a covalent bond to a hydrogen atom that is capable of being selectively broken or cleaved more rapidly than other bonds in a molecule under selected conditions, the bond is capable of being selectively broken or cleaved under conditions that substantially will not break or cleave other covalent bonds in the same molecule. Cleavage or lability of a bond may be described in terms of the half-life (t_{1/2}) of bond cleavage (the time required for half of the bonds to cleave). Unless otherwise indicated, a reversible bond of interest herein is a “physiologically reversible bond”, by which is meant that the bond is cleavable under conditions normally encountered or analogous to those encountered within a mammalian body. A physiologically reversible linkage is a linkage that comprises at least one physiologically reversible bond. In some embodiments, a physiologically reversible bond is reversible under mammalian intracellular conditions, which include chemical conditions such as pH, temperature, oxidative or reductive conditions or agents, and salt concentration found in or analogous to those found in mammalian cells. Mammalian intracellular conditions also include the presence of enzymatic activity normally present in a mammalian cell, such as from proteolytic or hydrolytic enzymes. Enzymatically labile bonds are cleaved by enzymes in the body, e.g., intracellular enzymes. pH labile bonds are cleaved at a pH less than or equal to 7.0. Examples of reversible bonds and linkages and their use to conjugate moieties to an inhibitory RNA (e.g., siRNA) are described in, e.g., US Pat. App. Pub. Nos. 20130281685 and 20150273081.

In some embodiments, a moiety comprises a protein transduction domain (PTD). Protein transduction domains are polypeptides or portions thereof that facilitate uptake of heterologous molecules attached to the domain (such heterologous molecules may be referred to as “cargo”). A protein transduction domain that is a peptide may be referred to as a cell penetrating peptide (CPP)). A number of protein transduction domains/peptides are known in the art. PTDs include a variety of naturally occurring or synthetic arginine-rich peptides. An arginine-rich peptide is a peptide that contains at least 30% arginine residues, e.g., at least 40%, 50%, 60%, or more. Examples of PTDs include TAT (at least amino acids 49-56), Antennopedia homeodomain, HSV VP22, and polyarginine. Such peptides may be a cationic, hydrophobic, or amphipathic peptide and may include non-standard amino acids and/or various modifications or variations such as use of circularly permuted, inverso, retro, retro-inverso, or peptidomimetic versions. The attachment of a PTD and a cargo may be covalent or noncovalent.

Exemplary PTDs that may be used are described in U.S. Pat. App. Pub. Nos. 20090093026, 20090093425, 20120142763, 20150238516, and 20160215022. A PTD may comprise two or more PTDs (e.g., between 2 and 10 PTDs), which may be the same or different. PTDs may be directly linked to one another or may be separated by a linking portion that may comprise one or more amino acids and/or one or more non-amino acid moieties, such as an alkyl chain or oligoethylene glycol moiety.

In some embodiments, an inhibitory RNA (e.g., siRNA) comprises or is physically associated with an anionic charge neutralizing moiety. An anionic charge neutralizing moiety refers to a molecule or chemical group that can reduce the overall net anionic charge of a nucleic acid with which it is physically associated. One or more anionic charge neutralizing molecules or groups can be associated with a nucleic acid wherein each independently contributes to a reduction of the anionic charge and or increase in cationic charge. By charge neutralized is meant that the anionic charge of the nucleic acid is reduced, neutralized or more cationic than the same nucleic acid in the absence of an anionic charge neutralizing molecule or group. Phosphodiester and/or phosphothioate protecting groups are examples of anionic charge neutralizing groups. In some embodiments, an inhibitory RNA (e.g., siRNA) comprises a protecting group at one or more positions that reduces the net anionic charge of a backbone that contains negatively charged groups (e.g., a phosphodiester or phosphorothioate backbone). In some embodiments, the negatively charged phosphodiester backbone is neutralized by synthesis with bioreversible phosphotriester protecting groups that are converted into charged phosphodiester bonds inside cells by the action of cytoplasmic thioesterases, resulting in an agent that is biologically active for inhibiting expression, e.g., an inhibitory RNA (e.g., siRNA) that can mediate RNAi. Such agents, which are sometimes referred to as short interfering ribonucleic neutrals (siRNNs) can therefore serve as siRNA prodrugs. It should be understood that the backbone need not be completely neutralized (i.e., uncharged). In some embodiments, between 5% and 100% of the phosphate groups are protected, e.g., 25%-50% or 50% to 75% or 75% to 100%. In certain embodiments at least 5, 6, 7, 8, 9, or 10 of the phosphate groups on one or both strands are protected. Examples of useful phosphodiester and/or phosphothioate protecting groups, methods of making them, and their use in nucleic acids (e.g., to generate RNAi agent prodrugs) are described in US Pat. App. Pub. Nos. 20110294869, 20090093425, 20120142763, and 20150238516. In various embodiments a siRNA may comprise any of the modifications described herein. For example, in some embodiments it may contain 2′ sugar modifications (e.g., 2′-F, 2′-O-Me). Furthermore, a siRNN may have any of the configurations or modification patterns described herein.

In some embodiments a moiety attached to an inhibitory RNA (e.g., siRNA) comprises a carbohydrate. Representative carbohydrates include mono-, di-, tri- and oligosaccharides containing from about 4, 5, 6, 7, 8, or 9 monosaccharide units. In certain embodiments the carbohydrate comprises galactose or a galactose derivative such as galactosamine, N-formyl-galactosamine, N-acetylgalactosamine, N-propionyl-galactosamine, N-n-butanoyl-galactosamine, and N-iso-butanoylgalactos-amine. In certain embodiments of particular interest the galactose derivative comprises N-acetylgalactosamine (GalNAc). In certain embodiments, the moiety comprises multiple instances of the galactose or galactose derivative, e.g., multiple N-acetylgalactosamine moieties, e.g., 3 GalNAc moieties. As used herein, the term “galactose derivative” includes both galactose and derivatives of galactose having affinity for the asialoglycoprotein receptor equal to or greater than that of galactose. The term “galactose cluster” refers to a structure comprising at least 2 galactose derivatives that are physically associated with each other, typically by being covalently attached to another moiety. In some embodiments, a galactose cluster has 2-10 (e.g., 6), or 2-4 (e.g., 3) terminal galactose derivatives. A terminal galactose derivative may be attached to another moiety through the C-1 carbon of the galactose derivative. In some embodiments two or more, e.g., three, galactose derivatives are attached to a moiety that serves as a branch point and that can be attached to an inhibitory RNA (e.g., siRNA). In some embodiments, a galactose derivative is linked to the moiety that serves as a branch point via a linker or spacer. In some embodiments, the moiety that serves as a branch point may be attached to an inhibitory RNA (e.g., siRNA) via a linker or spacer. For example, in some embodiments, a galactose derivative is attached to a branch point via a linker or spacer that comprises an amide, carbonyl, alkyl, oligoethylene glycol moiety, or combination thereof. In some embodiments the linkers or spacers attached to each galactose derivative are the same. In some embodiments, a galactose cluster has three terminal galactosamines or galactosamine derivatives (e.g., GalNAc) each having affinity for the asialoglycoprotein receptor. A structure in which 3 terminal GalNAc moieties are attached (e.g., through the C-1 carbons of the saccharides) to a moiety that serves as branch point may be referred to as tri-antennary N-acetylgalactosamine (GalNAc₃). In some embodiments, one or more monomeric units comprising a galactose derivative may be incorporated site-specifically into an inhibitory RNA (e.g., siRNA). Such galactose derivative-containing monomeric units may comprise a galactose derivative, e.g., GalNAc, attached to a nucleoside or to a non-nucleoside moiety. In some embodiments, at least 3 nucleoside-GalNAc monomers or at least 3 non-nucleoside-GalNAc monomers are incorporated site-specifically into an inhibitory RNA (e.g., siRNA). In some embodiments, such incorporation may occur during solid-phase synthesis using phosphoramidite chemistry or via postsynthetic conjugation. In some embodiments, the galactose derivative-containing monomeric units are joined via phosphodiester bonds to each other and/or to nucleosides of the inhibitory RNA (e.g., siRNA) that do not have a galactose derivative attached. In some embodiments 2, 3, or more galactose derivative-containing monomeric units are arranged consecutively, i.e., without any intervening units that lack a galactose derivative. In some embodiments a carbohydrate, e.g., a galactose cluster, e.g., tri-antennary N-acetylgalactosamine or two or more GalNAc-containing monomeric units, is present at the end of a strand, e.g., at the 3′ end of the sense strand or at the 5′ end of an antisense strand. Exemplary carbohydrates (e.g., galactose clusters), galactose derivative-containing monomeric units, carbohydrate-modified inhibitory RNAs, and methods of manufacture and use thereof are described in US Pat. App. Pub. Nos. 20090203135, 20090239814, 20110207799, 20120157509, 20150247143, US Pub. '124; Nair, J K, et al., J. Am. Chem. Soc. 136, 16958-16961 (2014); Matsuda, S., et al., ACS Chem. Biol. 10, 1181-1187 (2015); Rajeev, K., et al., ChemBioChem 16, 903-908 (2015); Migawa, MT., et al., Bioorg Med Chem Lett. 26 (9): 2194-7 (2016); Prakash, T P, et al., J Med Chem. 59 (6): 2718-33 (2016). Exemplary galactose clusters are depicted below.

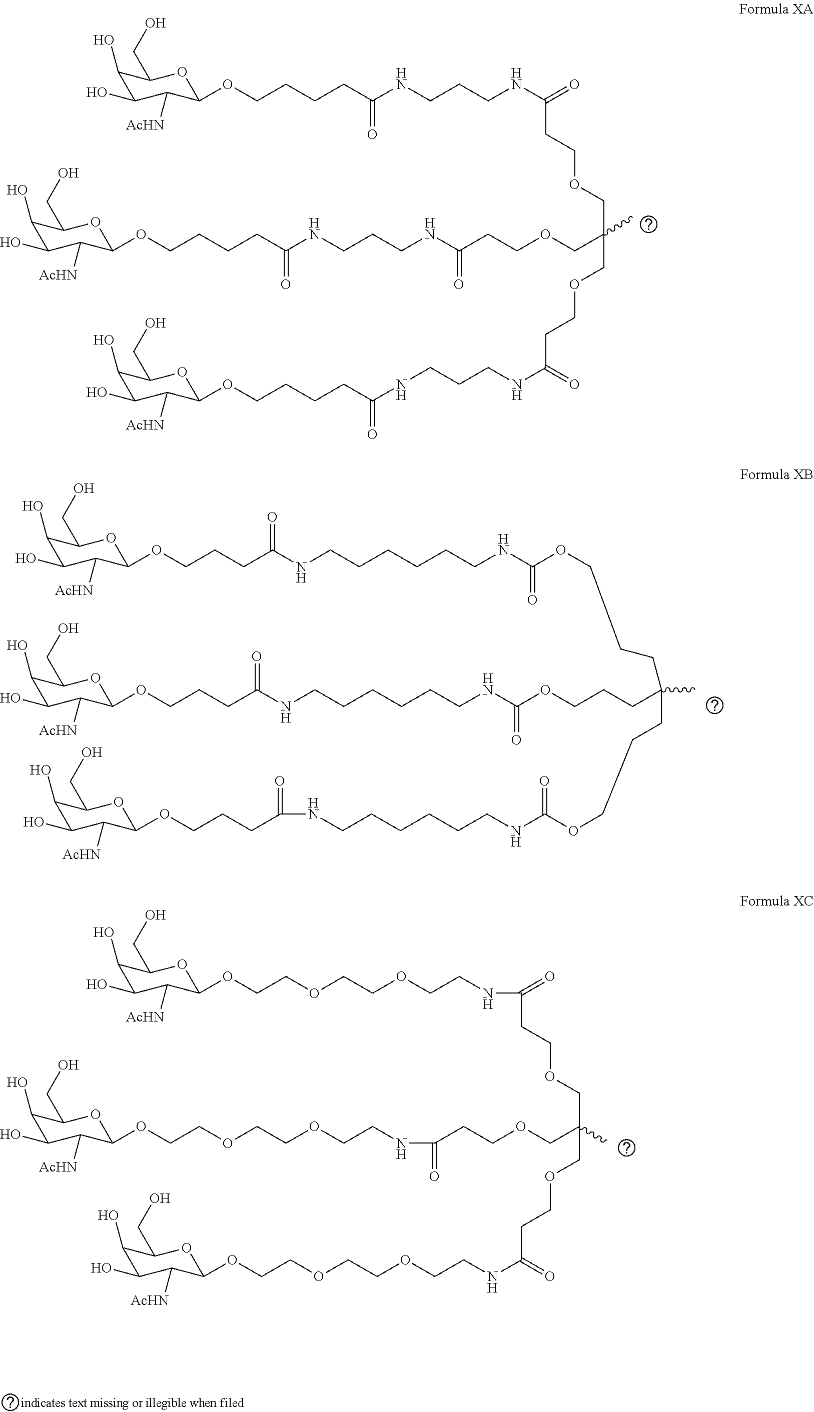

Additional GalNAc structures are depicted below (and can be synthesized as described in Sharma et al., Bioconjug. Chem. 29:2478-2488 (2018)):

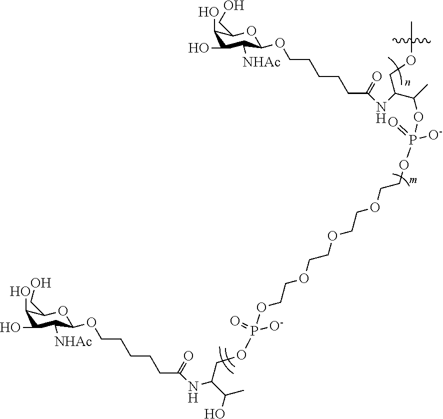

In some embodiments, m=0 and n=2. In some embodiments, m=1 and n=1. In some embodiments, m=1 and n=2. In some embodiments, m=1 and n=3.

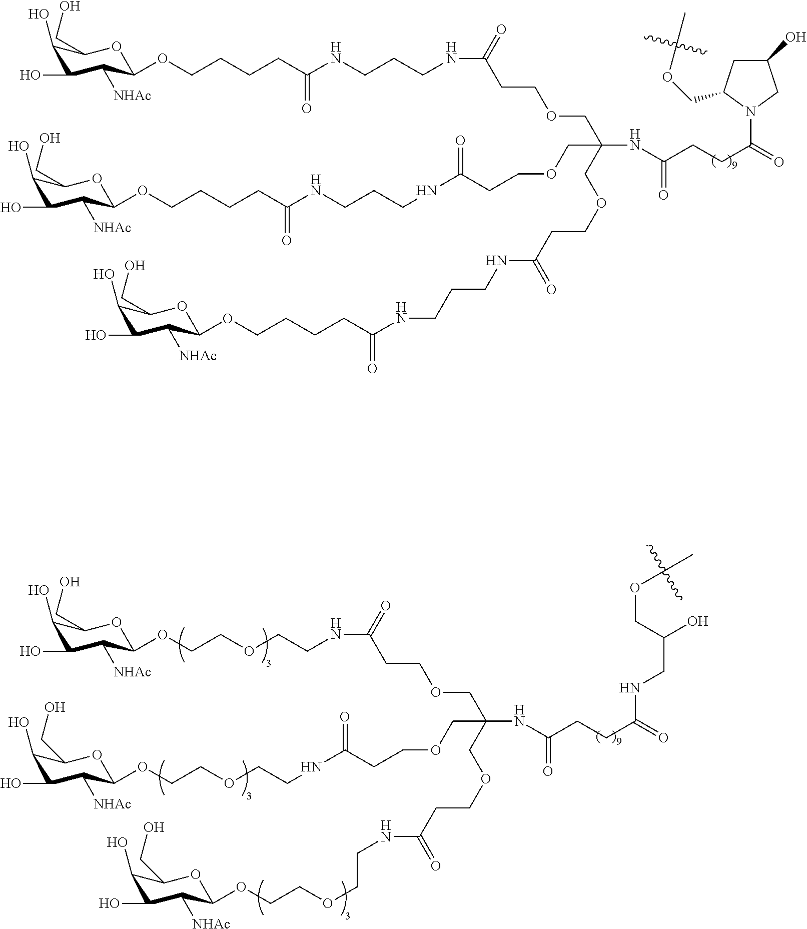

One of ordinary skill in the art appreciates that the structure of the linking moieties that connect each GalNAc to the branch point may vary. In some embodiments, an inhibitory RNA (e.g., siRNA) is conjugated to a GalNAc depicted below:

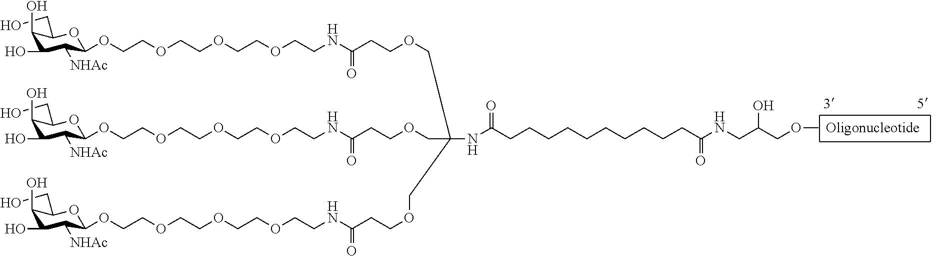

Formula XD (where said GalNAc can be conjugated at either strand (e.g., the sense strand) at the 3′ or 5′ end)

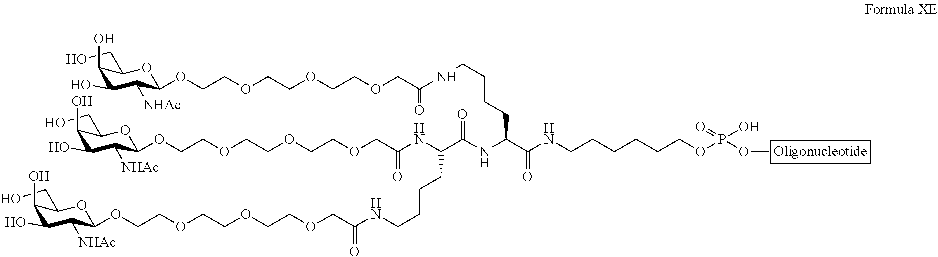

In some embodiments, an inhibitory RNA (e.g., siRNA, such as any of siRNAs: 1-57 listed in Tables 10 and 15, e.g., siRNAs 22, 32, and 53) is conjugated to a GalNAc ligand (e.g., a GalNAc of Formula XD or XE).

In some embodiments a GalNAc ligand (e.g., as shown in Formular XD or XE) is conjugated to the 3′-terminal nucleotide of the sense or antisense strand of an siRNA (e.g., any one of siRNAs: 1-57 e.g., siRNAs 22, 32, and 53). In some embodiments, a GalNAc ligand (e.g., as shown in Formula XD or XE) is conjugated to the 3′ position of the sugar on the 3′-terminal nucleotide of the sense or antisense strand of an siRNA.

In some embodiments a GalNAc ligand (e.g., as shown in Formular XD or XE) is conjugated to the 5′-terminal nucleotide of the sense or antisense strand of an siRNA (e.g., any one of siRNAs: 1-57 e.g., siRNAs 22, 32, and 53). In some embodiments a GalNAc ligand (e.g., as shown in Formula XD or XE) is conjugated to the 5′ position of the 5′-terminal nucleotide of the sense or antisense strand of an siRNA.

In some embodiments, when an inhibitory RNA (e.g., siRNAs of SEQ ID NOs: 1-57 e.g., siRNAs 22, 32, and 53) is conjugated to a ligand (e.g., a GalNAc ligand), the inhibitory RNA may not include a modification (e.g., a phosphorothioate bond “PS”) to the nucleotide(s) that is/are conjugated to the ligand.

In some embodiments, an siRNA (such as any of siRNAs: 1-57 e.g., siRNAs 22, 32, and 53) is conjugated to a GalNAc ligand (e.g., as shown in Formula XD or XE) at one terminus of either the sense or antisense strand. In some embodiments, the other three termini that are not conjugated to the GalNAc ligand contain a modification such as a phosphorothioate bond (“PS”). In some embodiments, a modification includes a PS bond between the two, three, or four 5′ or 3′-most nucleotides. In some embodiments, the terminus that is conjugated to a GalNAc ligand does not contain a phosphorothioate bond between the two, three or four 5′ or 3′-most nucleotides.

In some embodiments, an siRNA described herein can be conjugated to a galactose structure shown below:

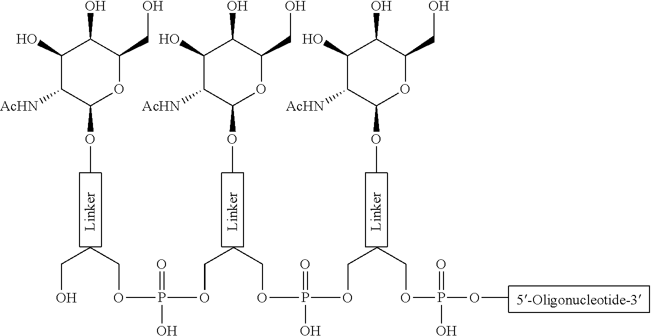

In some embodiments, the linker comprises an amide, carbonyl, alkyl, oligoethylene glycol moiety, or combination thereof.

In some embodiments, an siRNA described herein can be conjugated to a galactose structure shown below:

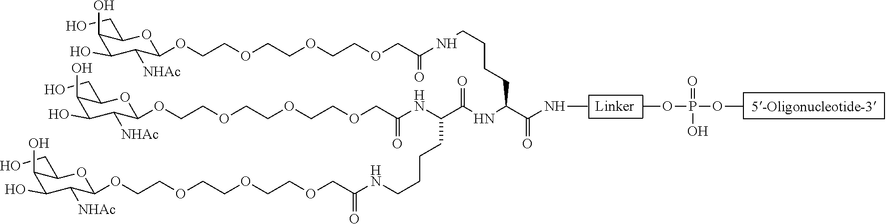

In some embodiments, the linker comprises an amide, carbonyl, alkyl, oligoethylene glycol moiety, or combination thereof.

Methods of synthesizing GalNAc ligands, methods of conjugating GalNAc ligands to inhibitory RNAs, and additional GalNAc ligands are known in the art and include, for example, those described in WO 2017/021385, WO 2017/178656, WO 2018/215391, WO 2019/145543, WO 2017/084987, WO 2017/055423, and WO 2012/083046, which are herein incorporated by reference in their entirety.

In some embodiments an inhibitory RNA (e.g., siRNA) is conjugated to a ligand as depicted below.

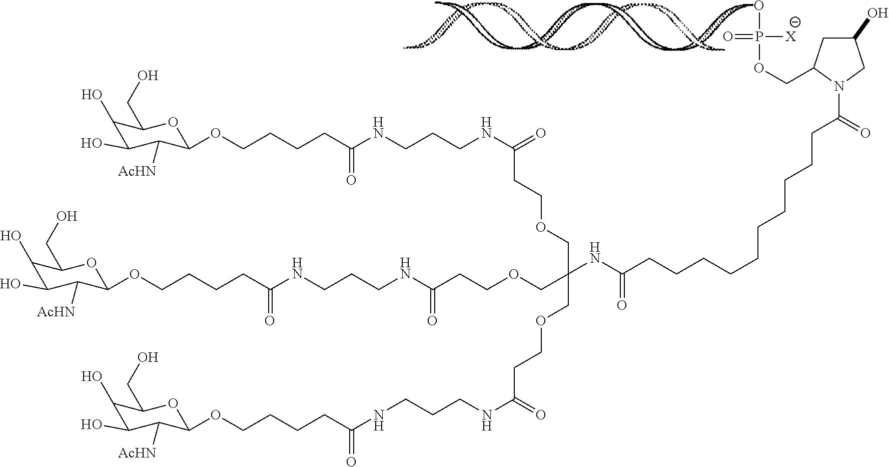

and, wherein X is O or S. In most embodiments, X is O. One of ordinary skill in the art will appreciate that the structure of the linking moiety that connects the galactose cluster to the phosphate group may vary.

In certain embodiments the moiety comprises a lipophilic moiety. In some embodiments the lipophilic moiety comprises a tocopherol, e.g., alpha-tocopherol. In some embodiments the lipophilic moiety comprises cholesterol. In some embodiments the lipophilic compound comprises an alkyl or heteroalkyl group. In some embodiments the lipophilic compound comprises palmitoyl, hexadec-8-enoyl, oleyl, (9E,12E)-octadeca-9,12-dienoyl, dioctanoyl, or C16-C20 acyl. In some embodiments the lipophilic moiety comprises at least 16 carbon atoms. In some embodiments the lipophilic moiety comprises —(CH)ₙ—NH—(C═O)—(CH)ₘ—CH₃. In some embodiments n and m are each independently between 1 and 20. In some embodiments n+m is at least 10, 12, 14, or 16. In some embodiments the lipophilic moiety is as shown below and/or is attached to a sugar moiety as shown below.

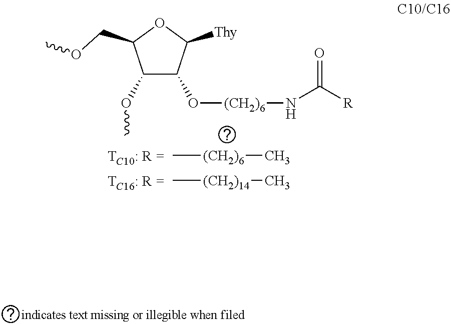

In general, a moiety may be attached at a terminus or internal subunit of an inhibitory RNA (e.g., siRNA). In some embodiments a moiety is attached to a modified subunit of the inhibitory RNA (e.g., siRNA). Those of ordinary skill in the art are aware of suitable methods to manufacture nucleic acids having moieties conjugated thereto. A nucleic acid strand comprising a modified nucleotide comprising a reactive functional group may be reacted with a moiety comprising a second reactive functional group, wherein the first and second reactive functional groups are capable of reacting with one another under conditions compatible with maintaining the structure of the nucleic acid strand. In some embodiments a moiety may be attached to a sense strand or an antisense strand prior to hybridization of the strand with the complementary antisense or sense strand, respectively. In some embodiments strands may be hybridized to form a duplex prior to incorporation of the moiety. In general, various methods of conjugation described herein may be used. See, e.g., Hermanson, G., Bioconjugate Techniques, 2nd ed., Academic Press, San Diego, 2008.

In some embodiments, an inhibitory RNA (e.g., siRNA) is a chimeric siRNA. “Chimeric” siRNAs as used herein, are siRNAs that contain two or more chemically distinct regions, each made up of at least one monomer unit, wherein the regions confer distinct properties on the compound. In some embodiments, at least one region is modified so as to confer upon the siRNA increased resistance to nuclease degradation, increased cellular uptake, and/or increased binding affinity for the target nucleic acid, and at least one additional region of the siRNA can serve as a substrate for enzymes (e.g., RNase H) capable of cleaving RNA: DNA or RNA: RNA hybrids. In some embodiments, at least one region of the siRNA can serve as a substrate for enzymes (e.g., RNase H) capable of cleaving RNA: DNA or RNA: RNA hybrids, and at least one region can inhibit translation by steric blocking.

In some embodiments, an inhibitory RNA (e.g., siRNA) described herein can be introduced to a target cell as an annealed duplex siRNA. In some embodiments, an inhibitory RNA (e.g., siRNA) described herein is introduced to a target cell as single stranded sense and antisense nucleic acid sequences that, once within the target cell, anneal to form an inhibitory RNA (e.g., siRNA) duplex. In some embodiments, the sense and antisense strands of the inhibitory RNA (e.g., siRNA) can be encoded by an expression vector (such as an expression vector described herein) that is introduced to the target cell. Upon expression within the target cell, the transcribed sense and antisense strands can anneal to form the inhibitory RNA (e.g., siRNA).

An inhibitory RNA (e.g., an siRNA or miRNA, or a vector comprising a nucleotide sequence encoding an siRNA or miRNA) described herein can be synthesized by standard methods known in the art, e.g., by use of an automated synthesizer. RNAs produced by such methodologies tend to be highly pure and to anneal efficiently to form inhibitory RNA (e.g., siRNA) duplexes. Following chemical synthesis, single stranded RNA molecules can be deprotected, annealed to form siRNAs, and purified (e.g., by gel electrophoresis or HPLC). Alternatively, standard procedures can be used for in vitro transcription of RNA from DNA templates, e.g., carrying one or more RNA polymerase promoter sequences (e.g., T7 or SP6 RNA polymerase promoter sequences). Protocols for preparation of siRNAs using T7 RNA polymerase are known in the art (see, e.g., Donze and Picard, Nucleic Acids Res. 2002; 30: e46; and Yu et al., Proc. Natl. Acad. Sci. USA 2002; 99:6047-6052). The sense and antisense transcripts can be synthesized in two independent reactions and annealed later, or they can be synthesized simultaneously in a single reaction.

An inhibitory RNA (e.g., an siRNA or miRNA) can also be formed within a cell by transcription of RNA from an expression construct introduced into the cell (see, e.g., Yu et al., Proc. Natl. Acad. Sci. USA 2002; 99:6047-6052). An expression construct for in vivo production of inhibitory RNA (e.g., siRNA) molecules can include one or more siRNA encoding sequences operably linked to elements necessary for the proper transcription of the siRNA encoding sequence(s), including, e.g., promoter elements and transcription termination signals. Preferred promoters for use in such expression constructs include the polymerase-III HI-RNA promoter (see, e.g., Brummelkamp et al., Science 2002; 296:550-553) and the U6 polymerase-III promoter (see, e.g., Sui et al., Proc. Natl. Acad. Sci. USA 2002; Paul et al., Nature Biotechnol. 2002; 20:505-508; and Yu et al., Proc. Natl. Acad. Sci. USA 2002; 99:6047-6052). An siRNA expression construct can further comprise one or more vector sequences that facilitate the cloning of the expression construct. Standard vectors that can be used include, e.g., pSilencer 2.0-U6 vector (Ambion Inc., Austin, Tex.).

### III. EXPRESSION VECTORS

In some embodiments, an inhibitory RNA described herein is delivered to a subject (e.g., to a cell of a subject, e.g., a liver cell of a subject) using an expression vector. Many forms of vectors can be used to deliver an inhibitory RNA described herein. Non-limiting examples of expression vectors include viral vectors (e.g., vectors suitable for gene therapy), plasmid vectors, bacteriophage vectors, cosmids, phagemids, artificial chromosomes, and the like.

In some embodiments, a nucleotide sequence encoding an inhibitory RNA described herein is integrated into a viral vector. Non-limiting examples of viral vectors include: retrovirus (e.g., Moloney murine leukemia virus (MMLV), Harvey murine sarcoma virus, murine mammary tumor virus, Rous sarcoma virus), adenovirus, adeno-associated virus, SV40-type virus, polyomavirus, Epstein-Barr virus, papilloma virus, herpes virus, vaccinia virus, and polio virus.

In vivo, many complement proteins, including C3, are synthesized primarily in the liver. As such, in some embodiments, hepatocytes are targeted for delivery of an inhibitory RNA described herein. Several classes of viral vectors have been shown competent for liver-targeted delivery of a gene therapy construct, including retroviral vectors (see, e.g., Axelrod et al., PNAS 87:5173-5177 (1990); Kay et al., Hum. Gene Ther. 3:641-647 (1992); Van den Driessche et al., PNAS 96:10379-10384 (1999); Xu et al., ASAIO J. 49:407-416 (2003); and Xu et al., PNAS 102:6080-6085 (2005)), lentiviral vectors (see, e.g., Mckay et al., Curr. Pharm. Des. 17:2528-2541 (2011); Brown et al., Blood 109:2797-2805 (2007); and Matrai et al., Hepatology 53:1696-1707 (2011)), adeno-associated viral (AAV) vectors (see, e.g., Herzog et al., Blood 91:4600-4607 (1998)), and adenoviral vectors (see, e.g., Brown et al., Blood 103:804-810 (2004) and Ehrhardt et al., Blood 99:3923-3930 (2002)).

Retroviruses are enveloped viruses that belong to the viral family Retroviridae. Once in a host's cell, the virus replicates by using a viral reverse transcriptase enzyme to transcribe its RNA into DNA. The retroviral DNA replicates as part of the host genome, and is referred to as a provirus. A selected nucleic acid can be inserted into a vector and packaged in retroviral particles using techniques known in the art. Protocols for the production of replication-deficient retroviruses are known in the art (see, e.g., Kriegler, M., Gene Transfer and Expression, A Laboratory Manual, W.H. Freeman Co., New York (1990) and Murry, E. J., Methods in Molecular Biology, Vol. 7, Humana Press, Inc., Cliffton, N.J. (1991)). The recombinant virus can then be isolated and delivered to cells of the subject either in vivo or ex vivo. A number of retroviral systems are known in the art, for example See U.S. Pat. Nos. 5,994,136, 6, 165,782, and 6,428,953. Retroviruses include the genus of Alpharetrovirus (e.g., avian leukosis virus), the genus of Betaretrovirus; (e.g., mouse mammary tumor virus) the genus of Deltaretrovirus (e.g., bovine leukemia virus and human T-lymphotropic virus), the genus of Epsilonretrovirus (e.g., Walleye dermal sarcoma virus), and the genus of Lentivirus.

In some embodiments, the retrovirus is a lentivirus of the Retroviridae family. In some examples, the lentivirus is, but is not limited to, human immunodeficiency viruses (HIV-1 and HIV-2), simian immunodeficiency virus (S1V), feline immunodeficiency virus (FIV), equine infections anemia (EIA), and visna virus.

In some embodiments, the vector is an adenovirus vector. Adenoviruses are a large family of viruses containing double stranded DNA. They replicate within the nucleus of a host cell, using the host's cell machinery to synthesize viral RNA, DNA and proteins.

In some embodiments, the viral vector is an adeno-associated virus (AAV) vector. AAV systems are generally well known in the art (see, e.g., Kelleher and Vos, Biotechniques, 17 (6): 1110-17 (1994); Cotten et al., P.N.A.S. U.S.A., 89 (13): 6094-98 (1992); Curiel, Nat Immun, 13 (2-3): 141-64 (1994); Muzyczka, Curr Top Microbiol Immunol, 158:97-129 (1992); and Asokan A, et al., Mol. Ther., 20 (4): 699-708 (2012)). Methods for generating and using recombinant AAV (rAAV) vectors are described, for example, in U.S. Pat. Nos. 5,139,941 and 4,797,368.

Several AAV serotypes have been characterized, including AAV1, AAV2, AAV3 (e.g., AAV3B), AAV4, AAV5, AAV6, AAV7, AAV8, AAV9, AAV10, and AAV11, as well as variants thereof. Generally, any AAV serotype may be used to deliver an inhibitory RNA described herein. However, the serotypes have different tropisms, e.g., they preferentially infect different tissues. In some embodiments, an AAV serotype is selected based on a liver tropism, found in at least serotypes AAV2, AAV3 (e.g., AAV3B), AAV5, AAV7, AAV8, and AAV9 (see, e.g., Shaoyong et al., Mol. Ther. 23:1867-1876 (2015)).

The AAV sequences of a rAAV vector typically comprise the cis-acting 5′ and 3′ inverted terminal repeat sequences (See, e.g., B. J. Carter, in “Handbook of Parvoviruses”, ed., P. Tijsser, CRC Press, pp. 155 168 (1990)). The ITR sequences are about 145 bp in length. In some embodiments, substantially the entire sequences encoding the ITRs are used in an rAAV vector, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J Virol., 70:520 532 (1996)). An example of an rAAV vector of the present disclosure is a “cis-acting” plasmid containing the transgene (e.g., nucleic acid encoding an inhibitory RNA described herein), in which the selected transgene sequence and associated regulatory elements are flanked by the 5′ and 3′ AAV ITR sequences. The AAV ITR sequences may be obtained from any known AAV, including presently identified mammalian AAV types.

In addition to the major elements identified above for an rAAV vector, the vector can also include conventional control elements operably linked to the transgene in a manner that permits its transcription, translation and/or expression in a cell transfected with the vector or infected with the virus produced by the disclosure. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A number of expression control sequences, including promoters that are native, constitutive, inducible and/or tissue-specific, are known in the art and may be included in a vector described herein. In some embodiments, operably linked coding sequences yield a functional RNA (e.g., miRNA or siRNA).

Examples of constitutive promoters include, without limitation, the retroviral Rous sarcoma virus (RSV) LTR promoter (optionally with the RSV enhancer), the cytomegalovirus (CMV) promoter (optionally with the CMV enhancer), the SV40 promoter, and the dihydrofolate reductase promoter. Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system, the ecdysone insect promoter, the tetracycline-repressible system, the tetracycline-inducible system, the RU486-inducible system and the rapamycin-inducible system. Still other types of inducible promoters which may be useful in this context are those which are regulated by a specific physiological state, e.g., temperature, acute phase, a particular differentiation state of the cell, or in replicating cells only. In another embodiment, a native promoter, or fragment thereof, for a transgene will be used. In a further embodiment, other native expression control elements, such as enhancer elements, polyadenylation sites or Kozak consensus sequences may also be used to mimic the native expression.

In some embodiments, regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. In some embodiments, the promoter is a chicken β-actin promoter, a pol II promoter, or a pol III promoter.

In some embodiments, an rAAV is designed for expressing an inhibitory RNA described herein in hepatocytes, and an rAAV includes one or more liver-specific regulatory elements, which substantially limit expression of the inhibitory RNA to hepatic cells. Generally, liver-specific regulatory elements can be derived from any gene known to be exclusively expressed in the liver. WO 2009/130208 identifies several genes expressed in a liver-specific fashion, including serpin peptidase inhibitor, clade A member 1, also known as α-antitrypsin (SERPINA1; GeneID 5265), apolipoprotein C-I (APOC1; GeneID 341), apolipoprotein C-IV (APOC4; GeneID 346), apolipoprotein H (APOH; GeneID 350), transthyretin (TTR; GeneID 7276), albumin (ALB; GeneID 213), aldolase B (ALDOB; GeneID 229), cytochrome P450, family 2, subfamily E, polypeptide 1 (CYP2E1; GeneID 1571), fibrinogen alpha chain (FGA; GeneID 2243), transferrin (TF; GeneID 7018), and haptoglobin related protein (HPR; GeneID 3250). In some embodiments, a viral vector described herein includes a liver-specific regulatory element derived from the genomic loci of one or more of these proteins. In some embodiments, a promoter may be the liver-specific promoter thyroxin binding globulin (TBG). Alternatively, other liver-specific promoters may be used (see, e.g., The Liver Specific Gene Promoter Database, Cold Spring Harbor, http://rulai.cshl.edu/LSPD/, such as, e.g., alpha 1 anti-trypsin (A1AT); human albumin (Miyatake et al., J. Virol. 71:5124 32 (1997)); humAlb; hepatitis B virus core promoter (Sandig et al., Gene Ther. 3:1002 9 (1996)); or LSP1. Additional vectors and regulatory elements are described in, e.g., Baruteau et al., J. Inherit. Metab. Dis. 40:497-517 (2017)).

In some embodiments, a viral vector (e.g., an rAAV vector) comprises a DNA sequence encoding an inhibitory RNA described herein.

In some embodiments, a vector (e.g., a viral vector) comprises one or more nucleotide sequences that encode more than one (e.g., 2, 3, 4, 5, or more) miRNAs or siRNAs comprising a nucleic acid strand that is complementary to a target portion of a C3 transcript, e.g., C3 mRNA (SEQ ID NO:75). In some embodiments, a vector comprises multiple nucleotide sequences, where each nucleotide sequence encodes a different inhibitory RNA described herein. In some embodiments, a vector comprises multiple nucleotide sequences encoding at least 2 different inhibitory RNAs, wherein at least two of the nucleotide sequences are copies of the same inhibitory RNA described herein.

In some embodiments, in addition to one or more sequences encoding one or more inhibitory RNAs described herein, a vector (e.g., a viral vector) comprises one or more additional nucleotide sequences encoding one or more C3 inhibitors, e.g., a C3 inhibitor described herein. For example, a C3 inhibitor can be a polypeptide inhibitor and/or a nucleic acid aptamer (see, e.g., U.S. Publ. No. 20030191084). Exemplary polypeptide inhibitors include a compstatin analog (e.g., a compstatin analog described herein that includes genetically encodable amino acids), an anti-C3 or anti-C3b antibody (e.g., scFv or single domain antibody, e.g., a nanobody), an enzyme that degrades C3 or C3b (see, e.g., U.S. Pat. No. 6,676,943), or a mammalian complement regulatory protein (e.g., CR1, DAF, MCP, CFH, CFI, C1 inhibitor (C1-INH), a soluble form of complement receptor 1 (sCR1), TP10 or TP20 (Avant Therapeutics), or portion thereof. Additional polypeptide inhibitors include mini-factor H (see, e.g., U.S. Publ. No. 20150110766), Efb protein or complement inhibitor (SCIN) protein from Staphylococcus aureus, or a variant or derivative or mimetic thereof (see, e.g., U.S. Publ. 20140371133).

In some embodiments, a polypeptide inhibitor is linked to a secretion signal sequence for secretion of the expressed polypeptide inhibitor from a host cell.

### IV. PRODUCTION OF EXPRESSION VECTORS

Methods for obtaining expression vectors, e.g., rAAVs, are known in the art. Typically, the methods involve culturing a host cell which contains a nucleic acid sequence encoding an AAV capsid protein or fragment thereof; a functional rep gene; a recombinant AAV vector composed of AAV inverted terminal repeats (ITRs) and a transgene; and/or sufficient helper functions to permit packaging of the recombinant AAV vector into the AAV capsid proteins.

The components to be cultured in a host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell that has been engineered to contain one or more of the required components using methods known to those of skill in the art. In some embodiments, such a stable host cell contains the required component(s) under the control of an inducible promoter. In other embodiments, the required component(s) may be under the control of a constitutive promoter. In other embodiments, a selected stable host cell may contain selected component(s) under the control of a constitutive promoter and other selected component(s) under the control of one or more inducible promoters. For example, a stable host cell may be generated that is derived from 293 cells (which contain E1 helper functions under the control of a constitutive promoter), but that contain the rep and/or cap proteins under the control of inducible promoters. Other stable host cells may be generated by one of skill in the art using routine methods.

Recombinant AAV vector, rep sequences, cap sequences, and helper functions required for producing an rAAV of the disclosure may be delivered to a packaging host cell using any appropriate genetic element (e.g., vector). A selected genetic element may be delivered by any suitable method known in the art, e.g., to those with skill in nucleic acid manipulation and include genetic engineering, recombinant engineering, and synthetic techniques (see, e.g., Sambrook et al, Molecular Cloning: A Laboratory Manual, Cold Spring Harbor Press, Cold Spring Harbor, N.Y.). Similarly, methods of generating rAAV virions are well known and any suitable method can be used with the present disclosure (see, e.g., K. Fisher et al, J. Virol., 70:520-532 (1993) and U.S. Pat. No. 5,478,745).

In some embodiments, recombinant AAVs may be produced using a triple transfection method (e.g., as described in U.S. Pat. No. 6,001,650). In some embodiments, recombinant AAVs are produced by transfecting a host cell with a recombinant AAV vector (comprising a transgene) to be packaged into AAV particles, an AAV helper function vector, and an accessory function vector. An AAV helper function vector encodes “AAV helper function” sequences (i.e., rep and cap), which function in trans for productive AAV replication and encapsidation. In some embodiments, the AAV helper function vector supports efficient AAV vector production without generating any detectable wild-type AAV virions (i.e., AAV virions containing functional rep and cap genes). Non-limiting examples of vectors suitable for use with the present disclosure include pHLP19 (see, e.g., U.S. Pat. No. 6,001,650) and pRep6cap6 vector (see, e.g., U.S. Pat. No. 6,156,303). An accessory function vector encodes nucleotide sequences for non-AAV derived viral and/or cellular functions upon which AAV is dependent for replication (i.e., “accessory functions”). Accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any known helper viruses such as adenovirus, herpesvirus (other than herpes simplex virus type-1), and vaccinia virus.

In some embodiments, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art (see, e.g., Graham et al. (1973) Virology, 52:456; Sambrook et al. (1989) Molecular Cloning, a laboratory manual, Cold Spring Harbor Laboratories, New York, Davis et al. (1986) Basic Methods in Molecular Biology, Elsevier; and Chu et al. (1981) Gene 13:197). Such techniques can be used to introduce one or more exogenous nucleic acids, such as a nucleotide integration vector and other nucleic acid molecules, into suitable host cells.

In some embodiments, a host cell is a mammalian cell. A host cell may be used as a recipient of an AAV helper construct, an AAV minigene plasmid, an accessory function vector, and/or other transfer DNA associated with the production of recombinant AAVs. The term includes the progeny of an original cell that has been transfected. Thus, a “host cell” as used herein may refer to a cell that has been transfected with an exogenous DNA sequence. It is understood that the progeny of a single parental cell may not necessarily be completely identical in morphology or in genomic or total DNA complement as the original parent, due to natural, accidental, or deliberate mutation.

Additional methods for generating and isolating AAV viral vectors suitable for delivery to a subject are described in, e.g., U.S. Pat. Nos. 7,790,449; 7,282,199; WO 2003/042397; WO 2005/033321, WO 2006/110689; and U.S. Pat. No. 7,588,772. In one system, a producer cell line is transiently transfected with a construct that encodes the transgene flanked by ITRs and a construct(s) that encodes rep and cap. In another system, a packaging cell line that stably supplies rep and cap is transiently transfected with a construct encoding the transgene flanked by ITRs. In each of these systems, AAV virions are produced in response to infection with helper adenovirus or herpesvirus, and rAAVs are separated from contaminating virus. Other systems do not require infection with helper virus to recover the AAV—the helper functions (i.e., adenovirus E1, E2a, VA, and E4 or herpesvirus UL5, UL8, UL52, and UL29, and herpesvirus polymerase) are also supplied, in trans, by the system. In such systems, helper functions can be supplied by transient transfection of the cells with constructs that encode the helper functions, or the cells can be engineered to stably contain genes encoding the helper functions, the expression of which can be controlled at the transcriptional or posttranscriptional level.

In yet another system, the transgene flanked by ITRs and rep/cap genes are introduced into insect host cells by infection with baculovirus-based vectors. Such production systems are known in the art (see generally, e.g., Zhang et al., 2009, Human Gene Therapy 20:922-929). Methods of making and using these and other AAV production systems are also described in U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

The foregoing methods for producing recombinant vectors are not meant to be limiting, and other suitable methods will be apparent to the skilled artisan.

### V. COMPOSITIONS AND ADMINISTRATION

Inhibitory RNAs (e.g., an siRNA or miRNA described herein), or a vector comprising a nucleotide sequence encoding an siRNA or miRNA described herein, can be used to treat a complement-mediated disease or disorder, e.g., subjects suffering from or susceptible to a complement-mediated disease or disorder described herein. The route and/or mode of administration of inhibitory RNAs described herein can vary depending upon the desired results. One with skill in the art, i.e., a physician, is aware that dosage regimens can be adjusted to provide the desired response, e.g., a therapeutic response. Methods of administration include, but are not limited to, intradermal, intramuscular, intraperitoneal, intravenous, subcutaneous, intranasal, epidural, oral, sublingual, intravaginal, transdermal, rectal, by inhalation, or topical. The mode of administration is left to the discretion of the practitioner.

In some embodiments, an inhibitory RNA described herein is administered systemically and is not administered locally (e.g., by suprachoroidal injection, subretinal injection, or intravitreal injection) to the eye. In some embodiments, an inhibitory RNA described herein is administered systemically and no additional complement inhibitors are administered (e.g., systemically to the subject or locally to the eye of the subject). In some embodiments, one or more additional complement inhibitors described herein are administered systemically and are not administered locally (e.g., by suprachoroidal injection, subretinal injection, or intravitreal injection) to the eye. In some embodiments, after systemic administration, an inhibitory RNA described herein does not penetrate or cross Bruch's membrane (e.g., does not substantially penetrate or cross Bruch's membrane). In some embodiments, an inhibitory RNA described herein (or a vector comprising a nucleotide sequence encoding an inhibitory RNA) does not comprise a moiety that targets the inhibitory RNA to an eye, that enhances update into the eye, and/or that increases transport across Bruch's membrane.

In some embodiments, systemic administration of an inhibitory RNA described herein to a subject results in a reduced level of C3 expression or activity (e.g., reduced level of one or more C3 activation products, e.g., C3a, C3b, and/or C3d) in the eye (e.g., vitreous humor, aqueous humor, retina, and/or retinal pigment epithelium of the eye) of the subject, e.g., relative to a control level of C3, C3a, C3b, and/or C3d (e.g., level of C3, C3a, C3b, and/or C3d in the eye (e.g., vitreous humor, aqueous humor, retina, and/or retinal pigment epithelium) of the subject prior to administration of an inhibitory RNA, level of C3, C3a, C3b, and/or C3d in the eye (e.g., vitreous humor, aqueous humor, retina, and/or retinal pigment epithelium) of a subject having a disorder described herein, and/or average level of C3, C3a, C3b, and/or C3d in the eye (e.g., vitreous humor, aqueous humor, retina, and/or retinal pigment epithelium) of a population of subjects having a disorder described herein). In some embodiments, systemic administration of an inhibitory RNA described herein to a subject reduces a measured level of C3 (and/or C3 activation products, e.g., C3a, C3b, and/or C3d) in or on microglia, astrocytes, myeloid cells, vascular cells, drusen or plaques of the eye of the subject, relative to a control level of C3 (and/or C3 activation products, e.g., C3a, C3b, and/or C3d) (e.g., level of C3 (and/or C3 activation products, e.g., C3a, C3b, and/or C3d) in or on microglia, astrocytes, myeloid cells, vascular cells, drusen or plaques of the eye of the subject prior to administration of an inhibitory RNA, level of C3 (and/or C3 activation products, e.g., C3a, C3b, and/or C3d) in or on microglia, astrocytes, myeloid cells, vascular cells, drusen and/or plaques of the eye of a subject having a disorder described herein, and/or average level of C3 (and/or C3 activation products, e.g., C3a, C3b, and/or C3d) in or on microglia, astrocytes, myeloid cells, vascular cells, drusen and/or plaques of the eye of a population of subjects having a disorder described herein). In some embodiments, systemic administration of an inhibitory RNA described herein reduces level of C3 (and/or C3 activation products, e.g., C3a, C3b, and/or C3d) in the eye of the subject (e.g., in the vitreous humor, aqueous humor, retina, and/or retinal pigment epithelium of the eye of the subject; and/or in microglia, astrocytes, myeloid cells, vascular cells, drusen and/or plaques of the eye of the subject) by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, relative to a control level of C3, C3a, C3b, and/or C3d. In some embodiments, level of C3 is C3 protein level. In some embodiments, level of C3 is C3 mRNA level.

The delivery of an inhibitory RNA described herein (e.g., siRNA) to a cell can be achieved in a number of different ways. In vivo delivery may be performed by administering a composition comprising an inhibitory RNA to a subject, e.g., by parenteral administration route, e.g., subcutaneous or intravenous or intramuscular administration.

In some embodiments an inhibitory RNA is associated with a delivery agent. “Delivery agent” refers to a substance or entity that is non-covalently or covalently associated with an inhibitory RNA or is co-administered with an inhibitory RNA and serves one or more functions that increase the stability and/or efficacy of the biologically active agent beyond that which would result if the biologically active agent was delivered (e.g., administered to a subject) in the absence of the delivery agent. For example, a delivery agent may protect an inhibitory RNA from degradation (e.g., in blood), may facilitate entry of an inhibitory RNA into cells or into a cellular compartment of interest (e.g., the cytoplasm), and/or may enhance associations with particular cells containing the molecular target to be modulated. Those of ordinary skill in the art are aware of numerous delivery agents that may be used to deliver inhibitory RNA, e.g., siRNAs. See Kanasty, R., et al. Nat Mater. 12 (11): 967-77 (2013) for review of some of these technologies. In some embodiments, e.g., for administering an inhibitory RNA systemically, the inhibitory RNA may be associated with a delivery agent such as a nanoparticle, a dendrimer, a polymer, liposomes, or a cationic delivery system. Without wishing to be bound by any theory, positively charged cationic delivery systems are believed to facilitate binding of a negatively charged inhibitory RNA and also enhance interactions at the negatively charged cell membrane to permit efficient uptake of an inhibitory RNA by the cell. Lipids (e.g., cationic lipids, or neutral lipids), dendrimers, or polymers may be bound to an inhibitory RNA or may form a vesicle or micelle that encapsulates an inhibitory RNA. Methods for making and administering complexes comprising a cationic agent and an inhibitory RNA are known in the art. In some embodiments it is particularly contemplated to use any of the delivery agents described in US Pub. 20160298124. In some embodiments, an inhibitory RNA forms a complex with cyclodextrin for systemic administration. In some embodiments an inhibitory RNA is administered in association with a lipid or lipid-containing particle. In some embodiments an inhibitory RNA is administered in association with a cationic polymer (which may be a polypeptide or a non-polypeptide polymer), a lipid, a peptide, PEG, cyclodextrin, or combination thereof, which may be in the form of a nanoparticle or microparticle. The lipid or peptide may be cationic. “Nanoparticle” refers to particles with lengths in two or three dimensions greater than 1 nanometer (nm) and smaller than about 150 nm e.g., 20 nm-50 nm or 50 nm-100 nm. “Microparticle” refers to particles with lengths in two or three dimensions greater than 150 nm and smaller than about 1000 nm. A nanoparticle may have a targeting moiety and/or cell-penetrating moiety or membrane active moiety covalently or noncovalently attached thereto. Nanoparticles, such as lipid nanoparticles, are described in, e.g., Tatiparti et al., Nanomaterials 7:77 (2017). Exemplary delivery agents, methods of manufacture and use in the delivery of inhibitory RNAs are described in U.S. Pat. Nos. 7,427,605; 8,158,601; 9,012,498; 9,415,109; 9,062,021; 9,402,816. In some embodiments it is contemplated to use delivery technology known in the art as “Smarticles”. In some embodiments it is contemplated to use delivery technology known in the art as “stable nucleic acid lipid particles” (SNALPs), wherein the nucleic acid to be delivered is encapsulated in a lipid bilayer containing a mixture of cationic and fusogenic lipids coated with also coated with a diffusible polyethylene glycol-lipid (PEG-lipid) conjugate that provides a neutral, hydrophilic exterior.

In some embodiments a delivery agent comprises one or more amino alcohol cationic lipids, such as those described in U.S. Pat. No. 9,044,512.

In some embodiments, a delivery agent comprises one or more amino acid lipids. Amino acid lipids are molecules containing an amino acid residue (e.g., arginine, homoarginine, norarginine, nor-norarginine, ornithine, lysine, homolysine, histidine, 1-methylhistidine, pyridylalanine, asparagine, N-ethylasparagine, glutamine, 4-aminophenylalanine, the N-methylated versions thereof, and side chain modified derivatives thereof) and one or more lipophilic tails. Exemplary amino acid lipids and their use to deliver nucleic acids are described in US Pat. App. Pub. No. 20110117125 and U.S. Pat. Nos. 8,877,729, 9,139,554, and 9,339,461. In some embodiments, membrane lytic poly (amido amine) polymers and polyconjugates such as those described in U.S. Pat. App. Pub. No. 20130289207 may be used. In some embodiments, a delivery agent comprises a lipopeptide compound comprising a central peptide and having lipophilic groups attached at each terminus. In some embodiments lipophilic groups can be derived from a naturally occurring lipid. In some embodiments a lipophilic group may comprise a C(1-22)alkyl, C(6-12) cycloalkyl, C(6-12) cycloalkyl-alkyl, C(3-18)alkenyl, C(3-18)alkynyl, C(1-5)alkoxy-C(1-5)alkyl, or a sphinganine, or (2R,3R)-2-amino-1,3-octadecanediol, icosasphinganine, sphingosine, phytosphingosine, or cis-4-sphingenine. The central peptide may comprise a cationic or amphipathic amino acid sequence. Examples of such lipopeptides and their use to deliver nucleic acids are described in, e.g., U.S. Pat. No. 9,220,785.

“Masking moiety” means a molecule or group that, when physically associated with another agent (e.g., a polymer), shields, inhibits or inactivates one or more properties (biophysical or biochemical characteristics) or activities of the agent. In some embodiments, a masking moiety may be attached covalently or noncovalently to an inhibitory RNA. A masking moiety may be reversible, meaning that it is attached to the inhibitory RNA that it masks via a reversible linkage. As will be appreciated by those of ordinary skill in the art, a sufficient number of masking moieties are linked to the inhibitory RNA to be masked to achieve a desired level of inactivation.

In some embodiments an inhibitory RNA is conjugated to a delivery agent that is a polymer. Useful delivery polymers include, e.g., poly (acrylate) polymers (see, e.g., US Pat. Pub. No. 20150104408), poly (vinyl ester) polymers (see., e.g., US Pat. Pub. No. 20150110732) and certain polypeptides. In some embodiments the delivery polymer is a reversibly masked membrane active polymer. In some embodiments the inhibitory RNA or polymer, or both, has a targeting moiety conjugated thereto. In some embodiments an inhibitory RNA or an inhibitory RNA-targeting moiety conjugate is co-administered with a delivery polymer but is not conjugated to the polymer. “Co-administered” in this context means that the inhibitory RNA and the delivery polymer are administered to the subject such that they are present in the subject during overlapping time periods. The inhibitory RNA-targeting moiety conjugate and the delivery polymer may be administered simultaneously or they may be delivered sequentially. For simultaneous administration, they may be mixed prior to administration. For sequential administration, either the inhibitory RNA or the delivery polymer may be administered first. The inhibitory RNA and the delivery polymer may be administered in the same composition or may be administered separately sufficiently close together in time such that cytoplasmic delivery of the inhibitory RNA to cells is enhanced relative to cytoplasmic delivery that would occur without administration of the polymer. In some embodiments the inhibitory RNA and the delivery polymer are administered no more than 15 minutes, 30 minutes, 60 minutes, or 120 minutes apart. In some embodiments the delivery polymer is a targeted, reversibly masked membrane active polymer. The polymer has a targeting moiety attached thereto that targets the polymer to cells to which enhanced cytoplasmic delivery of the inhibitory RNA is desired. The inhibitory RNA may be targeted to the same cells, optionally using the same targeting moiety, i.e., the inhibitory RNA may be administered as an inhibitory RNA-targeting moiety conjugate. As used herein, membrane active polymers are surface active, amphipathic polymers that are able to induce one or more of the following effects upon a biological membrane: an alteration or disruption of the membrane that allows non-membrane permeable molecules to enter a cell or cross the membrane, pore formation in the membrane, fission of membranes, or disruption or dissolving of the membrane. As used herein, a membrane, or cell membrane, comprises a lipid bilayer. The alteration or disruption of the membrane can be functionally defined by the polymer's activity in at least one the following assays: red blood cell lysis (hemolysis), liposome leakage, liposome fusion, cell fusion, cell lysis, and endosomal release. A membrane active polymer may enhance delivery of a polynucleotide to a cell by disrupting or destabilizing the plasma membrane or an internal vesicle membrane (such as an endosome or lysosome), e.g., by forming a pore in the membrane, or disrupting endosomal or lysosomal vesicles thereby permitting release of the contents of the vesicle into the cell cytoplasm. In some embodiments the targeted reversibly masked membrane active polymer is an endosomolytic polymer. Endosomolytic polymers are polymers that, in response to a change in pH, are able to cause disruption or lysis of an endosome or otherwise provide for release of a normally cell membrane impermeable compound, such as a polynucleotide or protein, from a cellular internal membrane-enclosed vesicle, such as an endosome or lysosome. In some embodiments the polymer is a reversibly modified amphipathic membrane active polyamine wherein reversible modification inhibits membrane activity, neutralizes the polyamine to reduce positive charge and form a near neutral charge polymer. The reversible modification may also provide cell-type specific targeting and/or inhibit non-specific interactions of the polymer. The polyamine may be reversibly modified through reversible modification of amines on the polyamine. The reversibly masked membrane active polymer is substantially not membrane active when masked but becomes membrane active upon unmasking. Masking moieties are generally covalently bound to the membrane active polymer through physiologically reversible linkages. By using physiologically reversible linkages, the masking moieties can be cleaved from the polymer in vivo, thereby unmasking the polymer and restoring activity of the unmasked polymer. By choosing an appropriate reversible linkage, the activity of the membrane active polymer is restored after the conjugate has been delivered or targeted to a desired cell type or cellular location. Reversibility of the linkages provides for selective activation of the membrane active polymer. The physiologically reversible bond is reversible under mammalian intracellular conditions, which include chemical conditions such as pH, temperature, oxidative or reductive conditions or agents, and salt concentration found in or analogous to those found in mammalian cells. In some embodiments a targeting moiety, e.g., an ASGPR targeting moiety may serve as a masking moiety. In some embodiments the ASGPR targeting moiety has a lipophilic moiety conjugated thereto. Exemplary targeting moieties (e.g., ASGPR targeting moieties), physiologically labile bonds (e.g., enzymatically labile bonds, pH labile bonds), masking moieties, membrane active polymers (e.g., endosmolytically active polymers), lipophilic moieties, RNAi agent-targeting moiety conjugates, delivery agent-targeting moiety conjugates, conjugates comprising an RNAi agent, targeting moiety, and delivery agent, and methods of delivering nucleic acids to cells (e.g., liver cells) are described in US Pat. App. Pub. Nos. 20130245091, 20130317079, 20120157509, 20120165393, 20120172412, 20120230938, 20140135380, 20140135381, 20150104408, and 20150110732. In some embodiments an inhibitory RNA is co-administered with a mellitin peptide, e.g., as described in US Pat. App. Pub. No. 20120165393. The inhibitory RNA, mellitin peptide, or both, may have a targeting moiety conjugated thereto, optionally via a reversible linkage. In some embodiments a masking moiety comprises a dipeptide-amidobenzyl-carbonate or disubstituted maleic anhydride masking moiety e.g., as described in US Pat. App. Pub. No. 20150110732.

In some embodiments an inhibitory RNA may be administered in “naked” form, i.e., administered in the absence of a delivery agent. The naked inhibitory RNA may be in a suitable buffer solution. The buffer solution may, for example, comprise acetate, citrate, prolamine, carbonate, or phosphate, or any combination thereof. In some embodiments the buffer solution is phosphate buffered saline (PBS). The pH and osmolarity of the buffer solution can be adjusted such that it is suitable for administering to a subject. In some embodiments an inhibitory RNA is administered not in physical association with a lipid or lipid-containing particle. In some embodiments an inhibitory RNA is administered not in physical association with a nanoparticle or microparticle. In some embodiments an inhibitory RNA is administered not in physical association with a cationic polymer. In some embodiments an inhibitory RNA is administered not in physical association with cyclodextrin. In some embodiments an inhibitory RNA administered in “naked” form comprises a targeting moiety.

Inhibitory RNAs (e.g., an siRNA or miRNA described herein), or a vector comprising a nucleotide sequence encoding an siRNA or miRNA described herein, can be incorporated into pharmaceutical compositions. Such pharmaceutical compositions are useful for, among other things, administration and delivery to a subject in vivo or ex vivo. In some embodiments, pharmaceutical compositions also contain a pharmaceutically acceptable carrier or excipient. Such excipients include any pharmaceutical agent, e.g., a pharmaceutical agent that does not itself induce an immune response harmful to the individual receiving the composition, and which may be administered without undue toxicity. As used herein the terms “pharmaceutically acceptable” and “physiologically acceptable” mean a biologically acceptable formulation, gaseous, liquid or solid, or mixture thereof, which is suitable for one or more routes of administration, in vivo delivery or contact. Pharmaceutically acceptable excipients include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles.

Pharmaceutical compositions may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding, free base forms. In some embodiments, a pharmaceutical composition may be a lyophilized powder.

Pharmaceutical compositions can include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions can be formulated to be compatible with a particular route of administration or delivery, as set forth herein or known to one of skill in the art. Thus, pharmaceutical compositions include carriers, diluents, or excipients suitable for administration by various routes.

Compositions suitable for parenteral administration can comprise aqueous and non-aqueous solutions, suspensions or emulsions of the active compound, which preparations are typically sterile and can be isotonic with the blood of the intended recipient. Non-limiting illustrative examples include water, buffered saline, Hanks' solution, Ringer's solution, dextrose, fructose, ethanol, animal, vegetable or synthetic oils. Aqueous injection suspensions may contain substances which increase the viscosity of the suspension, such as sodium carboxymethyl cellulose, sorbitol, or dextran. Additionally, suspensions of the active compounds may be prepared as appropriate oil injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility to allow for the preparation of highly concentrated solutions.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

After pharmaceutical compositions have been prepared, they may be placed in an appropriate container and labeled for treatment. Such labeling can include amount, frequency, and method of administration.

Pharmaceutical compositions and delivery systems appropriate for the compositions, methods and uses of the disclosure are known in the art (see, e.g., Remington: The Science and Practice of Pharmacy. 21st Edition. Philadelphia, PA. Lippincott Williams & Wilkins, 2005).

The disclosure also provides methods for introducing inhibitory RNAs (e.g., an siRNA or miRNA described herein), or a vector comprising a nucleotide sequence encoding an siRNA or miRNA described herein, into a cell or an animal. In some embodiments, such methods include contacting a subject (e.g., a cell or tissue of a subject) with, or administering to a subject (e.g., a subject such as a mammal), an inhibitory RNA described herein (or a vector comprising a nucleotide sequence encoding an inhibitory RNA described herein), such that the inhibitory RNA is expressed in the subject (e.g., in a cell or tissue of a subject). In another embodiment, a method includes providing cells of an individual (patient or subject such as a mammal) with an inhibitory RNA described herein (or a vector comprising a nucleotide sequence encoding an inhibitory RNA described herein), such that the inhibitory RNA is expressed in the individual.

Compositions of an inhibitory RNA described herein (or a vector (e.g., an rAAV vector) comprising a nucleotide sequence encoding a inhibitory RNA described herein) can be administered in a sufficient or effective amount to a subject in need thereof. Doses can vary and depend upon the type, onset, progression, severity, frequency, duration, or probability of the disease to which treatment is directed, the clinical endpoint desired, previous or simultaneous treatments, the general health, age, gender, race or immunological competency of the subject and other factors that will be appreciated by the skilled artisan. The dose amount, number, frequency or duration may be proportionally increased or reduced, as indicated by any adverse side effects, complications or other risk factors of the treatment or therapy and the status of the subject. The skilled artisan will appreciate the factors that may influence the dosage and timing required to provide an amount sufficient for providing a therapeutic or prophylactic benefit.

The dose to achieve a therapeutic effect, e.g., the dose in vector genomes/per kilogram of body weight (vg/kg) (e.g., in the case of vector-based delivery) or mg/kg of bodyweight (mg/kg), will vary based on several factors including, but not limited to: route of administration, the level of inhibitory RNA expression required to achieve a therapeutic effect, the specific disease treated, any host immune response to the viral vector, a host immune response to the heterologous inhibitory RNA, and the stability of the inhibitory RNA expressed. One skilled in the art can determine, for vector-based deliveries of the inhibitor RNAs, a rAAV/vector genome dose range to treat a patient having a particular disease or disorder based on the aforementioned factors, as well as other factors. Generally, doses will range from at least 1×10⁸, or more, for example, 1×10⁹, 1×10¹⁰, 1×10¹¹, 1×10¹², 1×10¹³, 1×10¹⁴, or more, vector genomes per kilogram (vg/kg) of the weight of the subject, to achieve a therapeutic effect.

In some embodiments compositions of an inhibitory RNA are administered to a subject in an amount that is between 0.01 mg/kg and 50 mg/kg. In some embodiments the inhibitory RNA composition is administered at a dose of about 0.01 mg/kg to about 10 mg/kg or about 0.5 mg/kg to about 15 mg/kg. In some embodiments, the inhibitory RNA composition is administered at a dose of about 10 mg/kg to about 30 mg/kg. In some embodiments, the inhibitory RNA composition is administered at a dose of about 0.5 mg/kg, about 1 mg/kg, about 1.5 mg/kg, about 2.0 mg/kg, about 2.5 mg/kg, about 3 mg/kg, about 3.5 mg/kg, about 4 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, or about 50 mg/kg. In some embodiments the amount is between 0.01 mg/kg and 0.1 mg/kg, between 0.01 mg/kg and 0.1 mg/kg, between 0.1 mg/kg and 1.0 mg/kg, between 1.0 mg/kg and 2.5 mg/kg, between 2.5 mg/kg and 5.0 mg/kg, between 5.0 mg/kg and 10 mg/kg, between 10 mg/kg and 20 mg/kg, between 20 mg/kg and 30 mg/kg, between 30 mg/kg and 40 mg/kg or between 40 mg/kg and 50 mg/kg. In some embodiments a fixed dose is administered. In some embodiments the dose is between 5 mg and 1.0 g, e.g., between 5 mg and 10 mg, between 10 mg and 20 mg, between 20 mg and 40 mg, between 40 mg and 80 mg, between 80 mg and 160 mg, between 160 mg and 320 mg, between 320 mg and 640 mg, between 640 mg and 1 g. In some embodiments the dose is about 1 mg, 5 mg, 10 mg, 25 mg, 50 mg, 100 mg, 150 mg, 200 mg, 250 mg, 300 mg, 350 mg, 400 mg, 450 mg, 500 mg, 600 mg, 700 mg, 800 mg, 900 mg, or 1000 mg. In some embodiments the dose is a daily dose. In some embodiments the dose is administered according to a dosing regimen with a dosing interval of at least 2 days, e.g., at least 7 days, e.g., about 2, 3, 4, 6, or 8 weeks. For example, in some embodiments, the inhibitory RNA composition is administered according to a dosing regimen with a dosing interval of at least 7 days. In some embodiments, the inhibitory RNA composition is administered daily, weekly, monthly, or every 2, 3, 4, 5, or 6 months or longer. In some embodiments, any of the doses and/or dosing regimens described herein are administered subcutaneously. In some embodiments, the inhibitory RNA composition is administered once and levels of inhibition are subsequently measured, and once the level of inhibition decreases to a certain level, a subsequent dose of the inhibitory composition is administered.

In some embodiments, a subject exhibits a sustained inhibition of C3, e.g., measured by C3 mRNA expression (e.g., in liver tissue, e.g., a liver biopsy) for a period of time that is at least 2 days, e.g., at least 7 days, e.g., about 2, 3, 4, 6, 8, 10, 12, 16, or 20 weeks post-administration. In some embodiments, a subject exhibits a reduce level of serum C3, and the reduced level of serum C3 is maintained for a period of time that is at least 2 days, e.g., at least 7 days, e.g., about 2, 3, 4, 6, 8, 10, 12, 16, or 20 weeks post-administration.

An effective amount or a sufficient amount can (but need not) be provided in a single administration, may require multiple administrations, and, can (but need not) be, administered alone or in combination with another composition (e.g., another complement inhibitor described herein). For example, the amount may be proportionally increased as indicated by the need of the subject, type, status and severity of the disease treated or side effects (if any) of treatment. Amounts considered effective also include amounts that result in a reduction of the use of another treatment, therapeutic regimen or protocol, such as administration of another complement inhibitor described herein.

Accordingly, pharmaceutical compositions of the disclosure include compositions wherein the active ingredients are contained in an effective amount to achieve the intended therapeutic purpose. Determining a therapeutically effective dose is well within the capability of a skilled medical practitioner using the techniques and guidance provided in the disclosure. Therapeutic doses can depend on, among other factors, the age and general condition of the subject, the severity of the complement-mediated disease or disorder, and the strength of the control sequences regulating the expression levels of an inhibitory RNA described herein. Thus, a therapeutically effective amount in humans will fall in a relatively broad range that may be determined by a medical practitioner based on the response of an individual patient to vector-based treatment. Pharmaceutical compositions may be delivered to a subject, so as to allow production of an inhibitory RNA described herein in vivo by gene- and or cell-based therapies or by ex-vivo modification of the patient's or donor's cells.

Methods and uses of the disclosure include delivery and administration systemically by any route, for example, by injection or infusion. Delivery of a pharmaceutical composition in vivo may generally be accomplished via injection using a conventional syringe, although other delivery methods such as convection-enhanced delivery can also be used (see, e.g., U.S. Pat. No. 5,720,720). For example, compositions may be delivered subcutaneously, epidermally, intradermally, intramucosally, intraperitoneally, intravenously, intra-pleurally, intraarterially, orally, intrahepatically, via the portal vein, or intramuscularly. Other modes of administration include oral and pulmonary administration, suppositories, and transdermal applications. A clinician specializing in the treatment of patients with complement-mediated disorders may determine the optimal route for administration of inhibitory RNAs (e.g., an siRNA or miRNA described herein), or a vector comprising a nucleotide sequence encoding an siRNA or miRNA described herein.

In some embodiments, an inhibitory RNA described herein (or a vector comprising a nucleotide sequence encoding an inhibitory RNA described herein) may be administered to a subject once daily, weekly, every 2, 3, or 4 weeks, or even at longer intervals. In some embodiments, an inhibitory RNA described herein (or a vector comprising a nucleotide sequence encoding inhibitory RNA described herein) may be administered according to a dosing regimen that includes (i) an initial administration that is once daily, weekly, every 2, 3, or 4 weeks, or even at longer intervals; followed by (ii) a period of no administration of, e.g., 1, 2, 3, 4, 5, 6, 8, or 10 months, or 1, 2, 3, 4, 5, 6, 7, 8, 9, or 10 years. In some embodiments a vector comprising a nucleotide sequence encoding an inhibitory RNA described herein may be administered (i) one or more times during an initial time period of up to 2, 4, or 6 weeks or less; followed by (ii) a period of no administration of, e.g., 1, 2, 3, 4, 5, 6, 7, 8, 9, or 10 years. In some embodiments, a subject is monitored before and/or following treatment for level of C3 expression and/or activity, e.g., as measured using an alternative pathway assay, a classical pathway assay, or both. Suitable assays are known in the art and include, e.g., a hemolysis assay. In some embodiments, a subject is treated, or is retreated, if a measured level of C3 expression and/or activity is more than 10%, 20%, 30%, 40%, 50%, 100%, 200%, or more, relative to measured level of C3 expression and/or activity in a control subject.

### VI. DISEASES, DISORDERS, AND CONDITIONS

In some embodiments, an inhibitory RNA described herein (or a vector comprising a nucleotide sequence encoding an inhibitory RNA described herein), alone or in combination with one or more additional complement inhibitors described herein, is systemically administered (e.g., subcutaneously or intravenously administered) to a subject for treatment of an eye disorder such as macular degeneration (e.g., age-related macular degeneration (AMD) and Stargardt macular dystrophy), diabetic retinopathy, glaucoma, or uveitis. In some embodiments, an inhibitory RNA described herein (or a vector comprising a nucleotide sequence encoding an inhibitory RNA described herein), alone or in combination with one or more additional complement inhibitors described herein, may be systemically administered (e.g., subcutaneously or intravenously) for treatment of a subject suffering from or at risk of AMD. In some embodiments the AMD is neovascular (wet) AMD. In some embodiments the AMD is dry AMD. As will be appreciated by those of ordinary skill in the art, dry AMD encompasses geographic atrophy (GA), intermediate AMD, and early AMD. In some embodiments, a subject with GA is treated in order to slow or halt progression of the disease. For example, in some embodiments, treatment of a subject with GA reduces the rate of retinal cell death. A reduction in the rate of retinal cell death may be evidenced by a reduction in the rate of GA lesion growth in patients treated with an inhibitory RNA described herein (or a vector comprising a nucleotide sequence encoding an inhibitory RNA described herein), alone or in combination with one or more additional complement inhibitors described herein, as compared with control (e.g., patients given a sham administration). In some embodiments, a subject has intermediate AMD. In some embodiments, a subject has early AMD. In some embodiments, a subject with intermediate or early AMD is treated in order to slow or halt progression of the disease. For example, in some embodiments, treatment of a subject with intermediate AMD may slow or prevent progression to an advanced form of AMD (neovascular AMD or GA). In some embodiments, treatment of a subject with early AMD may slow or prevent progression to intermediate AMD. In some embodiments an eye has both GA and neovascular AMD. In some embodiments an eye has GA but not wet AMD.

In some embodiments, a subject has an eye disorder characterized by macular degeneration, choroidal neovascularization (CNV), retinal neovascularization (RNV), ocular inflammation, or any combination of the foregoing. Macular degeneration, CNV, RNV, and/or ocular inflammation may be a defining and/or diagnostic feature of the disorder. Exemplary disorders that are characterized by one or more of these features include, but are not limited to, macular degeneration related conditions, diabetic retinopathy, retinopathy of prematurity, proliferative vitreoretinopathy, uveitis, keratitis, conjunctivitis, and scleritis. In some embodiments, a subject is in need of treatment for ocular inflammation. Ocular inflammation can affect a large number of eye structures such as the conjunctiva (conjunctivitis), cornea (keratitis), episclera, sclera (scleritis), uveal tract, retina, vasculature, and/or optic nerve. Evidence of ocular inflammation can include the presence of inflammation-associated cells such as white blood cells (e.g., neutrophils, macrophages) in the eye, the presence of endogenous inflammatory mediator(s), one or more symptoms such as eye pain, redness, light sensitivity, blurred vision and floaters, etc. Uveitis is a general term that refers to inflammation in the uvea of the eye, e.g., in any of the structures of the uvea, including the iris, ciliary body or choroid. Specific types of uveitis include iritis, iridocyclitis, cyclitis, pars planitis and choroiditis. In some embodiments, the eye disorder is an eye disorder characterized by optic nerve damage (e.g., optic nerve degeneration), such as glaucoma.

In some embodiments it is contemplated that a relatively short course of an inhibitory RNA described herein (or a vector comprising a nucleotide sequence encoding an inhibitory RNA described herein), alone or in combination with one or more additional complement inhibitors described herein, e.g., between 1 week and 6 weeks, e.g., about 2-4 week, may provide a long-lasting benefit. In some embodiments, a remission is achieved for a prolonged period of time, e.g., 1-3 months, 3-6 months, 6-12 months, 12-24 months, or more. In some embodiments a subject may be monitored and/or treated prophylactically before recurrence of symptoms. For example, a subject may be treated prior to or upon exposure to a triggering event. In some embodiments a subject may be monitored, e g., for an increase in a biomarker, e.g., a biomarker comprising an indicator of Th17 cells or Th17 cell activity, or complement activation, and may be treated upon increase in the level of such biomarker. See, e.g., PCT/US2012/043845 for further discussion.

### VII. COMBINATION THERAPY

In some aspects, methods of the present disclosure involve administering an inhibitory RNA described herein, alone or in combination with one or more additional complement inhibitors. In some embodiments, an inhibitory RNA is administered to a subject already receiving therapy with another complement inhibitor; in some embodiments, another complement inhibitor is administered to a subject receiving an inhibitory RNA. In some embodiments, both an inhibitory RNA and another complement inhibitor are administered to the subject.

In some embodiments, administration of an inhibitory RNA may allow for administering a reduced dosing regimen of (e.g., involving a smaller amount in an individual dose, reduced frequency of dosing, reduced number of doses, and/or reduced overall exposure to) a second complement inhibitor, as compared to administration of a second complement inhibitor as single therapy. Without wishing to be bound by any theory, in some embodiments a reduced dosing regimen of a second complement inhibitor may avoid one or more undesired adverse effects that could otherwise result.

In some aspects, systemic administration of an inhibitory RNA in combination with a second complement inhibitor can reduce the amount of C3 in the subject's blood sufficiently such that a reduced dosing regimen of an inhibitory RNA and/or the second complement inhibitor is required to achieve a desired degree of complement inhibition.

In some aspects, administration of an inhibitory RNA in combination with a second complement inhibitor can reduce the amount of C3 in the subject's blood sufficiently such that a reduced dosing regimen of an inhibitory RNA and/or the second complement inhibitor is required to achieve a desired level of, or a desired amount of improvement in, one or more signs, symptoms, biomarkers, or outcome measures, of a complement-mediated disorder.

In some embodiments such a reduced dose can be administered in a smaller volume, or using a lower concentration, or using a longer dosing interval, or any combination of the foregoing, as compared to administration of an inhibitory RNA or a second complement inhibitor as single therapy.

Any complement inhibitor, e.g., a complement inhibitor known in the art, can be administered in combination with an inhibitory RNA described herein. In some embodiments, a complement inhibitor is compstatin or a compstatin analog.

Compstatin is a cyclic peptide that binds to C3 and inhibits complement activation. U.S. Pat. No. 6,319,897 describes a peptide having the sequence Ile-[Cys-Val-Val-Gln-Asp-Trp-Gly-His-His-Arg-Cys]-Thr (SEQ ID NO: 1), with the disulfide bond between the two cysteines denoted by brackets. It will be understood that the name “compstatin” was not used in U.S. Pat. No. 6,319,897 but was subsequently adopted in the scientific and patent literature (see, e.g., Morikis, et al., Protein Sci., 7 (3): 619-27, 1998) to refer to a peptide having the same sequence as SEQ ID NO: 2 disclosed in U.S. Pat. No. 6,319,897, but amidated at the C terminus. The term “compstatin” is used herein consistently with such usage. Compstatin analogs that have higher complement inhibiting activity than compstatin have been developed. See, e.g., WO2004/026328 (PCT/US2003/029653), Morikis, D., et al., Biochem Soc Trans. 32 (Pt 1): 28-32, 2004, Mallik, B., et al., J. Med. Chem., 274-286, 2005; Katragadda, M., et al. J. Med. Chem., 49:4616-4622, 2006; WO2007062249 (PCT/US2006/045539); WO2007044668 (PCT/US2006/039397), WO/2009/046198 (PCT/US2008/078593); WO/2010/127336 (PCT/US2010/033345).

As used herein, the term “compstatin analog” includes compstatin and any complement inhibiting analog thereof. The term “compstatin analog” encompasses compstatin and other compounds designed or identified based on compstatin and whose complement inhibiting activity is at least 50% as great as that of compstatin as measured, e.g., using any complement activation assay accepted in the art or substantially similar or equivalent assays. Certain suitable assays are described in U.S. Pat. No. 6,319,897, WO2004/026328, Morikis, supra, Mallik, supra, Katragadda 2006, supra, WO2007062249 (PCT/US2006/045539); WO2007044668 (PCT/US2006/039397), WO/2009/046198 (PCT/US2008/078593); and/or WO/2010/127336 (PCT/US2010/033345). The assay may, for example, measure alternative or classical pathway-mediated erythrocyte lysis or be an ELISA assay. In some embodiments, an assay described in WO/2010/135717 (PCT/US2010/035871) is used.

Table 8 provides a non-limiting list of compstatin analogs useful in the present disclosure. The analogs are referred to in abbreviated form in the left column by indicating specific modifications at designated positions (1-13) as compared to the parent peptide, compstatin. Consistent with usage in the art, “compstatin” as used herein, and the activities of compstatin analogs described herein relative to that of compstatin, refer to the compstatin peptide amidated at the C-terminus. Unless otherwise indicated, peptides in Table 8 are amidated at the C-terminus. Bold text is used to indicate certain modifications. Activity relative to compstatin is based on published data and assays described therein (WO2004/026328, WO2007044668, Mallik, 2005; Katragadda, 2006). In certain embodiments, the peptides listed in Table 8 are cyclized via a disulfide bond between the two Cys residues when used in the therapeutic compositions and methods of the disclosure. Alternate means for cyclizing the peptides are also within the scope of the disclosure.

TABLE 8

| | | SEQ | |
|---|---|---|---|
| | | ID | Activity |
| Peptide | Sequence | NO: | over |
| Compstatin | H-ICVVQDWGHHRCT-CONH2 | 8 | compstatin* |
| Ac-compstatin | Ac-ICVVQDWGHHRCT-CONH2 | 9 | 3x more |
| Ac-V4Y/H9A | Ac-ICVYQDWGAHRCT-CONH2 | 10 | 14x more |
| Ac-V4W/H9A-OH | Ac-ICVWQDWGAHRCT-COOH | 11 | 27x more |
| Ac-V4W/H9A | Ac-ICVWQDWGAHRCT-CONH2 | 12 | 45x more |
| Ac-V4W/H9A/T13dT-OH | Ac-ICVWQDWGAHRCdT-COOH | 13 | 55x more |
| Ac-V4(2-Nal)/H9A | Ac-ICV(2-Nal)QDWGAHRCT-CONH2 | 14 | 99x more |
| AcV4(2-Nal)/H9A-OH | Ac-ICV(2-Nal)QDWGAHRCT-COOH | 15 | 38x more |
| AcV4(1-Nal)/H9A-OH | Ac-ICV(1-Nal)QDWGAHRCT-COOH | 16 | 30x more |
| Ac-V42lgl/H9A | Ac-ICV(2-IgI)QDWGAHRCT-CONH2 | 17 | 39x more |
| Ac-V42lgl/H9A-OH | Ac-ICV(2-IgI)QDWGAHRCT-COOH | 18 | 37x more |
| Ac-V4Dht/H9A-OH | Ac-ICVDhtQDWGAHRCT-COOH | 19 | 5x more |
| Ac-V4(Bpa)/H9A-OH | Ac-ICV(Bpa)QDWGAHRCT-COOH | 20 | 49x more |
| Ac-V4(Bpa)/H9A | Ac-ICV(Bpa)QDWGAHRCT-CONH2 | 21 | 86x more |
| Ac-V4(Bta)/H9A-OH | Ac-ICV(Bta)QDWGAHRCT-COOH | 22 | 65x more |
| Ac-V4(Bta)/H9A | Ac-ICV(Bta)QDWGAHRCT-CONH2 | 23 | 64x more |
| Ac-V4W/H9(2-Abu) | Ac-ICVWQDWG(2-Abu)HRCT-CONH2 | 24 | 64x more |
| +G/V4W/H9A+AN-OH | H-GICVWQDWGAHRCTAN-COOH | 25 | 38x more |
| Ac-V4(5fW)/H9A | Ac-ICV(5fW)QDWGAHRCT-CONH2 | 26 | 31x more |
| Ac-V4(5-MeW)/H9A | Ac-ICV(5-methyl-W)QDWGAHRCT-CONH2 | 27 | 67x more |
| Ac-V4(1-MeW)/H9A | Ac-ICV(1-methyl-W)QDWGAHRCT-CONH2 | 28 | 264x more |
| Ac-V4W/W7(5fW)/H9A | Ac-ICVWQD(5fW)GAHRCT-CONH2 | 29 | 121x more |
| Ac-V4(5fW)/W7(5fW)/H9A | Ac-ICV(5fW)QD(5fW)GAHRCT-CONH2 | 30 | NA |
| Ac-V4(5-MeW)/W7(5fW)H9A | Ac-ICV(5-methyl-W)QD(5fW)GAHRCT- | 31 | NA |
| | CONH2 | | |
| Ac-V4(1MeW)/W7(5fW)/H9A | Ac-ICV(1-methyl-W)QD(5fW)GAHRCT- | 32 | 264x more |
| | CONH2 | | |
| +G/V4(6fW)/W7(6fW)H9A+N- | H-GICV(6fW)QD(6fW)GAHRCTN-COOH | 33 | 126x more |
| OH | | | |
| Ac-V4(1-formyl-W)/H9A | Ac-ICV(1-formyl-W)QDWGAHRCT-CONH2 | 34 | 264x more |
| Ac-V4(5-methoxy-W)/H9A | Ac-ICV(1-methyoxy-W)QDWGAHRCT- | 35 | 76x more |
| | CONH2 | | |
| G/V4(5f-W)/WV7(5fW)/H9A+N- | H-GICV(5fW)QD(5fW)GAHRCTN-COOH | 36 | 112x more |
| OH | | | |

NA = not available

In certain embodiments of the compositions and methods of the disclosure, the compstatin analog has a sequence selected from sequences 9-36. In one embodiment, the compstatin analog has a sequence of SEQ ID NO: 28. As used herein, “L-amino acid” refers to any of the naturally occurring levorotatory alpha-amino acids normally present in proteins or the alkyl esters of those alpha-amino acids. The term “D-amino acid” refers to dextrorotatory alpha-amino acids. Unless specified otherwise, all amino acids referred to herein are L-amino acids.

In some embodiments, one or more amino acid(s) of a compstatin analog (e.g., any of the compstatin analogs disclosed herein) can be an N-alkyl amino acid (e.g., an N-methyl amino acid). For example, and without limitation, at least one amino acid within the cyclic portion of the peptide, at least one amino acid N-terminal to the cyclic portion, and/or at least one amino acid C-terminal to the cyclic portion may be an N-alkyl amino acid, e.g., an N-methyl amino acid. In some embodiments, for example, a compstatin analog comprises an N-methyl glycine, e.g., at the position corresponding to position 8 of compstatin and/or at the position corresponding to position 13 of compstatin. In some embodiments, one or more of the compstatin analogs in Table 8 contains at least one N-methyl glycine, e.g., at the position corresponding to position 8 of compstatin and/or at the position corresponding to position 13 of compstatin. In some embodiments, one or more of the compstatin analogs in Table 8 contains at least one N-methyl isoleucine, e.g., at the position corresponding to position 13 of compstatin. For example, a Thr at or near the C-terminal end of a peptide whose sequence is listed in Table 8 or any other compstatin analog sequence may be replaced by N-methyl Ile. As will be appreciated, in some embodiments the N-methylated amino acids comprise N-methyl Gly at position 8 and N-methyl Ile at position 13. In some embodiments, a compstatin analog (e.g., any one of the compstatin analogs listed in Table 8) comprises an isoleucine at position corresponding to position 3 of SEQ ID NO: 8, either instead of or in addition to one or more substitutions described herein. For example, in some embodiments, a compstatin analog comprises or consists of the sequence of any one of SEQ ID NOs: 8-36, where position 3 is an isoleucine. In some embodiments, a compstatin analog comprises or consists of the sequence of any one of SEQ ID NOs: 25, 33, or 36, where position 4 is an isoleucine. Additional compstatin analogs are described in, e.g., WO2019/166411.

Compstatin analogs may be prepared by various synthetic methods of peptide synthesis known in the art via condensation of amino acid residues, e.g., in accordance with conventional peptide synthesis methods, may be prepared by expression in vitro or in living cells from appropriate nucleic acid sequences encoding them using methods known in the art. For example, peptides may be synthesized using standard solid-phase methodologies as described in Malik, supra, Katragadda, supra, WO2004026328, and/or WO2007062249. Potentially reactive moieties such as amino and carboxyl groups, reactive functional groups, etc., may be protected and subsequently deprotected using various protecting groups and methodologies known in the art. See, e.g., “Protective Groups in Organic Synthesis”, 3rd ed. Greene, T. W. and Wuts, P. G., Eds., John Wiley & Sons, New York: 1999. Peptides may be purified using standard approaches such as reversed-phase HPLC. Separation of diasteriomeric peptides, if desired, may be performed using known methods such as reversed-phase HPLC. Preparations may be lyophilized, if desired, and subsequently dissolved in a suitable solvent, e.g., water. The pH of the resulting solution may be adjusted, e.g. to physiological pH, using a base such as NaOH. Peptide preparations may be characterized by mass spectrometry if desired, e.g., to confirm mass and/or disulfide bond formation. See, e.g., Mallik, 2005, and Katragadda, 2006.

A compstatin analog can be modified by addition of a molecule such as polyethylene glycol (PEG) to stabilize the compound, reduce its immunogenicity, increase its lifetime in the body, increase or decrease its solubility, and/or increase its resistance to degradation. Methods for pegylation are well known in the art (Veronese, F. M. & Harris, Adv. Drug Deliv. Rev. 54, 453-456, 2002; Davis, F. F., Adv. Drug Deliv. Rev. 54, 457-458, 2002); Hinds, K. D. & Kim, S.W. Adv. Drug Deliv. Rev. 54, 505-530 (2002; Roberts, M. J., Bentley, M. D. & Harris, J. M. Adv. Drug Deliv. Rev. 54, 459-476; 2002); Wang, Y. S. et al. Adv. Drug Deliv. Rev. 54, 547-570, 2002). A wide variety of polymers such as PEGs and modified PEGs, including derivatized PEGs to which polypeptides can conveniently be attached are described in Nektar Advanced Pegylation 2005-2006 Product Catalog, Nektar Therapeutics, San Carlos, CA, which also provides details of appropriate conjugation procedures.

In some embodiments, a compstatin analog of any of SEQ ID NOs: 9-36, is extended by one or more amino acids at the N-terminus, C-terminus, or both, wherein at least one of the amino acids has a side chain that comprises a reactive functional group such as a primary or secondary amine, a sulfhydryl group, a carboxyl group (which may be present as a carboxylate group), a guanidino group, a phenol group, an indole ring, a thioether, or an imidazole ring, which facilitate conjugation with a reactive functional group to attach a PEG to the compstatin analog. In some embodiments, the compstatin analog comprises an amino acid having a side chain comprising a primary or secondary amine, e.g., a Lys residue. For example, a Lys residue, or a sequence comprising a Lys residue, is added at the N-terminus and/or C-terminus of a compstatin analog described herein (e.g., a compstatin analog comprising any one of SEQ ID NOs: 9-36).

In some embodiments, the Lys residue is separated from the cyclic portion of the compstatin analog by a rigid or flexible spacer. The spacer may, for example, comprise a substituted or unsubstituted, saturated or unsaturated alkyl chain, oligo (ethylene glycol) chain, and/or other moieties, e.g., as described herein with regard to linkers. The length of the chain may be, e.g., between 2 and 20 carbon atoms. In other embodiments the spacer is a peptide. The peptide spacer may be, e.g., between 1 and 20 amino acids in length, e.g., between 4 and 20 amino acids in length. Suitable spacers can comprise or consist of multiple Gly residues, Ser residues, or both, for example. Optionally, the amino acid having a side chain comprising a primary or secondary amine and/or at least one amino acid in a spacer is a D-amino acid. Any of a variety of polymeric backbones or scaffolds could be used. For example, the polymeric backbone or scaffold may be a polyamide, polysaccharide, polyanhydride, polyacrylamide, polymethacrylate, polypeptide, polyethylene oxide, or dendrimer. Suitable methods and polymeric backbones are described, e.g., in WO98/46270 (PCT/US98/07171) or WO98/47002 (PCT/US98/06963). In one embodiment, the polymeric backbone or scaffold comprises multiple reactive functional groups, such as carboxylic acids, anhydride, or succinimide groups. The polymeric backbone or scaffold is reacted with the compstatin analogs. In one embodiment, the compstatin analog comprises any of a number of different reactive functional groups, such as carboxylic acids, anhydride, or succinimide groups, which are reacted with appropriate groups on the polymeric backbone. Alternately, monomeric units that could be joined to one another to form a polymeric backbone or scaffold are first reacted with the compstatin analogs and the resulting monomers are polymerized. In another embodiment, short chains are prepolymerized, functionalized, and then a mixture of short chains of different composition are assembled into longer polymers.

In some embodiments, a compstatin analog moiety is attached at each end of a linear PEG. A bifunctional PEG having a reactive functional group at each end of the chain may be used, e.g., as described herein. In some embodiments, the reactive functional groups are identical while in some embodiments different reactive functional groups are present at each end.

In general and compounds depicted herein, a polyethylene glycol moiety is drawn with the oxygen atom on the right side of the repeating unit or the left side of the repeating unit. In cases where only one orientation is drawn, the present disclosure encompasses both orientations (i.e., (CH₂CH₂O)ₙ and (OCH₂CH₂)ₙ) of polyethylene glycol moieties for a given compound or genus, or in cases where a compound or genus contains multiple polyethylene glycol moieties, all combinations of orientations are encompasses by the present disclosure.

In some embodiments a bifunctional linear PEG comprises a moiety comprising a reactive functional group at each of its ends. The reactive functional groups may be the same (homobifunctional) or different (heterobifunctional). In some embodiments the structure of a bifunctional PEG may be symmetric, wherein the same moiety is used to connect the reactive functional group to oxygen atoms at each end of the —(CH₂CH₂O)ₙ chain. In some embodiments different moieties are used to connect the two reactive functional groups to the PEG portion of the molecule. The structures of exemplary bifunctional PEGs are depicted below. For illustrative purposes, formulas in which the reactive functional group(s) comprise an NHS ester are depicted, but other reactive functional groups could be used.

In some embodiments, a bifunctional linear PEG is of formula A:

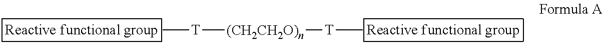

wherein each T and “Reactive functional group” is independently as defined below, and described in classes and subclasses herein, and n is as defined above and described in classes and subclasses herein.
   Each T is independently a covalent bond or a C₁₋₁₂ straight or branched, hydrocarbon chain wherein one or more carbon units of T are optionally and independently replaced by —O—, —S—, —N(R^{x})—, —C(O)—, —C(O)O—, —OC(O)—, —N(R^{x})C(O)—, —C(O)N(R^{x})—, —S(O)—, —S(O)₂—, —N(R^{x})SO₂—, or —SO₂N(R^{x})—; and
   each R^{x} is independently hydrogen or C₁₋₆ aliphatic.
   The Reactive functional group has the structure —COO—NHS.

Exemplary bifunctional PEGs of formula A include:

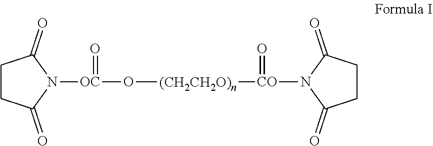

In some embodiments, a functional group (for example, an amine, hydroxyl, or thiol group) on a compstatin analog is reacted with a PEG-containing compound having a “reactive functional group” as described herein, to generate such conjugates. By way of example, Formula I can form compstatin analog conjugates having the structure:

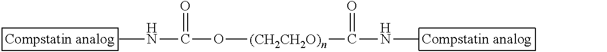

wherein,

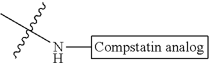 [SMILES: *NC]

represents the attachment point of an amine group on a compstatin analog. In certain embodiments, an amine group is a lysine side chain group.

In certain embodiments, the PEG component of such conjugates has an average molecular weight of about 5 kD, about 10 kD, about 15 kD, about 20 kD, about 30 kD, or about 40 kD. In certain embodiments, the PEG component of such conjugates has an average molecular weight of about 40 kD.

The term “bifunctional” or “bifunctionalized” is sometimes used herein to refer to a compound comprising two compstatin analog moieties linked to a PEG. Such compounds may be designated with the letter “BF”. In some embodiments a bifunctionalized compound is symmetrical. In some embodiments the linkages between the PEG and each of the compstatin analog moieties of a bifunctionalized compound are the same. In some embodiments, each linkage between a PEG and a compstatin analog of a bifunctionalized compound comprises a carbamate. In some embodiments, each linkage between a PEG and a compstatin analog of a bifunctionalized compound comprises a carbamate and does not comprise an ester. In some embodiments, each compstatin analog of a bifunctionalized compound is directly linked to a PEG via a carbamate. In some embodiments, each compstatin analog of a bifunctionalized compound is directly linked to a PEG via a carbamate, and the bifunctionalized compound has the structure:

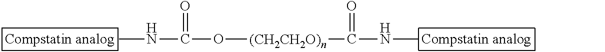

In some embodiments of formulae and embodiments described herein,

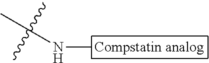 [SMILES: *NC]

represents point of attachment of a lysine side chain group in a compstatin analog having the structure:

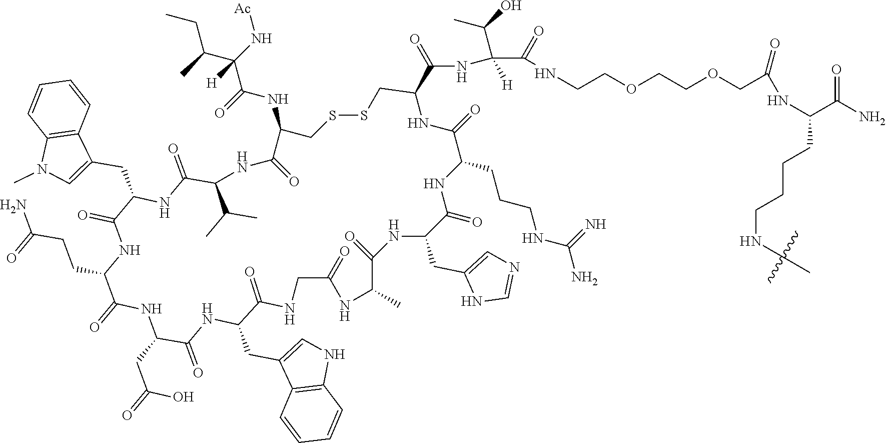 [SMILES: *NCCCC[C@H](NC(=O)COCCOCCNC(=O)[C@@H](NC(=O)[C@@H]1CSSC[C@H](NC(=O)[C@@H](NC(C)=O)[C@@H](C)CC)C(=O)N[C@@H](C(C)C)C(=O)N[C@@H](Cc2cn(C)c3ccccc23)C(=O)N[C@@H](CCC(N)=O)C(=O)N[C@@H](CC(=O)O)C(=O)N[C@@H](Cc2c[nH]c3ccccc23)C(=O)NCC(=O)N[C@@H](C)C(=O)N[C@@H](Cc2cnc[nH]2)C(=O)N[C@@H](CCCNC(=N)N)C(=O)N1)[C@@H](C)O)C(N)=O]

wherein the symbol “ [symbol] ” denotes the point of attachment of a chemical moiety to the remainder of a molecule or chemical formula.

PEGs comprising one or more reactive functional groups may, in some embodiments, be obtained from, e.g., NOF America Corp. White Plains, NY or BOC Sciences 45-16 Ramsey Road Shirley, NY 11967, USA, among others, or may be prepared using methods known in the art.

In some embodiments, a linker is used to connect a compstatin analog described herein and a PEG described herein. Suitable linkers for connecting a compstatin analog and a PEG are extensively described above and in classes and subclasses herein. In some embodiments, a linker has multiple functional groups, wherein one functional group is connected to a compstatin analog and another is connected to a PEG moiety. In some embodiments, a linker is a bifunctional compound. In some embodiments, a linker has the structure of NH₂(CH₂CH₂O)ₙCH₂C(═O)OH, wherein n is 1 to 1000. In some embodiments, a linker is 8-amino-3,6-dioxaoctanoic acid (AEEAc). In some embodiments, a linker is activated for conjugation with a polymer moiety or a functional group of a compstatin analog. For example, in some embodiments, the carboxyl group of AEEAc is activated before conjugation with the amine group of the side chain of a lysine group.

In some embodiments, a suitable functional group (for example, an amine, hydroxyl, thiol, or carboxylic acid group) on a compstatin analog is used for conjugation with a PEG moiety, either directly or via a linker. In some embodiments, a compstatin analog is conjugated through an amine group to a PEG moiety via a linker. In some embodiments, an amine group is the α-amino group of an amino acid residue. In some embodiments, an amine group is the amine group of the lysine side chain. In some embodiments, a compstatin analog is conjugated to a PEG moiety through the amino group of a lysine side chain (ε-amino group) via a linker having the structure of NH₂(CH₂CH₂O)ₙCH₂C(═O) OH, wherein n is 1 to 1000. In some embodiments, a compstatin analog is conjugated to the PEG moiety through the amino group of a lysine side chain via an AEEAc linker. In some embodiments, the NH₂(CH₂CH₂O)ₙCH₂C(═O) OH linker introduces a —NH(CH₂CH₂O)ₙCH₂C(—O)— moiety on a compstatin lysine side chain after conjugation. In some embodiments, the AEEAc linker introduces a —NH(CH₂CH₂O)₂CH₂C(—O)— moiety on a compstatin lysine side chain after conjugation.

In some embodiments, a compstatin analog is conjugated to a PEG moiety via a linker, wherein the linker comprises an AEEAc moiety and an amino acid residue. In some embodiments, a compstatin analog is conjugated to a PEG moiety via a linker, wherein the linker comprises an AEEAc moiety and a lysine residue. In some embodiments, the C-terminus of a compstatin analog is connected to the amino group of AEEAc, and the C-terminus of AEEAc is connected to a lysine residue. In some embodiments, the C-terminus of a compstatin analog is connected to the amino group of AEEAc, and the C-terminus of AEEAc is connected to the α-amino group of a lysine residue. In some embodiments, the C-terminus of a compstatin analog is connected to the amino group of AEEAc, the C-terminus of AEEAc is connected to the α-amino group of the lysine residue, and a PEG moiety is conjugated through the s-amino group of said lysine residue. In some embodiments, the C-terminus of the lysine residue is modified. In some embodiments, the C-terminus of the lysine residue is modified by amidation. In some embodiments, the N-terminus of a compstatin analog is modified. In some embodiments, the N-terminus of a compstatin analog is acetylated.

In certain embodiments a compstatin analog may be represented as M-AEEAc-Lys-B₂, wherein B₂ is a blocking moiety, e.g., NH₂, M represents any of SEQ ID NOs: 9-36, with the proviso that the C-terminal amino acid of any of SEQ ID NOs: 9-36 is linked via a peptide bond to AEEAc-Lys-B₂. The NHS moiety of a monofunctional or multifunctional (e.g., bifunctional) PEG reacts with the free amine of the lysine side chain to generate a monofunctionalized (one compstatin analog moiety) or multifunctionalized (multiple compstatin analog moieties) PEGylated compstatin analog. In various embodiments any amino acid comprising a side chain that comprises a reactive functional group may be used instead of Lys (or in addition to Lys). A monofunctional or multifunctional PEG comprising a suitable reactive functional group may be reacted with such side chain in a manner analogous to the reaction of NHS-ester activated PEGs with Lys.

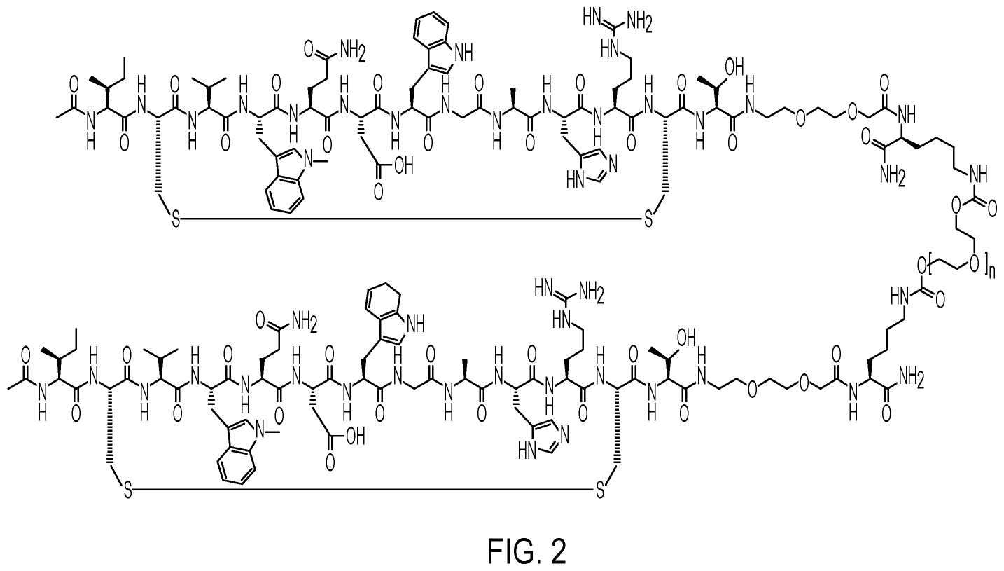

With regard to any of the above formulae and structures, it is to be understood that embodiments in which the compstatin analog component comprises any compstatin analog described herein, e.g., any compstatin analog of SEQ ID NOs; 9-36 are expressly disclosed. For example, and without limitation, a compstatin analog may comprise the amino acid sequence of SEQ ID NO: 28. An exemplary PEGylated compstatin analog in which the compstatin analog component comprises the amino acid sequence of SEQ ID NO: 28 is depicted in FIG. 2. It will be understood that the PEG moiety may have a variety of different molecular weights or average molecular weights in various embodiments, as described herein. In certain embodiments, a compstatin analog is pegcetacoplan (“APL-2”), having the structure of the compound of FIG. 2 with n of about 800 to about 1100 and a PEG having an average molecular weight of about 40 kD. Pegcetacoplan is also referred to as Poly (oxy-1,2-ethanediyl), α-hydro-ω-hydroxy-, 15,15′-diester with N-acetyl-L-isoleucyl-L-cysteinyl-L-valyl-1-methyl-L-tryptophyl-L-glutaminyl-L-α-aspartyl-L-tryptophylglycyl-L-alanyl-L-histidyl-L-arginyl-L-cysteinyl-L-threonyl-2-[2-(2-aminoethoxy) ethoxy]acetyl-N⁶-carboxy-L-lysinamide cyclic (2->12)-(disulfide); or O,O′-bis [(S²,S¹²-cyclo {N-acetyl-L-isoleucyl-L-cysteinyl-L-valyl-1-methyl-L-tryptophyl-L-glutaminyl-L-α-aspartyl-L-tryptophylglycyl-L-alanyl-L-histidyl-L-arginyl-L-cysteinyl-L-threonyl-2-[2-(2-aminoethoxy) ethoxy]acetyl-L-lysinamide})-N^{6.15}-carbonyl]polyethylene glycol (n=800-1100). Additional compstatin analogs are described in, e.g., WO 2012/155107 and WO 2014/078731.

In some embodiments, a compstatin analog described herein is administered twice weekly or every 3 days, at a dosage of about 800 mg to about 1200 mg, e.g., about 1060 mg to about 1100 mg, e.g., about 1070 mg to about 1090 mg, e.g., about 1075 mg to about 1085 mg, e.g., about 1080 mg, for about 4 weeks, about 8 weeks, about 12 weeks, about 16 weeks, about 20 weeks, about 24 weeks, about 28 weeks, about 32 weeks, about 36 weeks, about 40 weeks, about 44 weeks, about 48 weeks, about 52 weeks, about 1.2 years, 1.4 years, 1.6 years, 1.8 years, 2 years, 3 years, 4 years, 5 years, or longer.

In some embodiments, a composition comprising one or more inhibitory RNAs (e.g., an siRNA or miRNA described herein), or comprising a vector comprising a nucleotide sequence encoding an siRNA or miRNA described herein, is administered to a subject in combination with a compstatin analog, such that the compstatin analog and/or the inhibitory RNA composition is administered less frequently and/or at a lower dosage. In some embodiments, a composition comprising one or more inhibitory RNAs (e.g., an siRNA or miRNA described herein), or a vector comprising a nucleotide sequence encoding an siRNA or miRNA described herein, is administered to a subject in combination with a compstatin analog, such that the compstatin analog is administered once a week, once every 2 weeks, once a month, once every 2 months, 3 months, 4 months, 5 months, or longer, at a dosage of about 800 mg to about 1200 mg, e.g., about 1060 mg to about 1100 mg, e.g., about 1070 mg to about 1090 mg, e.g., about 1075 mg to about 1085 mg, e.g., about 1080 mg.

In some embodiments, a complement inhibitor is an antibody, e.g., an anti-C3 and/or anti-C5 antibody, or a fragment thereof. In some embodiments, an antibody fragment may be used to inhibit C3 or C5 activation. The fragmented anti-C3 or anti-C5 antibody may be Fab′, Fab′ (2), Fv, or single chain Fv. In some embodiments, the anti-C3 or anti-C5 antibody is monoclonal. In some embodiments, the anti-C3 or anti-C5 antibody is polyclonal. In some embodiments, the anti-C3 or anti-C5 antibody is de-immunized. In some embodiments the anti-C3 or anti-C5 antibody is a fully human monoclonal antibody. In some embodiments, the anti-C5 antibody is eculizumab. In some embodiments, a complement inhibitor is an antibody, e.g., an anti-C3 and/or anti-C5 antibody, or a fragment thereof.

In some embodiments, a complement inhibitor is a polypeptide inhibitor and/or a nucleic acid aptamer (see, e.g., U.S. Publ. No. 20030191084). Exemplary polypeptide inhibitors include an enzyme that degrades C3 or C3b (see, e.g., U.S. Pat. No. 6,676,943). Additional polypeptide inhibitors include mini-factor H (see, e.g., U.S. Publ. No. 20150110766), Efb protein or complement inhibitor (SCIN) protein from Staphylococcus aureus, or a variant or derivative or mimetic thereof (see, e.g., U.S. Publ. 20140371133).

A variety of other complement inhibitors can also be used in various embodiments of the disclosure. In some embodiments, the complement inhibitor is a naturally occurring mammalian complement regulatory protein or a fragment or derivative thereof. For example, the complement regulatory protein may be CR1, DAF, MCP, CFH, or CFI. In some embodiments, the complement regulatory polypeptide is one that is normally membrane-bound in its naturally occurring state. In some embodiments, a fragment of such polypeptide that lacks some or all of a transmembrane and/or intracellular domain is used. Soluble forms of complement receptor 1 (sCR1), for example, can also be used. For example the compounds known as TP10 or TP20 (Avant Therapeutics) can be used, C1 inhibitor (C1-INH) can also be used. In some embodiments a soluble complement control protein, e.g., CFH, is used.

Inhibitors of C1s can also be used. For example, U.S. Pat. No. 6,515,002 describes compounds (furanyl and thienyl amidines, heterocyclic amidines, and guanidines) that inhibit C1s. U.S. Pat. Nos. 6,515,002 and 7,138,530 describe heterocyclic amidines that inhibit C1s. U.S. Pat. No. 7,049,282 describes peptides that inhibit classical pathway activation. Certain of the peptides comprise or consist of WESNGQPENN (SEQ ID NO: 73) or KTISKAKGQPREPQVYT (SEQ ID NO: 74) or a peptide having significant sequence identity and/or three-dimensional structural similarity thereto. In some embodiments these peptides are identical or substantially identical to a portion of an IgG or IgM molecule. U.S. Pat. No. 7,041,796 discloses C3b/C4b Complement Receptor-like molecules and uses thereof to inhibit complement activation. U.S. Pat. No. 6,998,468 discloses anti-C2/C2a inhibitors of complement activation. U.S. Pat. No. 6,676,943 discloses human complement C3-degrading protein from Streptococcus pneumoniae.

All publications, patent applications, patents, and other references mentioned herein, including GenBank Accession Numbers, are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure is further illustrated by the following examples. The examples are provided for illustrative purposes only. They are not to be construed as limiting the scope or content of the disclosure in any way.

### VIII. EXEMPLIFICATION

### Example 1: Knockdown of C3 Expression in Hela Cells Using siRNAs

### Cell Culture

HeLa cells were obtained from ATCC (ATCC in partnership with LGC Standards, Wesel, Germany, cat. #ATCC-CRM-CCL-2) and cultured in HAM's F12 (#FG0815, Biochrom, Berlin, Germany), supplemented to contain 10% fetal calf serum (#1248D, Biochrom GmbH, Berlin, Germany), and 100U/ml Penicillin/100 μg/ml Streptomycin (#A2213, Biochrom GmbH, Berlin, Germany) at 37° C. in an atmosphere with 5% CO2 in a humidified incubator. For transfection of HeLa cells with siRNAs, cells were seeded at a density of 15,000 cells/well into 96-well tissue culture plates (#655180, GBO, Germany).

siRNAS

177 siRNAs were designed and synthesized to target different regions of the mRNA transcript. In this experiment, the sense strand of each siRNA contained 18 nucleotides identical to a target region sequence on the C3 transcript (SEQ ID NO: 75), and one additional adenine nucleotide at the 3′ end. Additionally, in this experiment, the antisense strand contained 18 nucleotides complementary to a target region sequence on the C3 transcript (SEQ ID NO: 75), and one additional uracil nucleotide at the 5′ end, and 2 additional uracil nucleotides at the 3′ end.

In this experiment, siRNAs contained modifications of the sense strand that included the following modification pattern:
xsxsXfxxxXfXfXfxxXfxxxxXfxa

Antisense strands included the following modification pattern:
usXfsxxxxxxxxxxxXfxxxxxsusu
where “x” represents any nucleotide; a lowercase letter represents a nucleotide modified with a 2′-O-Methyl group; “Xf” represents a nucleotide (“X” can be any nucleotide) modified with a 2′-Fluoro group. For example, “Af” represents an adenine nucleotide modified with a 2′-Fluoro group. An “s” represents a phosphorothioate bond.
   Transfection of siRNA and C3 Activity Assay Dual-Dose Experiments

Transfection of siRNA was carried out with Lipofectamine RNAiMax (Invitrogen/Life Technologies, Karlsruhe, Germany) according to manufacturer's instructions for reverse transfection. In this experiment, a dual dose screen was performed with siRNAs in quadruplicates at 10 nM and 0.5 nM, respectively. siRNAs targeting Aha1 served at the same time as an unspecific control for C3 target mRNA expression and as a positive control to analyze transfection efficiency with regards to Aha1 mRNA level. Firefly-Luciferase and Renilla-Luciferase was used as a mock transfection.

After 24h of incubation with siRNAs, medium was removed and cells were lysed in 150 μl Medium-Lysis Mixture (1 volume lysis mixture, 2 volumes cell culture medium) and then incubated at 53° C. for 30 minutes. bDNA assay (ThermoFisher QuantiGene RNA assays) was performed according to manufacturer's instructions with a probeset directed to human C3 (accession—#NM_000064 between base 106 and base 907 of the sequence) which had been designed by ThermoFisher Scientific, and synthesized by Metabion International AG, Planegg, Germany. Luminescence was read using 1420 Luminescence Counter (WALLAC VICTOR Light, Perkin Elmer, Rodgau-Jügesheim, Germany) following 30 minutes incubation at room temperature in the dark.

By hybridization with an Aha1 probeset, the other two target-unspecific controls (for firefly luciferase and Renilla luciferase) served as controls for Aha1 mRNA level. Transfection efficiency for each 96-well plate and both doses in the dual dose screen was calculated by relating the Aha1-level in wells with

Aha1-siRNA (normalized to GapDH) to Aha1-level obtained with controls. Transfection efficiency with siAha1 at the 10 nM dose was about 90%, and the transfection efficiency at the 0.5 nM dose was about 85%.

Activity of the siRNAs was measured by the lowest fluorescence or lowest percent mRNA concentration of the respective targets. For each well, the target mRNA level was normalized to the respective GAPDH mRNA level. The activity of a given siRNA was expressed as percent mRNA concentration of the respective target (normalized to GAPDH mRNA) in treated cells, relative to the target mRNA concentration (normalized to GAPDH mRNA) averaged across control wells.

Results from the dual dose screen of the top 24 siRNAs based on activity are shown in Table 9 below. The sequences for these siRNAs are shown in Table 10 below.

TABLE 9

| siRNA | % residual C3 | | % residual C3 | |
|---|---|---|---|---|
| ID | mRNA [10 nm] | SD | mRNA [0.5 nm] | SD |
| 1 | 17 | 3 | 40 | 13 |
| 2 | 22 | 2 | 75 | 4 |
| 3 | 23 | 10 | 57 | 12 |
| 4 | 25 | 3 | 63 | 4 |
| 5 | 27 | 4 | 63 | 8 |
| 6 | 27 | 5 | 62 | 3 |
| 7 | 27 | 4 | 83 | 4 |
| 8 | 28 | 2 | 79 | 7 |
| 9 | 28 | 3 | 55 | 6 |
| 10 | 28 | 4 | 53 | 3 |
| 11 | 28 | 5 | 76 | 2 |
| 12 | 29 | 5 | 56 | 3 |
| 13 | 29 | 8 | 94 | 18 |
| 14 | 30 | 3 | 79 | 8 |
| 15 | 31 | 5 | 65 | 4 |
| 16 | 32 | 3 | 47 | 10 |
| 17 | 32 | 8 | 80 | 12 |
| 18 | 34 | 6 | 58 | 9 |
| 19 | 35 | 5 | 57 | 19 |
| 20 | 38 | 4 | 63 | 6 |
| 21 | 39 | 4 | 72 | 2 |
| 22 | 39 | 6 | 59 | 3 |
| 23 | 39 | 10 | 72 | 6 |
| 24 | 42 | 9 | 62 | 4 |

TABLE 10

| siRNA | | |
|---|---|---|
| ID | Sense sequence (5′ to 3′) | Antisense sequence (5′ to 3′) |
| 1 | uscsAfacuCfAfCfcuGfuaauAfaa (SEQ ID NO: | usUfsuauuacaggugAfguugasusu (SEQ ID NO: |
| | 201) | 202) |
| 2 | asgsGfaugCfCfAfcuAfugucUfaa (SEQ ID NO: | usUfsagacauaguggCfauccususu (SEQ ID NO: |
| | 203) | 204) |
| 3 | csusUfgaaGfCfCfaaCfuacaUfga (SEQ ID NO: | usCfsauguaguuggcUfucaagsusu (SEQ ID NO: |
| | 205) | 206) |
| 4 | uscsCfaagCfCfUfugGfcucaAfua (SEQ ID NO: | usAfsuugagccaaggCfuuggasusu (SEQ ID NO: |
| | 207) | 208) |
| 5 | asgsUfcaaGfGfUfcuAfcgccUfaa (SEQ ID NO: | usUfsaggcguagaccUfugacususu (SEQ ID NO: |
| | 209) | 210) |
| 6 | asasAfcugUfGfGfcuGfuucgCfaa (SEQ ID NO: | usUfsgcgaacagccaCfaguuususu (SEQ ID NO: |
| | 211) | 212) |
| 7 | usgsAfgauCfUfGfuaCfcaggUfaa (SEQ ID NO: | usUfsaccugguacagAfucucasusu (SEQ ID NO: |
| | 213) | 214) |
| 8 | csusUfuguUfCfUfcaUfcucgCfua (SEQ ID NO: | usAfsgcgagaugagaAfcaaagsusu (SEQ ID NO: |
| | 215) | 216) |
| 9 | asusCfggaUfCfUfucAfccguCfaa (SEQ ID NO: | usUfsgacggugaagaUfccgaususu (SEQ ID NO: |
| | 217) | 218) |
| 10 | uscsAfacuUfCfCfucCfugcgAfaa (SEQ ID NO: | usUfsucgcaggaggaAfguugasusu (SEQ ID NO: |
| | 219) | 220) |
| 11 | csgsUfgcuGfCfCfcaGfuuucGfaa (SEQ ID NO: | usUfscgaaacugggcAfgcacgsusu (SEQ ID NO: |
| | 221) | 222) |
| 12 | asusAfggaAfCfAfccCfucauCfaa (SEQ ID NO: | usUfsgaugaggguguUfccuaususu (SEQ ID NO: |
| | 223) | 224) |
| 13 | usgsGfucaAfGfGfucUfucucUfca (SEQ ID NO: | usGfsagagaagaccuUfgaccasusu (SEQ ID NO: |
| | 225) | 226) |
| 14 | gscsAfacuCfCfAfacAfauuaCfca (SEQ ID NO: | usGfsguaauuguuggAfguugcsusu (SEQ ID NO: |
| | 227) | 228) |
| 15 | cscsUfuugUfCfAfucUfucggGfaa (SEQ ID NO: | usUfscccgaagaugaCfaaaggsusu (SEQ ID NO: |
| | 229) | 230) |
| 16 | csasAfugaCfUfUfugAfcgagUfaa (SEQ ID NO: | usUfsacucgucaaagUfcauugsusu (SEQ ID NO: |
| | 231) | 232) |
| 17 | ascsGfacuUfCfCfcaGfgcaaAfaa (SEQ ID NO: | usUfsuuugccugggaAfgucgususu (SEQ ID NO: |
| | 233) | 234) |
| 18 | gsasAfcagAfGfAfuaCfuacgGfua (SEQ ID NO: | usAfsccguaguaucuCfuguucsusu (SEQ ID NO: |
| | 235) | 236) |
| 19 | gsusUfucgAfGfGfucAfuaguGfga (SEQ ID NO: | usCfscacuaugaccuCfgaaacsusu (SEQ ID NO: |
| | 237) | 238) |
| 20 | asasUfgaaCfAfGfagAfuacuAfca (SEQ ID NO: | usGfsuaguaucucugUfucauususu (SEQ ID NO: |
| | 239) | 240) |
| 21 | asgsCfuaaAfAfGfacUfuugaCfua (SEQ ID NO: | usAfsgucaaagucuuUfuagcususu (SEQ ID NO: |
| | 241) | 242) |
| 22 | cscsAfacuAfCfAfugAfaccuAfca (SEQ ID NO: | usGfsuagguucauguAfguuggsusu (SEQ ID NO: |
| | 243) | 244) |
| 23 | csusAfcucUfGfUfugUfucgaAfaa (SEQ ID NO: | usUfsuucgaacaacaGfaguagsusu (SEQ ID NO: |
| | 245) | 246) |
| 24 | gsusGfcguUfGfGfcuCfaaugAfaa (SEQ ID NO: | usUfsucauugagccaAfcgcacsusu (SEQ ID NO: |
| | 247) | 248) |
| 25 | csusCfcugCfGfAfauGfgaccGfca (SEQ ID NO: | usGfscgguccauucgCfaggagsusu (SEQ ID NO: |
| | 249) | 250) |

### Dose Response Experiments

The top 12 siRNAs with the best activity at both doses were chosen to be tested in a dose response experiment (DRC). Dose-response experiments were done with siRNA in 10 concentrations transfected in quadruplicates, starting at 100 nM in 6-fold dilutions steps down to ˜10 fM. Mock transfected cells served as control in DRC experiments.

For each well, the target mRNA level was normalized to the respective GAPDH mRNA level. The activity of a given siRNA was expressed as percent mRNA concentration of the respective target (normalized to GAPDH mRNA) in treated cells, relative to the target mRNA concentration (normalized to GAPDH mRNA) averaged across mock transfected wells (DRCs).

IC50 and IC80 values from the DRC experiments and Max KD results from the dual dose experiments (10 nm dose) are shown in Table 11 below:

TABLE 11

| siRNA | | | Max KD |
|---|---|---|---|
| ID | IC50 | IC80 | (HeLa) |
| 9 | 0.751 | #N/A | 75% |
| 10 | 0.172 | #N/A | 77% |
| 23 | 0.775 | 45.873 | 79% |
| 3 | 0.646 | 4.035 | 84% |
| 22 | 0.246 | 442952.010 | 73% |
| 20 | 0.395 | #N/A | 71% |
| 18 | 0.564 | #N/A | 78% |
| 4 | 0.208 | 11.812 | 81% |
| 1 | 0.086 | 12.744 | 81% |
| 12 | 0.349 | #N/A | 77% |
| 5 | 0.445 | 14.092 | 81% |
| 16 | 0.087 | 6.186 | 83% |

### Example 2: Knockdown of C3 Expression in HepG2 Cells Using siRNAs

The dual dose experiments in Example 1 were repeated for the top 50 siRNAs based on their demonstrated activity in HepG2 cells (Example 1).

HepG2 cells were obtained from ATCC (ATCC in partnership with LGC Standards, Wesel, Germany, cat. #ATCC-HB-8065) and cultured in MEM Eagle (#M2279, Sigma-Aldrich, Germany), supplemented to contain 10% fetal calf serum (#1248D, Biochrom GmbH, Berlin, Germany), 1× non-essential amino acids (#K0293; Biochrom, Berlin, Germany), 4 mM L-Glutamine (#K0283, Biochrom, Berlin, Germany) and 100U/ml Penicillin/100 μg/ml Streptomycin (#A2213, Biochrom GmbH, Berlin, Germany) at 37° C. in an atmosphere with 5% CO2 in a humidified incubator.

Transfection of siRNA and C3 Activity Assay Dual-Dose Experiments

For transfection of HepG2 cells with siRNAs, cells were seeded at a density of 15,000 cells/well into collagen-coated 96-well tissue culture plates (#655150, GBO, Germany). Transfection of siRNA was carried out with Lipofectamine RNAiMax (Invitrogen/Life Technologies, Karlsruhe, Germany) according to manufacturer's instructions for reverse transfection directly after seeding. In this experiment, a dual dose screen was performed with siRNAs in quadruplicates at 10 nM and 0.1 nM, respectively. siRNAs targeting Aha1 served at the same time as an unspecific control for C3 target mRNA expression and as a positive control to analyze transfection efficiency with regards to Aha1 mRNA level. Firefly-Luciferase and Renilla-Luciferase was used as a mock transfection.

After 24h of incubation with siRNAs, medium was removed and cells were lysed in 150 μl Medium-Lysis Mixture (1 volume lysis mixture, 2 volumes cell culture medium) and then incubated at 53° C. for 30 minutes. bDNA assay (ThermoFisher QuantiGene RNA assays) was performed according to manufacturer's instructions with a probeset directed to human C3 (accession—#NM_000064 between base 106 and base 907 of the sequence) which had been designed by ThermoFisher Scientific, and synthesized by Metabion International AG, Planegg, Germany. Luminescence was read using 1420 Luminescence Counter (WALLAC VICTOR Light, Perkin Elmer, Rodgau-Jügesheim, Germany) following 30 minutes incubation at room temperature in the dark.

Aha1-siRNA served as an unspecific control for C3 mRNA expression and as a positive control to analyze the transfection efficiency by measuring Aha1 mRNA level by hybridization with an Aha1 probeset. The Aha-1 siRNA used had been formerly selected from a big set of candidate siRNAs, and is known to be very active in vitro and in vivo. Transfection efficiency for each 96-well plate was calculated by analysis of Aha1-knock-down with Aha1-siRNA (normalized to GapDH) compared to an unspecific control. Aha1-siRNA (normalized to GapDH) to Aha1-level obtained with controls. Transfection efficiency with siAha1 at the 10 nM dose was about 90%, and the transfection efficiency at the 0.5 nM dose was about 85%.

Activity of the siRNAs was measured by the fluorescence or percent mRNA concentration of the respective targets. For each well, the target mRNA level was normalized to the respective GAPDH mRNA level. The activity of a given siRNA was expressed as percent mRNA concentration of the respective target (normalized to GAPDH mRNA) in treated cells, relative to the target mRNA concentration (normalized to GAPDH mRNA) averaged across control wells.

Results from the dual dose screen of the top 12 siRNAs based on activity are shown in Tables 12 and 13 below, for the 10 nm and 0.1 nm dosages, respectively.

TABLE 12

| siRNA | KD @ | KD @ |
|---|---|---|
| ID | 10 nM | 0.1 nM |
| 4 | 94% | 66% |
| 22 | 93% | 67% |
| 1 | 92% | 68% |
| 3 | 92% | 52% |
| 16 | 92% | 67% |
| 9 | 91% | 63% |
| 18 | 90% | 37% |
| 5 | 89% | 55% |
| 10 | 88% | 84% |
| 20 | 88% | 44% |
| 12 | 87% | 53% |
| 25 | 87% | 36% |

TABLE 13

| siRNA | KD @ | KD @ |
|---|---|---|
| ID | 10 nM | 0.1 nM |
| 10 | 88% | 84% |
| 1 | 92% | 68% |
| 22 | 93% | 67% |
| 16 | 92% | 67% |
| 4 | 94% | 66% |
| 9 | 91% | 63% |
| 5 | 89% | 55% |
| 12 | 87% | 53% |
| 3 | 92% | 52% |
| 6 | 84% | 49% |
| 20 | 88% | 44% |
| 18 | 90% | 37% |

### Example 3: C3 Knockdown Expression in HepG2 Cells Using siRNAs with Various Modification Patterns

Modifications of the top 6 siRNAs from Examples 1 and 2 were tested.

### Cell Culture

HepG2 cells were obtained from ATCC (ATCC in partnership with LGC Standards, Wesel, Germany, cat. #ATCC-HB-8065) and cultured in MEM Eagle (#M2279, Sigma-Aldrich, Germany), supplemented to contain 10% fetal calf serum (#1248D, Biochrom GmbH, Berlin, Germany), 1× non-essential amino acids (#K0293; Biochrom, Berlin, Germany), 4 mM L-Glutamine (#K0283, Biochrom, Berlin, Germany) and 100U/ml Penicillin/100 μg/ml Streptomycin (#A2213, Biochrom GmbH, Berlin, Germany) at 37° C. in an atmosphere with 5% CO2 in a humidified incubator.

siRNAs

siRNAs were designed and synthesized based on the nucleotide sequence of the top siRNAs in terms of activity from Examples 1 and 2 (siRNA ID: 1, 4, 9, 10, 16, and 22) with different modification patterns. 5 “variants” were designed and synthesized for each of the top siRNA nucleotide sequences, and each duplex is identified as “variant1”, “variant2”, “variant3”, variant4”, “variant5” depending on which modifications were made. siRNAs from Examples 1 and 2 (siRNA ID: 1, 4, 9, 10, 16, and 22) are identified as “variant0”.

The following modification patterns (5′ to 3′) of the sense strand of each siRNA ID 1, 4, 9, 10, 16, and 22 were used:
xsxsXfxxxXfXfXfxxXfxxxxXfxa (pattern used in “variant0” and also in “variant1” and “variant5”)
XfsxsXfxXfxXfxXfxXfxXfxXfxXfxAf (pattern used in “variant2”, “variant3” and “variant4”)

The following modification patterns (5′ to 3′) of the antisense strand were used:
usXfsxxxxxxxxxxxXfxxxxxsusu (pattern used in “variant0”)
usXfsxxxxxxxxxxxXfxxxxxsxsx (pattern used in “variant1” and “variant3”; which is the same pattern as used in “variant0” except that the last two nucleotides are complementary to the C3 mRNA transcript, i.e., SEQ ID NO: 75)
usXfsxXfxXfxXfxXfxXfxXfxXfxXfxsxsx (pattern used in “variant2”)
usXfsxxxXfxxxxxxxXfxXfxxxsxsx (pattern used in “variant4” and “variant5”)

where “x” represents any nucleotide; a lowercase letter represents a nucleotide modified with a 2′-O-Methyl group; “Xf”′ represents a nucleotide (“X” can be any nucleotide) modified with a 2′-Fluoro group. For example, “Af” represents an adenine nucleotide modified with a 2′-Fluoro group. An “s” represents a phosphorothioate bond.

### Dose Response Experiments

Transfection of siRNA was carried out with Lipofectamine RNAiMax (Invitrogen/Life Technologies, Karlsruhe, Germany) according to manufacturer's instructions for reverse transfection.

Each siRNA was tested using dose response experiments (DRC) in HepG2 cells. Two additional siRNAs (siRNA ID 26 and 27), known to knockdown C3 expression, were used as positive controls.

Dose-response experiments were done with siRNA in 10 concentrations transfected in quadruplicates, starting at 100 nM in 6-fold dilutions steps down to ˜10 fM. Mock transfected cells served as negative controls.

For each well, the target mRNA level was normalized to the respective GAPDH mRNA level. The activity of a given siRNA was expressed as percent mRNA concentration of the respective target (normalized to GAPDH mRNA) in treated cells, relative to the target mRNA concentration (normalized to GAPDH mRNA) averaged across mock transfected wells (DRCs).

IC50, IC80 values, and Max KD from the DRC experiments are shown in Table 14 below. The sequences for these siRNAs are shown in Table 15 below.

TABLE 14

| siRNA ID | Description | IC₅₀ @ [nM] | IC₈₀ @ [nM] | Max KD |
|---|---|---|---|---|
| 27 | (+) Control | 0.004 | 0.027 | 89% |
| 26 | (+) Control | 0.006 | 0.062 | 86% |
| 32 | siRNA_1_Variant5 | 0.018 | 0.108 | 93% |
| 22 | siRNA_22_Variant0 | 0.054 | 0.475 | 91% |
| 53 | siRNA_22_Variant1 | 0.056 | 0.510 | 91% |
| 33 | siRNA_4_Variant1 | 0.075 | 0.601 | 91% |
| 47 | siRNA 10_Variant5 | 0.055 | 0.708 | 83% |
| 28 | siRNA_1_Variant1 | 0.001 | 0.711 | 95% |
| 38 | siRNA_9_Variant1 | 0.151 | 0.728 | 91% |
| 4 | siRNA_4_Variant0 | 0.179 | 0.885 | 91% |
| 56 | siRNA_22_Variant4 | 0.228 | 1.201 | 91% |
| 9 | siRNA_9_Variant0 | 0.210 | 1.325 | 90% |
| 37 | siRNA_4_Variant5 | 0.168 | 1.378 | 86% |
| 57 | siRNA_22_Variant5 | 0.029 | 1.381 | 91% |
| 54 | siRNA_22_Variant2 | 0.290 | 1.524 | 89% |
| 34 | siRNA_4_Variant2 | 0.324 | 1.641 | 86% |
| 39 | siRNA_9_Variant2 | 0.326 | 1.663 | 86% |
| 55 | siRNA_22_Variant3 | 0.265 | 1.736 | 89% |
| 52 | siRNA_16_Variant5 | 0.058 | 1.885 | 85% |
| 1 | siRNA_1_Variant0 | 0.345 | 2.198 | 84% |
| 40 | siRNA_9_Variant3 | 0.468 | 2.442 | 87% |
| 10 | siRNA_10_Variant0 | 0.024 | 2.799 | 90% |
| 42 | siRNA_9_Variant5 | 0.500 | 2.910 | 84% |
| 43 | siRNA_10_Variant1 | 0.096 | 3.219 | 82% |
| 16 | siRNA_16_Variant0 | 0.315 | 6.534 | 83% |
| 31 | siRNA_1_Variant4 | 0.343 | 7.640 | 81% |
| 48 | siRNA_16_Variant1 | 0.119 | 8.349 | 82% |
| 41 | siRNA_9_Variant4 | 1.397 | 11.098 | 84% |
| 29 | siRNA_1_Variant2 | 0.979 | #N/A | 68% |
| 30 | siRNA_1_Variant3 | — | — | 36% |
| 35 | siRNA_4_Variant3 | 0.622 | — | 55% |
| 36 | siRNA_4_Variant4 | 0.486 | — | 68% |
| 44 | siRNA_10_Variant2 | 0.386 | — | 76% |
| 45 | siRNA_10_Variant3 | 0.905 | — | 50% |
| 46 | siRNA_10_Variant4 | 0.540 | — | 63% |
| 49 | siRNA_16_Variant2 | — | — | 28% |
| 50 | siRNA_16_Variant3 | — | — | 16% |
| 51 | siRNA_16_Variant4 | — | — | 32% |

TABLE 15

| siRNA | | | |
|---|---|---|---|
| ID | siRNA desciption | Sense sequence (5′ to 3′) | Antisense sequence (5′ to 3′) |
| 26 | (+) Control | CAACUCACCUGUAAUAAAUUU (SEQ | pAUUUAUUACAGGUGAGUUGUU (SEQ |
| | | ID NO: 251) | ID NO: 252) |
| 27 | (+) Control | GAGCCGUUCUCUACAAUUAUU (SEQ | pUAAUUGUAGAGAACGGCUCUU (SEQ |
| | | ID NO: 253) | ID NO: 254) |
| 1 | siRNA | uscsAfacuCfAfCfcuGfuaauAfaa | usUfsuauuacaggugAfguugasusu (SEQ |
| | ID_1_Variant0 | (SEQ ID NO: 201) | ID NO: 202) |
| 28 | siRNA | uscsAfacuCfAfCfcuGfuaauAfaa | usUfsuauuacaggugAfguugasusc (SEQ |
| | ID_1_Variant1 | (SEQ ID NO: 201) | ID NO: 256) |
| 29 | siRNA | UfscsAfaCfuCfaCfcUfgUfaAfuAfaAf | usUfsuAfuUfaCfaGfgUfgAfgUfuGfasusc |
| | ID_1_Variant2 | (SEQ ID NO: 255) | (SEQ ID NO: 257) |
| 30 | siRNA_1_Variant3 | UfscsAfaCfuCfaCfcUfgUfaAfuAfaAf | usUfsuauuacaggugAfguugasusc (SEQ |
| | | (SEQ ID NO: 255) | ID NO: 256) |
| 31 | siRNA 1 Variant4 | UfscsAfaCfuCfaCfcUfgUfaAfuAfaAf | usUfsuauUfacaggugAfgUfugasusc (SEQ |
| | | (SEQ ID NO: 255) | ID NO: 258) |
| 32 | siRNA_1_Variant5 | uscsAfacuCfAfCfcuGfuaauAfaa (SEQ | usUfsuauUfacaggugAfgUfugasusc (SEQ |
| | | ID NO: 201) | ID NO: 258) |
| 4 | siRNA_4_Variant0 | uscsCfaagCfCfUfugGfcucaAfua (SEQ | usAfsuugagccaaggCfuuggasusu (SEQ |
| | | ID NO: 207) | ID NO: 208) |
| 33 | siRNA_4_Variant1 | uscsCfaagCfCfUfugGfcucaAfua (SEQ | usAfsuugagccaaggCfuuggasasc (SEQ |
| | | ID NO: 207) | ID NO: 260) |
| 34 | siRNA_4_Variant2 | UfscsCfaAfgCfcUfuGfgCfuCfaAfuAf | usAfsuUfgAfgCfcAfaGfgCfuUfgGfasasc |
| | | (SEQ ID NO: 259) | (SEQ ID NO: 261) |
| 35 | siRNA_4_Variant3 | UfscsCfaAfgCfcUfuGfgCfuCfaAfuAf | usAfsuugagccaaggCfuuggasasc (SEQ |
| | | (SEQ ID NO: 259) | ID NO: 260) |
| 36 | siRNA_4_Variant4 | UfscsCfaAfgCfcUfuGfgCfuCfaAfuAf | usAfsuugAfgccaaggCfuUfggasasc (SEQ |
| | | (SEQ ID NO: 259) | ID NO: 262) |
| 37 | siRNA_4_Variant5 | uscsCfaagCfCfUfugGfcucaAfua (SEQ | usAfsuugAfgccaaggCfuUfggasasc (SEQ |
| | | ID NO: 207) | ID NO: 262) |
| 9 | siRNA_9_Variant0 | asusCfggaUfCfUfucAfccguCfaa (SEQ | usUfsgacggugaagaUfccgaususu (SEQ |
| | | ID NO: 217) | ID NO: 218) |
| 38 | siRNA_9_Variant1 | asusCfggaUfCfUfucAfccguCfaa (SEQ | usUfsgacggugaagaUfccgausasg (SEQ |
| | | ID NO: 217) | ID NO: 263) |
| 39 | siRNA_9_Variant2 | AfsusCfgGfaUfcUfuCfaCfcGfuCfaAf | usUfsgAfcGfgUfgAfaGfaUfcCfgAfusasg |
| | | (SEQ ID NO: 264) | (SEQ ID NO: 265) |
| 40 | siRNA_9_Variant3 | AfsusCfgGfaUfcUfuCfaCfcGfuCfaAf | usUfsgacggugaagaUfccgausasg (SEQ |
| | | (SEQ ID NO: 264) | ID NO: 263) |
| 41 | siRNA_9_Variant4 | AfsusCfgGfaUfcUfuCfaCfcGfuCfaAf | usUfsgacGfgugaagaUfcCfgausasg (SEQ |
| | | (SEQ ID NO: 264) | ID NO: 266) |
| 42 | siRNA_9_Variant5 | asusCfggaUfCfUfucAfccguCfaa (SEQ | usUfsgacGfgugaagaUfcCfgausasg (SEQ |
| | | ID NO: 217) | ID NO: 266) |
| 10 | siRNA10_Variant0 | uscsAfacuUfCfCfucCfugcgAfaa (SEQ | usUfsucgcaggaggaAfguugasusu (SEQ |
| | | ID NO: 219) | ID NO: 220) |
| 43 | siRNA10_Variant1 | uscsAfacuUfCfCfucCfugcgAfaa (SEQ | usUfsucgcaggaggaAfguugascsg (SEQ |
| | | ID NO: 219) | ID NO: 267) |
| 44 | siRNA10_Variant2 | UfscsAfaCfuUfcCfuCfcUfgCfgAfaAf | usUfsuCfgCfaGfgAfgGfaAfgUfuGfascsg |
| | | (SEQ ID NO: 268) | (SEQ ID NO: 269) |
| 45 | siRNA10_Variant3 | UfscsAfaCfuUfcCfuCfcUfgCfgAfaAf | usUfsucgcaggaggaAfguugascsg (SEQ |
| | | (SEQ ID NO: 268) | ID NO: 267) |
| 46 | siRNA10_Variant4 | UfscsAfaCfuUfcCfuCfcUfgCfgAfaAf | usUfsucgCfaggaggaAfgUfugascsg (SEQ |
| | | (SEQ ID NO: 268) | ID NO: 270) |
| 47 | siRNA10_Variant5 | uscsAfacuUfCfCfucCfugcgAfaa (SEQ | usUfsucgCfaggaggaAfgUfugascsg (SEQ |
| | | ID NO: 219) | ID NO: 270) |
| 16 | siRNA_16_Variant0 | csasAfugaCfUfUfugAfcgagUfaa (SEQ | usUfsacucgucaaagUfcauugsusu (SEQ |
| | | ID NO: 231) | ID NO: 232) |
| 48 | siRNA_16_Variant1 | csasAfugaCfUfUfugAfcgagUfaa (SEQ | usUfsacucgucaaagUfcauugsgsa (SEQ |
| | | ID NO: 231) | ID NO: 271) |
| 49 | siRNA_16_Variant2 | CfsasAfuGfaCfuUfuGfaCfgAfgUfaAf | usUfsaCfuCfgUfcAfaAfgUfcAfuUfgsgsa |
| | | (SEQ ID NO: 272) | (SEQ ID NO: 273) |
| 50 | siRNA_16_Variant3 | CfsasAfuGfaCfuUfuGfaCfgAfgUfaAf | usUfsacucgucaaagUfcauugsgsa (SEQ |
| | | (SEQ ID NO: 272) | ID NO: 271) |
| 51 | siRNA_16_Variant4 | CfsasAfuGfaCfuUfuGfaCfgAfgUfaAf | usUfsacuCfgucaaagUfcAfuugsgsa (SEQ |
| | | (SEQ ID NO: 272) | ID NO: 274) |
| 52 | siRNA_16_Variant5 | csasAfugaCfUfUfugAfcgagUfaa (SEQ | usUfsacuCfgucaaagUfcAfuugsgsa (SEQ |
| | | ID NO: 231) | ID NO: 274) |
| 22 | siRNA_22_Variant0 | cscsAfacuAfCfAfugAfaccuAfca | usGfsuagguucauguAfguuggsusu (SEQ |
| | | (SEQ ID NO: 243) | ID NO: 244) |
| 53 | siRNA_22_Variant1 | cscsAfacuAfCfAfugAfaccuAfca | usGfsuagguucauguAfguuggscsu (SEQ |
| | | (SEQ ID NO: 243) | ID NO: 275) |
| 54 | siRNA_22_Variant2 | CfscsAfaCfuAfcAfuGfaAfcCfuAfcAf | usGfsuAfgGfuUfcAfuGfuAfgUfuGfgscsu |
| | | (SEQ ID NO: 276) | (SEQ ID NO: 277) |
| 55 | siRNA_22_Variant3 | CfscsAfaCfuAfcAfuGfaAfcCfuAfcAf | usGfsuagguucauguAfguuggscsu (SEQ |
| | | (SEQ ID NO: 276) | ID NO: 275) |
| 56 | siRNA_22_Variant4 | CfscsAfaCfuAfcAfuGfaAfcCfuAfcAf | usGfsuagGfuucauguAfgUfuggscsu (SEQ |
| | | (SEQ ID NO: 276) | ID NO: 278) |
| 57 | siRNA_22_Variant5 | cscsAfacuAfCfAfugAfaccuAfca | usGfsuagGfuucauguAfgUfuggscsu (SEQ |
| | | (SEQ ID NO: 243) | ID NO: 278) |

IC50 and IC80 values in Table 14 indicate that the modification patterns varied in performance. For example, siRNA ID: 32 (using modification patterns identified in “variant5”) had better activity (0.018 nM and 0.108 nM IC50 and IC80 values, respectively) than siRNA IDs: 1 (“variant0”), 28 (“variant1”), 29 (“variant2”), 30 (“variant3”), and 31 (“variant4”), even though each of these siRNAs are based on the same nucleotide sequence, and target the same region of the C3 transcript.

Additionally, the performance of the modification patterns (i.e., different variants) appeared to vary by siRNA nucleotide sequence (i.e., the target region of the C3 transcript). For example, “variant5” was shown to be the most effective in C3 knockdown in siRNAs based on the nucleotide sequence of siRNA 1 (see siRNA ID: 32 (variant5) compared to e.g., siRNA ID: 1 (variant0)), whereas in siRNAs based on the nucleotide sequence of siRNA 22, “variant0” appeared to be the most effective in C3 knockdown (see siRNA ID: 22 (variant0) compared to e.g., siRNA: 55 (variant3)).

### Example 4: In Vivo Evaluation of siRNA 58 in Non-Human Primates

siRNA Construct

The siRNA 53 from Example 3 was selected and further modified as described below to generate siRNA 58.

TABLE 16

| siRNA | | |
|---|---|---|
| ID | Sense sequence (5′ to 3′) | Antisense sequence (5′ to 3′) |
| 58 | cscsAfacuAfCfAfugAfaccuAfscsa (SEQ | usGfsuagguucauguAfguuggscsu |
| | ID NO: 325) | (SEQ ID NO: 275) |

Additionally, the siRNA was conjugated to the GalNAc structure shown below via a NHC6 linker at the 5′ end of the sense strand of siRNA 58.

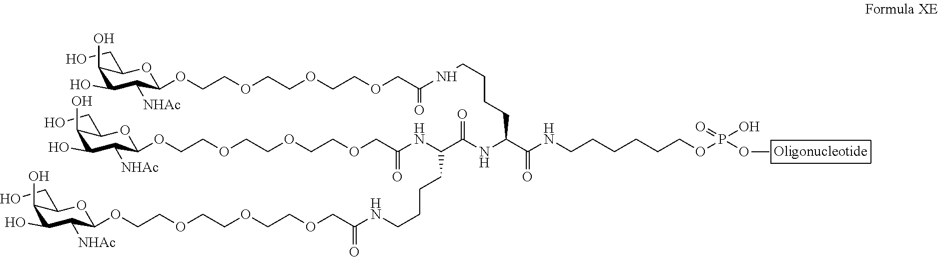

The modified siRNA (hereafter referred to as “siRNA 58”) was then evaluated in non-human primates.

### Study Design

Naive male cynomolgus monkeys (n=3 in each group) were subcutaneously (SC) administered a single dose of either 3 mg/kg. 10 mg/kg, or 30 mg/kg siRNA 58, or vehicle (phosphate buffered saline) on day 1.

Serum samples were scheduled to be collected on days −5, −1, 3, 8, 15, 22, 29, 40, 57, 67, 82, 97, 112, 127, 142, 157, 172 and 184 (where negative values correspond to days before injection with siRNA 58 or vehicle). The level of C3 protein in serum was measured using an ELISA assay. Additionally, serum samples were also analyzed for alternative complement pathway activity (AH50). Values at day −1 value were used as the baseline.

Liver needle biopsies were performed on days 15, 46 and 79. The level of C3 mRNA in the samples was measured using a quantitative PCR assay, C3 mRNA level was normalized to the level of ActB mRNA in these experiments.

### Results

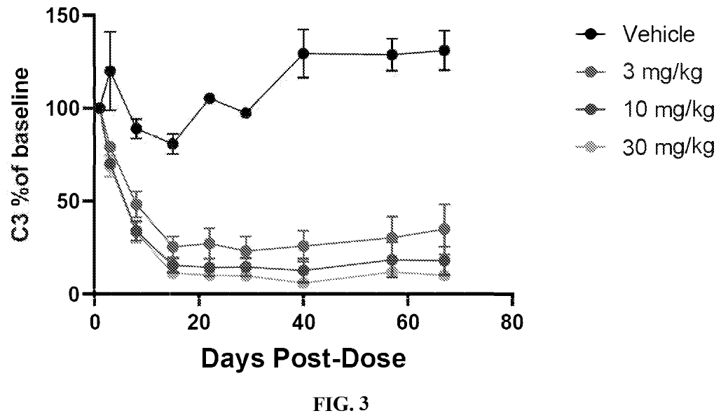

FIG. 3 presents the time course for the levels of serum C3 protein for up to 67 days post-dose for each group. The results indicate that a single SC dose of siRNA 58 reduced the level of serum C3 protein by 77% at the 3 mg/kg dose, by 85% at the 10 mg/kg dose, and by 90% at the 30 mg/kg dose by day 29, as compared to the baseline value, with reductions close to these levels evident by day 15. Additionally, the data in FIG. 3 show that reduction was sustained out to day 67.

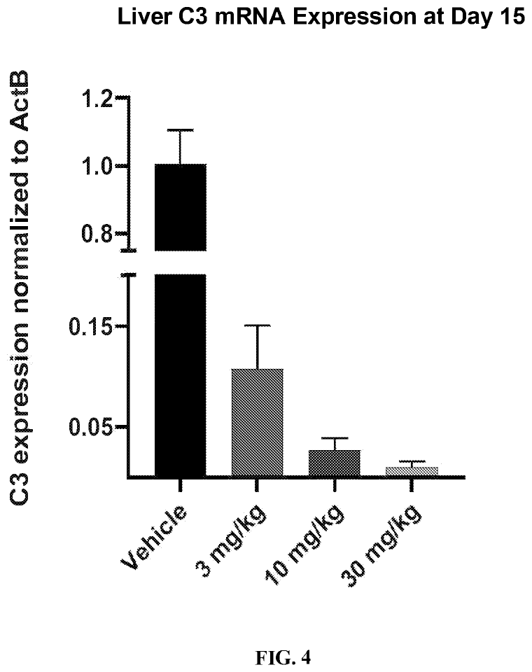

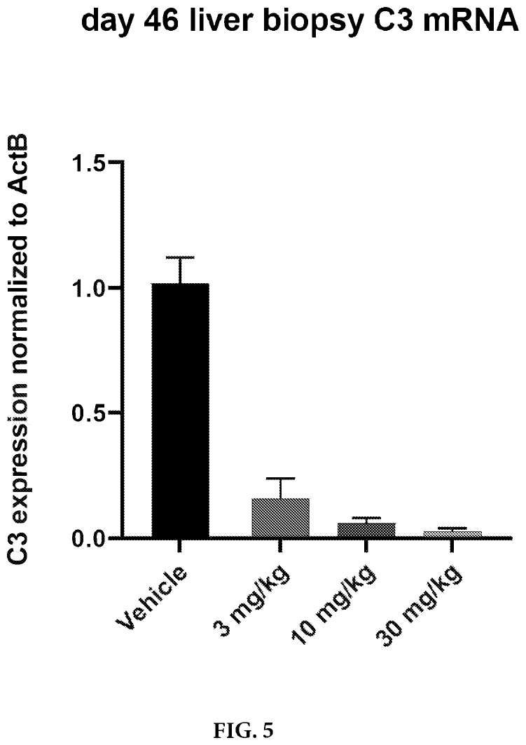

FIG. 4 shows that a single dose of siRNA 58 resulted in a reduction of liver C3 mRNA by 89% at the 3 mg/kg dose, 97% at the 10 mg/kg dose, and 99% at the 30 mg/kg dose, as compared to the vehicle control, by day 15. The reduction was sustained at day 46 (FIG. 5).

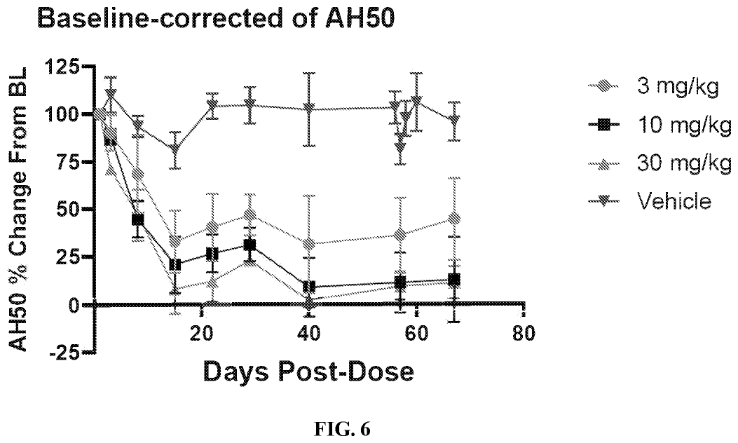

FIG. 6 presents a time course for the levels of alternative complement pathway (AH50) activity in serum collected up to day 67. The results indicate that a single SC dose of siRNA 58 reduced the level of alternative complement pathway activity by 65% at the 3 mg/kg dose, by 82% at the 10 mg/kg dose, and by 92% at the 30 mg/kg dose by day 29, as compared to the baseline value, with reductions reaching these levels by day 15. Additionally, the reduced activity was sustained at day 67, where activity was reduced by 68% at the 3 mg/kg dose, by 91% at the 10 mg/kg dose, and by 98% at the 30 mg/kg dose, as compared to the baseline value.

### Example 5: In Vivo Evaluation of siRNA 60 in Non-Human Primates

siRNA Construct

The siRNA 32 from Example 3 is selected and further modified as described below to generate siRNA 60.

TABLE 17

| siRNA | | |
|---|---|---|
| ID | Sense sequence (5′ to 3′) | Antisense sequence (5′ to 3′) |
| 60 | uscsAfacuCfAfCfcuGfuaauAfsasa (SEQ | usUfsuauUfacaggugAfgUfugasusc |
| | ID NO: 327) | (SEQ ID NO: 258) |

Additionally, the siRNA is conjugated to the GalNAc structure shown below via a NHC6 linker at the 5′ end of the sense strand of siRNA 60.

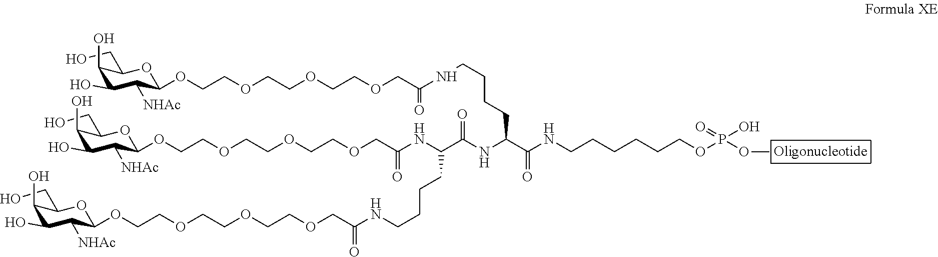

The modified siRNA (hereafter referred to as “siRNA 60”) is evaluated in non-human primates.

### Study Design

Naive male cynomolgus monkeys (n=3 in each group) are subcutaneously (SC) administered a single dose of either 3 mg/kg, 10 mg/kg, or 30 mg/kg siRNA 60, or vehicle (phosphate buffered saline) on day 1.

Serum samples are collected on days −5, −1, 3, 8, 15, 22, 29, 40, 57, 67, 82, 97, 112, 127, 142, 157, 172 and 184 (where negative values correspond to days before injection with siRNA 60 or vehicle). The level of C3 protein in serum is measured using an ELISA assay. Additionally, serum samples are also analyzed for alternative complement pathway activity (AH50). Values at day −1 value are used as the baseline.

Liver needle biopsies are performed on days 15, 46 and 79. The level of C3 mRNA in the samples is measured using a quantitative PCR assay, C3 mRNA level is normalized to the level of ActB mRNA in these experiments.

### Example 6: Off-Target Analysis and Safety of siRNA 58

The nucleotide sequences of the sense and antisense strands of siRNA 58 were analyzed for potential off-target activity (Lindow et al. 2012). The potential off-target activities for the sense and antisense strands were analyzed in mature human RNAs and primary human RNAs.

### Methods

The sequences analyzed were as follows:

Antisense (guide) strand:
(SEQ ID NO: 321)
5′-uguagguucauguaguuggcu-3′
Sense (passenger) strand:
(SEQ ID NO: 147)
5′-ccaacuacaugaaccuaca-3′

Antisense Strand (mature human RNAs): In order to identify potential off-target genes, similarity searches were performed using the Smith-Waterman gapped local alignment (sSearch) in the FASTA package (v36; Pearson 2000) with the following parameters:
-E E-value less than 5000. E corresponds to the number of search hits one can expect to see by chance when searching a database of this size; relatively high numbers are used to ensure all potential hybridization off-target sequences are detected.
-W (number of positions flanking alignments) is set to 5
-n for nucleic acids search
-f and -g for gap creation and extension penalties set to 1000 to avoid gapped alignments ssearch36 -n -W 5 -E 5000 -f 1000 -g 1000 query refMrna.fa

Antisense Strand (primary human RNAs): In order to identify genes with a potential off-target effect within the nucleus, the matching of the oligonucleotide sequence to primary RNAs (which are unspliced and contain introns) was probed. Specifically, these similarity searches were performed using the Smith-Waterman gapped local alignment (sSearch) in the FASTA package (v36; Pearson 2000) to the human genome (version hg38) with the following parameters:
-E E-value less than 5000. E corresponds to the number of search hits one can expect to see by chance when searching a database of this size; relatively high numbers are used to ensure all potential hybridization off-target sequences are detected.
-W (number of positions flanking alignments) is set to 5
-n for nucleic acids search
-f and -g for gap creation and extension penalties set to 1000 to avoid gapped alignments ssearch36 -n -W 5 -E 5000 -f 1000 -g 1000 query refGene.fa

Genomic alignment can identify the positions in the reference genome at which each sequence was found; however, the reference genome itself does not contain the locations of genes and gene bodies. Thus, to obtain information regarding which genes had the potential for hybridization, following alignment, sSearch genomic coordinates of hits were converted to a bed file format, and bedtools (v2.28.0) intersect was used to annotate the intragenic hits to their genes. Gene positions were obtained from the UCSC genome browser Table Viewer for Gencodev32 and hg38, from the “Gene and gene predictions table.” The following command was used for the annotation with bedtools:
bedtools intersect -a hitBedFile.bed -b geneBedFile.bed -wo
-a and -b notate inputs
-wo writes out the original positions of both hits and full annotations, needed for obtaining gene names

Sense strand (mature human RNAs): In order to identify potential off-target genes, similarity searches were performed using the Smith-Waterman gapped local alignment (sSearch) in the FASTA package (v36; Pearson 2000) with the following parameters:
-E E-value less than 5000. E corresponds to the number of search hits one can expect to see by chance when searching a database of this size; relatively high numbers are used to ensure all potential hybridization off-target sequences are detected.
-W (number of positions flanking alignments) is set to 5
-n for nucleic acids search
-f and -g for gap creation and extension penalties set to 1000 to avoid gapped alignments

The search was performed to look for mature RNA sequences with (1) longest uninterrupted complementarity over 11 (reduced from 13 due to shorter sense sequence) and with the number of matches over 16 (2 or one mismatches), and/or (2) 14 or more bp of uninterrupted complementarity with 16 matches (3 mismatches).

Sense Strand (primary human RNAs): In order to identify genes with potential off-target effect within the nucleus, the matching of the oligonucleotide sequence to primary RNAs (which are unspliced and contain introns) was probed. Specifically, these similarity searches were performed using the Smith-Waterman gapped local alignment (sSearch) in the FASTA package (v36; Pearson 2000) to the human genome (version hg38) with the following parameters:
-E E-value less than 5000. E corresponds to the number of search hits one can expect to see by chance when searching a database of this size; relatively high numbers are used to ensure all potential hybridization off-target sequences are detected.
-W (number of positions flanking alignments) is set to 5
-n for nucleic acids search
-f and -g for gap creation and extension penalties set to 1000 to avoid gapped alignments ssearch36 -n -W 5 -E 5000 -f 1000 -g 1000 query refGene.fa

To obtain information regarding which genes had the potential for off-target cleavage of their primary mRNAs by Argonaut, following alignment, sSearch genomic coordinates of hits were converted to a bed file format, and bedtools (v2.28.0) intersect was used to annotate the intragenic hits to their genes. Gene positions were obtained from the UCSC genome browser Table Viewer for Gencodev32 and hg38, from the “Gene and gene predictions table.” The following command was used for the annotation with bedtools:
bedtools intersect -a hitBedFile.bed -b geneBedFile.bed -wo
-a and -b notate inputs
-wo writes out the original positions of both hits and full annotations, needed for obtaining gene names

Gene Analysis: Genes identified based on the above searches were further analyzed in order to understand any potential safety risks. Gene expression in the main organs of oligonucleotide drug accumulation-liver and kidney-were evaluated. GTEx human tissue expression atlas (GTEx Consortium 2013) was used to calculate log 2 transformed Transcript per million (TPM) expression values for each gene in each subject in GTEx, and median values for each gene were obtained. Values: below 3 indicate very low expression, from 3-4 indicate low expression, from 4-6 indicate intermediate expression, and over 6 indicates high expression.

Next, searches for evidence of disease associations with full knockdown or partial knockdown of each target was performed. To examine evidence of disease in the presence of full knockdown of a target, the Online Mendelian Inheritance of Man database (OMIM; Hamosh et al. 2002) was used to find associations with rare germline mutations and human genetic disorders. Notably, most of the top gene off-target hits contain multiple mismatches that should severely dimmish RNAi efficiency. While most human autosomal genes are not dosage specific (Rice & McLysaght 2017), and inactivation of one allele does not lead to any phenotype, an additional search for evidence of dosage specific effects was performed using ClinGen (https://search.clinicalgenome org/), which collects information about both haploinsufficient and triplosensitivite dosage specific genes.

### Results:

Antisense Strand: The only perfect complementarity hit for the antisense strand of siRNA 58 is the target C3. The next best matches among primary and mature RNAs all had 3 or more mismatches, which would be expected to have significantly reduced hybridization-dependent off-target effects. Most of the top off-target candidates exhibited low expression in liver and kidney and/or no association with human genetic disorders. The only exception was RABL3, where a specific gain of function truncating mutation was associated with hereditary pancreatic cancer syndrome in a single family. Potential knockdown of RABL3 by siRNA 58 is not expected to mimic this gain-of-novel-function mutation.

Sense Strand: Among sequences complementary to the sense strand of siRNA 58, there was no perfect complementarity match among primary and mature human RNAs. Most of the genes with complementarity to the sense strand were neither appreciably expressed in human liver nor kidney, nor were they linked to known genetic disorders, including GPR173 with just a single mismatch to the sense oligonucleotide.

### Example 7: In Vivo Evaluation of siRNA 59 in Rats

siRNA Construct

The siRNA 33 from Example 3 was further modified as described below to generate siRNA 59.

TABLE 18

| siRNA | | |
|---|---|---|
| ID | Sense sequence (5′ to 3′) | Antisense sequence (5′ to 3′) |
| 59 | uscsCfaagCfCfUfugGfcucaAfsusa | usAfsuugagccaaggCfuuggasasc |
| | (SEQ ID NO: 326) | (SEQ ID NO: 260) |

Additionally, the siRNA was conjugated to the GalNAc structure shown below via a NHC6 linker at the 5′ end of the sense strand of siRNA 59.

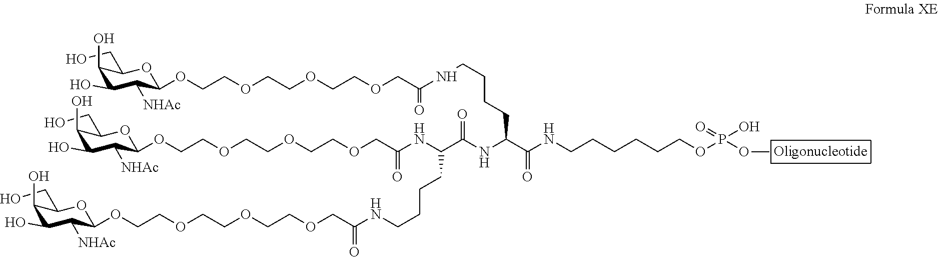

The modified siRNA (hereafter referred to as “siRNA 59”) was then evaluated in Sprague-Dawley rats.

Objectives: The objective of this study was to determine the plasma pharmacokinetics (PK) and limited tissue distribution of siRNA 59 at three dose levels, when administered as a single subcutaneous injection to Sprague-Dawley rats. The secondary objective of this study was to compare the pharmacodynamic effect of three dose levels of siRNA 59 administered as a single subcutaneous injection versus an equivalent dose administered over the course of three days (one administration per day).

### Methods

### Study Design:

TABLE 19
Experimental Study Design

| | | | | Pharmacodynamic | | Pharmacokinetic | | |
|---|---|---|---|---|---|---|---|---|
| | | Target | | Animals (PD) | | Animals (PK) | | |
| | | Dose | Route, | | Blood Collection | | Blood Collection | |
| Group | TA | (mg/kg) | Day | N | Time Points | N | Time Points | Necropsy |
| 1 | siRNA 59 | 3 mg/kg | SC, | 3 | Baseline^{A}, Day | 3 | 15(±5) minutes, | Day 3 - |
| | | | Day 1 | | 3, 8, 15, 22 and | | 1 hours (±10 min), | PK animals |
| 2 | | 10 mg/kg | SC, | 3 | 29 post-dose | 3 | 4 hours (±10 min), | Day 30 - |
| | | | Day 1 | | | | 8 hours (±20 min) | PD animals |
| 3 | | 30 mg/kg | SC, | 3 | | 3 | 24 hours (±20 min), | |
| | | | Day 1 | | | | 48 hours (±20 min), | |
| 4 | | 3 × 1 mg/kg | SC, | 3 | | — | and | |
| | | (3 mg/kg ^{B}) | Day 1-3 | | | | 72 hours (±20 min) | |
| 5 | | 3 × 3.3 mg/kg | SC, | 3 | | — | post dose | |
| | | (10 mg/kg ^{B}) | Day 1-3 | | | | | |
| 6 | | 3 × 10 mg/kg | SC, | 3 | | — | | |
| | | (30 mg/kg ^{B}) | Day 1-3 | | | | | |
| 7 | siRNA (−) | 3 × 10 mg/kg | SC, | 3 | | — | | |
| | control^{C} | (30 mg/kg ^{B}) | Day 1-3 | | | | | |
| 8 | Vehicle | n/a; dose | SC | 3 | | 3 | | |
| | | volume to | | | | | | |
| | | match highest | | | | | | |
| | | delivered to | | | | | | |
| | | TA group | | | | | | |

^{A}Baseline collection = one collection occurring between Day −5 and −1
^{B} Animals in Groups 4, 5, 6, and 7 will receive 3 daily doses of TA (or placebo) for the indicated total target dose of 3, 10, 30 and 30 mg/kg, respectively.
^{C}Negative control (siRNA that is not cross-reactive in rats and does not silence C3).
SC = subcutaneous;
PK = pharmacokinetic;
PD = pharmacodynamic;
TA = test article

Dosing: Animals from groups 1-3 and 8 were dosed with a single subcutaneous injection of PBS vehicle or 3, 10 or 30 mg/kg siRNA 59, formulated at a concentration of 0.6, 2 and 6 mg/ml in PBS, respectively, in a dosing volume of 5 ml. Animals from groups 4-7 were dosed with 3× daily subcutaneous injection of 10 mg/ml siRNA (−) control, or 1, 3.3 or 10 mg/ml siRNA 59, formulated at a concentration of 0.2, 0.6 and 2 mg/ml in PBS, respectively, in a dosing volume of 5 ml.

Sampling: For PD analyses, plasma and serum samples were collected at baseline and days 3, 8, 15, 22 and 29 post-dose. Plasma samples were collected at 15 minutes and 1, 4, 8, 24, 48 and 72 hours post-dose. Liver samples were collected at necropsy on days 3 and 30 post-dose.

Bioanalytical Methods: Plasma PD samples were analyzed for concentrations of C3 protein at Confluence Discovery Technologies (MO, USA) using a double antibody sandwich ELISA kit according to the manufacturer's instructions (Eagle Biosciences; plasma dilution of 1:10,000).

Serum PD samples were analyzed for alternative pathway (AP) complement activation at Confluence Discovery Technologies (MO, USA) using the AP Wieslab assay (Eagle Biosciences, Cat #COMPL AP330).

PD tissue samples were analyzed for C3 mRNA levels using a semi-validated RT-qPCR method at EpigenDx (MA, USA).

### Results

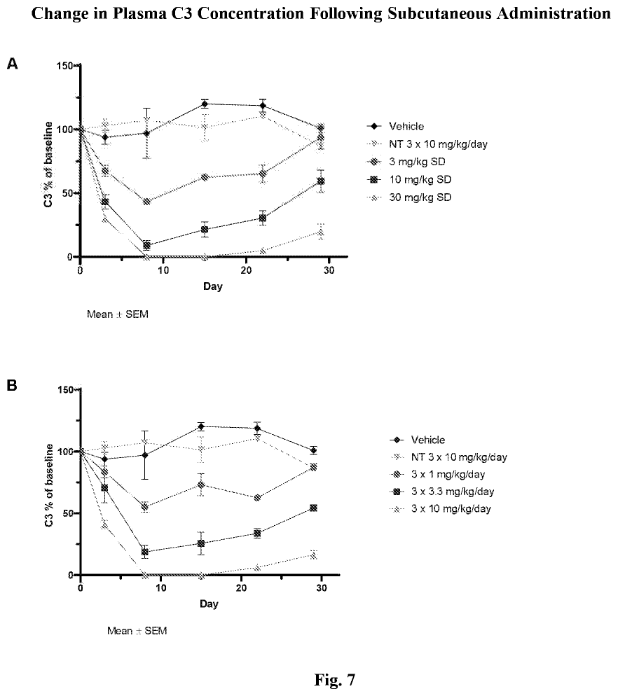

Circulating C3: Plasma was taken at 5 timepoints and assessed for changes in C3 concentration. A clear dose-dependent response to the siRNA was seen across the dose groups (FIG. 7). Maximal levels of C3 protein reduction was achieved by study day 8 for all dose groups. The plasma C3 concentration data were comparable between the single dose treatment and the QDx3 equivalent dose groups. Full recovery of plasma C3 protein to baseline levels were observed in the 3 mg/kg dose group only. The 10 and 30 mg/kg dose groups did not appear to have fully recovered to their respective baseline C3 protein levels by the final blood collection on day 29.

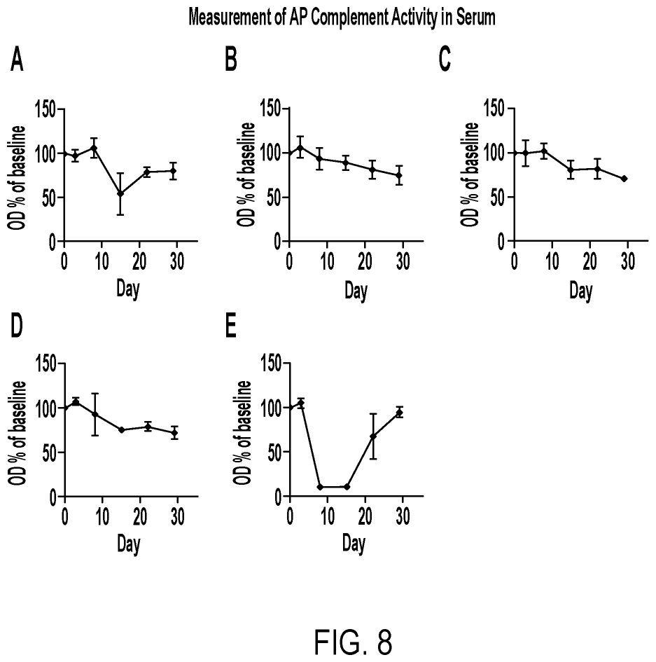

Alternative pathway complement activity: Sera from each animal treated with single dose siRNA 59 and vehicle, as well as the multi-dose siRNA (−) control-treated animals, were used in the AP Wieslab assay, an ELISA-based assay that measures formation of C5b-9 following alternative pathway activation, to assess complement activation (FIG. 8). FIG. 8 shows a measurement of alternative pathway activity, by detection and quantification of soluble C5b-9 complex using ELISA (measurement of optical density; OD), in serum from animals subcutaneously treated with vehicle (A); siRNA (−) control (B); siRNA 59 at 3 mg/kg (C); siRNA 59 at 10 mg/kg (D); and siRNA 59 at 30 mg/kg (E). Data represents mean±SEM (n=3). Serum AP complement activity was highly variable across the control groups. Serum from the high dose siRNA 59 group had a mean reduction in alternative pathway activity of approximately 90%, which persisted from day 8 to day 15 post-dose.

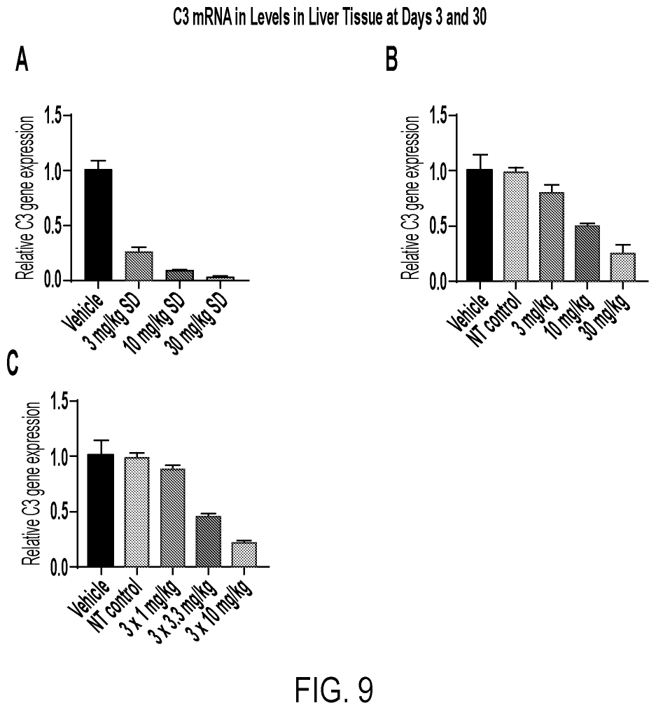

C3 transcript in liver tissue: At both terminal timepoints in the study, reduction in C3 mRNA in liver was observed to occur in a dose-dependent manner (FIG. 9). At 3 days following treatment, the level of liver C3 mRNA in the single dose PK animal groups was significantly reduced compared with C3 expression in the vehicle control. In animals treated with 30 mg/kg siRNA 59, the mean C3 expression in liver was at 3% of the gene expression in the vehicle treated arm, which was elevated to 25% by day 30. None of the treatment animals had fully recovered C3 expression by study termination at day 30.

Discussion: A clear, dose-dependent reduction of C3 protein in plasma and C3 mRNA in liver tissue was observed following subcutaneous administration of siRNA 59. Additionally, the observed dose-response was similar when siRNA 59 was administered as a single subcutaneous bolus and as a daily injection over the course of 3 days. By days 29 and 30, respectively, C3 plasma protein concentrations and mRNA expression in the liver had recovered in the low-dose groups only.

C3 plasma protein levels and liver gene expression were comparable at all timepoints between single dose groups and their multidose equivalents. No change in AP Wieslab activity was observed in any of the dose groups, except for serum from high-dose animals, despite the ˜90% reduction in C3 plasma protein concentration following a 10 mg/kg dose. No signs of distress or behavioral changes were noted in the animals from any treatment groups, and no loss in body weight was observed following TA administration, suggesting the doses used in this study were well-tolerated.

These results indicate that systemic circulating C3 protein can be silenced in a dose-dependent manner, following treatment with a GalNAc-tagged siRNA targeting C3.

### Example 8: Evaluation of C3 and C3a in Blood Fraction, Aqueous Humor (AH) and Vitreous Humor (VH) after Delivery of Liver-Targeted siRNA to Non-Human Primates

The present Example demonstrates, among other things, that protein concentrations of C3 and C3a in tissues such as plasma/serum and the AH and VH of non-human primates is reduced when a liver-targeted siRNA (which targets C3) is administered systemically. In this Example, a GalNAc-conjugated siRNA (“siRNA 58” from Example 4) was administered systemically to non-human primates. The specific materials and methods are detailed below.

### Study Design:

siRNA 58 or vehicle was administered to male cynomolgus monkeys as a single subcutaneous dose daily from days 1 to 4.

Serum/plasma samples were collected on Week 2, Week 4 and prior to terminal necropsy (Week 6). Samples of aqueous humor (AH) and vitreous humor (VH) was collected from each animal prior to necropsy. Tissue samples were collected on week 6 at necropsy.

The experimental design of this study is summarized in Table 20 below.

TABLE 20

| Experimental Design | | | | | Main |
|---|---|---|---|---|---|
| | | | Dose | Dose | Study |
| Group | Test | Dose Level | Volume* | Concentration | No. of |
| No. | Material | (mg/kg/dose) | (mL/kg) | (mg/mL) | Males |
| 1 | Vehicle | 0 | 2 | 0 | 4 |
| 2 | siRNA | 100 | 2 | 50 | 4 |

No. = Number
*Based on the most recent body weight measurement.

### Methods

Serum, AH, and VH C3 Analysis: Samples were analyzed for C3 protein concentration using an electrochemiluminescence detecting immunoassay (Mesoscale Delivery® (MSD); Cat #K151XYR-2) according to manufacturer's instruction. Prior to analysis, serum samples were diluted 1:10,000, AH samples were diluted 1:10, and VH samples were diluted 1:10. AH and VH samples were vortexed prior analysis.

Plasma, AH, and VH C3a Analysis: Samples were analyzed for C3a protein concentration using an enzyme-linked immunosorbent assay (ELISA) (Quidel®; Cat #A031) according to manufacturer's instruction. Prior to analysis, plasma samples were diluted 1:100 and AH and VH samples were diluted 1:5. AH and VH samples were vortexed prior analysis.

### Results

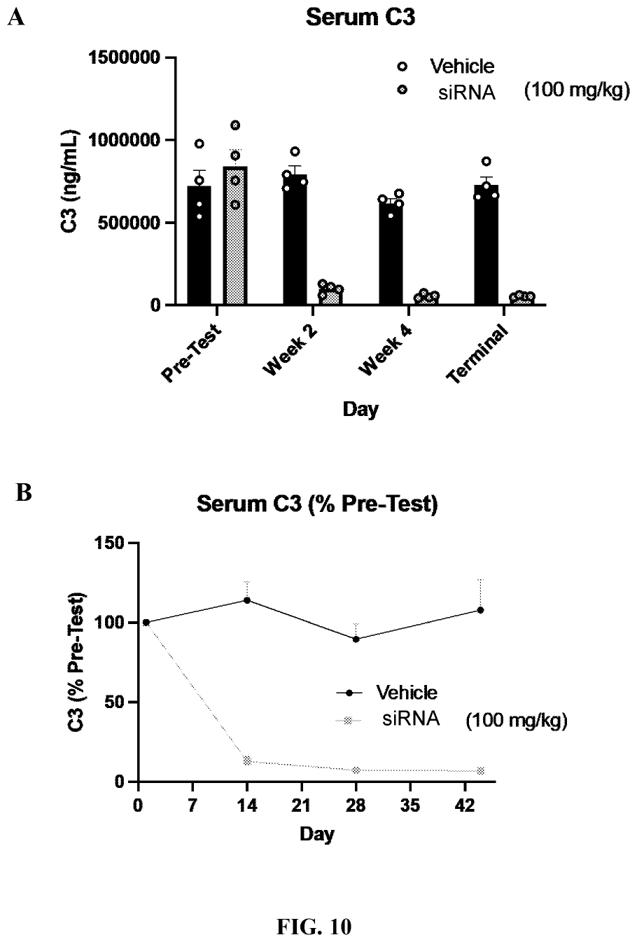

Serum C3 and Plasma C3a data: C3 levels in serum at the various timepoints following administration of siRNA or vehicle are shown in FIG. 10, panels A and B. FIG. 10, Panel A shows average serum C3 concentration (ng/ml). FIG. 10, Panel B shows average serum C3 reduction as a percent reduction from levels before siRNA or vehicle were administered (i.e., baseline levels).

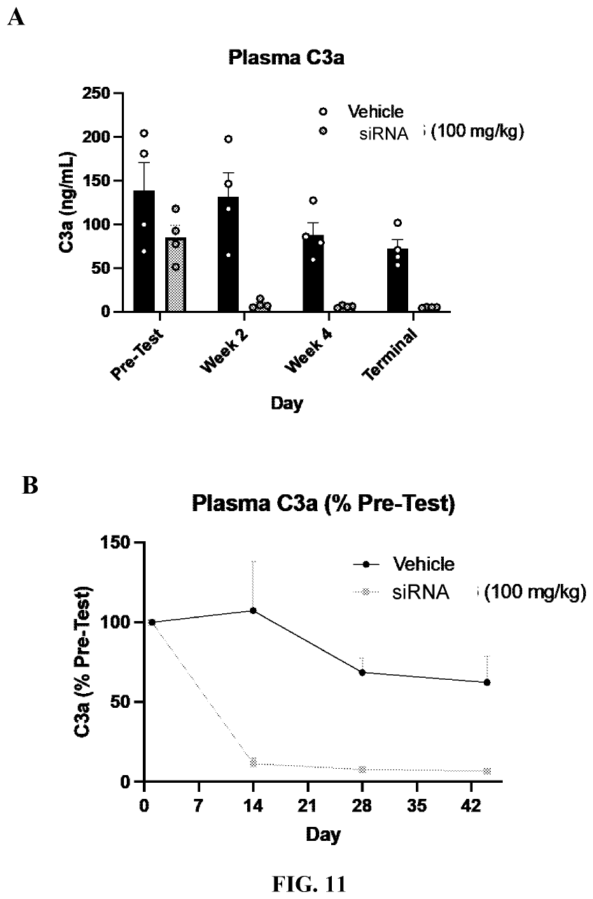

C3a levels in plasma at the various timepoints following administration of siRNA or vehicle are shown in FIG. 11, panels A and B. FIG. 11, Panel A shows average plasma C3a concentration (ng/ml). FIG. 11, Panel B shows average plasma C3a reduction as a percent reduction from levels before siRNA or vehicle were administered (i.e., baseline levels).

The results show a significant reduction in C3 and C3a protein in serum/plasma (i.e., the blood fraction) as a result of systemic administration of siRNA 58. The average reduction or knock down (KD) in protein concentration was between about 87-93% for both analytes C3 and C3a (see FIG. 10, panel B and Figure. 11, panel B).

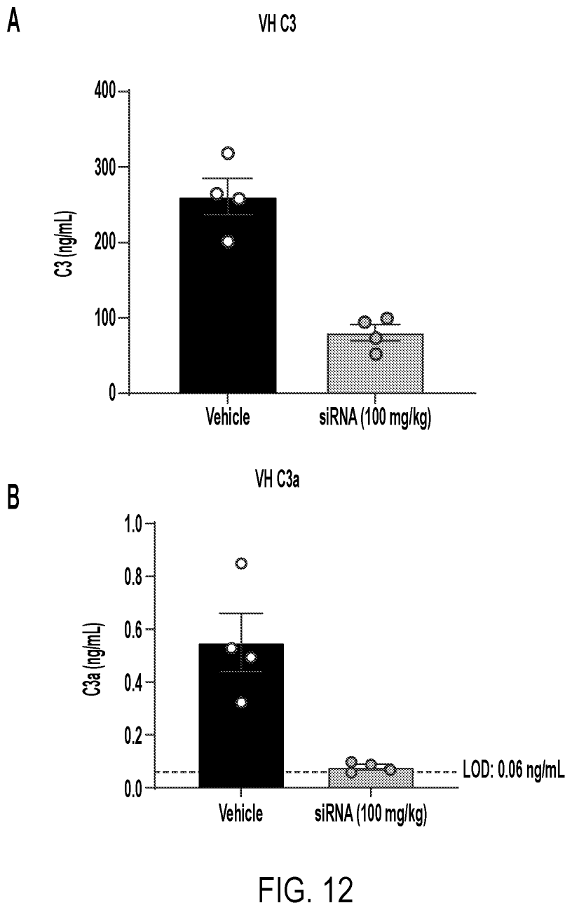

Vitreous Humor C3 and C3a data: C3 and C3a levels (ng/ml) in vitreous humor (VH) at Day 44 are shown in FIG. 12, panels A and B, respectively.

These results show that there was a significant reduction in both C3 and C3a levels in vitreous humor (VH) in animals dosed with siRNA 58 compared to animals dosed with vehicle. The average reduction in protein concentration (KD) was about 70% for C3 and about 83% for C3a when compared with baseline levels (before dosing). There was one animal in the treatment group receiving siRNA 58 where C3a values were reported below the limit of detection (LOD) or 0.06 ng/ml.

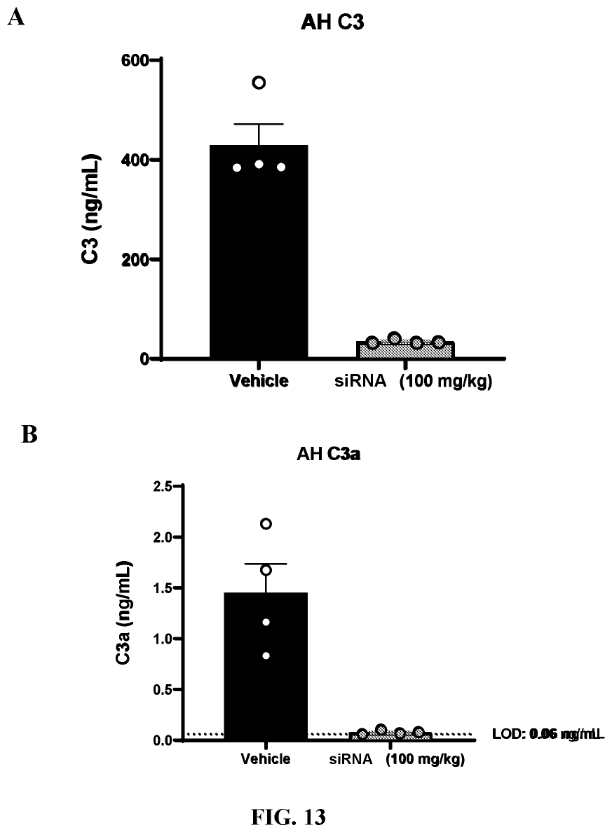

Aqueous Humor C3 and C3a data: C3 and C3a levels (ng/ml) in aqueous humor (AH) at Day 44 are shown in FIG. 13, panels A and B, respectively.

These results show that there was a significant reduction in both C3 and C3a protein levels in AH in animals dosed with siRNA 58 compared to animals dosed with vehicle. The average reduction in protein concentration (KD) was about 92% for C3 and about 94% for C3a when compared with baseline levels (before dosing). There was one animal in the treatment group receiving siRNA 58 where C3a values were reported below the limit of detection (LOD) or 0.06 ng/ml.

These data support the insight that systemic delivery of liver-targeted siRNA can reduce C3 and C3 activation products in ocular tissues, and be used to treat complement-mediated ocular disorders.

### IX. ADDITIONAL EXEMPLARY SEQUENCES

TABLE 21
Sense Sequence 5′ to 3′

CGUGGUCAAGGUCUUCUCUCU
ACGUGGUCAAGGUCUUCUCUA
UUUGACCUCAUGGUGUUCGUG

TABLE 22
Sense Sequence 5′ to 3′

GGAGAAUUGCUUCAUACAAAA
UGUUAAAUGGCUGAUCCUGGA
GACAGACAAGACCAUCUACAC
CCAGACAGACAAGACCAUCUA
CCAGAUCCACUUCACCAAGAA
UUGACCUCAUGGUGUUCGUGA
CCCCUUCGAGGUCACAGUAAU
AUGAACAAAACUGUGGCUGUU
AGACAGACAAGACCAUCUACA
CCAGAUCCACUUCACCAAGAC
AGGGAUCUGUGUGGCAGACCA
GACAAGACCAUCUACACCCCU
GCUGAGGAGAAUUGCUUCAUA
ACGUGGUCAAGGUCUUCUCUC
GGAUCUGUGUGGCAGACCCCU
ACAGACAAGACCAUCUACACA
AUCCAGACAGACAAGACCAUU
CUCCGUGUGGGUGGACGUCAA
UCCAGACAGACAAGACCAUCU
AGGGAUCUGUGUGGCAGACCC
CAAGAAAGGGAUCUGUGUGGA

TABLE 23
Sense Sequence 5′ to 3′

UGACCUCAUGGUGUUCGUGAU
GCAGCUAAAAGACUUUGACUU
CAUCCAGACAGACAAGACCAU
ACAGACAAGACCAUCUACACC
AUCCAGACAGACAAGACCAUC
UUUGACCUCAUGGUGUUCGUU
GGAUGCCAAGAACACUAUGAU
AAGAAAGGGAUCUGUGUGGCA
CAAGAAAGGGAUCUGUGUGGC
UACGUGGUCAAGGUCUUCUCU
CAGUUUCGAGGUCAUAGUGGA
CGUGCCGGAAGGAAUCAGAAU
GAAAGGGAUCUGUGUGGCAGA
GACAGACAAGACCAUCUACAA
UGACCUCAUGGUGUUCGUGAC
UGUAAUAAAUUCGACCUCAAG
AACUACAUGAACCUACAGAGA
AGAGAGAAGACCUUGACCACGUA
UAGAGAAGACCUUGACCACGUAG
CACGAACACCAUGAGGUCAAAGG
UUUUGUAUGAAGCAAUUCUCCUC
UCCAGGAUCAGCCAUUUAACAGC
GUGUAGAUGGUCUUGUCUGUCUG
UAGAUGGUCUUGUCUGUCUGGAU
UUCUUGGUGAAGUGGAUCUGGUA
UCACGAACACCAUGAGGUCAAAG
AUUACUGUGACCUCGAAGGGGUC
AACAGCCACAGUUUUGUUCAUUC
UGUAGAUGGUCUUGUCUGUCUGG
GUCUUGGUGAAGUGGAUCUGGUA
UGGUCUGCCACACAGAUCCCUUU
AGGGGUGUAGAUGGUCUUGUCUG
UAUGAAGCAAUUCUCCUCAGCAC
GAGAGAAGACCUUGACCACGUAG
AGGGGUCUGCCACACAGAUCCCU
UGUGUAGAUGGUCUUGUCUGUCU
AAUGGUCUUGUCUGUCUGGAUGA
UUGACGUCCACCCACACGGAGUC
AGAUGGUCUUGUCUGUCUGGAUG
GGGUCUGCCACACAGAUCCCUUU
UCCACACAGAUCCCUUUCUUGUC
AUCACGAACACCAUGAGGUCAAA
AAGUCAAAGUCUUUUAGCUGCAG
AUGGUCUUGUCUGUCUGGAUGAA
GGUGUAGAUGGUCUUGUCUGUCU
GAUGGUCUUGUCUGUCUGGAUGA
AACGAACACCAUGAGGUCAAAGG
AUCAUAGUGUUCUUGGCAUCCUG
UGCCACACAGAUCCCUUUCUUGU
GCCACACAGAUCCCUUUCUUGUC
AGAGAAGACCUUGACCACGUAGG
UCCACUAUGACCUCGAAACUGGG
AUUCUGAUUCCUUCCGGCACGAC
UCUGCCACACAGAUCCCUUUCUU
UUGUAGAUGGUCUUGUCUGUCUG
GUCACGAACACCAUGAGGUCAAA
CUUGAGGUCGAAUUUAUUACAGG
UCUCUGUAGGUUCAUGUAGUUGG

TABLE 24

| Sense Sequence | Antisense Sequence |
|---|---|
| 5′ to 3′ | 5′ to 3′ |
| CGGGUACCUCUUCAUCCAGAU | AUCUGGAUGAAGAGGUACCCGCU |
| GUACCUCUUCAUCCAGACAGU | ACUGUCTGGAUGAAGAGGUACCC |
| UACCUCUUCAUCCAGACAGAU | AUCUGUCUGGAUGAAGAGGUACC |
| CAUCCAGACAGACAAGACCAU | AUGGUCTUGUCUGUCUGGAUGAA |
| CCAGACAGACAAGACCAUCUU | AAGAUGGUCUUGUCUGUCUGGAU |
| AGACAGACAAGACCAUCUACU | AGUAGATGGUCUUGUCUGUCUGG |
| GACAGACAAGACCAUCUACAU | AUGUAGAUGGUCUUGUCUGUCUG |
| AUUCCGGAACUCGUCAACAUU | AAUGUUGACGAGUUCCGGAAUGU |
| CACUGAGUUUGAGGUGAAGGU | ACCUUCACCUCAAACUCAGUGGA |
| ACUGAGUUUGAGGUGAAGGAU | AUCCUUCACCUCAAACUCAGUGG |
| GCCCAGUUUCGAGGUCAUAGU | ACUAUGACCUCGAAACUGGGCAG |
| AAUUCUACUACAUCUAUAACU | AGUUAUAGAUGUAGUAGAAUUUC |
| UCCCUACCAGAUCCACUUCAU | AUGAAGTGGAUCUGGUAGGGAGA |
| CCCUACCAGAUCCACUUCACU | AGUGAAGUGGAUCUGGUAGGGAG |
| CUACCAGAUCCACUUCACCAU | AUGGUGAAGUGGAUCUGGUAGGG |
| UACCAGAUCCACUUCACCAAU | AUUGGUGAAGUGGAUCUGGUAGG |
| CCAGAUCCACUUCACCAAGAU | AUCUUGGUGAAGUGGAUCUGGUA |
| GGGCAACUCCAACAAUUACCU | AGGUAATUGUUGGAGUUGCCCAC |
| GCAACUCCAACAAUUACCUGU | ACAGGUAAUUGUUGGAGUUGCCC |
| CAACUCCAACAAUUACCUGCU | AGCAGGTAAUUGUUGGAGUUGCC |
| CAACAAUUACCUGCAUCUCUU | AAGAGATGCAGGUAAUUGUUGGA |
| CAAGAUCCGCUACUACACCUU | AAGGUGTAGUAGCGGAUCUUGGC |
| CGUGCUGAAUAAGAAGAACAU | AUGUUCTUCUUAUUCAGCACGAA |
| GUGCUGAAUAAGAAGAACAAU | AUUGUUCUUCUUAUUCAGCACGA |
| ACUGACGCAGAGUAAGAUCUU | AAGAUCTUACUCUGCGUCAGUUU |
| UGCAGAAGAGAACAUCGUUUU | AAAACGAUGUUCUCUUCUGCAAU |
| CCUCAAGGUCACCAUAAAACU | AGUUUUAUGGUGACCUUGAGGUC |
| CUCAAGGUCACCAUAAAACCU | AGGUUUUAUGGUGACCUUGAGGU |
| UCAAGGUCACCAUAAAACCAU | AUGGUUUUAUGGUGACCUUGAGG |
| GAUGCCAAGAACACUAUGAUU | AAUCAUAGUGUUCUUGGCAUCCU |
| AGGAUGCCACUAUGUCUAUAU | AUAUAGACAUAGUGGCAUCCUGG |
| CAAGGUCUACGCCUAUUACAU | AUGUAAUAGGCGUAGACCUUGAC |
| AAGGUCUACGCCUAUUACAAU | AUUGUAAUAGGCGUAGACCUUGA |
| AGGUCUACGCCUAUUACAACU | AGUUGUAAUAGGCGUAGACCUUG |

TABLE 25

| Sense Sequence | Antisense Sequence |
|---|---|
| 5′ to 3′ | 5′ to 3′ |
| ACGGUCAUGGUCAACAUUGAU | AUCAAUGUUGACCAUGACCGUCC |
| AGAUCCGAGCCUACUAUGAAU | AUUCAUAGUAGGCUCGGAUCUUC |
| GAUCCGAGCCUACUAUGAAAU | AUUUCAUAGUAGGCUCGGAUCUU |
| AAUUCUACUACAUCUAUAACU | AGUUAUAGAUGUAGUAGAAUUUC |
| UGGGCAACUCCAACAAUUACU | AGUAAUUGUUGGAGUUGCCCACG |
| GGCAACUCCAACAAUUACCUU | AAGGUAAUUGUUGGAGUUGCCCA |
| CGUGUUCGUGCUGAAUAAGAU | AUCUUAUUCAGCACGAACACGCC |
| UGUUCGUGCUGAAUAAGAAGU | ACUUCUUAUUCAGCACGAACACG |
| GUUCGUGCUGAAUAAGAAGAU | AUCUUCUUAUUCAGCACGAACAC |
| UUCGUGCUGAAUAAGAAGAAU | AUUCUUCUUAUUCAGCACGAACA |
| ACUGACGCAGAGUAAGAUCUU | AAGAUCUUACUCUGCGUCAGUUU |
| UCCGAGCCGUUCUCUACAAUU | AAUUGUAGAGAACGGCUCGGAUU |
| CCGAGCCGUUCUCUACAAUUU | AAAUUGUAGAGAACGGCUCGGAU |
| CGAGCCGUUCUCUACAAUUAU | AUAAUUGUAGAGAACGGCUCGGA |
| GAGCCGUUCUCUACAAUUACU | AGUAAUUGUAGAGAACGGCUCGG |
| UUCCUUGAAGCCAACUACAUU | AAUGUAGUUGGCUUCAAGGAAGU |
| GCCUCUUCUUAACAAAUUUCU | AGAAAUUUGUUAAGAAGAGGCCC |
| CCACAGCCAAAGAUAAGAACU | AGUUCUUAUCUUUCGCUGUGGUC |
| CUACUGCAGCUAAAAGACUUU | AAAGUCUUUUAGCUGCAGUAGGG |
| UCGUGCGUUGGCUCAAUGAAU | AUUCAUUGAGCCAACOGACGACG |
| GUGCGUUGGCUCAAUGAACAU | AUGUUCAUUGAGCCAACGCACGA |
| AGCCUUGGCUCAAUACCAAAU | AUUUGGUAUUGAGCCAAGGCUUG |
| AACUCACCUGUAAUAAAUUCU | AGAAUUUAUUACAGGUGAGUUGA |
| ACUCACCUGUAAUAAAUUCGU | ACGAAUUUAUUACAGGUGAGUUG |
| CUCACCUGUAAUAAAUUCGAU | AUCGAAUUUAUUACAGGUGAGUU |
| GACCUCAAGGUCACCAUAAAU | AUUUAUGGUGACCUUGAGGUCGA |
| CCUCAAGGUCACCAUAAAACU | AGUUUUAUGGUGACCUUGAGGUC |
| CUCAAGGUCACCAUAAAACCU | AGGUUUUAUGGUGACCUUGAGGU |
| UCAAGGUCACCAUAAAACCAU | AUGGUUUUAUGGUGACCUUGAGG |
| GAUGCCAAGAACACUAUGAUU | AAUCAUAGUGUUCUUGGCAUCCU |
| AGGAUGCCACUAUGUCUAUAU | AUAUAGACAUAGUGGCAUCCUGG |
| CAAGGUCUACGCCUAUUACAU | AUGUAAUAGGCGUAGACCUUGAC |
| AAGGUCUACGCCUAUUACAAU | AUUGUAAUAGGCGUAGACCUUGA |
| AGGUCUACGCCUAUUACAACU | AGUUGUAAUAGGCGUAGACCUUG |

TABLE 26

| Sense Sequence | Antisense Sequence |
|---|---|
| 5′ to 3′ | 5′ to 3′ |
| AGACAGACAAGACCAUCUACU | AGUAGAUGGUCUUGUCUGUCUGG |
| ACAGACAAGACCAUCUACACU | AGUGUAGAUGGUCUUGUCUGUCU |
| UGGGACAUUCCGGAACUCGUU | AACGAGUUCCGGAAUGUCCCAAG |
| AUUCCGGAACUCGUCAACAUU | AAUGUUGACGAGUUCCGGAAUGU |
| AGAUCCGAGCCUACUAUGAAU | AUUCAUAGUAGGCUCGGAUCUUC |
| GCAGGUCUUCUCCACUGAGUU | AACUCAGUGGAGAAGACCUGCUG |
| GCCCAGUUUCGAGGUCAUAGU | ACUAUGACCUCGAAACUGGGCAG |
| CCAGUUUCGAGGUCAUAGUGU | ACACUAUGACCUCGAAACUGGGC |
| AAUUCUACUACAUCUAUAACU | AGUUAUAGAUGUAGUAGAAUUUC |
| ACUGCCUUUGUCAUCUUCGGU | ACCGAAGAUGACAAAGGCAGUUC |
| CUCAUGGUGUUCGUGACGAAU | AUUCGUCACGAACACCAUGAGGU |
| UGGGCAACUCCAACAAUUACU | AGUAAUUGUUGGAGUUGCCCACG |
| GGGCAACUCCAACAAUUACCU | AGGUAAUUGUUGGAGUUGCCCAC |
| GCAACUCCAACAAUUACCUGU | ACAGGUAAUUGUUGGAGUUGCCC |
| GUCAACUUCCUCCUGCGAAUU | AAUUCGCAGGAGGAAGUUGACGU |
| AACUGACGCAGAGUAAGAUCU | AGAUCUUACUCUGCGUCAGUUUG |
| UGCAGAAGAGAACAUCGUUUU | AAAACGAUGUUCUCUUCUGCAAU |
| GAAGAGAACAUCGUUUCCCGU | ACGGGAAACGAUGUUCUCUUCUG |
| AAGAGAACAUCGUUUCCCGAU | AUCGGGAAACGAUGUUCUCUUCU |
| AGAACAUCGUUUCCCGAAGUU | AACUUCGGGAAACGAUGUUCUCU |
| GAACAUCGUUUCCCGAAGUGU | ACACUUCGGGAAACGAUGUUCUC |
| AACAUCGUUUCCCGAAGUGAU | AUCACUUCGGGAAACGAUGUUCU |
| CGGACAAGAAAGGGAUCUGUU | AACAGAUCCCUUUCUUGUCCGAC |
| GGACAAGAAAGGGAUCUGUGU | ACACAGAUCCCUUUCUUGUCCGA |
| GACAAGAAAGGGAUCUGUGUU | AACACAGAUCCCUUUCUUGUCCG |
| ACAAGAAAGGGAUCUGUGUGU | ACACACAGAUCCCUUUCUUGUCC |

TABLE 27

| Sense Sequence | Antisense Sequence |
|---|---|
| 5′ to 3′ | 5′ to 3′ |
| CAAGAAAGGGAUCUGUGUGGU | ACCACACAGAUCCCUUUCUUGUC |
| AAGAAAGGGAUCUGUGUGGCU | AGCCACACAGAUCCCUUUCUUGU |
| GAAAGGGAUCUGUGUGGCAGU | ACUGCCACACAGAUCCCUUUCUU |
| CUUCGAGGUCACAGUAAUGCU | AGCAUUACUGUGACCUCGAAGGG |
| UUCGAGGUCACAGUAAUGCAU | AUGCAUUACUGUGACCUCGAAGG |
| GGCUACCCUACUCUGUUGUUU | AAACAACAGAGUAGGGUAGCCGC |
| CUACCCUACUCUGUUGUUCGU | ACGAACAACAGAGUAGGGUAGCC |
| UACCCUACUCUGUUGUUCGAU | AUCGAACAACAGAGUAGGGUAGC |
| ACCCUACUCUGUUGUUCGAAU | AUUCGAACAACAGAGUAGGGUAG |
| CCCUACUCUGUUGUUCGAAAU | AUUUCGAACAACAGAGUAGGGUA |
| CCUACUCUGUUGUUCGAAACU | AGUUUCGAACAACAGAGUAGGGU |
| CUACUCUGUUGUUCGAAACGU | ACGUUUCGAACAACAGAGUAGGG |
| UACUCUGUUGUUCGAAACGAU | AUCGUUUCGAACAACAGAGUAGG |
| ACUCUGUUGUUCGAAACGAGU | ACUCGUUUCGAACAACAGAGUAG |
| CUCUGUUGUUCGAAACGAGCU | AGCUCGUUUCGAACAACAGAGUA |
| CCGUUCUCUACAAUUACCGGU | ACCGGUAAUUGUAGAGAACGGCU |
| AACAAAACUGUGGCUGUUCGU | ACGAACAGCCACAGUUUUGUUCA |
| GGUCAUCGCUGUGCAUUACCU | AGGUAAUGCACAGCGAUGACCGU |
| GUCAUCGCUGUGCAUUACCUU | AAGGUAAUGCACAGCGAUGACCG |
| UGCAUUACCUGGAUGAAACGU | ACGUUUCAUCCAGGUAAUGCACA |
| CGUGGUCAAGGUCUUCUCUCU | AGAGAGAAGACCUUGACCACGUA |
| CGGCCUUUGUUCUCAUCUCGU | ACGAGAUGAGAACAAAGGCCGUG |
| GGCCUUUGUUCUCAUCUCGCU | AGCGAGAUGAGAACAAAGGCCGU |
| GCCUUUGUUCUCAUCUCGCUU | AAGCGAGAUGAGAACAAAGGCCG |
| UUCCUUGAAGCCAACUACAUU | AAUGUAGUUGGCUUCAAGGAAGU |
| UGCAGCUAAAAGACUUUGACU | AGUCAAAGUCUUUUACCUGCAGU |
| UCGUGCGUUGGCUCAAUGAAU | AUUCAUUGAGCCAACGCACGACG |

TABLE 28

| Sense Sequence | Antisense Sequence |
|---|---|
| 5′ to 3′ | 5′ to 3′ |
| CGUGCGUUGGCUCAAUGAACU | AGUUCAUUGAGCCAACGCACGAC |
| CAAUGAACAGAGAUACUACGU | ACGUAGUAUCUCUGUUCAUUGAG |
| AAUGAACAGAGAUACUACGGU | ACCGUAGUAUCUCUGUUCAUUGA |
| AUGAACAGAGAUACUACGGUU | AACCGUAGUAUCUCUGUUCAUUG |
| CCAAGCCUUGGCUCAAUACCU | AGGUAUUGAGCCAAGGCUUGGAA |
| CAAGCCUUGGCUCAAUACCAU | AUGGUAUUGAGCCAAGGCUUGGA |
| CCACCGUAUCCACUGGGAAUU | AAUUCCCAGUGGAUACGGUGGGU |
| ACCAAGGAAAAUGAGGGUUUU | AAAACCCUCAUUUUCCUUGGUCU |
| AAGGAAAAUGAGGGUUUCACU | AGUGAAACCCUCAUUUUCCUUGG |
| AACUCACCUGUAAUAAAUUCU | AGAAUUUAUUACAGGUGAGUUGA |
| ACUCACCUGUAAUAAAUUCGU | ACGAAUUUAUUACAGGUGAGUUG |
| CUCACCUGUAAUAAAUUCGAU | AUCGAAUUUAUUACAGGUGAGUU |
| ACCUGUAAUAAAUUCGACCUU | AAGGUCGAAUUUAUUACAGGUGA |
| UAAUAAAUUCGACCUCAAGGU | ACCUUGAGGUCGAAUUUAUUACA |
| ACCUCAAGGUCACCAUAAAAU | AUUUUAUGGUGACCUUGAGGUCG |
| CCUCAAGGUCACCAUAAAACU | AGUUUUAUGGUGACCUUGAGGUC |
| CUCAAGGUCACCAUAAAACCU | AGGUUUUAUGGUGACCUUGAGGU |
| AGGAUGCCAAGAACACUAUGU | ACAUAGUGUUCUUGGCAUCCUGA |
| CAGAUACAUCUCCAAGUAUGU | ACAUACUUGGAGAUGUAUCUGUC |
| UGGACAAAGCCUUCUCCGAUU | AAUCGGAGAAGGCUUUGUCCAGC |
| AGGAACACCCUCAUCAUCUAU | AUAGAUGAUGAGGGUGUUCCUAU |
| GGAACACCCUCAUCAUCUACU | AGUAGAUGAUGAGGGUGUUCCUA |
| AACACCCUCAUCAUCUACCUU | AAGGUAGAUGAUGAGGGUGUUCC |
| CUUUAAUGUAGAGCUUAUCCU | AGGAUAAGCUCUACAUUAAAGUA |
| UUUAAUGUAGAGCUUAUCCAU | AUGGAUAAGCUCUACAUUAAAGU |
| UCAAGGUCUACGCCUAUUACU | AGUAAUAGGCGUAGACCUUGACU |
| CAAGGUCUACGCCUAUUACAU | AUGUAAUAGGCGUAGACCUUGAC |

TABLE 29

| Sense Sequence | Antisense Sequence |
|---|---|
| 5′ to 3′ | 5′ to 3′ |
| AAGGUCUACGCCUAUUACAAU | AUUGUAAUAGGCGUAGACCUUGA |
| AGGUCUACGCCUAUUACAACU | AGUUGUAAUAGGCGUAGACCUUG |
| UCUACGCCUAUUACAACCUGU | ACAGGUUGUAAUAGGCGUAGACC |
| ACCUGUCCAAUGACUUUGACU | AGUCAAAGUCAUUGGACAGCUGA |
| GCUGUCCAAUGACUUUGACGU | ACGUCAAAGUCAUUGGACAGCUG |
| AGCAUGGUUGUCUUUGGGUGU | ACACCCAAAGACAACCAUGCUCU |
| AAUAAGAAGAACAAACUGACA | UGUCAGUUUGUUCUUCUUAUUCA |
| AAUAAGAAGAACAAGCUGACA | UGUCAGCUUGUUCUUCUUAUUCA |
| AACACCCUCAUCAUCUACCUU | AAGGUAGAUGAUGAGGGUGUUCC |

TABLE 30

| Sense Sequence | Antisense Sequence |
|---|---|
| 5′ to 3′ | 5′ to 3′ |
| GUGCUGAAUAAGAAGAACAAU | AUUGUUCUUCUUAUUCAGCACGA |
| UGCUGAAUAAGAAGAACAAAU | AUUUGUUCUUCUUAUUCAGCACG |
| GCUGAAUAAGAAGAACAAACU | AGUUUGUUCUUCUUAUUCAGCAC |
| CUGAAUAAGAAGAACAAACUU | AAGUUUGUUCUUCUUAUUCAGCA |
| UGAAUAAGAAGAACAAACUGU | ACAGUUUGUUCUUCUUAUUCAGC |
| GAAUAAGAAGAACAAACUGAU | AUCAGUUUGUUCUUCUUAUUCAG |
| AAUAAGAAGAACAAACUGACU | AGUCAGUUUGUUCUUCUUAUUCA |
| AUAAGAAGAACAAACUGACGU | ACGUCAGUUUGUUCUUCUUAUUC |
| UAAGAAGAACAAACUGACGCU | AGCGUCAGUUUGUUCUUCUUAUU |
| AAGAAGAACAAACUGACGCAU | AUGCGUCAGUUUGUUCUUCUUAU |
| AGAAGAACAAACUGACGCAGU | ACUGCGUCAGUUUGUUCUUCUUA |
| GAAGAACAAACUGACGCAGAU | AUCUGCGUCAGUUUGUUCUUCUU |
| AAGAACAAACUGACGCAGAGU | ACUCUGCGUCAGUUUGUUCUUCU |
| AGAACAAACUGACGCAGAGUU | AACUCUGCGUCAGUUUGUUCUUC |
| GAACAAACUGACGCAGAGUAU | AUACUCUGCGUCAGUUUGUUCUU |
| AACAAACUGACGCAGAGUAAU | AUUACUCUGCGUCAGUUUGUUCU |
| ACAAACUGACGCAGAGUAAGU | ACUUACUCUGCGUCAGUUUGUUC |
| CAAACUGACGCAGAGUAAGAU | AUCUUACUCUGCGUCAGUUUGUU |
| AAACUGACGCAGAGUAAGAUU | AAUCUUACUCUGCGUCAGUUUGU |
| AACUGACGCAGAGUAAGAUCU | AGAUCUUACUCUGCGUCAGUUUG |
| CUGACGCAGAGUAAGAUCUGU | ACAGAUCUUACUCUGCGUCAGUU |
| UGACGCAGAGUAAGAUCUGGU | ACCAGAUCUUACUCUGCGUCAGU |
| GACGCAGAGUAAGAUCUGGGU | ACCCAGAUCUUACUCUGCGUCAG |
| ACGCAGAGUAAGAUCUGGGAU | AUCCCAGAUCUUACUCUGCGUCA |
| UGAGCAUGUCGGACAAGAAAU | AUUUCUUGUCCGACAUGCUCACA |
| GAGCAUGUCGGACAAGAAAGU | ACUUUCUUGUCCGACAUGCUCAC |
| AGCAUGUCGGACAAGAAAGGU | ACCUUUUUGUCCGACAUGCUCA |
| GCAUGUCGGACAAGAAAGGGU | ACCCUUUCUUGUCCGACAUGCUC |
| CAUGUCGGACAAGAAAGGGAU | AUCCCUUUCUUGUCCGACAUGCU |

TABLE 31

| Sense Sequence | Antisense Sequence |
|---|---|
| 5′ to 3′ | 5′ to 3′ |
| AUGUCGGACAAGAAAGGGAUU | AAUCCCUUUCUUGUCCGACAUGC |
| UGUCGGACAAGAAAGGGAUCU | AGAUCCCUUUCUUGUCCGACAUG |
| GUCGGACAAGAAAGGGAUCUU | AAGAUCCCUUUCUUGUCCGACAU |
| UCGGACAAGAAAGGGAUCUGU | ACAGAUCCCUUUCUUGUCCGACA |
| CGGACAAGAAAGGGAUCUGUU | AACAGAUCCCUUUCUUGUCCGAC |
| GGACAAGAAAGGGAUCUGUGU | ACACAGAUCCCUUUCUUGUCCGA |
| GACAAGAAAGGGAUCUGUGUU | AACACAGAUCCCUUUCUUGUCCG |
| ACAAGAAAGGGAUCUGUGUGU | ACACACAGAUCCCUUUCUUGUCC |
| CAAGAAAGGGAUCUGUGUGGU | ACCACACAGAUCCCUUUCUUGUC |
| AAGAAAGGGAUCUGUGUGGCU | AGCCACACAGAUCCCUUUCUUGU |
| AGAAAGGGAUCUGUGUGGCAU | AUGCCACACAGAUCCCUUUCUUG |
| GAAAGGGAUCUGUGUGGCAGU | ACUGCCACACAGAUCCCUUUCUU |
| AAAGGGAUCUGUGUGGCAGAU | AUCUGCCACACAGAUCCCUUUCU |
| AAGGGAUCUGUGUGGCAGACU | AGUCUGCCACACAGAUCCCUUUC |
| AGGGAUCUGUGUGGCAGACCU | AGGUCUGCCACACAGAUCCCUUU |
| GGGAUCUGUGUGGCAGACCCU | AGGGUCUGCCACACAGAUCCCUU |
| GAAAUCCGAGCCGUUCUCUAU | AUAGAGAACGGCUCGGAUUUCCA |
| AAAUCCGAGCCGUUCUCUACU | AGUAGAGAACGGCUCGGAUUUCC |
| AAUCCGAGCCGUUCUCUACAU | AUGUAGAGAACGGCUCGGAUUUC |
| AUCCGAGCCGUUCUCUACAAU | AUUGUAGAGAACGGCUCGGAUUU |
| AGCCGUUCUCUACAAUUACCU | AGGUAAUUGUAGAGAACGGCUCG |
| GCCGUUCUCUACAAUUACCGU | ACGGUAAUUGUAGAGAACGGCUC |
| CCGUUCUCUACAAUUACCGGU | ACCGGUAAUUGUAGAGAACGGCU |
| CGUUCUCUACAAUUACCGGCU | AGCCGGUAAUUGUAGAGAACGGC |
| GUUCUCUACAAUUACCGGCAU | AUGCCGGUAAUUGUAGAGAACGG |
| UUCUCUACAAUUACCGGCAGU | ACUGCCGGUAAUUGUAGAGAACG |
| UCUCUACAAUUACCGGCAGAU | AUCUGCCGGUAAUUGUAGAGAAC |
| GGCUGACCGCCUACGUGGUCU | AGACCACGUAGGCGGUCAGCCAG |
| GCUGACCGCCUACGUGGUCAU | AUGACCACGUAGGCGGUCAGCCA |
| CUGACCGCCUACGUGGUCAAU | AUUGACCACGUAGGGGUCAGCC |
| UGACCGCCUACGUGGUCAAGU | ACUUGACCACGUAGGCGGUCAGC |
| GACCGCCUACGUGGUCAAGGU | ACCUUGACCACGUAGGCGGUCAG |

TABLE 32

| Sense Sequence | Antisense Sequence |
|---|---|
| 5′ to 3′ | 5′ to 3′ |
| ACCGCCUACGUGGUCAAGGUU | AACCUUGACCACGUAGGCGGUCA |
| CCGCCUACGUGGUCAAGGUCU | AGACCUUGACCACGUAGGCGGUC |
| CGCCUACGUGGUCAAGGUCUU | AAGACCUUGACCACGUAGGCGGU |
| GCCUACGUGGUCAAGGUCUUU | AAAGACCUUGACCACGUAGGCGG |
| CCUACGUGGUCAAGGUCUUCU | AGAAGACCUUGACCACGUAGGCG |
| CUACGUGGUCAAGGUCUUCUU | AAGAAGACCUUGACCACGUAGGC |
| UACGUGGUCAAGGUCUUCUCU | AGAGAAGACCUUGACCACGUAGG |
| ACGUGGUCAAGGUCUUCUCUU | AAGAGAAGACCUUGACCACGUAG |
| CGUGGUCAAGGUCUUCUCUCU | AGAGAGAAGACCUUGACCACGUA |
| GUGGUCAAGGUCUUCUCUCUU | AAGAGAGAAGACCUUGACCACGU |
| UGGUCAAGGUCUUCUCUCUGU | ACAGAGAGAAGACCUUGACCACG |
| GGUCAAGGUCUUCUCUCUGGU | ACCAGAGAGAAGACCUUGACCAC |
| GUCAAGGUCUUCUCUCUGGCU | AGCCAGAGAGAAGACCUUGACCA |
| UCAAGGUCUUCUCUCUGGCUU | AAGCCAGAGAGAAGACCUUGACC |
| CAAGGUCUUCUCUCUGGCUGU | ACAGCCAGAGAGAAGACCUUGAC |
| AAGGUCUUCUCUCUGGCUGUU | AACAGCCAGAGAGAAGACCUUGA |
| AGGUCUUCUCUCUGGCUGUCU | AGACAGCCAGAGAGAAGACCUUG |
| GGUCUUCUCUCUGGCUGUCAU | AUGACAGCCAGAGAGAAGACCUU |
| GUCUUCUCUCUGGCUGUCAAU | AUUGACAGCCAGAGAGAAGACCU |
| UCUUCUCUCUGGCUGUCAACU | AGUUGACAGCCAGAGAGAAGACC |
| UAAAGCAGGAGACUUCCUUGU | ACAAGGAAGUCUCCUGCUUUAGU |
| AAAGCAGGAGACUUCCUUGAU | AUCAAGGAAGUCUCCUGCUUUAG |
| AAGCAGGAGACUUCCUUGAAU | AUUCAAGGAAGUCUCCUGCUUUA |
| AGCAGGAGACUUCCUUGAAGU | ACUUCAAGGAAGUCUCCUGCUUU |
| GCAGGAGACUUCCUUGAAGCU | AGCUUCAAGGAAGUCUCCUGCUU |
| CAGGAGACUUCCUUGAAGCCU | AGGCUUCAAGGAAGUCUCCUGCU |
| AGGAGACUUCCUUGAAGCCAU | AUGGCUUCAAGGAAGUCUCCUGC |
| GGAGACUUCCUUGAAGCCAAU | AUUGGCUUCAAGGAAGUCUCCUG |
| GAGACUUCCUUGAAGCCAACU | AGUUGGCUUCAAGGAAGUCUCCU |
| AGAGCGGGUACCUCUUCAUCU | AGAUGAAGAGGUACCCGCUCUGC |
| GAGCGGGUACCUCUUCAUCCU | AGGAUGAAGAGGUACCCGCUCUG |
| AGCGGGUACCUCUUCAUCCAU | AUGGAUGAAGAGGUACCCGCUCU |

TABLE 33

| Sense Sequence | Antisense Sequence |
|---|---|
| 5′ to 3′ | 5′ to 3′ |
| GCGGGUACCUCUUCAUCCAGU | ACUGGAUGAAGAGGUACCCGCUC |
| CGGGUACCUCUUCAUCCAGAU | AUCUGGAUGAAGAGGUACCCGCU |
| GGGUACCUCUUCAUCCAGACU | AGUCUGGAUGAAGAGGUACCCGC |
| GGUACCUCUUCAUCCAGACAU | AUGUCUGGAUGAAGAGGUACCCG |
| GUACCUCUUCAUCCAGACAGU | ACUGUCUGGAUGAAGAGGUACCC |
| UACCUCUUCAUCCAGACAGAU | AUCUGUCUGGAUGAAGAGGUACC |
| ACCUCUUCAUCCAGACAGACU | AGUCUGUCUGGAUGAAGAGGUAC |
| CCUCUUCAUCCAGACAGACAU | AUGUCUGUCUGGAUGAAGAGGUA |
| CUCUUCAUCCAGACAGACAAU | AUUGUCUGUCUGGAUGAAGAGGU |
| UCUUCAUCCAGACAGACAAGU | ACUUGUCUGUCUGGAUGAAGAGG |
| CUUCAUCCAGACAGACAAGAU | AUCUUGUCUGUCUGGAUGAAGAG |
| UUCAUCCAGACAGACAAGACU | AGUCUUGUCUGUCUGGAUGAAGA |
| UCAUCCAGACAGACAAGACCU | AGGUCUUGUCUGUCUGGAUGAAG |
| CAUCCAGACAGACAAGACCAU | AUGGUCUUGUCUGUCUGGAUGAA |
| AUCCAGACAGACAAGACCAUU | AAUGGUCUUGUCUGUCUGGAUGA |
| UCCAGACAGACAAGACCAUCU | AGAUGGUCUUGUCUGUCUGGAUG |
| CCAGACAGACAAGACCAUCUU | AAGAUGGUCUUGUCUGUCUGGAU |
| CAGACAGACAAGACCAUCUAU | AUAGAUGGUCUUGUCUGUCUGGA |
| GACAGACAAGACCAUCUACAU | AUGUAGAUGGUCUUGUCUGUCUG |
| CAGACAAGACCAUCUACACCU | AGGUGUAGAUGGUCUUGUCUGUC |
| AGACAAGACCAUCUACACCCU | AGGGUGUAGAUGGUCUUGUCUGU |
| GACAAGACCAUCUACACCCCU | AGGGGUGUAGAUGGUCUUGUCUG |
| ACAAGACCAUCUACACCCCUU | AAGGGGUGUAGAUGGUCUUGUCU |
| GGCCAGUGGAAGAUCCGAGCU | AGCUCGGAUCUUCCACUGGCCCA |
| GCCAGUGGAAGAUCCGAGCCU | AGGCUCGGAUCUUCCACUGGCCC |
| CCAGUGGAAGAUCCGAGCCUU | AAGGCUCGGAUCUUCCACUGGCC |
| CAGUGGAAGAUCCGAGCCUAU | AUAGGCUCGGAUCUUCCACUGGC |
| AGUGGAAGAUCCGAGCCUACU | AGUAGGCUCGGAUCUUCCACUGG |
| GUGGAAGAUCCGAGCCUACUU | AAGUAGGCUCGGAUCUUCCACUG |
| UGGAAGAUCCGAGCCUACUAU | AUAGUAGGCUCGGAUCUUCCACU |
| GGAAGAUCCGAGCCUACUAUU | AAUAGUAGGCUCGGAUCUUCCAC |
| GAAGAUCCGAGCCUACUAUGU | ACAUAGUAGGCUCGGAUCUUCCA |

TABLE 34

| Sense Sequence | Antisense Sequence |
|---|---|
| 5′ to 3′ | 5′ to 3′ |
| AAGAUCCGAGCCUACUAUGAU | AUCAUAGUAGGCUCGGAUCUUCC |
| AUCCGAGCCUACUAUGAAAAU | AUUUUCAUAGUAGGCUCGGAUCU |
| UCCGAGCCUACUAUGAAAACU | AGUUUUCAUAGUAGGCUCGGAUC |
| CCGAGCCUACUAUGAAAACUU | AAGUUUUCAUAGUAGGCUCGGAU |
| CGAGCCUACUAUGAAAACUCU | AGAGUUUUCAUAGUAGGCUCGGA |
| GAGCCUACUAUGAAAACUCAU | AUGAGUUUUCAUAGUAGGCUCGG |
| AGCCUACUAUGAAAACUCACU | AGUGAGUUUUCAUAGUAGGCUCG |
| GCCUACUAUGAAAACUCACCU | AGGUGAGUUUUCAUAGUAGGCUC |
| CCUACUAUGAAAACUCACCAU | AUGGUGAGUUUUCAUAGUAGGCU |
| CUACUAUGAAAACUCACCACU | AGUGGUGAGUUUUCAUAGUAGGC |
| UACUAUGAAAACUCACCACAU | AUGUGGUGAGUUUUCAUAGUAGG |
| CCUACAGAGAAAUUCUACUAU | AUAGUAGAAUUUCUCUGUAGGCU |
| CUACAGAGAAAUUCUACUACU | AGUAGUAGAAUUUCUCUGUAGGC |
| UACAGAGAAAUUCUACUACAU | AUGUAGUAGAAUUUCUCUGUAGG |
| ACAGAGAAAUUCUACUACAUU | AAUGUAGUAGAAUUUCUCUGUAG |
| CAGAGAAAUUCUACUACAUCU | AGAUGUAGUAGAAUUUCUCUGUA |
| AGAGAAAUUCUACUACAUCUU | AAGAUGUAGUAGAAUUUCUCUGU |
| GAGAAAUUCUACUACAUCUAU | AUAGAUGUAGUAGAAUUUCUCUG |
| AGAAAUUCUACUACAUCUAUU | AAUAGAUGUAGUAGAAUUUCUCU |
| GAAAUUCUACUACAUCUAUAU | AUAUAGAUGUAGUAGAAUUUCUC |
| AAAUUCUACUACAUCUAUAAU | AUUAUAGAUGUAGUAGAAUUUCU |
| AUUCUACUACAUCUAUAACGU | ACGUUAUAGAUGUAGUAGAAUUU |
| UUCUACUACAUCUAUAACGAU | AUCGUUAUAGAUGUAGUAGAAUU |
| UCUACUACAUCUAUAACGAGU | ACUCGUUAUAGAUGUAGUAGAAU |
| CUACUACAUCUAUAACGAGAU | AUCUCGUUAUAGAUGUAGUAGAA |
| UACUACAUCUAUAACGAGAAU | AUUCUCGUUAUAGAUGUAGUAGA |
| ACUACAUCUAUAACGAGAAGU | ACUUCUCGUUAUAGAUGUAGUAG |
| CUACAUCUAUAACGAGAAGGU | ACCUUCUCGUUAUAGAUGUAGUA |
| UACAUCUAUAACGAGAAGGGU | ACCCUUCUCGUUAUAGAUGUAGU |
| ACAUCUAUAACGAGAAGGGCU | AGCCCUUCUCGUUAUAGAUGUAG |
| CAUCUAUAACGAGAAGGGCCU | AGGCCCUUCUCGUUAUAGAUGUA |
| CCUCUCCCUACCAGAUCCACU | AGUGGAUCUGGUAGGGAGAGGUC |

TABLE 35

| Sense Sequence | Antisense Sequence |
|---|---|
| 5′ to 3′ | 5′ to 3′ |
| CUCUCCCUACCAGAUCCACUU | AAGUGGAUCUGGUAGGGAGAGGU |
| UCUCCCUACCAGAUCCACUUU | AAAGUGGAUCUGGUAGGGAGAGG |
| CUCCCUACCAGAUCCACUUCU | AGAAGUGGAUCUGGUAGGGAGAG |
| UCCCUACCAGAUCCACUUCAU | AUGAAGUGGAUCUGGUAGGGAGA |
| CCCUACCAGAUCCACUUCACU | AGUGAAGUGGAUCUGGUAGGGAG |
| CCUACCAGAUCCACUUCACCU | AGGUGAAGUGGAUCUGGUAGGGA |
| CUACCAGAUCCACUUCACCAU | AUGGUGAAGUGGAUCUGGUAGGG |
| UACCAGAUCCACUUCACCAAU | AUUGGUGAAGUGGAUCUGGUAGG |
| ACCAGAUCCACUUCACCAAGU | ACUUGGUGAAGUGGAUCUGGUAG |
| CCAGAUCCACUUCACCAAGAU | AUCUUGGUGAAGUGGAUCUGGUA |
| CAGAUCCACUUCACCAAGACU | AGUCUUGGUGAAGUGGAUCUGGU |
| AGAUCCACUUCACCAAGACAU | AUGUCUUGGUGAAGUGGAUCUGG |
| GAUCCACUUCACCAAGACACU | AGUGUCUUGGUGAAGUGGAUCUG |
| AUCCACUUCACCAAGACACCU | AGGUGUCUUGGUGAAGUGGAUCU |
| UUUGACCUCAUGGUGUUCGUU | AACGAACACCAUGAGGUCAAAGG |
| UGACCUCAUGGUGUUCGUGAU | AUCACGAACACCAUGAGGUCAAA |
| AGGGCGUGUUCGUGCUGAAUU | AAUUCAGCACGAACACGCCCUUG |
| GGGCGUGUUCGUGCUGAAUAU | AUAUUCAGCACGAACACGCCCUU |
| GGCGUGUUCGUGCUGAAUAAU | AUUAUUCAGCACGAACACGCCCU |
| GCGUGUUCGUGCUGAAUAAGU | ACUUAUUCAGCACGAACACGCCC |
| GUGUUCGUGCUGAAUAAGAAU | AUUCUUAUUCAGCACGAACACGC |
| UCGUGCUGAAUAAGAAGAACU | AGUUCUUCUUAUUCAGCACGAAC |
| CGUGCUGAAUAAGAAGAACAU | AUGUUCUUCUUAUUCAGCACGAA |
| AGACUUCCUUGAAGCCAACUU | AAGUUGGCUUCAAGGAAGUCUCC |
| GACUUCCUUGAAGCCAACUAU | AUAGUUGGCUUCAAGGAAGUCUC |
| ACUUCCUUGAAGCCAACUACU | AGUAGUUGGCUUCAAGGAAGUCU |
| CUUCCUUGAAGCCAACUACAU | AUGUAGUUGGCUUCAAGGAAGUC |
| UCCUUGAAGCCAACUACAUGU | ACAUGUAGUUGGCUUCAAGGAAG |
| CCUUGAAGCCAACUACAUGAU | AUCAUGUAGUUGGCUUCAAGGAA |
| CUUGAAGCCAACUACAUGAAU | AUUCAUGUAGUUGGCUUCAAGGA |
| UUGAAGCCAACUACAUGAACU | AGUUCAUGUAGUUGGCUUCAAGG |
| UGAAGCCAACUACAUGAACCU | AGGUUCAUGUAGUUGGCUUCAAG |

TABLE 36

| Sense Sequence | Antisense Sequence |
|---|---|
| 5′ to 3′ | 5′ to 3′ |
| GAAGCCAACUACAUGAACCUU | AAGGUUCAUGUAGUUGGCUUCAA |
| AAGCCAACUACAUGAACCUAU | AUAGGUUCAUGUAGUUGGCUUCA |
| AGCCAACUACAUGAACCUACU | AGUAGGUUCAUGUAGUUGGCUUC |
| GCCAACUACAUGAACCUACAU | AUGUAGGUUCAUGUAGUUGGCUU |
| CCAACUACAUGAACCUACAGU | ACUGUAGGUUCAUGUAGUUGGCU |
| CAACUACAUGAACCUACAGAU | AUCUGUAGGUUCAUGUAGUUGGC |
| AACUACAUGAACCUACAGAGU | ACUCUGUAGGUUCAUGUAGUUGG |
| ACUACAUGAACCUACAGAGAU | AUCUCUGUAGGUUCAUGUAGUUG |
| CUACAUGAACCUACAGAGAUU | AAUCUCUGUAGGUUCAUGUAGUU |
| UACAUGAACCUACAGAGAUCU | AGAUCUCUGUAGGUUCAUGUAGU |
| ACAUGAACCUACAGAGAUCCU | AGGAUCUCUGUAGGUUCAUGUAG |
| CAUGAACCUACAGAGAUCCUU | AAGGAUCUCUGUAGGUUCAUGUA |
| AUGAACCUACAGAGAUCCUAU | AUAGGAUCUCUGUAGGUUCAUGU |
| UGAACCUACAGAGAUCCUACU | AGUAGGAUCUCUGUAGGUUCAUG |
| GAACCUACAGAGAUCCUACAU | AUGUAGGAUCUCUGUAGGUUCAU |
| AACCUACAGAGAUCCUACACU | AGUGUAGGAUCUCUGUAGGUUCA |
| GGCCCUACUGCAGCUAAAAGU | ACUUUUAGCUGCAGUAGGGCCAA |
| GCCCUACUGCAGCUAAAAGAU | AUCUUUUAGCUGCAGUAGGGCCA |
| CCCUACUGCAGCUAAAAGACU | AGUCUUUUAGCUGCAGUAGGGCC |
| CCUACUGCACCUAAAAGACUU | AAGUCUUUUAGCUGCAGUAGGGC |
| UACUGCAGCUAAAAGACUUUU | AAAAGUCUUUUAGCUGCAGUAGG |
| ACUGCAGCUAAAAGACUUUGU | ACAAAGUCUUUUAGCUGCAGUAG |
| CUGCAGCUAAAAGACUUUGAU | AUCAAAGUCUUUUAGCUGCAGUA |
| UGCAGCUAAAAGACUUUGACU | AGUCAAAGUCUUUUAGCUGCAGU |
| GCAGCUAAAAGACUUUGACUU | AAGUCAAAGUCUUUUAGCUGCAG |
| CAGCUAAAAGACUUUGACUUU | AAAGUCAAAGUCUUUUAGCUGCA |
| AGCUAAAAGACUUUGACUUUU | AAAAGUCAAAGUCUUUUAGCUGC |
| GCUAAAAGACUUUGACUUUGU | ACAAAGUCAAAGUCUUUUAGCUG |
| GUGCCUCCCGUCGUGGGUUGU | ACAACGCACGACGGGAGGCACAA |
| UGCCUCCCGUCGUGCGUUGGU | ACCAACGCACGACGGGAGGCACA |
| GCCUCCCGUCGUGCGUUGGCU | AGCCAACGCACGACGGGAGGCAC |
| CCUCCCGUCGUGCGUUGGCUU | AAGCCAACGCACGACGGGAGGCA |

TABLE 37

| Sense Sequence | Antisense Sequence |
|---|---|
| 5′ to 3′ | 5′ to 3′ |
| CUCCGGUGGUGCGUUGGCUCU | AGAGCCAACGCACGACGGGAGGC |
| UCCCGUCGUGCGUUGGCUCAU | AUGAGCCAACGCACGACGGGAGG |
| CCCGUCGUGCGUUGGCUCAAU | AUUGAGCCAACGCACGACGGGAG |
| CCGUCGUGCGUUGGCUCAAUU | AAUUGAGCCAACGCACGACGGGA |
| CGUCGUGCGUUGGCUCAAUGU | ACAUUGAGCCAACGCACGACGGG |
| GUCGUGCGUUGGCUCAAUGAU | AUCAUUGAGCCAACGCACGACGG |
| CGUGCGUUGGCUCAAUGAACU | AGUUCAUUGAGCCAACGCACGAC |
| UGCGUUGGCUCAAUGAACAGU | ACUGUUCAUUGAGCCAACGCACG |
| GCGUUGGCUCAAUGAACAGAU | AUCUGUUCAUUGAGCCAACGCAC |
| CGUUGGCUCAAUGAACAGAGU | ACUCUGUUCAUUGAGCCAACGCA |
| GUUGGCUCAAUGAACAGAGAU | AUCUCUGUUCAUUGAGCCAACGC |
| UUGGCUCAAUGAACAGAGAUU | AAUCUCUGUUCAUUGAGCCAACG |
| UGGCUCAAUGAACAGAGAUAU | AUAUCUCUGUUCAUUGAGCCAAC |
| GGCUCAAUGAACAGAGAUACU | AGUAUCUCUGUUCAUUGAGCCAA |
| GCUCAAUGAACAGAGAUACUU | AAGUAUCUCUGUUCAUUGAGCCA |
| CUCAAUGAACAGAGAUACUAU | AUAGUAUCUCUGUUCAUUGAGCC |
| UCAAUGAACAGAGAUACUACU | AGUAGUAUCUCUGUUCAUUGAGC |
| CAAUGAACAGAGAUACUACGU | ACGUAGUAUCUCUGUUCAUUGAG |
| AAUGAACAGAGAUACUACGGU | ACCGUAGUAUCUCUGUUCAUUGA |
| AUGAACAGAGAUACUACGGUU | AACCGUAGUAUCUCUGUUCAUUG |
| GCAGUCAAGGUCUACGCCUAU | AUAGGCGUAGACCUUGACUGCUC |
| CAGUCAAGGUCUACGCCUAUU | AAUAGGCGUAGACCUUGACUGCU |
| AGUCAAGGUCUACGCCUAUUU | AAAUAGGCGUAGACCUUGACUGC |
| GUCAAGGUCUACGCCUAUUAU | AUAAUAGGGGUAGACCUUGACUG |
| UCAAGGUCUACGCCUAUUACU | AGUAAUAGGCGUAGACCUUGACU |
| GGUCUACGCCUAUUACAACCU | AGGUUGUAAUAGGCGUAGACCUU |
| GUCUACGCCUAUUACAACCUU | AAGGUUGUAAUAGGCGUAGACCU |
| UCUACGCCUAUUACAACCUGU | ACAGGUUGUAAUAGGCGUAGACC |
| CUACGCCUAUUACAACCUGGU | ACCAGGUUGUAAUAGGCGUAGAC |
| UACGCCUAUUACAACCUGGAU | AUCCAGGUUGUAAUAGGCGUAGA |
| ACGCCUAUUACAACCUGGAGU | ACUCCAGGUUGUAAUAGGCGUAG |
| CGCCUAUUACAACCUGGAGGU | ACCUCCAGGUUGUAAUAGGGGUA |

TABLE 38

| Sense Sequence | Antisense Sequence |
|---|---|
| 5′ to 3′ | 5′ to 3′ |
| GCUGAGGAGAAUUGCUUCAUU | AAUGAAGCAAUUCUCCUCAGCAC |
| GCCAGGAGUGGACUAUGUGUU | AACACAUAGUCCACUCCUGGCUC |
| CCAGGAGUGGACUAUGUGUAU | AUACACAUAGUCCACUCCUGGCU |
| CAGGAGUGGACUAUGUGUACU | AGUACACAUAGUCCACUCCUGGC |
| AGGAGUGGACUAUGUGUACAU | AUGUACACAUAGUCCACUCCUGG |
| GGAGUGGACUAUGUGUACAAU | AUUGUACACAUAGUCCACUCCUG |
| GAGUGGACUAUGUGUACAAGU | ACUUGUACACAUAGUCCACUCCU |
| AGUGGACUAUGUGUACAAGAU | AUCUUGUACACAUAGUCCACUCC |
| GUGGACUAUGUGUACAAGACU | AGUCUUGUACACAUAGUCCACUC |
| UGGACUAUGUGUACAAGACCU | AGGUCUUGUACACAUAGUCCACU |
| ACUAUGUGUACAAGACCCGAU | AUCGGGUCUUGUACACAUAGUCC |
| CUAUGUGUACAAGACCCGACU | AGUCGGGUCUUGUACACAUAGUC |

TABLE 39

| Sense Sequence | Antisense Sequence |
|---|---|
| 5′ to 3′ | 5′ to 3′ |
| AGAGCGGGUACCUCUUCAUCU | AGAUGAAGAGGUACCCGCUCUGC |
| GAGCGGGUACCUCUUCAUCCU | AGGAUGAAGAGGUACCCGCUCUG |
| AGCGGGUACCUCUUCAUCCAU | AUGGAUGAAGAGGUACCCGCUCU |
| GCGGGUACCUCUUCAUCCAGU | ACUGGATGAAGAGGUACCCGCUC |
| CGGGUACCUCUUCAUCCAGAU | AUCUGGAUGAAGAGGUACCCGCU |
| GGGUACCUCUUCAUCCAGACU | AGUCUGGAUGAAGAGGUACCCGC |
| GGUACCUCUUCAUCCAGACAU | AUGUCUGGAUGAAGAGGUACCCG |
| GUACCUCUUCAUCCAGACAGU | ACUGUCTGGAUGAAGAGGUACCC |
| UACCUCUUCAUCCAGACAGAU | AUCUGUCUGGAUGAAGAGGUACC |
| ACCUCUUCAUCCAGACAGACU | AGUCUGTCUGGAUGAAGAGGUAC |
| CCUCUUCAUCCAGACAGACAU | AUGUCUGUCUGGAUGAAGAGGUA |
| CUCUUCAUCCAGACAGACAAU | AUUGUCTGUCUGGAUGAAGAGGU |
| UCUUCAUCCAGACAGACAAGU | ACUUGUCUGUCUGGAUGAAGAGG |
| CUUCAUCCAGACAGACAAGAU | AUCUUGTCUGUCUGGAUGAAGAG |
| UUCAUCCAGACAGACAAGACU | AGUCUUGUCUGUCUGGAUGAAGA |
| UCAUCCAGACAGACAAGACCU | AGGUCUTGUCUGUCUGGAUGAAG |
| CAUCCAGACAGACAAGACCAU | AUGGUCTUGUCUGUCUGGAUGAA |
| AUCCAGACAGACAAGACCAUU | AAUGGUCUUGUCUGUCUGGAUGA |

TABLE 40

| Sense Sequence | Antisense Sequence |
|---|---|
| 5′ to 3′ | 5′ to 3′ |
| UCCAGACAGACAAGACCAUCU | AGAUGGTCUUGUCUGUCUGGAUG |
| CCAGACAGACAAGACCAUCUU | AAGAUGGUCUUGUCUGUCUGGAU |
| CAGACAGACAAGACCAUCUAU | AUAGAUGGUCUUGUCUGUCUGGA |
| GACAGACAAGACCAUCUACAU | AUGUAGAUGGUCUUGUCUGUCUG |
| CAGACAAGACCAUCUACACCU | AGGUGUAGAUGGUCUUGUCUGUC |
| ACAAGACCAUCUACACCCCUU | AAGGGGTGUAGAUGGUCUUGUCU |
| GGCCAGUGGAAGAUCCGAGCU | AGCUCGGAUCUUCCACUGGCCCA |
| GCCAGUGGAAGAUCCGAGCCU | AGGCUCGGAUCUUCCACUGGCCC |
| CCAGUGGAAGAUCCGAGCCUU | AAGGCUCGGAUCUUCCACUGGCC |
| CAGUGGAAGAUCCGAGCCUAU | AUAGGCTCGGAUCUUCCACUGGC |
| AGUGGAAGAUCCGAGCCUACU | AGUAGGCUCGGAUCUUCCACUGG |
| GUGGAAGAUCCGAGCCUACUU | AAGUAGGCUCGGAUCUUCCACUG |
| UGGAAGAUCCGAGCCUACUAU | AUAGUAGGCUCGGAUCUUCCACU |
| GGAAGAUCCGAGCCUACUAUU | AAUAGUAGGCUCGGAUCUUCCAC |
| GAAGAUCCGAGCCUACUAUGU | ACAUAGTAGGCUCGGAUCUUCCA |
| AAGAUCCGAGCCUACUAUGAU | AUCAUAGUAGGCUCGGAUCUUCC |
| AUCCGAGCCUACUAUGAAAAU | AUUUUCAUAGUAGGCUCGGAUCU |
| UCCGAGCCUACUAUGAAAACU | AGUUUUCAUAGUAGGCUCGGAUC |
| CCGAGCCUACUAUGAAAACUU | AAGUUUTCAUAGUAGGCUCGGAU |
| CGAGCCUACUAUGAAAACUCU | AGAGUUTUCAUAGUAGGCUCGGA |
| GAGCCUACUAUGAAAACUCAU | AUGAGUTUUCAUAGUAGGCUCGG |
| GCCUACUAUGAAAACUCACCU | AGGUGAGUUUUCAUAGUAGGCUC |
| CCUACUAUGAAAACUCACCAU | AUGGUGAGUUUUCAUAGUAGGCU |
| CUACUAUGAAAACUCACCACU | AGUGGUGAGUUUUCAUAGUAGGC |
| UACUAUGAAAACUCACCACAU | AUGUGGTGAGUUUUCAUAGUAGG |
| CCUACAGAGAAAUUCUACUAU | AUAGUAGAAUUUCUCUGUAGGCU |
| CUACAGAGAAAUUCUACUACU | AGUAGUAGAAUUUCUCUGUAGGC |
| UACAGAGAAAUUCUACUACAU | AUGUAGTAGAAUUUCUCUGUAGG |
| ACAGAGAAAUUCUACUACAUU | AAUGUAGUAGAAUUUCUCUGUAG |
| CAGAGAAAUUCUACUACAUCU | AGAUGUAGUAGAAUUUCUCUGUA |
| AGAGAAAUUCUACUACAUCUU | AAGAUGTAGUAGAAUUUCUCUGU |
| GAGAAAUUCUACUACAUCUAU | AUAGAUGUAGUAGAAUUUCUCUG |
| AGAAAUUCUACUACAUCUAUU | AAUAGATGUAGUAGAAUUUCUCU |
| GAAAUUCUACUACAUCUAUAU | AUAUAGAUGUAGUAGAAUUUCUC |

TABLE 41

| Sense Sequence | Antisense Sequence |
|---|---|
| 5′ to 3′ | 5′ to 3′ |
| AAAUUCUACUACAUCUAUAAU | AUUAUAGAUGUAGUAGAAUUUCU |
| AUUCUACUACAUCUAUAACGU | ACGUUATAGAUGUAGUAGAAUUU |
| UUCUACUACAUCUAUAACGAU | AUCGUUAUAGAUGUAGUAGAAUU |
| UCUACUACAUCUAUAACGAGU | ACUCGUTAUAGAUGUAGUAGAAU |
| CUACUACAUCUAUAACGAGAU | AUCUCGTUAUAGAUGUAGUAGAA |
| UACUACAUCUAUAACGAGAAU | AUUCUCGUUAUAGAUGUAGUAGA |
| ACUACAUCUAUAACGAGAAGU | ACUUCUCGUUAUAGAUGUAGUAG |
| CUACAUCUAUAACGAGAAGGU | ACCUUCTCGUUAUAGAUGUAGUA |
| UACAUCUAUAACGAGAAGGGU | ACCCUUCUCGUUAUAGAUGUAGU |
| ACAUCUAUAACGAGAAGGGCU | AGCCCUTCUCGUUAUAGAUGUAG |
| CAUCUAUAACGAGAAGGGCCU | AGGCCCTUCUCGUUAUAGAUGUA |
| CCUCUCCCUACCAGAUCCACU | AGUGGATCUGGUAGGGAGAGGUC |
| CUCUCCCUACCAGAUCCACUU | AAGUGGAUCUGGUAGGGAGAGGU |
| UCUCCCUACCAGAUCCACUUU | AAAGUGGAUCUGGUAGGGAGAGG |
| CUCCCUACCAGAUCCACUUCU | AGAAGUGGAUCUGGUAGGGAGAG |
| UCCCUACCAGAUCCACUUCAU | AUGAAGTGGAUCUGGUAGGGAGA |
| CCCUACCAGAUCCACUUCACU | AGUGAAGUGGAUCUGGUAGGGAG |
| CCUACCAGAUCCACUUCACCU | AGGUGAAGUGGAUCUGGUAGGGA |
| CUACCAGAUCCACUUCACCAU | AUGGUGAAGUGGAUCUGGUAGGG |
| UACCAGAUCCACUUCACCAAU | AUUGGUGAAGUGGAUCUGGUAGG |
| ACCAGAUCCACUUCACCAAGU | ACUUGGTGAAGUGGAUCUGGUAG |
| CCAGAUCCACUUCACCAAGAU | AUCUUGGUGAAGUGGAUCUGGUA |
| CAGAUCCACUUCACCAAGACU | AGUCUUGGUGAAGUGGAUCUGGU |
| AGAUCCACUUCACCAAGACAU | AUGUCUTGGUGAAGUGGAUCUGG |
| GAUCCACUUCACCAAGACACU | AGUGUCTUGGUGAAGUGGAUCUG |
| AUCCACUUCACCAAGACACCU | AGGUGUCUUGGUGAAGUGGAUCU |
| UUUGACCUCAUGGUGUUCGUU | AACGAACACCAUGAGGUCAAAGG |
| UGACCUCAUGGUGUUCGUGAU | AUCACGAACACCAUGAGGUCAAA |
| AGGGCGUGUUCGUGCUGAAUU | AAUUCAGCACGAACACGCCCUUG |
| GGGCGUGUUCGUGCUGAAUAU | AUAUUCAGCACGAACACGCCCUU |
| GGCGUGUUCGUGCUGAAUAAU | AUUAUUCAGCACGAACACGCCCU |
| GCGUGUUCGUGCUGAAUAAGU | ACUUAUTCAGCACGAACACGCCC |
| GUGUUCGUGCUGAAUAAGAAU | AUUCUUAUUCAGCACGAACACGC |
| UCGUGCUGAAUAAGAAGAACU | AGUUCUTCUUAUUCAGCACGAAC |

TABLE 42

| Sense Sequence | Antisense Sequence |
|---|---|
| 5′ to 3′ | 5′ to 3′ |
| CGUGCUGAAUAAGAAGAACAU | AUGUUCTUCUUAUUCAGCACGAA |
| GUGCUGAAUAAGAAGAACAAU | AUUGUUCUUCUUAUUCAGCACGA |
| UGCUGAAUAAGAAGAACAAAU | AUUUGUTCUUCUUAUUCAGCACG |
| GCUGAAUAAGAAGAACAAACU | AGUUUGTUCUUCUUAUUCAGCAC |
| CUGAAUAAGAAGAACAAACUU | AAGUUUGUUCUUCUUAUUCAGCA |
| UGAAUAAGAAGAACAAACUGU | ACAGUUTGUUCUUCUUAUUCAGC |
| GAAUAAGAAGAACAAACUGAU | AUCAGUTUGUUCUUCUDAUUCAG |
| AAUAAGAAGAACAAACUGACU | AGUCAGTUUGUUCUUCUUAUUCA |
| AUAAGAAGAACAAACUGACGU | ACGUCAGUUUGUUCUUCUUAUUC |
| UAAGAAGAACAAACUGACGCU | AGCGUCAGUUUGUUCUUCUUAUU |
| AAGAAGAACAAACUGACGCAU | AUGCGUCAGUUUGUUCUUCUUAU |
| AGAAGAACAAACUGACGCAGU | ACUGCGTCAGUUUGUUCUUCUUA |
| GAAGAACAAACUGACGCAGAU | AUCUGCGUCAGUUUGUUCUUCUU |
| AAGAACAAACUGACGCAGAGU | ACUCUGCGUCAGUUUGUUCUUCU |
| AGAACAAACUGACGCAGAGUU | AACUCUGCGUCAGUUUGUUCUUC |
| GAACAAACUGACGCAGAGUAU | AUACUCTGCGUCAGUUUGUUCUU |
| AACAAACUGACGCAGAGUAAU | AUUACUCUGCGUCAGUUUGUUCU |
| ACAAACUGACGCAGAGUAAGU | ACUUACTCUGCGUCAGUUUGUUC |
| CAAACUGACGCAGAGUAAGAU | AUCUUACUCUGCGUCAGUUUGUU |
| AAACUGACGCAGAGUAAGAUU | AAUCUUACUCUGCGUCAGUUUGU |
| AACUGACGCAGAGUAAGAUCU | AGAUCUTACUCUGCGUCAGUUUG |
| CUGACGCAGAGUAAGAUCUGU | ACAGAUCUUACUCUGCGUCAGUU |
| UGACGCAGAGUAAGAUCUGGU | ACCAGATCUUACUCUGCGUCAGU |
| GACGCAGAGUAAGAUCUGGGU | ACCCAGAUCUUACUCUGCGUCAG |
| ACGCAGAGUAAGAUCUGGGAU | AUCCCAGAUCUUACUCUGCGUCA |
| UGAGCAUGUCGGACAAGAAAU | AUUUCUTGUCCGACAUGCUCACA |
| GAGCAUGUCGGACAAGAAAGU | ACUUUCTUGUCCGACAUGCUCAC |
| AGCAUGUCGGACAAGAAAGGU | ACCUUUCUUGUCCGACAUGCUCA |
| GCAUGUCGGACAAGAAAGGGU | ACCCUUTCUUGUCCGACAUGCUC |
| CAUGUCGGACAAGAAAGGGAU | AUCCCUTUCUUGUCCGACAUGCU |
| AUGUCGGACAAGAAAGGGAUU | AAUCCCTUUCUUGUCCGACAUGC |
| UGUCGGACAAGAAAGGGAUCU | AGAUCCCUUUCUUGUCCGACAUG |
| GUCGGACAAGAAAGGGAUCUU | AAGAUCCCUUUCUUGUCCGACAU |
| UCGGACAAGAAAGGGAUCUGU | ACAGAUCCCUUUCUUGUCCGACA |

TABLE 43

| Sense Sequence | Antisense Sequence |
|---|---|
| 5′ to 3′ | 5′ to 3′ |
| CGGACAAGAAAGGGAUCUGUU | AACAGATCCCUUUCUUGUCCGAC |
| GGACAAGAAAGGGAUCUGUGU | ACACAGAUCCCUUUCUUGUCCGA |
| GACAAGAAAGGGAUCUGUGUU | AACACAGAUCCCUUUCUUGUCCG |
| ACAAGAAAGGGAUCUGUGUGU | ACACACAGAUCCCUUUCUUGUCC |
| CAAGAAAGGGAUCUGUGUGGU | ACCACACAGAUCCCUUUCUUGUC |
| AAGAAAGGGAUCUGUGUGGCU | AGCCACACAGAUCCCUUUCUUGU |
| GAAAGGGAUCUGUGUGGCAGU | ACUCCCACACAGAUCCCUUUCUU |
| AAAGGGAUCUGUGUGGCAGAU | AUCUGCCACACAGAUCCCUUUCU |
| AAGGGAUCUGUGUGGCAGACU | AGUCUGCCACACAGAUCCCUUUC |
| AGGGAUCUGUGUGGCAGACCU | AGGUCUGCCACACAGAUCCCUUU |
| GGGAUCUGUGUGGCAGACCCU | AGGGUCTGCCACACAGAUCCCUU |
| GAAAUCCGAGCCGUUCUCUAU | AUAGAGAACGGCUCGGAUUUCCA |
| AAAUCCGAGCCGUUCUCUACU | AGUAGAGAACGGCUCGGAUUUCC |
| AAUCCGAGCCGUUCUCUACAU | AUGUAGAGAACGGCUCGGAUUUC |
| AUCCGAGCCGUUCUCUACAAU | AUUGUAGAGAACGGCUCGGAUUU |
| AGCCGUUCUCUACAAUUACCU | AGGUAATUGUAGAGAACGGCUCG |
| GCCGUUCUCUACAAUUACCGU | ACGGUAAUUGUAGAGAACGGCUC |
| CCGUUCUCUACAAUUACCGGU | ACCGGUAAUUGUAGAGAACGGCU |
| CGUUCUCUACAAUUACCGGCU | AGCCGGTAAUUGUAGAGAACGGC |
| GUUCUCUACAAUUACCGGCAU | AUGCCGGUAAUUGUAGAGAACGG |
| UUCUCUACAAUUACCGGCAGU | ACUGCCGGUAAUUGUAGAGAACG |
| UCUCUACAAUUACCGGCAGAU | AUCUGCCGGUAAUUGUAGAGAAC |
| GGCUGACCGCCUACGUGGUCU | AGACCACGUAGGCGGUCAGCCAG |
| GCUGACCGCCUACGUGGUCAU | AUGACCACGUAGGCGGUCAGCCA |
| CUGACCGCCUACGUGGUCAAU | AUUGACCACGUAGGCGGUCAGCC |
| UGACCGCCUACGUGGUCAAGU | ACUUGACCACGUAGGCGGUCAGC |
| GACCGCCUACGUGGUCAAGGU | ACCUUGACCACGUAGGCGGUCAG |
| ACCGCCUACGUGGUCAAGGUU | AACCUUGACCACGUAGGCGGUCA |
| CCGCCUACGUGGUCAAGGUCU | AGACCUTGACCACGUAGGCGGUC |
| CGCCUACGUGGUCAAGGUCUU | AAGACCTUGACCACGUAGGCGGU |
| GCCUACGUGGUCAAGGUCUUU | AAAGACCUUGACCACGUAGGCGG |
| CCUACGUGGUCAAGGUCUUCU | AGAAGACCUUGACCACGUAGGCG |
| CUACGUGGUCAAGGUCUUCUU | AAGAAGACCUUGACCACGUAGGC |
| UACGUGGUCAAGGUCUUCUCU | AGAGAAGACCUUGACCACGUAGG |

TABLE 44

| Sense Sequence | Antisense Sequence |
|---|---|
| 5′ to 3′ | 5′ to 3′ |
| ACGUGGUCAAGGUCUUCUCUU | AAGAGAAGACCUUGACCACGUAG |
| CGUGGUCAAGGUCUUCUCUCU | AGAGAGAAGACCUUGACCACGUA |
| GUGGUCAAGGUCUUCUCUCUU | AAGAGAGAAGACCUUGACCACGU |
| UGGUCAAGGUCUUCUCUCUGU | ACAGAGAGAAGACCUUGACCACG |
| GGUCAAGGUCUUCUCUCUGGU | ACCAGAGAGAAGACCUUGACCAC |
| GUCAAGGUCUUCUCUCUGGCU | AGCCAGAGAGAAGACCUUGACCA |
| UCAAGGUCUUCUCUCUGGCUU | AAGCCAGAGAGAAGACCUUGACC |
| CAAGGUCUUCUCUCUGGCUGU | ACAGCCAGAGAGAAGACCUUGAC |
| AAGGUCUUCUCUCUGGCUGUU | AACAGCCAGAGAGAAGACCUUGA |
| AGGUCUUCUCUCUGGCUGUCU | AGACAGCCAGAGAGAAGACCUUG |
| GGUCUUCUCUCUGGCUGUCAU | AUGACAGCCAGAGAGAAGACCUU |
| GUCUUCUCUCUGGCUGUCAAU | AUUGACAGCCAGAGAGAAGACCU |
| UCUUCUCUCUGGCUGUCAACU | AGUUGACAGCCAGAGAGAAGACC |
| UAAAGCAGGAGACUUCCUUGU | ACAAGGAAGUCUCCUGCUUUAGU |
| AAAGCAGGAGACUUCCUUGAU | AUCAAGGAAGUCUCCUGCUUUAG |
| AAGCAGGAGACUUCCUUGAAU | AUUCAAGGAAGUCUCCUGCUUUA |
| AGCAGGAGACUUCCUUGAAGU | ACUUCAAGGAAGUCUCCUGCUUU |
| AGGAGACUUCCUUGAAGCCAU | AUGGCUTCAAGGAAGUCUCCUGC |
| GGAGACUUCCUUGAAGCCAAU | AUUGGCTUCAAGGAAGUCUCCUG |
| GAGACUUCCUUGAAGCCAACU | AGUUGGCUUCAAGGAAGUCUCCU |
| AGACUUCCUUGAAGCCAACUU | AAGUUGGCUUCAAGGAAGUCUCC |
| GACUUCCUUGAAGCCAACUAU | AUAGUUGGCUUCAAGGAAGUCUC |
| ACUUCCUUGAAGCCAACUACU | AGUAGUTGGCUUCAAGGAAGUCU |
| CUUCCUUGAAGCCAACUACAU | AUGUAGTUGGCUUCAAGGAAGUC |
| UCCUUGAAGCCAACUACAUGU | ACAUGUAGUUGGCUUCAAGGAAG |
| CCUUGAAGCCAACUACAUGAU | AUCAUGTAGUUGGCUUCAAGGAA |
| CUUGAAGCCAACUACAUGAAU | AUUCAUGUAGUUGGCUUCAAGGA |
| UUGAAGCCAACUACAUGAACU | AGUUCATGUAGUUGGCUUCAAGG |
| UGAAGCCAACUACAUGAACCU | AGGUUCAUGUAGUUGGCUUCAAG |
| GAAGCCAACUACAUGAACCUU | AAGGUUCAUGUAGUUGGCUUCAA |
| AAGCCAACUACAUGAACCUAU | AUAGGUTCAUGUAGUUGGCUUCA |
| AGCCAACUACAUGAACCUACU | AGUAGGTUCAUGUAGUUGGCUUC |
| GCCAACUACAUGAACCUACAU | AUGUAGGUUCAUGUAGUUGGCUU |
| CCAACUACAUGAACCUACAGU | ACUGUAGGUUCAUGUAGUUGGCU |

TABLE 45

| Sense Sequence | Antisense Sequence |
|---|---|
| 5′ to 3′ | 5′ to 3′ |
| CAACUACAUGAACCUACAGAU | AUCUGUAGGUUCAUGUAGUUGGC |
| AACUACAUGAACCUACAGAGU | ACUCUGTAGGUUCAUGUAGUUGG |
| ACUACAUGAACCUACAGAGAU | AUCUCUGUAGGUUCAUGUAGUUG |
| CUACAUGAACCUACAGAGAUU | AAUCUCTGUAGGUUCAUGUAGUU |
| UACAUGAACCUACAGAGAUCU | AGAUCUCUGUAGGUUCAUGUAGU |
| ACAUGAACCUACAGAGAUCCU | AGGAUCTCUGUAGGUUCAUGUAG |
| CAUGAACCUACAGAGAUCCUU | AAGGAUCUCUGUAGGUUCAUGUA |
| AUGAACCUACAGAGAUCCUAU | AUAGGATCUCUGUAGGUUCAUGU |
| UGAACCUACAGAGAUCCUACU | AGUAGGAUCUCUGUAGGUUCAUG |
| GAACCUACAGAGAUCCUACAU | AUGUAGGAUCUCUGUAGGUUCAU |
| AACCUACAGAGAUCCUACACU | AGUGUAGGAUCUCUGUAGGUUCA |
| GGCCCUACUGCAGCUAAAAGU | ACUUUUAGCUGCAGUAGGGCCAA |
| GCCCUACUGCAGCUAAAAGAU | AUCUUUTAGCUGCAGUAGGGCCA |
| CCCUACUGCAGCUAAAAGACU | AGUCUUTUAGCUGCAGUAGGGCC |
| CCUACUGCAGCUAAAAGACUU | AAGUCUTUUAGCUGCAGUAGGGC |
| UACUGCAGCUAAAAGACUUUU | AAAAGUCUUUUAGCUGCAGUAGG |
| ACUGCAGCUAAAAGACUUUGU | ACAAAGTCUUUUAGCUGCAGUAG |
| CUGCAGCUAAAAGACUUUGAU | AUCAAAGUCUUUUAGCUGCAGUA |
| UGCACCUAAAAGACUUUGACU | AGUCAAAGUCUUUUAGCUCCAGU |
| GCAGCUAAAAGACUUUGACUU | AAGUCAAAGUCUUUUAGCUGCAG |
| CAGCUAAAAGACUUUGACUUU | AAAGUCAAAGUCUUUUAGCUGCA |
| AGCUAAAAGACUUUGACUUUU | AAAAGUCAAAGUCUUUUAGCUGC |
| GCUAAAAGACUUUGACUUUGU | ACAAAGTCAAAGUCUUUUAGCUG |
| GUGCCUCCCGUCGUGCGUUGU | ACAACGCACGACGGGAGGCACAA |
| UGCCUCCCGUCGUGCGUUGGU | ACCAACGCACGACGGGAGGCACA |
| GCCUCCCGUCGUGCGUUGGCU | AGCCAACGCACGACGGGAGGCAC |
| CCUCCCGUCGUGCGUUGGCUU | AAGCCAACGCACGACGGGAGGCA |
| CUCCGGUCGUGCGUUGGCUCU | AGAGCCAACGCACGACGGGAGGC |
| UCCCGUCGUGCGUUGGCUCAU | AUGAGCCAACGCACGACGGGAGG |
| CCCGUCGUGCGUUGGCUCAAU | AUUGAGCCAACGCACGACGGGAG |
| CCGUCGUGCGUUGGCUCAAUU | AAUUGAGCCAACGCACGACGGGA |
| CGUCGUGCGUUGGCUCAAUGU | ACAUUGAGCCAACGCACGACGGG |
| GUCGUGCGUUGGCUCAAUGAU | AUCAUUGAGCCAACGCACGACGG |
| CGUGCGUUGGCUCAAUGAACU | AGUUCATUGAGCCAACGCACGAC |

TABLE 46

| Sense Sequence | Antisense Sequence |
|---|---|
| 5′ to 3′ | 5′ to 3′ |
| UGCGUUGGCUCAAUGAACAGU | ACUGUUCAUUGAGCCAACGCACG |
| GCGUUGGCUCAAUGAACAGAU | AUCUGUTCAUUGAGCCAACGCAC |
| CGUUGGCUCAAUGAACAGAGU | ACUCUGTUCAUUGAGCCAACGCA |
| GUUGGCUCAAUGAACAGAGAU | AUCUCUGUUCAUUGAGCCAACGC |
| UUGGCUCAAUGAACAGAGAUU | AAUCUCTGUUCAUUGAGCCAACG |
| UGGCUCAAUGAACAGAGAUAU | AUAUCUCUGUUCAUUGAGCCAAC |
| GGCUCAAUGAACAGAGAUACU | AGUAUCTCUGUUCAUUGAGCCAA |
| GCUCAAUGAACAGAGAUACUU | AAGUAUCUCUGUUCAUUGAGCCA |
| CUCAAUGAACAGAGAUACUAU | AUAGUATCUCUGUUCAUUGAGCC |
| UCAAUGAACAGAGAUACUACU | AGUAGUAUCUCUGUUCAUUGAGC |
| CAAUGAACAGAGAUACUACGU | ACGUAGTAUCUCUGUUCAUUGAG |
| AAUGAACAGAGAUACUACGGU | ACCGUAGUAUCUCUGUUCAUUGA |
| AUGAACAGAGAUACUACGGUU | AACCGUAGUAUCUCUGUUCAUUG |
| GAGCAGUCAAGGUCUACGGCU | AGGCGUAGACCUUGACUGCUCCA |
| AGCAGUCAAGGUCUACGCCUU | AAGGCGTAGACCUUGACUGCUCC |
| GCAGUCAAGGUCUACGCCUAU | AUAGGCGUAGACCUUGACUGCUC |
| CAGUCAAGGUCUACGCCUAUU | AAUAGGCGUAGACCUUGACUGCU |
| AGUCAAGGUCUACGCCUAUUU | AAAUAGGCGUAGACCUUGACUGC |
| GUCAAGGUCUACGCCUAUUAU | AUAAUAGGCGUAGACCUUGACUG |
| UCAAGGUCUACGCCUAUUACU | AGUAAUAGGCGUAGACCUUGACU |
| GGUCUACGCCUAUUACAACCU | AGGUUGTAAUAGGCGUAGACCUU |
| GUCUACGCCUAUUACAACCUU | AAGGUUGUAAUAGGCGUAGACCU |
| UCUACGCCUAUUACAACCUGU | ACAGGUTGUAAUAGGCGUAGACC |
| CUACGCCUAUUACAACCUGGU | ACCAGGTUGUAAUAGGCGUAGAC |
| UACGCCUAUUACAACCUGGAU | AUCCAGGUUGUAAUAGGCGUAGA |
| ACGCCUAUUACAACCUGGAGU | ACUCCAGGUUGUAAUAGGCGUAG |
| CGCCUAUUACAACCUGGAGGU | ACCUCCAGGUUGUAAUAGGCGUA |
| GCUGAGGAGAAUUGCUUCAUU | AAUGAAGCAAUUCUCCUCAGCAC |
| GCCAGGAGUGGACUAUGUGUU | AACACATAGUCCACUCCUGGCUC |
| CCAGGAGUGGACUAUGUGUAU | AUACACAUAGUCCACUCCUGGCU |
| CAGGAGUGGACUAUGUGUACU | AGUACACAUAGUCCACUCCUGGC |
| AGGAGUGGACUAUGUGUACAU | AUGUACACAUAGUCCACUCCUGG |
| GGAGUGGACUAUGUGUACAAU | AUUGUACACAUAGUCCACUCCUG |
| GAGUGGACUAUGUGUACAAGU | ACUUGUACACAUAGUCCACUCCU |

TABLE 47

| Sense Sequence | Antisense Sequence |
|---|---|
| 5′ to 3′ | 5′ to 3′ |
| AGUGGACUAUGUGUACAAGAU | AUCUUGTACACAUAGUCCACUCC |
| GUGGACUAUGUGUACAAGACU | AGUCUUGUACACAUAGUCCACUC |
| UGGACUAUGUGUACAAGACCU | AGGUCUTGUACACAUAGUCCACU |
| ACUAUGUGUACAAGACCCGAU | AUCGGGTCUUGUACACAUAGUCC |
| CUAUGUGUACAAGACCCGACU | AGUCGGGUCUUGUACACAUAGUC |

TABLE 48

| Sense Sequence | Antisense Sequence |
|---|---|
| 5′ to 3′ | 5′ to 3′ |
| CCAGACAGACAAGACCAUCUU | AAGAUGGUCUUGUCUGUCUGGAU |
| CCAGACAGACAAGACCAUCUU | AAGATGGUCUUGUCUGUCUGGAU |
| CCAGACAGACAAGACCAUCUU | AAGATGGUCUUGUCUGUCUGGAU |
| CCAGACAGACAAGACCAUCUU | AAGATGGUCUUGUCUGUCUGGCU |
| CCAGACAGACAAGACCAUCUU | AAGATGGUCUUGUCUGUCUGGCC |
| AGACAGACAAGACCAUCUU | AAGATGGUCUUGUCUGUCUGG |
| CCAGACAGACAAGACCAUCUU | AAGATGGUCUUGUCUGUCUGGCU |
| CCAGACAGACAAGACCAUCUU | AAGATGGUCUUGUCUGUCUGGCU |
| GACUUCCUUGAAGCCAACUAU | AUAGUUGGCUUCAAGGAAGUCUC |
| GACUUCCUUGAAGCCAACUAU | AUAGTUGGCUUCAAGGAAGUCUC |
| GACUUCCUUGAAGCCAACUAU | AUAGTUGGCUUCAAGGAAGUCCC |
| GACUUCCUUGAAGCCAACUAU | AUAGTUGGCUUCAAGGAAGUCCC |
| CUUCCUUGAAGCCAACUAU | AUAGTUGGCUUCAAGGAAGUC |
| GACUUCCUTGAAGCCAACUAU | AUAGTUGGCUUCAAGGAAGUCCC |
| GACUUCCUTGAAGCCAACUAU | AUAGTUGGCUUCAAGGAAGUCCC |
| GACUUCCUTGAAGCCAACUAU | AUAGTUGGCUUCAAGGAAGUCCC |
| GACUUCCUTGAAGCCAACUAU | AUAGTUGGCUUCAAGGAAGUCCC |
| AGCCAACUACAUGAACCUACU | AGUAGGTUCAUGUAGUUGGCUUC |
| AGCCAACUACAUGAACCUACU | AGUAGGTUCAUGUAGUUGGCUUC |
| AGCCAACUACAUGAACCUACU | AGUAGGTUCAUGUAGUUGGCUUC |
| AGCCAACUACAUGAACCUACU | AGUAGGTUCAUGUAGUUGGCUUC |
| AGCCAACUACAUGAACCUACU | AGUAGGTUCAUGUAGUUGGCUCC |
| CCAACUACAUGAACCUACU | AGUAGGTUCAUGUAGUUGGCU |
| AGCCAACUACAUGAACCUACU | AGUAGGTUCAUGUAGUUGGCUCC |
| AGCCAACUACAUGAACCUACU | AGUAGGTUCAUGUAGUUGGCUCC |
| AGCCAACUACAUGAACCUACU | AGUAGGUUCAUGUAGUUGGCUCC |
| AGCCAACUACAUGAACCUACU | AGUAGGTUCAUGUAGUUGGCUCC |
| AGCCAACUACAUGAACCUACU | AGUAGGTUCAUGUAGUUGGCUUC |
| GAGAACCAGAAACAAUGCCAU | AUGGCATUGUUUCUGGUUCUCUU |
| GAGAACCAGAAACAAUGCCAU | AUGGCATUGUUUCUGGUUCUCUU |
| GAGAACCAGAAACAAUGCCAU | AUGGCATUGUUUCUGGUUCUCUU |

TABLE 49

| Sense Sequence | Antisense Sequence |
|---|---|
| 5′ to 3′ | 5′ to 3′ |
| GAGAACCAGAAACAAUGCCAU | AUGGCATUGUUUCUGGUUCUCCU |
| GAACCAGAAACAAUGCCAU | AUGGCATUGUUUCUGGUUCUC |
| GAGAACCAGAAACAAUGCCAU | ATGGCATUGUUUCUGGUUCUCCU |
| GAGAACCAGAAACAAUGCCAU | AUGGCATUGUUUCUGGUUCUCCU |
| GAGAACCAGAAACAAUGCCAU | AUGGCATUGUUUCUGGUUCUCCU |
| GAGAACCAGAAACAAUGCCAU | AUGGCATUGUUUCUGGUUCUCCU |
| ACAGACAAGACCAUCUACACU | AGUGUAGAUGGUCUUGUCUGUCU |
| ACAGACAAGACCAUCUACACU | AGUGUAGAUGGUCUUGUCUGUCU |
| ACAGACAAGACCAUCUACACU | AGUGTAGAUGGUCUUGUCUGUGC |
| AGACAAGACCAUCUACACU | AGUGTAGAUGGUCUUGUCUGU |
| ACAGACAAGACCAUCUACACU | AGUGTAGAUGGUCUUGUCUGUCU |
| ACAGACAAGACCAUCUACACU | AGUGTAGAUGGUCUUGUCUGUCU |
| ACAGACAAGACCAUCUACACU | AGUGTAGAUGGUCUUGUCUGUCU |
| AGAUCCGAGCCUACUAUGAAU | AUUCAUAGUAGGCUCGGAUCUUC |
| AGAUCCGAGCCUACUAUGAAU | AUUCAUAGUAGGCUCGGAUCUUC |
| AGAUCCGAGCCUACUAUGAAU | AUUCAUAGUAGGCUCGGAUCUCC |
| AUCCGAGCCUACUAUGAAU | AUUCAUAGUAGGCUCGGAUCU |
| AGAUCCGAGCCUACUAUGAAU | AUUCAUAGUAGGCUCGGAUCUUC |
| AGAUCCGAGCCUACUAUGAAU | AUUCAUAGUAGGCUCGGAUCUUC |
| AGAUCCGAGCCUACUAUGAAU | AUUCAUAGUAGGCUCGGAUCUUC |
| AGAUCCGAGCCUACUAUGAAU | AUUCAUAGUAGGCUCGGAUCUUC |
| CCGAGCCGUUCUCUACAAUUU | AAAUUGUAGAGAACGGCUCGGAU |
| CCGAGCCGUUCUCUACAAUUU | AAAUUGUAGAGAACGGCUCGGAU |
| CCGAGCCGUUCUCUACAAUUU | AAAUUGUAGAGAACGGCUCGGAU |
| CCGAGCCGUUCUCUACAAUUU | AAAUTGTAGAGAACGGCUCGGAU |
| CCGAGCCGUUCUCUACAAUUU | AAAUTGTAGAGAACGGCUCGGAU |
| CCGAGCCGUUCUCUACAAUUU | AAAUTGTAGAGAACGGCUCGGGC |
| GAGCCGUUCUCUACAAUUU | AAAUTGTAGAGAACGGCUCGG |
| CCGAGCCGTUCUCUACAAUUU | AAAUTGTAGAGAACGGCUCGGGC |
| CCGAGCCGTUCUCUACAAUUU | AAAUTGTAGAGAACGGCUCGGGC |
| CCGAGCCGTUCUCUACAAUUU | AAAUTGTAGAGAACGGCUCGGGC |
| CGAGCCGUUCUCUACAAUUAU | AUAAUUGUAGAGAACGGCUCGGA |
| CGAGCCGUUCUCUACAAUUAU | AUAAUUGUAGAGAACGGCUCGGA |
| CGAGCCGUUCUCUACAAUUAU | AUAAUUGUAGAGAACGGCUCGGA |

TABLE 50

| Sense Sequence | Antisense Sequence |
|---|---|
| 5′ to 3′ | 5′ to 3′ |
| CGAGCCGUUCUCUACAAUUAU | AUAAUUGUAGAGAACGGCUCGGC |
| CGAGCCGUUCUCUACAAUUAU | AUAATUGUAGAGAACGGCUCGGC |
| AGCCGUUCUCUACAAUUAU | AUAATUGUAGAGAACGGCUCG |
| CGAGCCGUUCUCUACAAUUAU | AUAAUUGUAGAGAACGGCUCGGC |
| CGAGCCGUUCUCUACAAUUAU | AUAAUUGUAGAGAACGGCUCGGC |
| CGAGCCGUTCTCUACAAUUAU | AUAAUUGUAGAGAACGGCUCGGC |
| CGAGCCGUTCTCUACAAUUAU | AUAAUUGUAGAGAACGGCUCGGC |
| GAGCCGUUCUCUACAAUUACU | AGUAAUUGUAGAGAACGGCUCGG |
| GAGCCGUUCUCUACAAUUACU | AGUAAUUGUAGAGAACGGCUCGG |
| GAGCCGUUCUCUACAAUUACU | AGUAAUUGUAGAGAACGGCUCGG |
| GAGCCGUUCUCUACAAUUACU | AGUAAUUGUAGAGAACGGCUCCU |
| GCCGUUCUCUACAAUUACU | AGUAAUUGUAGAGAACGGCUC |
| GAGCCGUUCUCUACAAUUACU | AGUAAUUGUAGAGAACGGCUCGG |
| GAGCCGUUCUCUACAAUUACU | AGUAAUUGUAGAGAACGGCUCGG |
| GAGCCGTUCUCUACAAUUACU | AGUAAUUGUAGAGAACGGCUCGG |
| CAAGCCUUGGCUCAAUACCAU | AUGGUAUUGAGCCAAGGCUUGGA |
| CAAGCCUUGGCUCAAUACCAU | AUGGUAUUGAGCCAAGGCUUGGA |
| CAAGCCUUGGCUCAAUACCAU | AUGGUAUUGAGCCAAGGCUUGGC |
| CAAGCCUUGGCUCAAUACCAU | AUGGTATUGAGCCAAGGCUUGGC |
| AGCCUUGGCUCAAUACCAU | AUGGTATUGAGCCAAGGCUUG |
| CAAGCCUUGGCUCAAUACCAU | AUGGTATUGAGCCAAGGCUUGGC |
| CAAGCCUUGGCUCAAUACCAU | AUGGTATUGAGCCAAGGCUUGGC |
| ACUCACCUGUAAUAAAUUCGU | ACGAAUUUAUUACAGGUGAGUUG |
| ACUCACCUGUAAUAAAUUCGU | ACGAAUUUAUUACAGGUGAGUUG |
| ACUCACCUGUAAUAAAUUCGU | ACGAAUTUAUUACAGGUGAGUUG |
| ACUCACCUGUAAUAAAUUCGU | ACGAAUUUAUUACAGGUGAGUCC |
| UCACCUGUAAUAAAUUCGU | ACGAAUUUAGUACAGGUGAGU |
| ACUCACCUGUAAUAAAUUCGU | ACGAAUUUAUUACAGGUGAGUUG |
| ACUCACCUGUAAUAAAUUCGU | ACGAAUUUAUUACAGGUGAGUUG |
| ACUCACCUGUAAUAAAUUCGU | ACGAAUUUAUUACAGGUGAGUUG |
| ACUCACCUGUAAUAAAUUCGU | ACGAAUUUAUUACAGGUGAGUUG |
| GAUGCCAAGAACACUAUGAUU | AAUCAUAGUGUUCUUGGCAUCCU |
| GAUGCCAAGAACACUAUGAUU | AAUCAUAGUGUUCUUGGCAUCCU |
| GAUGCCAAGAACACUAUGAUU | AAUCAUAGUGUUCUUGGCAUCCU |

TABLE 51

| Sense Sequence | Antisense Sequence |
|---|---|
| 5′ to 3′ | 5′ to 3′ |
| GAUGCCAAGAACACUAUGAUU | AAUCAUAGUGUUCUUGGCAUCCU |
| GAUGCCAAGAACACUAUGAUU | AAUCAUAGUGUUCUUGGCAUCGG |
| UGCCAAGAACACUAUGAUU | AAUCAUAGUGUUCUUGGCAUC |
| GAUGCCAAGAACACUAUGAUU | AAUCAUAGUGUUCUUGGCAUCCU |
| GAUGCCAAGAACACUAUGAUU | AAUCAUAGUGUUCUUGGCAUCCU |
| GAUGCCAAGAACACUAUGAUU | AAUCAUAGUGUUCUUGGCAUCCU |
| GAUGCCAAGAACACUAUGAUU | AAUCAUAGUGUUCUUGGCAUCCU |
| GAUGCCAAGAACACUAUGAUU | AAUCAUAGUGUUCUUGGCAUCCU |
| UCAAGGUCUACGCCUAUUACU | AGUAAUAGGCGUAGACCUUGACU |
| UCAAGGUCUACGCCUAUUACU | AGUAAUAGGCGUAGACCUUGACU |
| UCAAGGUCUACGCCUAUUACU | AGUAAUAGGCGUAGACCUUGACU |
| UCAAGGUCUACGCCUAUUACU | AGUAAUAGGCGUAGACCUUGACC |
| AAGGUCUACGCCUAUUACU | AGUAAUAGGCGUAGACCUUGA |
| UCAAGGUCUACGCCUAUUACU | AGUAAUAGGCGUAGACCUUGACU |
| UCAAGGUCUACGCCUAUUACU | AGUAAUAGGCGUAGACCUUGACU |
| UCAAGGUCUACGCCUAUUACU | AGUAAUAGGCGUAGACCUUGACU |
| UCAAGGUCTACGCCUAUUACU | AGUAAUAGGCGUAGACCUUGACU |
| UCAAGGUCTACGCCUAUUACU | AGUAAUAGGCGUAGACCUUGACU |
| AAUUCUACUACAUCUAUAACU | AGUUAUAGAUGUAGUAGAAUUUC |
| AAUUCUACUACAUCUAUAACU | AGUUAUAGAUGUAGUAGAAUUUC |
| AAUUCUACUACAUCUAUAACU | AGUUAUAGAUGUAGUAGAAUUUC |
| AAUUCUACUACAUCUAUAACU | AGUUAUAGAUGTAGUAGAAUUUC |
| AAUUCUACUACAUCUAUAACU | AGUUAUAGAUGUAGUAGAAUUGG |
| AAUUCUACUACAUCUAUAACU | AGUUAUAGAUGTAGUAGAAUUGG |
| AAUUCUACUACAUCUAUAACU | AGUUAUAGAUGUAGUAGAAUU |
| AAUUCUACUACAUCUAUAACU | AGUUAUAGAUGTAGUAGAAUU |
| AAUUCUACUACAUCUAUAACU | AGUUAUAGAUGUAGUAGAAUUUC |
| AAUUCUACUACAUCUAUAACU | AGUUAUAGAUGUAGUAGAAUUGG |
| AAUUCUACUACAUCUAUAACU | AGUUAUAGAUGTAGUAGAAUUGG |
| AAUUCUACUACAUCUAUAACU | AGUUAUAGAUGUAGUAGAAUU |
| AAUUCUACUACAUCUAUAACU | AGUUAUAGAUGTAGUAGAAUU |
| CAGAGAAAUUCUACUACAUCU | AGAUGUAGUAGAAUUUCUCUGUA |
| CAGAGAAAUUCUACUACAUCU | AGAUGUAGUAGAAUUUCUCUGUC |
| CAGAGAAAUUCUACUACAUCU | AGAUGUAGUAGAAUUUCUCUGUC |

TABLE 52

| Sense Sequence | Antisense Sequence |
|---|---|
| 5′ to 3′ | 5′ to 3′ |
| AGAGAAAUUCUACUACAUCUU | AAGAUGTAGUAGAAUUUCUCUGU |
| AGAGAAAUUCUACUACAUCUU | AAGATGTAGUAGAAUUUCUCUGU |
| AGAGAAAUUCUACUACAUCUU | AAGATGUAGUAGAAUUUCUCUGU |
| AGAGAAAUUCUACUAUAUCUU | AAGATATAGUAGAAUUUCUCUGU |
| AGAAAUUCUACUACAUCUAUU | AAUAGATGUAGUAGAAUUUCUCU |
| AGAAAUUCUACUACAUCUAUU | AAUAGATGUAGTAGAAUUUCUCU |
| AGAAAUUCUACUACAUCUAUU | AAUAGAUGUAGTAGAAUUUCUCU |
| AGAAAUUCUACUACAUCUAUU | AAUAGATGUAGTAGAAUUUCUCU |
| CAAGGUCUUCUCUCUGGCUGU | ACAGCCAGAGAGAAGACCUUGAC |
| CAAGGUCUUCUCUCUGGCUGU | ACAGCCAGAGAGAAGACCUUGGC |
| CAAGGUCUUCUCUCUGGCUGU | ACAGCCAGAGAGAAGACCUUGGC |
| CAAGGUCUUCUCUCUGGCUGU | ACAGCCAGAGAGAAGACCUUGGC |
| AGGAGACUUCCUUGAAGCCAU | AUGGCUTCAAGGAAGUCUCCUGC |
| AGGAGACUUCCUUGAAGCCAU | AUGGCUTCAAGGAAGUCUCCUGC |
| AGGAGACUUCCUUGAAGCCAU | AUGGCUUCAAGGAAGUCUCCUGC |
| GGAGACUUCCUUGAAGCCAAU | AUUGGCTUCAAGGAAGUCUCCUG |
| GGAGACUUCCUUGAAGCCAAU | AUUGGCTUCAAGGAAGUCUCCUG |
| GGAGACUUCCUUGAAGCCAAU | AUUGGCUUCAAGGAAGUCUCCUG |
| CCUACAGAGAAAUUCUACUAU | AUAGUAGAAUUUCUCUGUAGGCU |
| CCUACAGAGAAAUUCUACUAU | AUAGTAGAAUUUCUCUGUAGGCU |
| CCUACAGAGAAAUUCUACUAD | AUAGTAGAAUUUCUCUGUAGGCU |
| ACAGAGAAAUUCUACUACAUU | AAUGUAGUAGAAUUUCUCUGUAG |
| ACAGAGAAAUUCUACUACAUU | AAUGTAGUAGAAUUUCUCUGUGG |
| ACAGAGAAAUUCUACUACAUU | AAUGTAGUAGAAUUUCUCUGUGG |
| GAGAAAUUCUACUACAUCUAU | AUAGAUGUAGUAGAAUUUCUCUG |
| GAGAAAUUCUACUACAUCUAU | AUAGAUGUAGUAGAAUUUCUCUG |
| GAGAAAUUCUACUACAUCUAU | AUAGAUGUAGUAGAAUUUCUCUG |
| AGAAAUUCUACUACAUCUAUU | AAUAGAUGUAGUAGAAUUUCUCU |
| AGAAAUUCUACUACAUCUAUU | AAUAGAUGUAGTAGAAUUUCUCU |
| AGAAAUUCUACUACAUCUAUU | AAUAGATGUAGTAGAAUUUCUCU |
| GAAAUUCUACUACAUCUAUAU | AUAUAGAUGUAGUAGAAUUUCUC |
| GAAAUUCUACUACAUCUAUAU | AUAUAGAUGUAGUAGAAUUUCUC |
| GAAAUUCUACUACAUCUAUAU | AUAUAGAUGUAGUAGAAUUUCUC |
| ACCCUACUCUGUUGUUCGAAU | AUUCGAACAACAGAGUAGGGUAG |

TABLE 53

| Sense Sequence | Antisense Sequence |
|---|---|
| 5′ to 3′ | 5′ to 3′ |
| ACCCUACUCUGUUGUUCGAAU | AUUCGAACAACAGAGUAGGGUGG |
| ACCCUACUCUGUUGUUCGAAU | AUUCGAACAACAGAGUAGGGUGG |
| CAAGGUCUUCUCUCUGGCUGU | ACAGCCAGAGAGAAGACCUUGAC |
| CAAGGUCUUCUCUCUGGCUGU | ACAGCCAGAGAGAAGACCUUGGC |
| CAAGGUCUUCUCUCUGGCUGU | ACAGCCAGAGAGAAGACCUUGGC |
| UGGCUCAAUGAACAGAGAUAU | AUAUCUCUGUUCAUUGAGCCAAC |
| UGGCUCAAUGAACAGAGAUAU | AUAUCUCUGUUCAUUGAGCCAGC |
| UGGCUCAAUGAACAGAGAUAU | AUAUCUCUGUUCAUUGAGCCAGC |
| UGGCUCAAUGAACAGAGAUAU | AUAUCUCUGUUCAUUGAGCCAGC |
| GCUGAGGAGAAUUGCUUCAUU | AAUGAAGCAAUUCUCCUCAGCAC |
| GCUGAGGAGAAUUGCUUCAUU | AAUGAAGCAAUUCUCCUCAGCGC |
| GCUGAGGAGAAUUGCUUCAUU | AAUGAAGCAAUUCUCCUCAGCGC |

TABLE 54

| Sense Sequence | Antisense Sequence |
|---|---|
| 5′ to 3′ | 5′ to 3′ |
| CGGGUACCUCUUCAUCCAGAU | AUCUGGAUGAAGAGGUACCCGCU |
| GUACCUCUUCAUCCAGACAGU | ACUGUCTGGAUGAAGAGGUACCC |
| UACCUCUUCAUCCAGACAGAU | AUCUGUCUGGAUGAAGAGGUACC |
| CAUCCAGACAGACAAGACCAU | AUGGUCTUGUCUGUCUGGAUGAA |
| CCAGACAGACAAGACCAUCUU | AAGAUGGUCUUGUCUGUCUGGAU |
| AGACAGACAAGACCAUCUACU | AGUAGATGGUCUUGUCUGUCUGG |
| GACAGACAAGACCAUCUACAU | AUGUAGAUGGUCUUGUCUGUCUC |
| AUUCCGGAACUCGUCAACAUU | AAUGUUGACGAGUUCCGGAAUGU |
| CACUGAGUUUGAGGUGAAGGU | ACCUUCACCUCAAACUCAGUGGA |
| ACUGAGUUUGAGGUGAAGGAU | AUCCUUCACCUCAAACUCAGUGG |
| GCCCAGUUUCGAGGUCAUAGU | ACUAUGACCUCGAAACUGGGCAG |
| AAUUCUACUACAUCUAUAACU | AGUUAUAGAUGUAGUAGAAUUUC |
| UCCCUACCAGAUCCACUUCAU | AUGAAGTGGAUCUGGUAGGGAGA |
| CCCUACCAGAUCCACUUCACU | AGUGAAGUGGAUCUGGUAGGGAG |
| CUACCAGAUCCACUUCACCAU | AUGGUGAAGUGGAUCUGGUAGGG |
| UACCAGAUCCACUUCACCAAU | AUUGGUGAAGUGGAUCUGGUAGG |
| CCAGAUCCACUUCACCAAGAU | AUCUUGGUGAAGUGGAUCUGGUA |
| GGGCAACUCCAACAAUUACCU | AGGUAATUGUUGGAGUUGCGCAC |
| GCAACUCCAACAAUUACCUGU | ACAGGUAAUUGUUGGAGUUGCCC |
| CAACUCCAACAAUUACCUGCU | AGCAGGTAAUUGUUGGAGUUGCC |
| CAACAAUUACCUGCAUCUCUU | AAGAGATGCAGGUAAUUGUUGGA |
| CAAGAUCCGCUACUACACCUU | AAGGUGTAGUAGOGGAUCUUGGC |
| CGUGCUGAAUAAGAAGAACAU | AUGUUCTUCUUAUUCAGCACGAA |
| GUGCUGAAUAAGAAGAACAAU | AUUGUUCUUCUUAUUCAGCACGA |
| ACUGACGCAGAGUAAGAUCUU | AAGAUCTUACUCUGCGUCAGUUU |

TABLE 55

| Sense Sequence | Antisense Sequence |
|---|---|
| 5′ to 3′ | 5′ to 3′ |
| UGCAGAAGAGAACAUCGUUUU | AAAACGAUGUUCUCUUCUGCAAU |
| CAUGUCGGACAAGAAAGGGAU | AUCCCUTUCUUGUCCGACAUGCU |
| AUGUCGGACAAGAAAGGGAUU | AAUCCCTUUCUUGUCCGACAUGC |
| GUCGGACAAGAAAGGGAUCUU | AAGAUCCCUUUCUUGUCCGACAD |
| CGGACAAGAAAGGGAUCUGUU | AACAGATCCCUUUCUUGUCCGAC |
| ACAGUAAUGCAGGACUUCUUU | AAAGAAGUCCUGCAUUACUGUGA |
| AGUAAUGCAGGACUUCUUCAU | AUGAAGAAGUCCUGCAUUACUGU |
| GUAAUGCAGGACUUCUUCAUU | AAUGAAGAAGUCCUGCALUACUG |
| CUACCCUACUCUGUUGUUCGU | ACGAACAACAGAGUAGGGUAGCC |
| UACCCUACUCUGUUGUUCGAU | AUCGAACAACAGAGUAGGGUAGC |
| ACCCUACUCUGUUGUUCGAAU | AUUCGAACAACAGAGUAGGGUAG |
| CCCUACUCUGUUGUUCGAAAU | AUUUCGAACAACACAGUAGGGUA |
| CCUACUCUGUUGUUCGAAACU | AGUUUCGAACAACAGAGUAGGGU |
| CUACUCUGUUGUUCGAAACGU | ACGUUUCGAACAACAGAGUAGGG |
| UACUCUGUUGUUCGAAACGAU | AUCGUUTCGAACAACAGAGUAGG |
| ACUCUGUUGUUCGAAACGAGU | ACUCGUTUCGAACAACAGAGUAG |
| CCGUUCUCUACAAUUACCGGU | ACCGGUAAUUGUAGAGAACGGCU |
| GGUGGAACUACUCCACAAUCU | AGAUUGTGGAGUAGUUCCACCCU |
| CCGAGUCUGAGACCAGAAUUU | AAAUUCTGGUCUCAGACUCGGUG |
| GUGCAUUACCUGGAUGAAACU | AGUUUCAUCCAGGUAAUGCACAG |
| CUACGUGGUCAAGGUCUUCUU | AAGAAGACCUUGACCACGUAGGC |
| CGUGGUCAAGGUCUUCUCUCU | AGAGAGAAGACCUUGACCACGUA |
| GUGGUCAAGGUCUUCUCUCUU | AAGAGAGAAGACCUUGACCACGU |
| CGUGAUACACCAAGAAAUGAU | AUCAUUTCUUGGUGUAUCACGGG |
| CGGCCUUUGUUCUCAUCUCGU | ACGAGATGAGAACAAAGGCCGUG |
| GACUUCCUUGAAGCCAACUAU | AUAGUUGGCUUCAAGGAAGUCUC |

TABLE 56

| Sense Sequence | Antisense Sequence |
|---|---|
| 5′ to 3′ | 5′ to 3′ |
| UUCCUUGAAGCCAACUACAUU | AAUGUAGUUGGCUUCAAGGAAGU |
| AAGCCAACUACAUGAACCUAU | AUAGGUTCAUGUAGUUGGCUUCA |
| AGCCAACUACAUGAACCUACU | AGUAGGTUCAUGUAGUUGGCUUC |
| CAACUACAUGAACCUACAGAU | AUCUGUAGGUUCAUGUAGUUGGC |
| UUCUGACCACAGCCAAAGAUU | AAUCUUTGGCUGUGGUCAGAAAU |
| UGCAGCUAAAAGACUUUGACU | AGUCAAAGUCUUUUAGCUGCAGU |
| CGUGCGUUGGCUCAAUGAACU | AGUUCATUGAGCCAACGCACGAC |
| UUCAUGGUGUUCCAAGCCUUU | AAAGGCTUGGAACACCAUGAAGG |
| CUGCGAUCAGAAGAGACCAAU | AUUGGUCUCUUCUGAUCGCAGGA |
| UGCGAUCAGAAGAGACCAAGU | ACUUGGTCUCUUCUGAUCGCAGG |
| ACCAAGGAAAAUGAGGGUUUU | AAAACCCUCAUUUUCCUUGGUCU |
| AAGGAAAAUGAGGGUUUCACU | AGUGAAACCCUCAUUUUCCUUGG |
| ACUCACCUGUAAUAAAUUCGU | ACGAAUTUAUUACAGGUGAGUUG |
| CUCACCUGUAAUAAAUUCGAU | AUCGAATUUAUUACAGGUGAGUU |
| ACCUGUAAUAAAUUCGACCUU | AAGGUCGAAUUUAUUACAGGUGA |
| CCUGUAAUAAAUUCGACCUCU | AGAGGUCGAAUUUAUUACAGGUG |
| CUGUAAUAAAUUCGACCUCAU | AUGAGGTCGAAUUUAUUACAGGU |
| ACCUCAAGGUCACCAUAAAAU | AUUUUATGGUGACCUUGAGGUCG |
| CCUCAAGGUCACCAUAAAACU | AGUUUUAUGGUGACCUUGAGGUC |
| UCAAGGUCACCAUAAAACCAU | AUGGUUTUAUGGUGACCUUGAGG |
| CAGAUACAUCUCCAAGUAUGU | ACAUACTUGGAGAUGUAUCUGUC |
| UGGACAAAGCCUUCUCCGAUU | AAUCGGAGAAGGCUUUGUCCAGC |
| UCUAGCUUUCAAAGUUCACCU | AGGUGAACUUUGAAAGCUAGACA |
| CUAGCUUUCAAAGUUCACCAU | AUGGUGAACUUUGAAAGCUAGAC |
| AGUCAAGGUCUACGCCUAUUU | AAAUAGGCGUAGACCUUGACUGC |
| CAAGGUCUACGCCUAUUACAU | AUGUAATAGGCGUAGACCUUGAC |

TABLE 57

| Sense Sequence | Antisense Sequence |
|---|---|
| 5′ to 3′ | 5′ to 3′ |
| AAGGUCUACGCCUAUUACAAU | AUUGUAAUAGGCGUAGACCUUGA |
| AGGUCUACGCCUAUUACAACU | AGUUGUAAUAGGCGUAGACCUUG |
| GUCUACGCCUAUUACAACCUU | AAGGUUGUAAUAGGCGUAGACCU |
| UCUACGCCUADUACAACCUGU | ACAGGUTGUAAUAGGCGUAGACC |
| GGAGUGGACUAUGUGUACAAU | AUUGUACACAUAGUCCACUCCUG |
| AGUGGACUAUGUGUACAAGAU | AUCUUGTACACAUAGUCCACUCC |
| GUGGACUAUGUGUACAAGACU | AGUCUUGUACACAUAGUCCACUC |
| UGGACUAUGUGUACAAGACCU | AGGUCUTGUACACAUAGUCCACU |
| ACCUGUCCAAUGACUUUGACU | AGUCAAAGUCAUUGGACAGCUGA |
| GCUGUCCAAUGACUUUGACGU | ACGUCAAAGUCAUUGGACAGCUG |
| GAGAACCAGAAACAAUGCCAU | AUGGCATUGUUUCUGGUUCUCUU |

TABLE 58

| Sense Sequence | Antisense Sequence |
|---|---|
| 5′ to 3′ | 5′ to 3′ |
| ACGGUCAUGGUCAACAUUGAU | AUCAAUGUUGACCAUGACCGUCC |
| AGAUCCGAGCCUACUAUGAAU | AUUCAUAGUAGGCUCGGAUCUUC |
| GAUCCGAGCCUACUAUGAAAU | AUUUCAUAGUAGGCUCGGAUCUU |
| AAUUCUACUACAUCUAUAACU | AGUUAUAGAUGUAGUAGAAUUUC |
| UGGGCAACUCCAACAAUUACU | AGUAAUUGUUGGAGUUGCCCACG |
| GGCAACUCCAACAAUUACCUU | AAGGUAAUUGUUGGAGUUGCCCA |
| CGUGUUCGUGCUGAAUAAGAU | AUCUUAUUCAGCACGAACACGCC |
| UGUUCGUGCUGAAUAAGAAGU | ACUUCUUAUUCAGCACGAACACG |
| GUUCGUGCUGAAUAAGAAGAU | AUCUUCUUAUUCAGCACGAACAC |
| UUCGUGCUGAAUAAGAAGAAU | AUUCUUCUUAUUCAGCACGAACA |
| ACUGACGCAGAGUAAGAUCUU | AAGAUCUUACUCUGCGUCAGUUU |
| UCCGAGCCGUUCUCUACAAUU | AAUUGUAGAGAACGGCUCGGAUU |
| CCGAGCCGUUCUCUACAAUUU | AAAUUGUAGAGAACGGCUCGGAU |
| CGAGCCGUUCUCUACAAUUAU | AUAAUUGUAGAGAACGGCUCGGA |
| GAGCGGUUCUCUACAAUUACU | AGUAAUUGUAGAGAACGGCUCGG |
| UUCCUUGAAGCCAACUACAUU | AAUGUAGUUGGCUUCAAGGAAGU |
| GCCUCUUCUUAACAAAUUUCU | AGAAAUUUGUUAAGAAGAGGCCC |
| CCACAGCCAAAGADAAGAACU | AGUUCUUAUCUUUGGCUGUGGUC |
| CUACUGCAGCUAAAAGACUUU | AAAGUCUUUUAGCUGCAGUAGCG |
| UCGUGCGUUGGCUCAAUGAAU | AUUCAUUGAGCCAACGCACGACG |
| GUGCGUUGGCUCAAUGAACAU | AUGUUCAUUGAGCCAACGCACGA |
| AGCCUUGGCUCAAUACCAAAU | AUUUGGUAUUGAGCCAAGGCUUG |
| AACUCACCUGUAAUAAAUUCU | AGAAUUUAUUACAGGUGAGUUGA |
| ACUCACCUGUAAUAAAUUCGU | ACGAAUUUAUUACAGGUGAGUUG |
| CUCACCUGUAAUAAAUUCGAU | AUCGAAUUUAUUACAGGUGAGUU |

TABLE 59

| Sense Sequence | Antisense Sequence |
|---|---|
| 5′ to 3′ | 5' to 3' |
| GACCUCAAGGUCACCAUAAAU | AUUUAUGGUGACCUUGAGGUCGA |
| CCUCAAGGUCACCAUAAAACU | AGUUUUAUGGUGACCUUGAGGUC |
| CUCAAGGUCACCAUAAAACCU | AGGUUUUAUGGUGACCUUGAGGU |
| UCAAGGUCACCAUAAAACCAU | AUGGUUUUAUGGUGACCUUGAGG |
| GAUGCCAAGAACACUAUGAUU | AAUCAUAGUGUUCUUGGCAUCCU |
| AGGAUGCCACUAUGUCUAUAU | AUAUAGACAUAGUGGCAUCCUGG |
| CAAGGUCUACGCCUAUUACAU | AUGUAAUAGGCGUAGACCUUGAC |
| AAGGUCUACGCCUAUUACAAU | AUUGUAAUAGGCGUAGACCUUGA |
| AGGUCUACGCCUAUUACAACU | AGUUGUAAUAGGCGUAGACCUUG |

TABLE 60

| Sense Sequence | Antisense Sequence |
|---|---|
| 5′ to 3′ | 5' to 3' |
| AGACAGACAAGACCAUCUACU | AGUAGAUGGUCUUGUCUGUCUGG |
| ACAGACAAGACCAUCUACACU | AGUGUAGAUGGUCUUGUCUGUCU |
| UGGGACAUUCCGGAACUCGUU | AACGAGUUCCGGAAUGUCCCAAG |
| AUUCCGGAACUCGUCAACAUU | AAUGUUGACGAGUUCCGGAAUGU |
| AGAUCCGAGCCUACUAUGAAU | AUUCAUAGUAGGCUCGGAUCUUC |
| GCAGGUCUUCUCCACUGAGUU | AACUCAGUGGAGAAGACCUGCUG |
| GCCCAGUUUCGAGGUCAUAGU | ACUAUGACCUCGAAACUGGGCAG |
| CCAGUUUCGAGGUCAUAGUGU | ACACUAUGACCUCGAAACUGGGC |
| AAUUCUACUACAUCUAUAACU | AGUUAUAGAUGUAGUAGAAUUUC |
| ACUGCCUUUGUCAUCUUCGGU | ACCGAAGAUGACAAAGGCAGUUC |
| CUCAUGGUGUUCGUGACGAAU | AUUCGUCACGAACACCAUGAGGU |
| UGGGCAACUCCAACAAUUACU | AGUAAUUGUUGGAGUUGCCCACG |
| GGGCAACUCCAACAAUUACCU | AGGUAAUUGUUGGAGUUGCCCAC |
| GCAACUCCAACAAUUACCUGU | ACAGGUAAUUGUUGGAGUUGCCC |
| GUCAACUUCCUCCUGCGAAUU | AAUUCGCAGGAGGAAGUUGACGU |
| AACUGACGCAGAGUAAGAUCU | AGAUCUUACUCUGCGUCAGUUUG |
| UGCAGAAGAGAACAUCGUUUU | AAAACGAUGUUCUCUUCUGCAAU |
| GAAGAGAACAUCGUUUCCCGU | ACGGGAAACGAUGUUCUCUUCUG |
| AAGAGAACAUCGUUUCCCGAU | AUCGGGAAACGAUGUUCUCUUCU |
| AGAACAUCGUUUCCCGAAGUU | AACUUCGGGAAACGAUGUUCUCU |
| GAACAUCGUUUCCCGAAGUGU | ACACUUCGGGAAACGAUGUUCUC |
| AACAUCGUUUCCCGAAGUGAU | AUCACUUCGGGAAACGAUGUUCU |
| CGGACAAGAAAGGGAUCUGUU | AACAGAUCCCUUUCUUGUCCGAC |
| GGACAAGAAAGGGAUCUGUGU | ACACAGAUCCCUUUCUUGUCCGA |
| GACAAGAAAGGGAUCUGUGUU | AACACAGAUCCCUUUCUUGUCCG |

TABLE 61

| Sense Sequence | Antisense Sequence |
|---|---|
| 5′ to 3′ | 5' to 3' |
| ACAAGAAAGGGAUCUGUGUGU | ACACACAGAUCCCUUUCUUGUCC |
| CAAGAAAGGGAUCUGUGUGGU | ACCACACAGAUCCCUUUCUUGUC |
| AAGAAAGGGAUCUGUGUGGCU | AGCCACACAGAUCCCUUUCUUGU |
| GAAAGGGAUCUGUGUGGCAGU | ACUGCCACACAGAUCCCUUUCUU |
| CUUCGAGGUCACAGUAAUGCU | AGCAUUACUGUGACCUCGAAGGG |
| UUCGAGGUCACAGUAAUGCAU | AUGCAUUACUGUGACCUCGAAGG |
| GGCUACCCUACUCUGUUGUUU | AAACAACAGAGUAGGGUAGCCGC |
| CUACCCUACUCUGUUGUUCGU | ACGAACAACAGAGUAGGGUAGCC |
| UACCCUACUCUGUUGUUCGAU | AUCGAACAACAGAGUAGGGUAGC |
| ACCCUACUCUGUUGUUCGAAU | AUUCGAACAACAGAGUAGGGUAG |
| CCCUACUCUGUUGUUCGAAAU | AUUUCGAACAACAGAGUAGGGUA |
| CCUACUCUGUUGUUCGAAACU | AGUUUCGAACAACAGAGUAGGGU |
| CUACUCUGUUGUUCGAAACGU | ACGUUUCGAACAACAGAGUAGGG |
| UACUCUGUUGUUCGAAACGAU | AUCGUUUCGAACAACAGAGUAGG |
| ACUCUGUUGUUCGAAACGAGU | ACUCGUUUCGAACAACAGAGUAG |
| CUCUGUUGUUCGAAACGAGCU | AGCUCGUUUCGAACAACAGAGUA |
| CCGUUCUCUACAAUUACCGGU | ACCGGUAAUUGUAGAGAACGGCU |
| AACAAAACUGUGGCUGUUCGU | ACGAACAGCCACAGUUUUGUUCA |
| GGUCAUCGCUGUGCAUUACCU | AGGUAAUGCACAGCGAUGACCGU |
| GUCAUCGCUGUGCAUUACCUU | AAGGUAAUGCACAGCGAUGACCG |
| UGCAUUACCUGGAUGAAACGU | ACGUUUCAUCCAGGUAAUGCACA |
| CGUGGUCAAGGUCUUCUCUCU | AGAGAGAAGACCUUGACCACGUA |
| GGCCUUUGUUCUCAUCUCGCU | ACGAGAUGAGAACAAAGGCCGUG |
| GCCUUUGUUCUCAUCUCGCUU | AGCGAGAUGAGAACAAAGGCCGU |
| UUCCUUGAAGCCAACUACAUU | AAGCGAGAUGAGAACAAAGGCCG |
| ACAAGAAAGGGAUCUGUGUGU | AAUGUAGUUGGCUUCAAGGAAGU |

TABLE 62

| Sense Sequence | Antisense Sequence |
|---|---|
| 5′ to 3′ | 5' to 3' |
| UGCAGCUAAAAGACUUUGACU | AGUCAAAGUCUUUUAGCUGCAGU |
| UCGUGCGUUGGCUCAAUGAAU | AUUCAUUGAGCCAACGCACGACG |
| CGUGGGUUGGCUCAAUGAACU | AGUUCAUUGAGCCAACGCACGAC |
| CAAUGAACAGAGAUACUACGU | ACGUAGUAUCUCUGUUCAUUGAG |
| AAUGAACAGAGAUACUACGGU | ACCGUAGUAUCUCUGUUCAUUGA |
| AUGAACAGAGAUACUACGGUU | AACCGUAGUAUCUCUGUUCAUUG |
| CCAAGCCUUGGCUCAAUACCU | AGGUAUUGAGCCAAGGCUUGGAA |
| CAAGCCUUGGCUCAAUACCAU | AUGGUAUUGAGCCAAGGCUUGGA |
| CCACCGUAUCCACUGGGAAUU | AAUUCCCAGUGGAUACGGUGGGU |
| ACCAAGGAAAAUGAGGGUUUU | AAAACCCUCAUUUUCCUUGGUCU |
| AAGGAAAAUGAGGGUUUCACU | AGUGAAACCCUCAUUUUCCUUGG |
| AACUCACCUGUAAUAAAUUCU | AGAAUUUAUUACAGGUCAGUUGA |
| ACUCACCUGUAAUAAAUUCGU | ACGAAUUUAUUACAGGUGAGUUG |
| CUCACCUGUAAUAAAUUCGAU | AUCGAAUUUAUUACAGGUGAGUU |
| ACCUGUAAUAAAUUCGACCUU | AAGGUCGAAUUUAUUACAGGUGA |
| UAAUAAAUUCGACCUCAAGGU | ACCUUGAGGUCGAAUUUAUUACA |
| ACCUCAAGGUCACCAUAAAAU | AUUUUAUGGUGACCUUGAGGUCG |
| CCUCAAGGUCACCAUAAAACU | AGUUUUAUGGUGACCUUGAGGUC |
| CUCAAGGUCACCAUAAAACCU | AGGUUUUAUGGUGACCUUGAGGU |
| AGGAUGCCAAGAACACUAUGU | ACAUAGUGUUCUUGGCAUCCUGA |
| CAGAUACAUCUCCAAGUAUGU | ACAUACUUGGAGAUGUAUCUGUC |
| UGGACAAAGCCUUCUCCGAUU | AAUCGGAGAAGGCUUUGUCCAGC |
| AGGAACACCCUCAUCAUCUAU | AUAGAUGAUGAGGGUGUUCCUAU |
| GGAACACCCUCAUCAUCUACU | AGUAGAUGAUGAGGGUGUUCCUA |
| AACAGCCUCAUCAUCUACCUU | AAGGUAGAUGAUGAGGGUGUUCC |
| CUUUAAUGUAGAGCUUAUCCU | AGGAUAAGCUCUACAUUAAAGUA |

TABLE 63

| Sense Sequence | Antisense Sequence |
|---|---|
| 5′ to 3′ | 5' to 3' |
| UUUAAUGUAGAGCUUAUCCAU | AUGGAUAAGCUCUACAUUAAAGU |
| UCAAGGUCUACGCCUAUUACU | AGUAAUAGGCGUAGACCUUGACU |
| CAAGGUCUACGCCUAUUACAU | AUGUAAUAGGCGUAGACCUUGAC |
| AAGGUCUACGCCUAUUACAAU | AUUGUAAUAGGCGUAGACCUUGA |
| AGGUCUACGCCUAUUACAACU | AGUUGUAAUAGGCGUAGACCUUG |
| UCUACGGCUAUUACAACCUGU | ACAGGUUGUAAUAGGCGUAGACC |
| ACCUGUCCAAUGACUUUGACU | AGUCAAAGUCAUUGGACAGCUGA |
| GCUCUCCAAUGACUUUGACGU | ACGUCAAAGUCAUUGGACAGGUG |
| AGCAUGGUUGUCUUUGGGUGU | ACACCCAAAGACAACCAUGCUCU |
| AAUAAGAAGAACAAACUGACA | UGUCAGUUUGUUCUUCUUAUUCA |
| AAUAAGAAGAACAAGCUGACA | UGUCAGCUUGUUCUUCUUAUUCA |
| AACACCCUCAUCAUCUACCUU | AAGGUAGAUGAUGAGGGUGUUCC |

TABLE 64

| Sense Sequence | Antisense Sequence |
|---|---|
| 5′ to 3′ | 5' to 3' |
| GUGGUCAAGGUCUUCUCUCUA | UAGAGAGAAGACCUUGACCACGU |
| UACCUCUUCAUCCAGACAGAA | UUCUGUCUGGAUGAAGAGGUACC |
| CCAGAUCCACUUCACCAAGAA | UUCUUGGUGAAGUGGAUCUGGUA |
| CGGGUACCUCUUCAUCCAGAA | UUCUGGAUGAAGAGGUACCGGCU |
| GUACCUCUUCAUCCAGACAGA | UCUGUCUGGAUGAAGAGGUACCC |
| CAUCCAGACAGACAAGACCAA | UUCGUCUUGUCUGUCUGGAUGAA |
| CCAGACAGACAAGACCAUCUA | UAGAUGGUCUUGUCUGUCUGGAU |
| AGACAGACAAGACCAUCUACA | UGUAGAUGGUCUUGUCUGUCUGG |
| ACAGACAAGACCAUCUACACA | UGUGUAGAUGGUCUUGUCUGUCU |
| GACACACAAGACCAUCUACAA | UUGUAGAUGGUCUUGUCUGUCUG |
| ACGGUCAUGGUCAACAUUGAA | UUCAAUGUUGACCAUGACCGUCC |
| UGGGACAUUCCGGAACUCGUA | UACGAGUUCCGGAAUGUCGCAAG |
| AUUCGGGAACUCGUCAACAUA | UAUGUUGACGAGUUCCGGAAUGU |
| AGAUCCGAGCCUACUAUGAAA | UUUCAUAGUAGGCUCGGAUCUUC |
| GAUCCGAGCCUACUAUGAAAA | UUUUCAUAGUAGGCUCGGAUCUU |
| GCAGGUCUUCUCCACUGAGUA | UACUCAGUGGAGAAGACCUGCUG |
| CACUGAGUUUGAGGUGAAGGA | UCCUUCACCUCAAACUCAGUGGA |
| ACUGAGUUUGAGGUGAAGGAA | UUCCUUCACCUCAAACUCAGUGG |
| GCCCAGUUUCGAGGUCAUAGA | UCUAUGACCUCGAAACUGGGCAG |

TABLE 65

| Sense Sequence | Antisense Sequence |
|---|---|
| 5′ to 3′ | 5' to 3' |
| CCAGUUUCGAGGUCAUAGUGA | UCACUAUGACCUCGAAACUGGGC |
| AAUUCUACUACAUCUAUAACA | UGUUAUAGAUGUAGUAGAAUUUC |
| ACUGGCUUUGUCAUCUUCGGA | UCCGAAGAUGACAAAGGCAGUUC |
| UCCCUACCAGAUCCACUUCAA | UUGAAGUGGAUCUGGUAGGGAGA |
| CCCUACCAGAUCCACUUCACA | UGUGAAGUGGAUCUGGUAGGGAG |
| CUACCAGAUCCACUUCACCAA | UUGGUGAAGUGGAUCUGGUAGGG |
| UACCAGAUCCACUUCACCAAA | UUUGGUGAAGUGGAUCUGGUAGG |
| CUCAUGGUGUUCGUGACGAAA | JUUCGUCACGAACACCAUGAGGU |
| UGGGCAACUCCAACAAUUACA | UGUAAUUGUUGGAGUUGGCCACG |
| GGGCAACUCCAACAAUUACCA | UGGUAAUUGUUGGAGUUGGCCAC |
| GGCAACUCCAACAAUUACCUA | UAGGUAAUUGUUGGAGUUGCCCA |
| GCAACUCCAACAAUUACCUGA | UCAGGUAAUUGUUGGAGUUGCGC |
| CAACUCCAACAAUUACCUGCA | UGCAGGUAAUUGUUGGAGUUGCC |
| CAACAAUUACCUGCAUCUCUA | UAGAGAUGCAGGUAAUUGUUGGA |
| GUCAACUUCCUCCUGCGAAUA | UAUUCGCAGGAGGAAGUUCACGU |
| CAAGAUCCGCUACUACACCUA | UAGGUGUAGUAGCGGAUCUUGGC |
| CGUGUUCGUGCUGAAUAAGAA | UUCUUAUUCAGCACGAACACGCC |
| UGUUCGUGCUGAAUAAGAAGA | UCUUCUUAUUCAGCACGAACACG |
| GUUCGUGCUGAAUAAGAAGAA | UUCUUCUUAUUCAGCACGAACAC |
| UUCGUGCUGAAUAAGAAGAAA | UUUCUUCUUAUUCAGCACGAACA |
| CGUGCUGAAUAAGAAGAACAA | UUGUUCUUCUUAUUCAGCACGAA |
| GUGCUGAAUAAGAAGAACAAA | UUUGUUCUUCUUAUUCAGCACGA |
| AAUAAGAAGAACAAACUGACA | UGUCAGUUUGUUCUUCUUAUUCA |
| AACUGACGCAGAGUAAGAUCA | UGAUCUUACUCUGCGUCAGUUUG |
| ACUGACGCAGAGUAAGAUCUA | UAGAUCUUACUCUGCGUCAGUUU |
| UGCAGAAGAGAACAUCGUUUA | UAAACGAUGUUCUCUUCUGCAAU |

TABLE 66

| Sense Sequence | Antisense Sequence |
|---|---|
| 5′ to 3′ | 5' to 3' |
| GAAGAGAACAUCGUUUCCCGA | UCGGGAAACGAUGUUCUCUUCUG |
| AAGAGAACAUCGUUUCCCGAA | UUCGGGAAACGAUGUUCUCUUCU |
| AGAACAUCGUUUCCCGAAGUA | UACUUCGGGAAACGAUGUUCUCU |
| GAACAUCGUUUCCCGAAGUGA | UCACUUCGGGAAACGAUGUUCUC |
| AACAUCGUUUCCCGAAGUGAA | UUCACUUCGGGAAACGAUGUUCU |
| CAUGUGGGACAAGAAAGGGAA | UUCCCUUUCUUGUCCGACAUGCU |
| AUGUCGGACAAGAAAGGGAUA | UAUCCCUUUCUUGUCCGACAUGC |
| GUCGGACAAGAAAGGGAUCUA | UAGAUCCCUUUCUUGUCCGACAU |
| CGGACAAGAAAGGGAUCUGUA | UACAGAUCCCUUUCUUGUCCGAC |
| GGACAAGAAAGGGAUCUGUGA | UCACAGAUCCCUUUCUUGUCCGA |
| GACAAGAAAGGGAUCUGUGUA | UACACAGAUCCCUUUCUUGUCCG |
| ACAAGAAAGGGAUCUGUGUGA | UCACACAGAUCCCUUUCUUGUCC |
| CAAGAAAGGGAUCUGUGUGGA | UCCACACAGAUCCCUUUCUUGUC |
| AAGAAAGGGAUCUGUGUGGCA | UGCCACACAGAUCCCUUUCUUGU |
| GAAAGGGAUCUGUGUGGCAGA | UCUGCCACACAGAUCGCUUUCUU |
| CUUCGAGGUCACAGUAAUGCA | UGCAUUACUGUGACCUCGAAGGG |
| UUCGAGGUCACAGUAAUGCAA | UUGCAUUACUGUGACCUCGAAGG |
| ACAGUAAUGCAGGACUUCUUA | UAAGAAGUCCUGCAUUACUGUGA |
| AGUAAUGCAGGACUUCUUCAA | UUGAAGAAGUCCUGCAUUACUGU |
| GUAAUGCAGGACUUCUUCAUA | UAUGAAGAAGUCCUGCAUUACUG |
| GGCUACCCUACUCUGUUGUUA | UAACAACAGAGUAGGGUAGCCGC |
| CUACCCUACUCUGUUGUUCGA | UCGAACAACAGAGUAGGGUAGCC |
| UACCCUACUCUGUUGUUGGAA | UUCGAACAACAGAGUAGGGUAGC |
| ACCCUACUCUGUUGUUCGAAA | UUUCGAACAACAGAGUAGGGUAG |
| CCCUACUCUGUUGUUCGAAAA | UUUUCGAACAACAGAGUAGGGUA |
| CCUACUCUGUUGUUCGAAACA | UGUUUCGAACAACAGAGUAGGGU |

TABLE 67

| Sense Sequence | Antisense Sequence |
|---|---|
| 5′ to 3′ | 5' to 3' |
| CUACUCUGUUGUUCGAAACGA | UCGUUUCGAACAACAGAGUAGGG |
| UACUCUGUUGUUCCAAACGAA | UUCGUUUCGAACAACAGAGUAGG |
| ACUCUGUUGUUCGAAACGAGA | UCUCGUUUCGAACAACAGAGUAG |
| CUCUGUUGUUCGAAACGAGCA | UGCUCGUUUCGAACAACAGAGUA |
| UCCGAGCCGUUCUCUACAAUA | UAUUGUAGAGAACGGCUCGGAUU |
| CCGAGCCGUUCUCUACAAUUA | UAAUUGUAGAGAACGGCUCGGAU |
| CGAGCCGUUCUCUACAAUUAA | UUAAUUGUAGAGAACGGCUCGGA |
| GAGCCGUUCUCUACAAUUACA | UGUAAUUGUAGAGAACGGCUCGG |
| CCGUUCUCUACAAUUACCGGA | UCCGGUAAUUGUAGAGAACGGCU |
| GGUGGAACUACUCCACAAUCA | UGAUUGUGGAGUAGUUCCACCCU |
| AACAAAACUGUGGCUGUUCGA | UCGAACAGCCACAGUUUUGUUCA |
| CCGAGUCUGAGACCAGAAUUA | UAAUUCUGGUCUCAGACUCGGUG |
| GGUCAUCGCUGUGCAUUACCA | UGGUAAUGCACAGGGAUGACGGU |
| GUCAUCGCUGUGCAUUACCUA | UAGGUAAUGCACAGCGAUGACCG |
| GUCCAUUACCUGGAUGAAACA | UGUUUCAUCCAGGUAAUGCACAG |
| UGCAUUACCUGGAUGAAACGA | UCGUUUCAUCCAGGUAAUGCACA |
| CUACGUGGUCAAGGUCUUCUA | UAGAAGACCUUGACCACGUAGGC |
| CGUGGUCAAGGUCUUCUCUCA | UGAGAGAAGACCUUGACCACGUA |
| CGUGAUACACCAAGAAAUGAA | UUCAUUUCUUGGUGUAUCACGGG |
| CGGCCUUUGUUCUCAUCUCGA | UCGAGAUGAGAACAAAGGCCGUG |
| GGCCUUUGUUCUCAUCUGGCA | UGCGAGAUGAGAACAAAGGCCGU |
| GCCUUUGUUCUCAUCUCGCUA | UAGCGAGAUGAGAACAAAGGCCG |
| GACUUCCUUGAAGCCAACUAA | UUAGUUGGCUUCAAGGAAGUCUC |
| UUCCUUGAAGCCAACUACAUA | UAUGUAGUUGGCUUCAAGGAAGU |
| AAGCCAACUACAUGAACCUAA | UUAGGUUCAUGUAGUUGGCUUCA |
| AGCCAACUACAUGAACCUACA | UGUAGGUUCAUGUAGUUGGCUUC |

TABLE 68

| Sense Sequence | Antisense Sequence |
|---|---|
| 5′ to 3′ | 5' to 3' |
| CAACUACAUGAACCUACAGAA | UUCUGUAGGUUCAUGUAGUUGGC |
| GCCUCUUCUUAACAAAUUUCA | UGAAAUUUGUUAAGAAGAGGCCC |
| UUCUGACCACAGCCAAAGAUA | UAUCUUUGGCUGUGGUCAGAAAU |
| CCACAGCCAAAGAUAAGAACA | UGUUCUUAUCUUUGGCUGUGGUC |
| CUACUGCAGCUAAAAGACUUA | UAAGUCUUUUAGCUGCAGUAGGG |
| UGCAGCUAAAAGACUUUGACA | UGUCAAAGUCUUUUAGCUGCAGU |
| UCGUCCGUUGGCUCAAUGAAA | UUUCAUUGAGCCAACGCACGACG |
| CGUGCGUUGGCUCAAUGAACA | UGUUCAUUGAGCCAACGCACGAC |
| GUGCGUUGGCUCAAUGAACAA | UUGUUCAUUGAGCCAACGCACGA |
| CAAUGAACAGAGAUACUACGA | UCGUAGUAUCUCUGUUCAUUGAG |
| AAUGAACAGAGAUACUACGGA | UCCGUAGUAUCUCUGUUCAUUGA |
| AUGAACAGAGAUACUACGGUA | UACCGUAGUAUCUCUGUUCAUUG |
| UUCAUGGUGUUCCAAGCCUUA | UAAGGCUUGGAACACCAUGAAGG |
| CCAAGCCUUGGCUCAAUACCA | UGGUAUUGAGCCAAGGCUUGGAA |
| CAAGCCUUGGCUCAAUACCAA | UUGGUAUUGAGCCAAGGCUUGGA |
| AGCCUUGGCUCAAUACCAAAA | UUUUGGUAUUGAGCCAAGGCUUG |
| CCACCGUAUCCACUGGGAAUA | UAUUCCCAGUGGAUACGGUGGGU |
| CUGCGAUCAGAAGAGACCAAA | UUUGGUCUCUUCUGAUCGCAGGA |
| UGCGAUCAGAAGAGACCAAGA | UCUUGGUCUCUUCUGAUCGCAGG |
| CCAAGGAAAAUGAGGGUUUCA | UGAAACCCUCAUUUUCCUUGGUC |
| AAGGAAAAUGAGGGUUUCACA | UGUGAAACCCUCAUUUUCCUUGG |
| AACUCACCUGUAAUAAAUUCA | UGAAUUUAUUACAGGUGAGUUGA |
| ACUCACCUGUAAUAAAUUCGA | UCGAAUUUAUUACAGGUGAGUUG |
| CUCACCUGUAAUAAAUUCGAA | UUCGAAUUUAUUACAGGUGAGUU |
| ACCUGUAAUAAAUUCGACCUA | UAGGUCGAAUUUAUUACAGGUGA |
| CCUGUAAUAAAUUCGACCUCA | UGAGGUCGAAUUUAUUACAGGUG |

TABLE 69

| Sense Sequence | Antisense Sequence |
|---|---|
| 5′ to 3′ | 5' to 3' |
| CUGUAAUAAAUUCGACCUCAA | UUGAGGUCGAAUUUAUUACAGGU |
| UAAUAAAUUCGACCUCAAGGA | UCCUUGAGGUCGAAUUUAUUACA |
| GACCUCAAGGUCACCAUAAAA | UUUUAUGGUGACCUUGAGGUCGA |
| ACCUCAAGGUCACCAUAAAAA | UUUUUAUGGUGACCUUGAGGUCG |
| CCUCAAGGUCACCAUAAAACA | UGUUUUAUGGUGACCUUGAGGUC |
| CUCAAGGUCACCAUAAAACCA | UGGUUUUAUGGUGACCUUGAGGU |
| UCAAGGUCACCAUAAAACCAA | UUGGUUUUAUGGUGACCUUGAGG |
| AGGAUGCCAAGAACACUAUGA | UCAUAGUGUUCUUGGCAUCCUGA |
| GAUGCCAAGAACACUAUGAUA | UAUCAUAGUGUUCUUGGCAUCCU |
| AGGAUGCCACUAUGUCUAUAA | UUAUAGACAUAGUGGCAUCCUGG |
| CAGAUACAUCUCCAAGUAUGA | UCAUACUUGGAGAUGUAUCUGUC |
| UGGACAAAGCCUUCUCCGAUA | UAUCGGAGAAGGCUUUGUCCAGC |
| AGGAACACCCUCAUCAUCUAA | UUAGAUGAUGAGGGUGUUCCUAU |
| GGAACACCCUCAUCAUCUACA | UGUAGAUGAUGAGGGUGUUCCUA |
| AACACCCUCAUCAUCUACCUA | UAGGUAGAUGAUGAGGGUGUUCC |
| UCUAGCUUUCAAAGUUCACCA | UGGUGAACUUUGAAAGCUAGACA |
| CUAGCUUUCAAAGUUCACCAA | UUGGUGAACUUUGAAAGCUAGAC |
| CUUUAAUGUAGAGCUUAUCCA | UGGAUAAGCUCUACAUUAAAGUA |
| UUUAAUGUAGAGCUUAUCCAA | UUGGAUAAGCUCUACAUUAAAGU |
| AGUCAAGGUCUACGCCUAUUA | UAAUAGGCGUAGACCUUGACUGC |
| UCAAGGUCUACGCCUAUUACA | UGUAAUAGGCGUAGACCUUGACU |
| CAAGGUCUACGCCUAUUACAA | UUGUAAUAGGCGUAGACCUUGAC |
| AAGGUCUACGCCUAUUACAAA | UUUGUAAUAGGCGUAGACCUUGA |
| AGGUCUACGCCUAUUACAACA | UGUUGUAAUAGGCGUAGACCUUG |
| GUCUACGCCUAUUACAACCUA | UAGGUUGUAAUAGGCGUAGACCU |
| UCUACGCCUAUUACAACCUGA | UCAGGUUGUAAUAGGCGUAGACC |

TABLE 70

| Sense Sequence | Antisense Sequence |
|---|---|
| 5′ to 3′ | 5' to 3' |
| GGAGUGGACUAUGUGUACAAA | UUUGUACACAUAGUCCACUCCUG |
| AGUGGACUAUGUGUACAAGAA | UUCUUGUACACAUAGUCCACUCC |
| GUGGACUAUGUGUACAAGACA | UGUCUUGUACACAUAGUCCACUC |
| UGGACUAUGUGUACAAGACCA | UGGUCUUGUACACAUAGUCCACU |
| ACCUGUCCAAUGACUUUGACA | UGUCAAAGUCAUUGGACAGCUGA |
| GCUGUCCAAUGACUUUGACGA | UCGUCAAAGUCAUUGGACAGCUG |
| GAGAACCAGAAACAAUGGCAA | UUGGCAUUGUUUCUGGUUCUCGU |
| AGCAUGGUUGUCULUGGGUGA | UCACCCAAAGACAACCAUGCUCU |

TABLE 71
sIRNA Sense Sequence

AAACUGACGCAGAGUAAGA
AAAGAUCAACUCACCUGUA
AACAAGCUCUGCCGUGAUG
AAGAAACACUACCUCAUGU
PAGAAAUGAUUGGUGGAUU
AAGAUCAACUCACCUGUAA
ACAAAGCCUUCUCCGAUAG
ACACCUACCUGAUCAUGAA
ACAGGGAGUUCAAGUCAGA
ACAUGAACCUACAGAGAUC
ACGAAGAGAACCAGAAACA
ACGAAUGCCAAGACGAAGA
ACGGAACAACAACGAGAAA
ACGGAGAAGCGAAUGGACA
ACGUUGAGGACUUGAAAGA
AGAAAUGAUUGGUGGAUUA
AGAACACUAUGAUCCUUGA
AGACACAGAUGACCUGAAG
AGACAGACAAGACCAUCUA
AGACGAAGAGAACCAGAAA
AGAUGACCCUGAAGAUAGA
AGGAUGGAAAGCUGAACAA
AGGGAGUUCAAGUCAGAAA
AGGUUCAGCUGUCCAAUGA
AGUCACAGCUGAAGGAAAA
AGUGGAGCCUACAGAGAAA
AGUUUGAGGUGAAGGAGUA
AUUACAACCUGGAGGAAAG
CAACAACGAGAAAGACAUG
CAACAGGGAGUUCAAGUCA
CAACCCAGCUCUGCCUUUG
CAACUCACCUGUAAUAAAU
CAAGAACACUAUGAUCCUU
CAAGAUCACCCACCGUAUC
CAAGGUCACCCUGGAAGAA
CACCAAUACUUUAAUGUAG
CAGAAGACCUGGUGGGGAA
CAGAAGAGAACAUCGUUUC
CAGAAGAGACCAAGGAAAA
CAGAAGCCCUUGAGCAUCA
CAGAUCCACUUCACCAAGA
CAGAUGACCCUGAAGAUAG
CAGCAGCGCACGUUCAUCA
CAGCAUCAGUAAAGCAGGA
CAGUCAAGGUCUACGCCUA
CAGUCACAGCUGAAGGAAA
CAUCAUUGCAGAAGAGAAC
CCAACUACAUGAACCUACA
CCAAGACACCCAAGUACGU
CCAAGGARAAUGAGGGUUU
CCAAGGAGCUGCGCAAGUG
CCAAGUAUGAGCUGGACAA
CCAAUGACUUUGACGAGUA
CCAAUGGUGUUGACAGAUA
CCACCAACCACAUGGGCAA
CCACUGAGUUUGAGGUGAA
CCCUGAAGCUGGAGGAGAA
CCGGAAGGAAUCAGAAUGA
CCGUGAAGGAGUGCAGAAA
CCGUGAUACACCAAGAAAU
CCGUGUGGGUGGACGUCAA
CCUACAGCACCGUGGGCAA
CCUACCUGAUCAUGAACAA
CCUCAAGCGCAUUCCGAUU
CGAAACGAGCAGGUGGAAA
CGAAGCUCAUGAAUAUAUU
CGAAGUGAGUUCCCAGAGA
CGGAAAAGGAGGAUGGAAA
CGGAAGGCAUCCCGGUCAA
CGGAGAAGCGAAUGGACAA
CGGCUACCCUACUCUGUUG
CGGGCAGUGGGAAGGAUUA
CGGUCAUCGCUGUGCAUUA
CUACAUCUAUAACGAGAAG
CUACAUGAACCUACAGAGA
CUACGAAGCUCAUGAAUAU
CUGAAUAAGAAGAACAAAC
CUGCAGGAGGCUAAAGAUA
CUGGAGCAGUCAAGGUCUA
CUUGAAAGAGCCACCGAAA
GAAACGAGCAGGUGGAAAU
GAAACGGAGCAGUGGGAGA
GAACAACAACGAGAAAGAC
GAACAAGCUCUGCCGUGAU
GAACAGAACAUGAUCGGCA
GAACAUCGUUUCCCGAAGU
GAACCAGCUUGGCGUCUUG
GAACGUUGAGGACUUGAAA
GAACUGAACCUUGAUGUGU
GAACUGCCUUUGUCAUCUU
GAAGAAACACUACCUCAUG
GAAGAGAACCAGAAACAAU
GAAGAUCCGAGCCUACUAU
GAAGGAAUCAGAAUGAACA
GAAGUUCGGCCUAGAGAAG
GAAUGGACAAAGUCGGCAA
GACAAAGCCUUCUCCGAUA
GACCACAGCCAAAGAUAAG
GACCAGAAUUCUCCUGCAA
GAGAAGUUCGGCCUAGAGA
GAGAAUUGCUUCAUACAAA
GAGCCGUUCUCUACAAUUA
GAGGAGAAUUGCUUCAUAC
GAGGAUGACUGUCUAGCUU
GAGUGCAGAAAGAGGACAU
GAGUGGACUAUGUGUACAA
GAUAGGAACACCCUCAUCA
GAUCAACUCACCUGUAAUA
GAUCAGAAGAGACCAAGGA
GAUCCGAGCCUACUAUGAA
GCAACAAGUUCGUGACCGU
GCAGAAGAGAACAUCGUUU
GCAGAUGACCCUGAAGAUA
GCAGCUGGCCUUCAGACAA
GCAGGAGGCUAAAGAUAUU
GCCAAGACGAAGAGAACCA
GCCAGAAGCCCUUGAGCAU
GCGCAUUCCGAUUGAGGAU
GCGCCUUCACCGAGAGCAU
GCGUGUUCGUGCUGAAUAA
GCUCUGCCGUGAUGAACUG
GGAACAACAACGAGAAAGA
GGAACACCCUCAUCAUCUA
GGAACUGCCUUUGUCAUCU
GGAAGAAAGUGGAGGGAAC
GGAAGAUCCGAGCCUACUA
GGAAGGAAUCAGAAUGAAC
GGAAUCAGAAUGAACAAAA
GGACAAAGUCGGCAAGUAC
GGACAAGGCCUGUGAGCCA
GGACAAGGUCUCACACUCU
GGACAGCAGCGCACGUUCA
GGACAUAUCCAUGAUGACU
GGACAUCAUUGCAGAAGAG
GGACCCAAGUGGUGGAGAA
GGACGAAUGCCAAGACGAA
GGACGGUCAUGGUCAACAU
GGAGAAGCGAAUGGACAAA
GGAGAAUUGCUUCAUACAA
GGAGAGAAGCCCAACCUCA
GGAGGGAACUGCCUUUGUC
GCAGUAACCUGGAUGAGGA
GGAUGACUGUCUAGCUUUC
GGAUGCCACUAUGUCUAUA
GGAUJACGCCGGUGUCUUC
GGCAGAACCAAGAGCUCAA
GGCAGCAGAUGACCCUGAA
GGCCAAUGGUGUUGACAGA
GGCCUCUUCUUAACAAAUU
GGCUCAAUGAACAGAGAUA
GGGAAGAAAGUGGAGGGAA
GGGAGACCCUCAACGUCAA
GGUCAUCGCUGUGCAUUAC
GGUCUUCUCCACUGAGUUU
GUAAGCAGCUCUACAACGU
GUAAUAAAUUCGACCUCAA
GUGAAGGAGUGCAGAAAGA
GUGACAAUGUACCAUGCUA
GUGACAUGGUGCAGGCAGA
GUGCAUUACCUGGAUGAAA
GUGCUGAAUAAGAAGAACA
GUGGGCAACUCCAACAAUU
GUUCACCAAUACUUUAAUG
UCAACUCACCUGUAAUAAA
UCAAUGAACAGAGAUACUA
UCACAGUAAUGCAGGACUU
UCACCAAGACACCCAAGUA
UCAGAAGAGACCAAGGAAA
UCUACGAAGCUCAUGAAUA
UCUGGGACGUGGUGGAGAA
UGAAGCAGCUGGCCAAUGG
UGAAGCUGGAGGAGAAGAA
UGACAAUGUACCAUGCUAA
UGACCACAGCCAAAGAUAA
UGAGGAGAAUUGCUUCAUA
UGCAGGAAGUGGAAGUCAA
UGCUAAGGCCAAAGAUCAA
UGGAAGAACGGCUGGACAA
UGGCUUUGCUCCAGACACA
UGUGGAACGUUGAGGACUU
UUUCAAAGUUCACCAAUAC

TABLE 72
Targeting Sequence 5' to 3'

| | | | | | | |
|---|---|---|---|---|---|---|
| AAT | CCG | AGC | CGT | TCT | CTA | CAA |
| AAC | AAG | CTC | TGC | CGT | GAT | GAA |
| AAT | GGA | CAA | AGT | CGG | CAA | GTA |
| AAC | TAG | ATG | AAC | CTA | CAG | AGA |
| AAA | AAG | CGG | CCA | GTC | AGA | AGA |
| AAT | GAT | TGG | TGG | ATT | ACG | GAA |
| AAG | TCG | TCG | TTG | TCC | GTT | CCA |
| AAA | TGA | TTG | GTG | GAT | TAC | GGA |
| AAT | TAC | CGG | CAG | AAC | CAA | GAG |
| AAA | TGG | AAT | CTC | TAG | GAA | GCT |
| AAT | GAA | CAG | AGA | TAC | TAG | GGT |
| AAG | CCT | TGG | CTC | AAT | ACC | AAA |
| AAG | CGC | ATT | CCG | ATT | GAG | GAT |
| AAT | GGA | ATC | TCT | ACG | AAG | CTG |
| AAC | CTC | ATC | GCC | ATC | GAC | TCC |

TABLE 73
SEQUENCE

ccatggtgctgggacagtgc
aggtgggtcccatggcgccg
ttagtagcaggagcagcagg
ctgttggacccactttgtag
gtcgtgggcctccagcacca
gtaacagtgactggaacatc
ctgccctggcctctccccgg
actggacagcactagttttt
cccagcagcatgaatgcagc
ctgagggcctgttccccagc
tgaactccctgttggctggg
tctgtctggatgaagaggta
tcttgtctgtctggatgaag
gatggtctcgtctgtctgga
gtgtagatggtcttgtctgt
ttgtggttgacggtgaagat
cacgtactccttcacctcaa
aatttctctgtaggctccac
agaggaacctggcggtgctg
tcgccatcctggatcccgaa
tcaatcggaatgcgcttgag
gacttccccaccaggtgttc
tgcctgcaccatgtcactgc
ttggtgaagtggatctggta
ggtgtcttggtgaagtggat
caccatgaggtcaaagggca
tcacgaacaccatgaggtca
ggctggagagccatcagggt
ctgcctccgagcgctcctgc
ttgttggagttgcccacggt
tgtacgtagcactgagagat
caggtcctggccgggctctc
cagcgtgtagtacgccacca
tattcagcacgaacacgccc
ccgcagctctgtgatgtagt
caggcccaggtggctggccc
tcgtagcgattccatttttc
cccacgtggtgatggagtct
ccctttcttgtccgacatgc
cacacagatccctttcttgt
ggtctgccacacagatccct
ttcctgcaggccggtcttta
acggcagccttgacttccac
acttggtcactgaggtctgc
caggagaattctggtctccg
ccttgcaggagaattctggt
gggtcccttgcaggagaatt
tccaggtaatgcacagcgat
gccagctgccgggtgtaccc
tgaaggccagctgctgggtg
aagaccttgaccacgtaggc
agagcgaagaccttgaccac
agtcgatggcgatgaggttg
gggcttctgcttctccagga
ttcttggtgtatcacgggcg
ccaccaatcatttcttggtg
tcgcaaatatctttagcctc
gctgttgacctgctcctcgc
cagtgtaggatctctgtagg
cttctctttggctgtggtca
taccagggtcctcccagcgg
gcataggatgtggcctccac
tggaccaccctgacggtggc
gtattgagccaaggcttgga
ggtgatcttggagctgcggc
gttcttggcatcctgaggcc
gcaaagccagtcatcatgga
tgtgtctggagcaaagccag
gaaggctttgtccagctcat
ggtagatgatgcgggtgttc
cccagagcgtgagaccttgc
ccttttccggatggtagaac
ccacatgaggtagtgtttct
tcggaggagagcccccacat
tcaggccagtgctccaccca

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A method of treating a complement-mediated eye disorder, the method comprising systemically administering to a subject an siRNA that targets C3 mRNA in the subject.

2. The method of claim 1, wherein the siRNA comprises a liver-targeting moiety.

3. The method of claim 2, wherein the liver-targeting moiety is a GalNAc moiety.

4. The method of claim 1, wherein the siRNA comprises an antisense strand comprising a sequence listed in Tables 2A, 2B, 4, 5, 6, 10, 15, 16, 17, or 18 and/or comprises a sense strand comprising a sequence listed in Tables 1, 3A, 3B, 10, 15, 16, 17, or 18.

5. The method of claim 1, wherein the siRNA comprises an antisense strand and a sense strand, wherein the antisense strand is complementary to a nucleotide sequence that is at least 90% identical to any one of SEQ ID NOs: 76-100 and/or the sense strand comprises a nucleotide sequence that is at least 90% identical to any one of SEQ ID NOs: 76-100.

6-14. (canceled)

15. The method of claim 5, wherein one or both of the sense stand and the antisense strand comprises at least one modified nucleotide.

16. The method of claim 15, wherein the at least one modified nucleotide comprises a nucleotide that includes a 2′-O-Methyl group, a nucleotide that includes a 2′-Fluoro group, and/or a phosphorothioate bond with an adjacent nucleotide.

17-19. (canceled)

20. The method of claim 5, wherein the sense strand comprises the nucleotide sequence of any one of SEQ ID NOs: 76-100, 126-150, 201, 203, 205, 207, 209, 211, 213, 215, 217, 219, 221, 223, 225, 227, 229, 231, 233, 235, 237, 239, 241, 243, 245, 247, 249, 255, 259, 264, 268, 272, 276, 325, 326, and 327.

21. The method of claim 5, wherein the antisense strand comprises the nucleotide sequence of any one of SEQ ID NOs: 101-125, 151-200, 202, 204, 206, 208, 210, 212, 214, 216, 218, 220, 222, 224, 226, 228, 230, 232, 234, 236, 238, 240, 242, 244, 246, 248, 250, 252, 254, 256, 257, 258, 260, 261, 262, 263, 265, 266, 267, 269, 270, 271, 273, 274, 275, 277, 278, and 300-324.

22. (canceled)

23. The method of claim 1, wherein the siRNA comprises at least one ligand attached to one or more of the 5′ end of the sense strand, the 3′ end of the sense strand, the 5′ end of the antisense strand, and the 3′ end of the antisense strand.

24. The method of claim 23, wherein the ligand comprises at least one GalNAc moiety.

25-26. (canceled)

27. The method of claim 1, wherein after the administration of the siRNA, a level of C3 transcript or C3 protein in the subject or in a biological sample from the subject is reduced relative to a level before the administration of the siRNA.

28. (canceled)

29. The method of claim 1, wherein the siRNA is administered intravenously or subcutaneously to the subject.

30-34. (canceled)

35. The method of claim 1, wherein the eye disorder is geographic atrophy or intermediate AMD.

36. A method of inhibiting or reducing, relative to a control, level of complement C3 in the eye of a subject, the method comprising systemically administering to the subject an siRNA that targets C3 mRNA in the subject.

37. The method of claim 36, wherein the siRNA comprises a liver-targeting moiety.

38. The method of claim 37, wherein the liver-targeting moiety is a GalNAc moiety.

39. The method of claim 36, wherein the siRNA comprises an antisense strand comprising a sequence listed in Tables 2A, 2B, 4, 5, 6, 10, 15, 16, 17, or 18 and/or comprises a sense strand comprising a sequence listed in Tables 1, 3A, 3B, 10, 15, 16, 17, or 18.

40. The method of claim 36, wherein the siRNA comprises an antisense strand and a sense strand, wherein the antisense strand is complementary to a nucleotide sequence that is at least 90% identical to any one of SEQ ID NOs: 76-100 and/or the sense strand comprises a nucleotide sequence that is at least 90% identical to any one of SEQ ID NOs: 76-100.

41. The method of claim 40, wherein the antisense strand is complementary to a nucleotide sequence comprising a sequence that differs by no more than 1, 2, 3, or 4 nucleotides from any one of SEQ ID NOs: 76-100 and/or the sense strand comprises a nucleotide sequence that differs by no more than 1, 2, 3, or 4 nucleotides from any one of SEQ ID NOs: 76-100.

42-49. (canceled)

50. The method of claim 40, wherein one or both of the sense stand and the antisense strand comprises at least one modified nucleotide.

51. The method of claim 50, wherein the at least one modified nucleotide comprises a nucleotide that includes a 2′-O-Methyl group, a nucleotide that includes a 2′-Fluoro group, and/or a phosphorothioate bond with an adjacent nucleotide.

52-54. (canceled)

55. The method of claim 40, wherein the sense strand comprises the nucleotide sequence of any one of SEQ ID NOs: 76-100, 126-150, 201, 203, 205, 207, 209, 211, 213, 215, 217, 219, 221, 223, 225, 227, 229, 231, 233, 235, 237, 239, 241, 243, 245, 247, 249, 255, 259, 264, 268, 272, 276, 325, 326, and 327.

56. The method of claim 40, wherein the antisense strand comprises the nucleotide sequence of any one of SEQ ID NOs: 101-125, 151-200, 202, 204, 206, 208, 210, 212, 214, 216, 218, 220, 222, 224, 226, 228, 230, 232, 234, 236, 238, 240, 242, 244, 246, 248, 250, 252, 254, 256, 257, 258, 260, 261, 262, 263, 265, 266, 267, 269, 270, 271, 273, 274, 275, 277, 278, and 300-324.

57. (canceled)

58. The method of claim 36, wherein the siRNA further comprises at least one ligand attached to one or more of the 5′ end of the sense strand, the 3′ end of the sense strand, the 5′ end of the antisense strand, and the 3′ end of the antisense strand.

59. The method of claim 58, wherein the ligand comprises at least one GalNAc moiety.

60. The method of claim 59, wherein the ligand comprises three GalNAc moieties.

61.-64. (canceled)

65. The method of claim 36, wherein the subject suffers from a complement-mediated eye disorder.

66. The method of claim 65, wherein the eye disorder is geographic atrophy or intermediate AMD.

67. (canceled)
